(19) United States
(12) Patent Application Publication
(10) Pub. No.: US 2021/0261538 A1
(43) Pub. Date: Aug. 26, 2021

(54) PROTEIN ARGININE METHYLTRANSFERASE 5 (PRMT5) DEGRADATION / DISRUPTION COMPOUNDS AND METHODS OF USE

(71) Applicant: ICAHN SCHOOL OF MEDICINE AT MOUNT SINAI, New YOrk, NY (US)

(72) Inventors: Jian Jin, New York, NY (US); Jing Liu, Oradell, NJ (US); Yudao Shen, New York, NY (US); Ernesto Guccione, New York, NY (US); Martin Walsh, New York, NY (US); Almudena Bosch, New York, NY (US); Megan Schwarz, New York, NY (US)

(21) Appl. No.: 16/970,305

(22) PCT Filed: Feb. 22, 2019
(86) PCT No.: PCT/US2019/019123

§ 371 Date: Aug. 14, 2020

Related U.S. Application Data
(60) Provisional application No. 62/634,039, filed on Feb. 22, 2018.

(51) Int. Cl. C07D 417/14; C07D 401/14

(52) U.S. Cl. CPC C07D 417/14; C07D 401/14

(57) ABSTRACT

Disclosed are protein arginine methyltransferase 5 (PRMT5) degradation/disruption compounds including a PRMT5 ligand, a degradation/disruption tag and a linker, and methods for use of such compounds in the treatment of PRMT5-mediated diseases. The PRMT5 degraders disclosed herein offer a novel mechanism for treating PRMT5-mediated diseases compared to small molecule inhibitors of PRMT5 activity.

### CROSS-REFERENCE TO RELATED APPLICATION

This application claims the benefit of U.S. Provisional Application No. 62/634,039, filed on Feb. 22, 2018. The entire contents of the foregoing are incorporated herein by reference.

### TECHNICAL FIELD

This disclosure relates to bivalent compounds (e.g., bi-functional compounds, e.g., bi functional small molecule compounds) which degrade and/or disrupt protein arginine methyltransferase 5 (PRMT5), compositions comprising one or more of the bivalent compounds, and methods of use thereof for the treatment of PRMT5-mediated diseases in a subject in need thereof. The disclosure also relates to methods for designing such bivalent compounds.

### BACKGROUND OF THE INVENTION

Protein arginine methyltransferases (PRMTs) catalyze an important post-translational modification in eukaryotic cells, arginine methylation. Significant efforts have been spent attempting to develop small molecule inhibitors of the methyltransferase activity of protein arginine methyltransferace 5 (PRMT5) because overexpression of PRMT5 is associated with several human malignancies, including lymphomas, melanoma, adenocarcinoma, pancreas, prostate, lung cancer, breast cancer, colorectal, and ovarian cancer. PRMT5 is one of nine protein arginine methyltransferases that transfer the methyl group from the cofactor S-5′-adenosyl-L-methionine (SAM) to arginine residues of a variety of histone and non-histone proteins. Methylation of protonated arginine guanidium moieties (positively charged at physiological conditions) increases their bulkiness and alters their charge distribution, hydrophobicity, and hydrogen bond formation potential, thus affecting their protein- and nucleic acid-binding activity and ultimately their physiological function (Wei et al., 2014). Dysregulation of PRMTs has been linked to a variety of human diseases, such as pulmonary diseases, cardiovascular disease, diabetes, renal disease, Huntington's disease, Alzheimer's disease, asthma, and verities of cancer (Hu et al., 2016).

Nine PRMTs have been identified. Based on their product specificity, they are grouped into three categories, type I, type II and type III. Type I PRMTs (PRMT1-4, PRMT6 and PRMT5) catalyze arginine mono- and asymmetric dimethylation. Type II PRMTs (PRMT5 and PRMT5) catalyze arginine mono- and symmetric dimethylation. Type III PRMT (PRMT7) catalyzes arginine monomethylation only (Kaniskan et al., 2015). Protein arginine methyltransferase 5 (PRMT5) is the predominate type II PRMT and the major enzyme for arginine symmetric dimethylation.

PRMT5 methylates a variety of histone substrates in vivo, including H2AR3, H4R3, H3R2, and H3R8, which are associated with transcriptional regulatory processes. PRMT5 also methylates many non-histone proteins, including SmD3, NF-κB, p53, E2F-1, Raf, and RPS 10. Through the regulation of these non-histone targets, PRMT5 plays important roles in processes including RNA splicing, transcription, signaling pathway, and ribosome biogenesis. The substrate specificity of PRMT5 is regulated by its binding partners, including Blimp1, RioK1, pICLn, MBD/NuRD, and MEP50. The most common PRMT5 partner is MEP50, a member of the WD40 family of proteins, which is required for PRMT5 enzymatic activity and is likely present in every PRMT5-containing complex in vivo.

However, traditional catalytic inhibition of PRMT5 has not been an optimal solution for treating PRMT5 overexpression. First, cancer cells frequently develop resistance to small molecule inhibitors through mutations in the active site that overcome pharmacological inhibition. Second, most proteins have functions in addition to the (catalytic) activity targeted by small molecule inhibitors. For example, methyltransferases form complexes with other proteins through protein-protein interactions, and bind DNA directly at transcriptional promoter sites. Studies have shown that treating cancer cells with the enzymatic inhibitor EPZ015666 alone failed to optimally inhibit cancer cell proliferation. (Jin, 2016; Kryukov, 2016).

PRMT5 overexpression has been associated with multiple human malignancies, including lymphomas, melanoma, adenocarcinoma, pancreatic cancer, prostate cancer, lung cancer, breast cancer, colorectal cancer, and ovarian cancer. For example, overexpression of PRMT5 has been reported in human chronic myelogenous leukemia (CML) leukemia stem cells (LSCs). PRMT5 knockdown or inhibition dramatically prolonged survival in a murine model of BCR-ABL-driven CML and impaired the in vivo self-renewal capacity of transplanted CML LSCs (Jin et al., 2016). PRMT5 expression levels are significantly higher in gastric cancer (GC) tissues than the corresponding adjacent normal tissues. Knockdown of PRMT5 decreased the proliferation, invasion and migration of a GC cell line (Kanda et al., 2016). PRMT5 overexpression in patient multiple myeloma (MM) cells is associated with decreased progression-free survival and overall survival. Genetic knockdown of PRMT5 or inhibition of PRMT5 significantly inhibited the growth of patient MM cells (Gulla et al., 2017). PRMT5 promotes prostate cancer cell growth through androgen receptor (AR) upregulation. Knockdown of PRMT5 or inhibition of PRMT5 decreases the AR expression and suppresses the proliferation of AR-positive, but not AR-negative, prostate cancer cells (Deng et al., 2017). PRMT5 has been reported as a key mediator of glioblastoma (GBM) growth. PRMT5 knockdown or inhibition potently suppressed in vivo GBM tumors, including patient-derived xenografts (Braun et al., 2017). PRMT5 overexpression in hepatocellular carcinoma (HCC) tissues is associated with advanced disease stage and adverse prognosis. PRMT5 knockdown significantly decreased the proliferation, invasion, and migration of HCC cell lines (Shimizu et al., 2017). PRMT5 is highly expressed in pancreatic ductal adenocarcinoma (PDAC) and colorectal cancer (CRC). PRMT5 promotes cancer progression through the activation of NF-κB, while shRNA knockdown had opposite effect (Prabhu et al., 2017).

Significant efforts have been made to the development of therapeutics capable of inhibiting the methyltransferase activity of PRMT5. A number of PRMT5 inhibitors have been published, including EPZ015666, GSK591, GSK3326595 (EPZ015938), BLL-1, HLCL-61, and LLY-283 and PF-06855800. Several compounds including GSK3326595, are being investigated in phase I clinical trials in patients with solid tumors and non-Hodgkin's lymphoma.

Recent studies demonstrated that deletion of MTAP in cancer cells confers enhanced dependency on PRMT5 (Kryukov et al., 2016; Marjon et al., 2016; Mavrakis et al., 2016).

Genetic PRMT5 knockdown significantly inhibited the growth of MTAP-deleted cells, while PRMT5 pharmacological inhibition with PRMT5 inhibitor EPZ015666 did not lead to a similar anti-proliferation effect.

Unlike traditional enzyme inhibitors, which only inhibit the catalytic activity of the target enzyme, the PRMT5 degraders disclosed herein, including proteolysis-targeted chimeras (PROTACs), bind and induce degradation of PRMT5, thus eliminating any scaffolding functions of PRMT5 in addition to eliminating its enzymatic activity. The PRMT5 degraders disclosed herein are bivalent compounds, including a PRMT5 ligand conjugated to a degradation/disruption tag.

The PRMT5 degraders disclosed herein offer a novel mechanism for treating PRMT5-mediated diseases. In particular, the ability of the degraders to target PRMT5 for degradation, as opposed to merely inhibiting PRMT5's catalytic activity, is expected to overcome resistance, regardless of whether the drugs that were used in a prior treatment or whether acquired resistance was caused by gene mutation, amplification or otherwise.

In an aspect, this disclosure provides a method of treating PRMT5-mediated diseases, the method including administering one or more PRMT5 degraders to a subject who has a PRMT5-mediated disease, the PRMT5 degraders being bivalent compounds including a PRMT5 ligand conjugated to a degradation/disruption tag. The PRMT5-mediated diseases may be a disease resulted from PRMT5 amplification. The PRMT5-mediated diseases can have elevated PRMT5 enzymatic activity relative to a wild-type tissue of the same species and tissue type. Non-limiting examples of PRMT5-mediated diseases include acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), a hematopoietic cancer (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL), follicular lymphoma, chronic lymphocytic leukemia/small lymphocytic lymphoma (CLL/SLL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (e.g., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MPH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g. seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva). The PRMT5-mediated cancer can include, e.g., a relapsed cancer. The PRMT5-mediated cancer can, e.g., be refractory to one or more previous treatments.

### SUMMARY OF THE INVENTION

The present disclosure relates generally to bivalent compounds (e.g., bi-functional compounds, e.g., bi-functional small molecule compounds) which degrade and/or disrupt PRMT5 and to methods for the treatment of PRMT5-mediated diseases (i.e., a disease which depends on PRMT5; overexpresses PRMT5; depends on PRMT5 activity; or includes elevated levels of PRMT5 activity relative to a wild-type tissue of the same species and tissue type). It is important to note, because the PRMT5 degraders/disruptors have dual functions (enzyme inhibition plus protein degradation/disruption), the bivalent compounds of the present disclosure can be significantly more effective therapeutic agents than currently available PRMT5 inhibitors, which inhibit the enzymatic activity of PRMT5, but do not affect PRMT5 protein levels. The present disclosure further provides methods for identifying PRMT5 degraders/disruptors as described herein.

More specifically, the present disclosure provides a bivalent compound including a PRMT5 ligand conjugated to a degradation/disruption tag.

In some aspects, the PRMT5 degraders/disruptors have the form “PI-linker-EL”, as shown below:

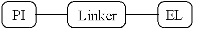

wherein PI (protein of interest) comprises a PRMT5 ligand (e.g., a PRMT5 inhibitor) and EL (E3 ligase) comprises a degradation/disruption tag (e.g., E3 ligase ligand). Exemplary PRMT5 ligands (PI), exemplary degradation/disruption tags (EL), and exemplary linkers (Linker) are illustrated below:

### PRMT5 Ligands

In one aspect, the PRMT5 Ligand (PI) comprises:

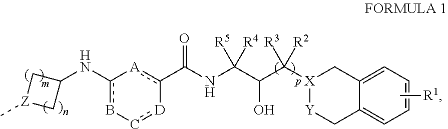

wherein
A, B, C, and D are independently a bond, CR⁶, NR⁷, N, O, or S;
X and Z are independently CR⁷, CR⁸, or N;
Y is a bond, CR⁸, CR⁹, N, or NR¹⁰,
R¹, R², R³, R⁴, R⁵, R⁶, R⁷, R⁸, R⁹, and R¹⁰ are independently hydrogen, halogen, optionally substituted C₁-C₈ alkyl, optionally substituted C₁-C₈ alkoxy, and optionally substituted C₁-C₈ alkoxyalkyl;
m and n are independently 0, 1, 2, 3, or 4; and
p is 0 or 1.

In some embodiments with respect to FORMULA 1,

the “Linker” moiety of the bivalent compound is attached to Z;
A, B, C, and D are independently a bond, CR⁶, NR⁷, N, O, or S;
X and Z are independently CR⁸, or N;
Y is a bond, CR⁹, or NR¹⁰,
R¹, R², R³, R⁴, R⁵, R⁶, R⁷, R⁸, R⁹, and R¹⁰ are independently hydrogen, halogen, optionally substituted C₁-C₈ alkyl, optionally substituted C₁-C₈ alkoxy, and optionally substituted C₁-C₈ alkoxyalkyl;
m and n are independently 0, 1, 2, 3, or 4; and
p is 0 or 1.

In some embodiments with respect to FORMULA 1,

A, B, C, and D are independently a bond, CR⁶, N, O, or S;
X and Z are independently CR⁷ or N;
Y is a bond, CR⁸, N, or NR¹⁰,
R¹, R², R³, R⁴, R⁵, R⁶, R⁷, and R⁸ are independently hydrogen, halogen, C₁-C₈ alkyl, C₁-C₈ alkoxy, and C₁-C₈ alkoxyalkyl;
m and n are independently 0-3; and
p is 0 or 1.

In another embodiment, with respect to FORMULA 1, A and C are CH; B is N; D is optionally selected from CH or N.

In another embodiment, with respect to FORMULA 1, X and Z are N.

In another embodiment, with respect to FORMULA 1, Y is a bond or CH₂.

In another embodiment, with respect to FORMULA 1, R¹, R², R³, R⁴, R⁵, R⁶, R⁷, R⁸, R⁹, and R¹⁰ are independently selected from hydrogen and halogen.

In another embodiment, with respect to FORMULA 1, m and n are independently selected from 1 and 2.

In another embodiment, with respect to FORMULA 1, p is 1.

In another aspect, the PRMT5 Ligand (PI) comprises:

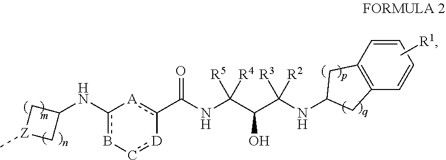

wherein,
A, B, C, and D are independently selected from a bond, CR⁶, NR⁷, N, O, and S;
Z is independently selected from CR⁷, CR⁸ and N;
R¹, R², R³, R⁴, R⁵, R⁶, R⁷, and R⁸ are independently selected from hydrogen, halogen, optionally substituted C₁-C₈ alkyl, optionally substituted C₁-C₈ alkoxy, and optionally substituted C₁-C₈ alkoxyalkyl; and
m, n, p, and q are independently selected from 0, 1, 2, 3, and 4.

In some embodiments, with respect to FORMULA 2,

A, B, C, and D are independently a bond, CR⁶, N, O, or S;
Z is independently CR⁷, or N;
R¹, R², R³, R⁴, R⁵, R⁶, R⁷, and R⁸ are independently hydrogen, halogen, C₁-C₈ alkyl, C₁-C₈ alkoxy, or C₁-C₈ alkoxyalkyl; and
m, n, and p are 0-3.

In some embodiments, with respect to FORMULA 2,

the “Linker” moiety of the bivalent compound is attached to Z;
A, B, C, and D are independently selected from a bond, CR⁶, NR⁷, N, O, and S;
Z is independently selected from CR⁸ and N;
R¹, R², R³, R⁴, R⁵, R⁶, R⁷, and R⁸ are independently selected from hydrogen, halogen, optionally substituted C₁-C₈ alkyl, optionally substituted C₁-C₈ alkoxy, and optionally substituted C₁-C₈ alkoxyalkyl; and
m, n, p, and q are independently selected from 0, 1, 2, 3, and 4.

In some embodiments, with respect to FORMULA 2, A and C are CH; B is N; D is optionally selected from CH and N.

In some embodiments, with respect to FORMULA 2, Z is N.

In some embodiments, with respect to FORMULA 2, R¹, R², R³, R⁴, R⁵, R⁶, R⁷, and R⁸ are independently selected from hydrogen and halogen.

In some embodiments, with respect to FORMULA 2, m, n, p and q are independently selected from 1 and 2.

In another aspect, the PRMT5 Ligand (PI) comprises:

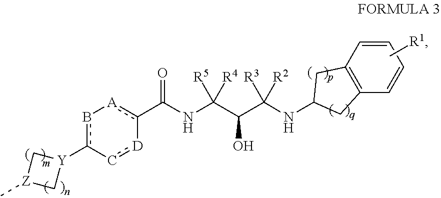

wherein
the “Linker” moiety of the bivalent compound is attached to Z;
A, B, C, and D are independently selected from a bond, CR⁶, NR⁷, N, O, or S;
Y and Z are independently selected from CR⁸ or N;
R¹, R², R³, R⁴, R⁵, R⁶, R⁷, and R⁸ are independently selected from hydrogen, halogen, optionally substituted C₁-C₈ alkyl, optionally substituted C₁-C₈ alkoxy, and optionally substituted C₁-C₈ alkoxyalkyl; and
m, n, p, and q are independently selected from 0, 1, 2, 3, and 4.

In some embodiments, with respect to FORMULA 3, A and C are CH; B is N; D is optionally selected from CH or N.

In some embodiments, with respect to FORMULA 3, Y and Z independently selected from CH and N.

In some embodiments, with respect to FORMULA 3, R¹, R², R³, R⁴, R⁵, R⁶, R⁷, and R⁸ are independently selected from hydrogen and halogen.

In some embodiments, with respect to FORMULA 3, m, n, p and q are independently selected from 1 and 2.

In the formulas above, the reference to a “bond” means that the respective letter A, B, C or D refers to the absence of an atom or moiety, and there is a bond between adjacent atoms in the structure.

In another aspect, the PRMT5 Ligand (PI) comprises:

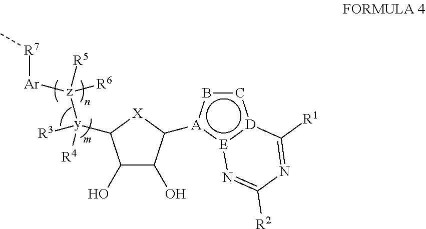

wherein
the “Linker” moiety of the bivalent compound is attached to R⁷;
X is selected from CH₂ and O;
Y and Z are selected from null, C, O, and S;
A, B, C, D, and E are independently selected from null, CR⁸, CR⁸═CR⁹, CNR¹⁰R¹¹, CNR¹⁰C(O)R¹¹, C NR⁸C(O)NR¹⁰R¹¹, CNR⁸SOR¹⁰, CNR⁸SO₂R¹⁰, NR¹⁰, N, N═N, CR⁸═N, O, and S, wherein
R⁸, R⁹, R¹⁰, and R¹¹ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈ alkylamino, optionally substituted C₁-C₈alkylaminoC₁-C₈alkyl, optionally substituted 3-10 membered cycloalkyl, optionally substituted 4-10 membered heterocyclyl;
   R¹ is selected from hydrogen, halogen, cyano, nitro, OR¹², SR¹², NR¹³R¹⁴, COR¹², CO₂R¹²,
C(O)NR¹³R¹⁴, SOR¹², SO₂R¹²SO₂NR¹³R¹⁴, NR¹²C(O)R¹³, NR¹²C(O)NR¹³R¹⁴, NR¹²SOR¹³, NR¹²SO₂R¹³, optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl, optionally substituted aryl, and optionally substituted heteroaryl, wherein
R¹², R¹³, and R¹⁴ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹² and R¹³, R¹³ and R¹⁴ together with the atom to which they are connected form an optionally substituted 4-10 membered heterocyclyl ring;
   R², R³, R⁴, R⁵ and R⁶ are independently selected from null, hydrogen, halogen, OR¹⁵, NR¹⁶R¹⁷, optionally substituted C₁-C₈ alkyl, optionally substituted C₂-C₈ alkenyl, optionally substituted C₂-C₈ alkynyl, optionally substituted 3-10 membered cycloalkyl, and optionally substituted 4-10 membered heterocyclyl, wherein
R¹⁵, R¹⁶, and R¹⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted C₂-C₈ alkenyl, optionally substituted C₂-C₈ alkynyl, or
R¹⁶ and R¹⁷ together with the atom to which they are connected form an optionally substituted 4-10 membered heterocyclyl ring;
   R⁷ is selected from null, OR¹⁸, SR¹⁸, NR¹⁸R¹⁹, OC(O)R¹⁸, OC(O)OR¹⁸, OCONR¹⁸R¹⁹, C(O)R¹⁸, C(O)OR¹⁸, CONR¹⁸R¹⁹, S(O)R¹⁸, S(O)₂R¹⁸, SO₂NR¹⁸R¹⁹, NR²⁰C(O)OR¹⁸, NR²⁰C(O)R¹⁸, NR²⁰C(O)NR¹⁸R¹⁹, NR²⁰S(O)R¹⁸, NR²⁰S(O)₂R¹⁸, NR²⁰S(O)₂NR¹⁸R¹⁹, optionally substituted C₁-C₈ alkylene, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹⁸ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹⁹ and R²⁰ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl; or
R¹⁸ and R¹⁹, R¹⁸ and R²⁰, R¹⁹ and R²⁰ together with the atom to which they are connected form a 4-20 membered heterocyclyl ring;
   Ar is selected from null, aryl and heteroaryl, each of which is substituted with R⁷ and optionally substituted with one or more substituents independently selected from hydrogen, halogen, oxo, CN, NO₂, OR²¹, SR²¹, NR²¹, R²², OCOR²¹, OCO₂R²¹, OCONR²¹R²², COR²¹, CO₂R²¹, CONR²¹R²², SOR²¹, SO₂R²¹, SO₂NR²¹R²², NR²³CO₂R²¹, NR²³COR²¹, NR²³C(O)NR²¹R²², NR²³SOR²¹, NR²³SO₂R²¹, NR²³SO₂NR²¹R²², optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R²¹, R²² and R²³ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R²¹ and R²², R²¹ and R²³ together with the atom to which they are connected form a 4-20 membered heterocyclyl ring; and
   m and n are independently selected from 0 and 1.

In some embodiments, FORMULA 4 is FORMULA 4A:

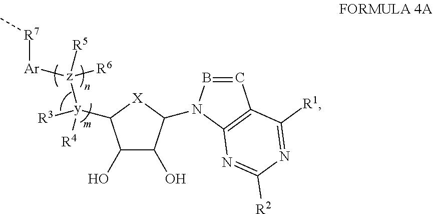

wherein
the definitions of X, Y, Z, B, C, R¹, R², R³, R⁴, R⁵, R⁶, R⁷ and Ar are the same as FORMULA 4.

In some embodiments, FORMULA 4 is FORMULA 4B:

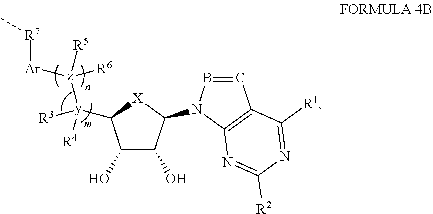

wherein
the definitions of X, Y, Z, B, C, R¹, R², R³, R⁴, R⁵, R⁶, R⁷ and Ar are the same as FORMULA 4.

In some embodiments, FORMULA 4 is FORMULAE 4C, 4D and 4E:

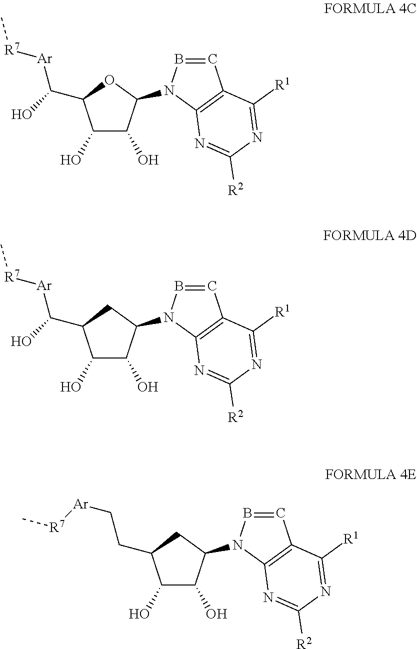

wherein
the definitions of B, C, R¹, R², R⁷ and Ar are the same as FORMULA 4.

In some embodiments, with respect to FORMULA 4 and FORMULAS 4A-4E,

B is selected from CH and N;
C is selected from CR⁸, CNR¹⁰R¹¹, CNR¹⁰C(O)R¹¹, C NR⁸C(O)NR¹⁰R¹¹, CNR⁸SOR¹⁰, CNR⁸SO₂R¹⁰, and N, wherein
R⁸, R¹⁰, and R¹¹ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkylamino, optionally substituted 3-10 membered cycloalkyl, optionally substituted 4-10 membered heterocyclyl;
   R¹ is selected from NR¹³R¹⁴, NR¹²C(O)R¹³, NR¹²C(O)NR¹³R¹⁴, NR¹²SOR¹³, NR¹²SO₂R¹³, optionally substituted C₁-C₈ alkyl, optionally substituted 3-10 membered cycloalkyl, optionally substituted 4-10 membered heterocyclyl, wherein

R¹², R¹³ and R¹⁴ are independently selected from hydrogen, optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl, optionally substituted aryl, and optionally substituted heteroaryl, or

R¹³ and R¹⁴ together with the atom to which they are connected form an optionally substituted 4-10 membered heterocyclyl ring;

R² is selected from hydrogen, methyl, and NH₂;
R⁷ is selected from null, OR¹⁸, SR¹⁸, NR¹⁸R¹⁹, C(O)R¹⁸, C(O)OR¹⁸, CONR¹⁸R¹⁹, S(O)R¹⁸, S(O)₂R¹⁸, SO₂NR¹⁸R¹⁹, NR²⁰C(O)OR¹⁸, NR²⁰C(O)R¹⁸, NR²⁰C(O)NR¹⁸R¹⁹, NR²⁰S(O)R¹⁸, NR²⁰S(O)₂R¹⁸, NR²⁰S(O)₂NR¹⁸R¹⁹, optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹⁸ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹⁹ and R²⁰ are independently selected from optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl, optionally substituted aryl, and optionally substituted heteroaryl; or
R¹⁸ and R¹⁹, R¹⁸ and R²⁰, R¹⁹ and R²⁰ together with the atom to which they are connected form a 4-20 membered heterocyclyl ring;
   Ar is selected from null, aryl and heteroaryl, each of which is substituted with R⁷ and optionally substituted with one or more substituents independently selected from hydrogen, halogen, oxo, CN, NO₂, OR²¹, SR²¹, NR²¹R²², OCOR²¹, OCO₂R²¹, OCONR²¹R²², COR²¹, CO₂R²¹, CONR²¹R²², SOR²¹, SO₂R²¹, SO₂NR²¹R²², NR²³CO₂R²¹, NR²³COR²¹, NR²³C(O)NR²¹R²², NR²³SOR²¹, NR²³SO₂R²¹, NR²³SO₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R²¹, R²² and R²³ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R²¹ and R²², R²¹ and R²³ together with the atom to which they are connected form a 4-20 membered heterocyclyl ring.

In some embodiments, FORMULA 4 is FORMULA 4F:

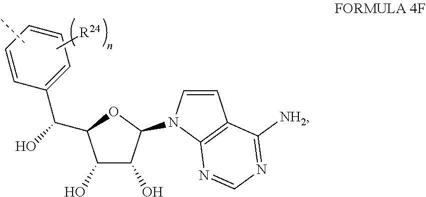

wherein
each R²⁴ is independently selected from null, hydrogen, halogen, oxo, CN, NO₂, OR²⁵, SR²⁵, NR²⁵R²⁶, OCOR²⁵, OCO₂R²⁵, OCONR²⁵R²⁶, COR²⁵, CO₂R²⁵, CONR²⁵R²⁶, SOR²⁵, SO₂R²⁵, SO₂NR²⁵R²⁶, NR²⁷CO₂R²⁵, NR²⁷COR²⁵, NR²⁷C(O)NR²⁵R²⁶, NR²⁷SOR²⁵, NR²⁷SO₂R²⁵, NR²⁷SO₂NR²⁵R²⁶, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R²⁵, R²⁶ and R²⁷ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted C₃-C₈ cycloalkoxy, optionally substituted 4-10 membered heterocyclyl, optionally substituted aryl, and optionally substituted heteroaryl, or
R²⁵ and R²⁶, R²⁵ and R²⁷ together with the atom to which they are connected form a 4-20 membered heterocyclyl ring; and
   n is independently selected from 0, 1, 2, 3, and 4.

In some embodiments, FORMULA 4 is FORMULA 4G:

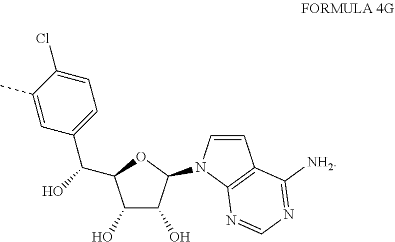

In another aspect, the PRMT5 Ligand (PI) comprises:

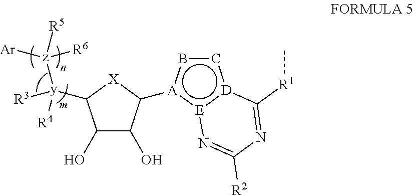

wherein
the “Linker” moiety of the bivalent compound is attached to R¹;
X is selected from CH₂ and O;
Y and Z are selected from null, C, O, and S;
A, B, C, D, and E are independently selected from null, CR⁷, CR⁷═CR⁸, CNR⁹R¹⁰, CNR⁹C(O)R¹⁰, CNR⁸C(O)NR⁹R¹⁰, CNR⁷SOR⁹, CNR⁷SO₂R⁹, NR⁹, N, N═N, CR⁷═N, O, and S, wherein
R⁷, R⁸, R⁹ and R¹⁹ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered cycloalkyl, optionally substituted 4-10 membered heterocyclyl;
   R¹ is selected from null, OR¹¹, SR¹¹, NR¹¹R¹², OC(O)R¹¹, OC(O)OR¹¹, OCONR¹¹R¹², C(O)R¹¹, C(O)OR¹¹, CONR¹¹R¹², S(O)R¹¹, S(O)₂R¹¹, SO₂NR¹¹R¹², NR¹³, C(O)OR¹¹, NR¹³C(O)R¹¹, NR¹³C(O)NR¹¹R¹², NR¹³S(O)R¹¹, NR¹³S(O)₂R¹¹, NR¹³S(O)₂NR¹¹R¹² optionally substituted C₁-C₈ alkylene, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹¹ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹² and R¹³ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl; or
R¹¹ and R¹², R¹¹ and R¹³, R¹² and R¹³ together with the atom to which they are connected form a 4-20 membered heterocyclyl ring;
   R², R³, R⁴, R⁵ and R⁶ are independently selected from hydrogen, halogen, OR¹⁴, NR¹⁵R¹⁶ optionally substituted C₁-C₈ alkyl, optionally substituted C₂-C₈ alkenyl, optionally substituted C₂-C₈ alkynyl, optionally substituted 3-10 membered cycloalkyl, and optionally substituted 4-10 membered heterocyclyl, wherein
R¹⁴, R¹⁵ and R¹⁶ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl; or
R¹⁵ and R¹⁶ together with the atom to which they are connected form an optionally substituted 4-10 membered heterocyclyl ring;
   Ar is selected from aryl and heteroaryl, each of which is optionally substituted with one or more substituents independently selected from hydrogen, halogen, oxo, CN, NO₂, OR¹⁷, SR¹⁷, NR¹⁷R¹⁸, OCOR¹⁷, OCO₂R¹⁷, OCONR¹⁷R¹⁸, COR¹⁷, CO₂R¹⁷, CONR¹⁷R¹⁸, SOR¹⁷, SO₂R¹⁷, SO₂NR¹⁷R¹⁸, NR¹⁹CO₂R¹⁷, NR¹⁹COR¹⁷, NR¹⁹C(O)NR¹⁷R¹⁸, NR¹⁹SOR¹⁷, NR¹⁹SO₂R¹⁷, NR¹⁹SO₂NR¹⁷R¹⁸, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R¹⁷, R¹⁸ and R¹⁹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹⁷ and R¹⁸, R¹⁷ and R¹⁹ together with the atom to which they are connected form a 4-20 membered heterocyclyl ring; and
   m and n are independently selected from 0 and 1.

In some embodiments, the FORMULA 5 is FORMULA 5A:

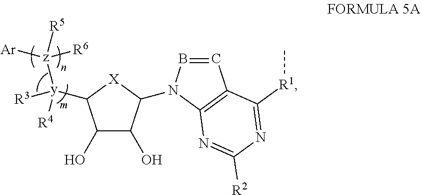

wherein
the definitions of X, Y, Z, B, C, R¹, R², R³, R⁴, R⁵, R⁶ and Ar are the same as FORMULA 5.

In some embodiments, the FORMULA 5 is FORMULA 5B:

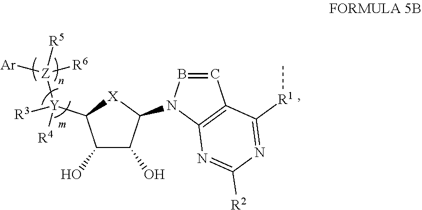

wherein
the definitions of X, Y, Z, B, C, R¹, R², R³, R⁴, R⁵, R⁶ and Ar are the same as FORMULA 5.

In some embodiments, the FORMULA 5 is FORMULAE 5C, 5D, and 5E:

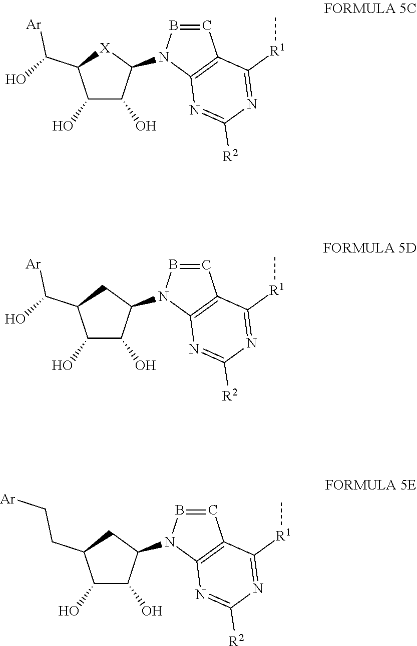

wherein
the definitions of B, C, R¹, R² and Ar are the same as FORMULA 5.

In some embodiments, with respect to FORMULAS 5C-5E,

B is selected from CH and N;
C is selected from CR⁸, CNR¹⁰R¹¹, CNR¹⁰C(O)R¹¹, C NR⁸C(O)NR¹⁰R¹¹, CNR⁸SOR¹⁰, CNR⁸SO₂R¹⁰, and N, wherein
R⁸, R¹⁰, and R¹¹ are independently selected from hydrogen, optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl;
   R¹ is selected from null, OR¹¹, SR¹¹, NR¹¹R¹², OC(O)R¹¹, OC(O)OR¹¹, OCONR¹¹R¹², C(O)R¹¹, C(O)OR¹¹, CONR¹¹R¹², S(O)R¹¹, S(O)₂R¹¹, SO₂NR¹¹R¹², NR¹³, C(O)OR¹¹, NR¹³C(O)R¹¹, NR¹³C(O)NR¹¹R¹², NR¹³S(O)R¹¹, NR¹³S(O)₂R¹¹, NR¹³S(O)₂NR¹¹R¹² optionally substituted C₁-C₈ alkylene, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹¹ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹² and R¹³ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl; or
R¹¹ and R¹², R¹¹ and R¹³, R¹² and R¹³ together with the atom to which they are connected form a 4-20 membered heterocyclyl ring;
   R² is selected from hydrogen, methyl, and NH₂; and
   Ar is selected from aryl and heteroaryl, each of which is optionally substituted with one or more substituents independently selected from hydrogen, halogen, oxo, CN, NO₂, OR¹⁷, SR¹⁷, NR¹⁷R¹⁸, OCOR¹⁷, OCO₂R¹⁷, OCONR¹⁷R¹⁸, COR¹⁷, CO₂R¹⁷, CONR¹⁷R¹⁸, SOR¹⁷, SO₂R¹⁷, SO₂NR¹⁷R¹⁸, NR¹⁹CO₂R¹⁷, NR¹⁹COR¹⁷, NR¹⁹C(O)NR¹⁷R¹⁸, NR¹⁹SOR¹⁷, NR¹⁹SO₂R¹⁷, NR¹⁹SO₂NR¹⁷R¹⁸, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R¹⁷, R¹⁸ and R¹⁹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹⁷ and R¹⁸, R¹⁷ and R¹⁹ together with the atom to which they are connected form a 4-20 membered heterocyclyl ring.

In some embodiments, the FORMULA 5 is FORMULA 5F:

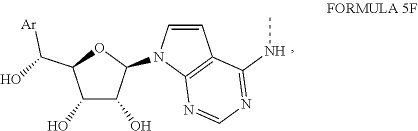

wherein
the definitions of Ar is the same as FORMULA 5.

In addition, the PRMT5 ligand can be a PRMT5 inhibitor, such as, EPZ015666 (Chan-Penebre et al., 2015), GSK591 (Kaniskan et al., 2017), GSK3326595 (EPZ015938) (Kaniskan et al., 2017), BLL-1 (CPD 5) (Alinari et al., 2015), HLCL-61 (Tarighat et al., 2016), LLY-283 (Kaniskan et al., 2017), PF-06855800 (Mcalpine et al., 2018) and/or analogs thereof.

In some aspects, the PRMT5 ligand can be, e.g.,

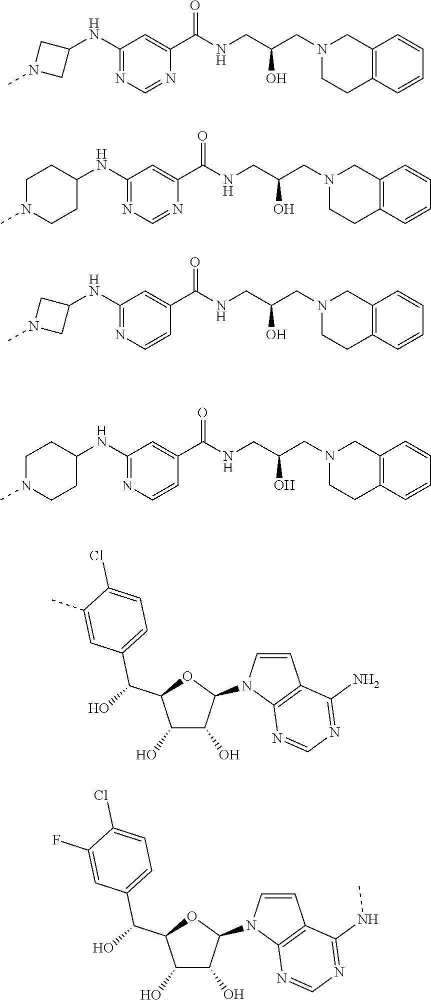

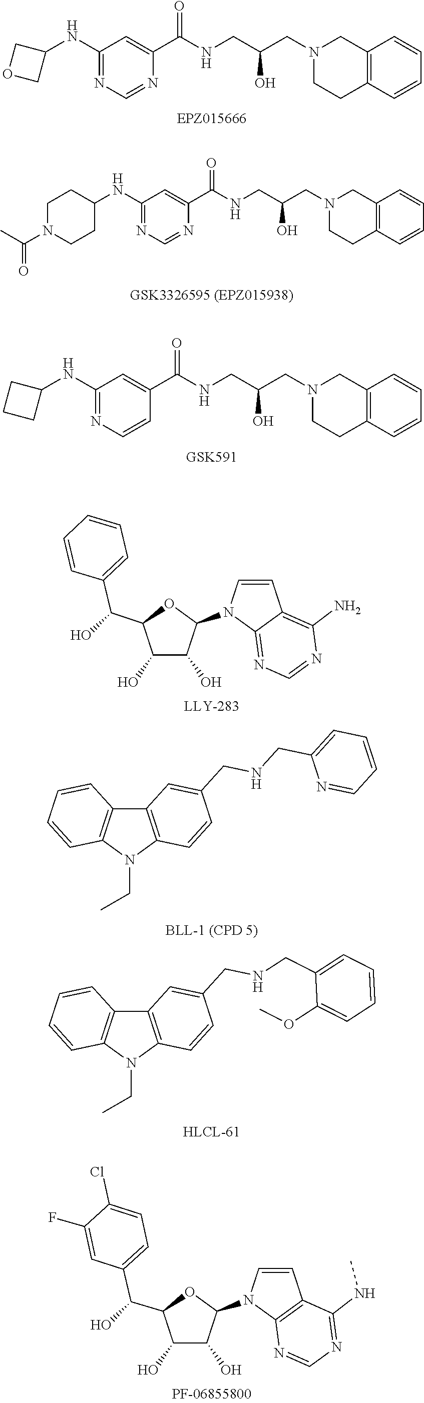

### Degradation/Disruption Tags

In some aspects, the Degradation/Disruption tag (EL) comprises any one of FORMULA 6A-6D:

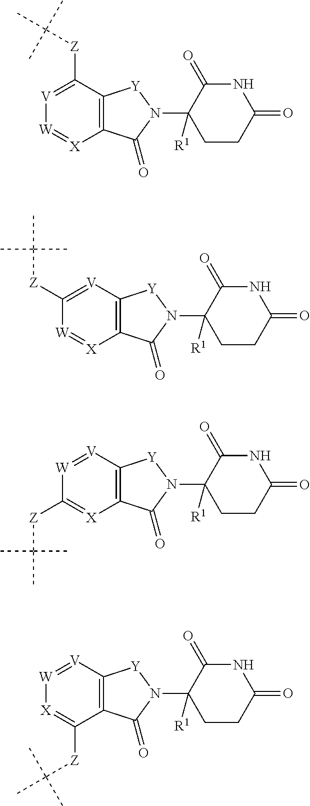

wherein
V, W, and X are independently selected from CR² and N;
Y is selected from CO, CH₂, and N═N;
Z is selected from CH₂, NH, and O;
R¹ is selected from hydrogen, methyl, fluoro, C1-C5 alkyl, and halogen; and
R² is hydrogen, halogen, or C1-C5 alkyl.

In certain embodiments, with respect to FORMULAS 6A-6D,

V, W, and X are independently selected from CR² and N;
Y is selected from CO and CH₂;
Z is selected from CH₂, NH, and O;
R¹ is selected from hydrogen, methyl, and fluoro; and
R² is hydrogen, halogen, or C₁-C₅ alkyl.

In certain embodiments, with respect to FORMULAS 6A-6D,

V, W, and X are independently selected from CR² or N;
Y is selected from CO, CH₂, N═N;
Z is selected from CH₂, NH, or O;
R¹ is selected from hydrogen, C₁-C₅ alkyl and halogen; and
R² is hydrogen, halogen, or C₁-C₅ alkyl;

In some aspects, the Degradation/Disruption tag (EL) comprises:

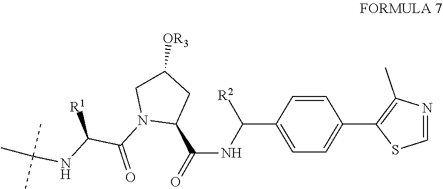

wherein
R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-8 membered cycloalkyl, optionally substituted 4-8 membered heterocyclyl, optionally substituted C₂-C₈ alkenyl, and optionally substituted C₂-C₈ alkynyl;
R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₈ cycloalkyl, optionally substituted C(O)(4-8 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₈ cycloalkyl, optionally substituted C(O)O(4-8 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₈ cycloalkyl, optionally substituted C(O)N(4-8 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In some aspects, the Degradation/Disruption tags (EL) comprises:

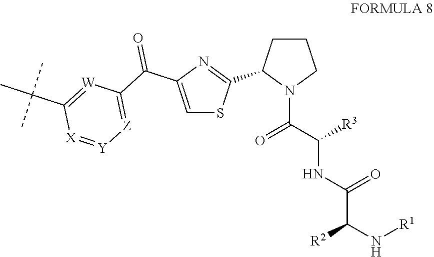

wherein
V, W, X, and Z are independently selected from CR⁴ and N; and
R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted 3-8 membered cycloalkyl, optionally substituted 4-8 membered heterocyclyl, optionally substituted C₂-C₈ alkenyl, and optionally substituted C₂-C₈ alkynyl.

In some aspects, the degradation/disruption tag can be, e.g., pomalidomide (Fischer et al., 2014), thalidomide (Fischer et al., 2014), lenalidomide (Fischer et al., 2014), VH032 (Galdeano et al., 2014; Maniaci et al., 2017), adamantine (Xie et al., 2014), 1-((4,4,5,5,5-pentafluoropentyl)sulfinyl)nonane (E. Wakeling, 1995), nutlin-3a (Vassilev et al., 2004), RG7112 (Vu et al., 2013), RG7338, AMG 232 (Sun et al., 2014), AA-115 (Aguilar et al., 2017), bestatin (Hiroyuki Suda et al., 1976), MV1 (Varfolomeev et al., 2007), LCL161 (Weisberg et al., 2010), and/or analogs thereof.

In some aspects, the degradation/disruption tag can be, e.g., one of the following structures:

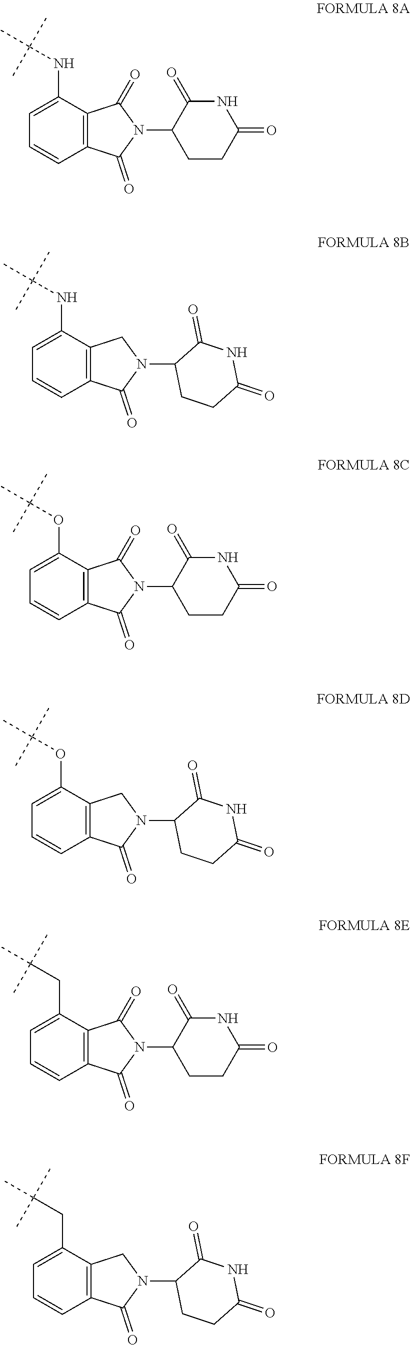

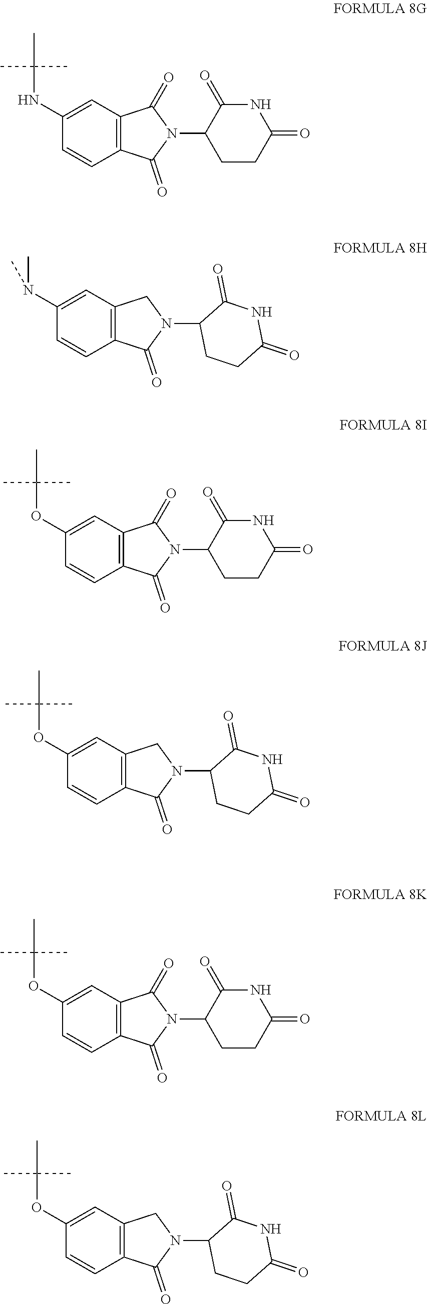

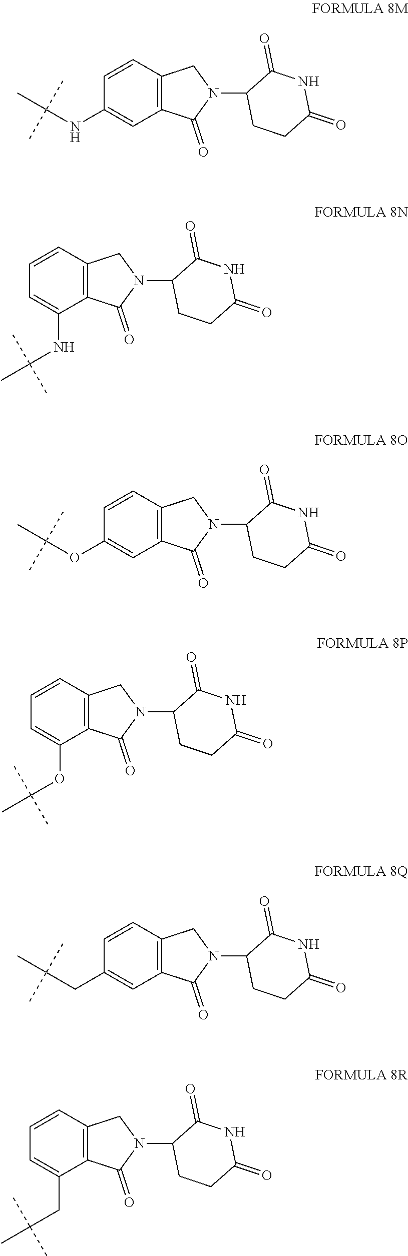

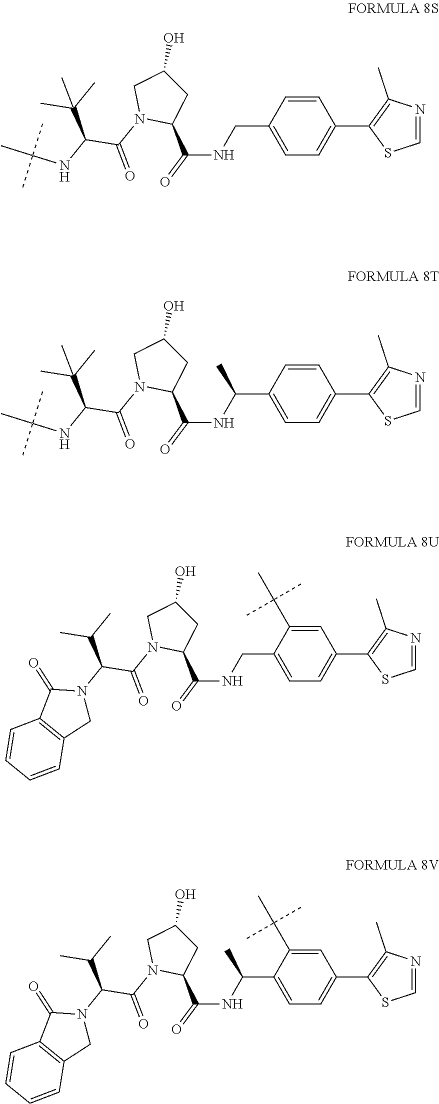

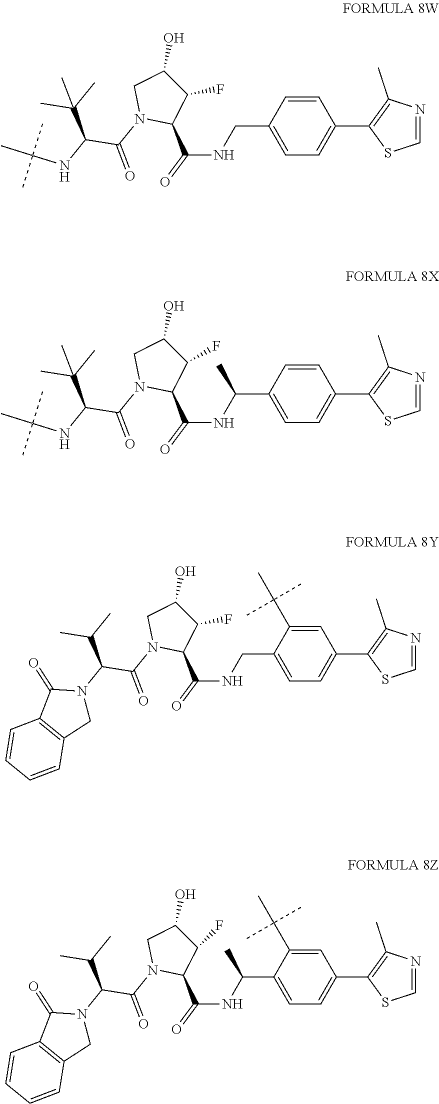

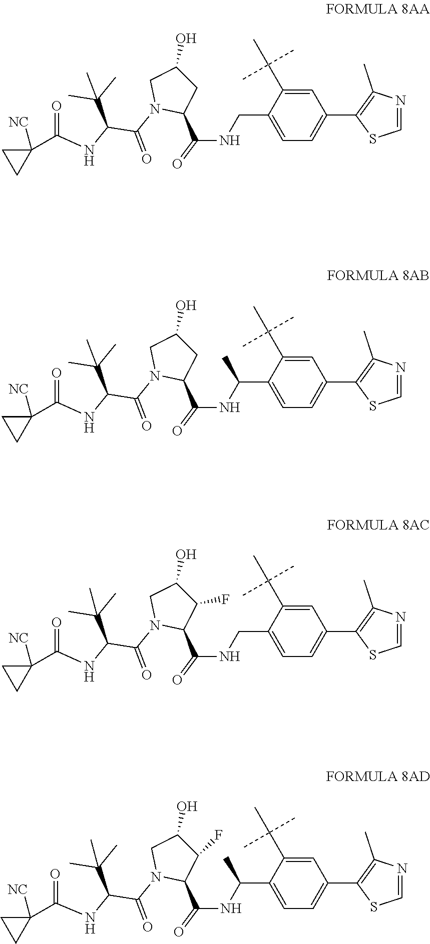

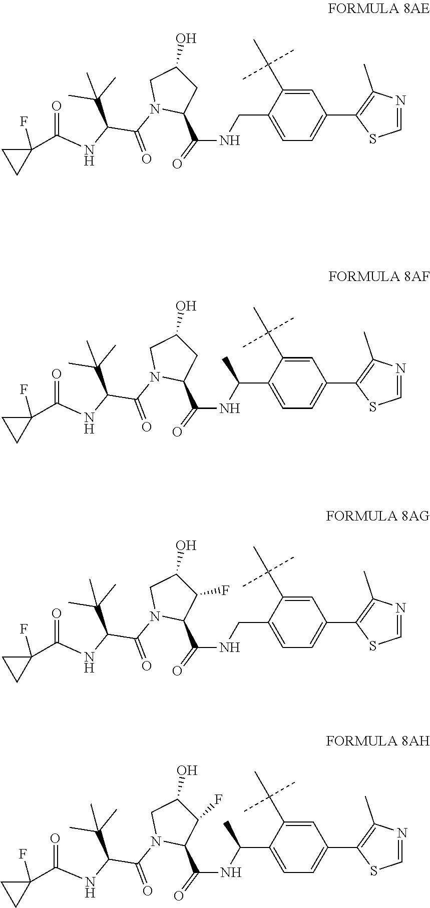

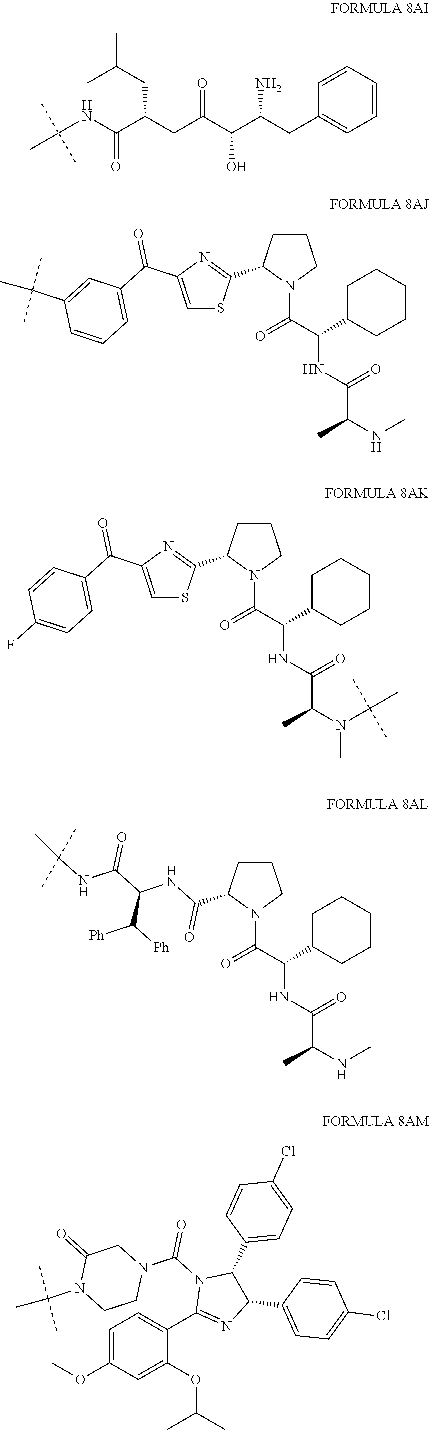

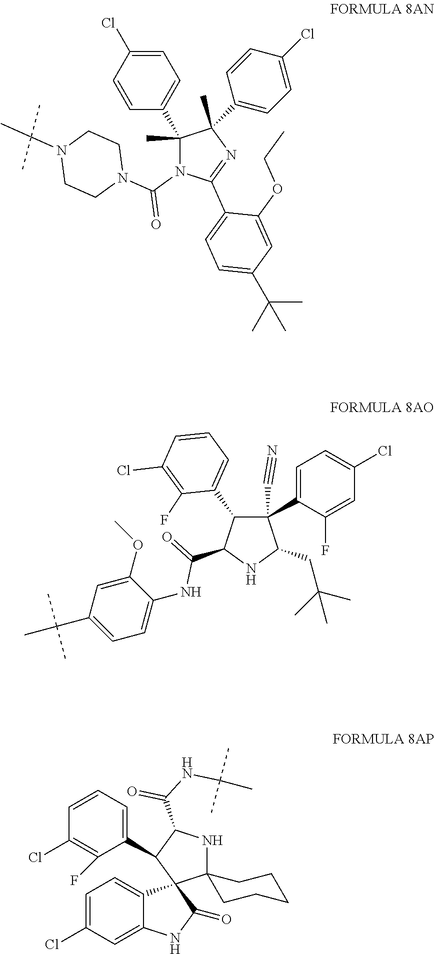

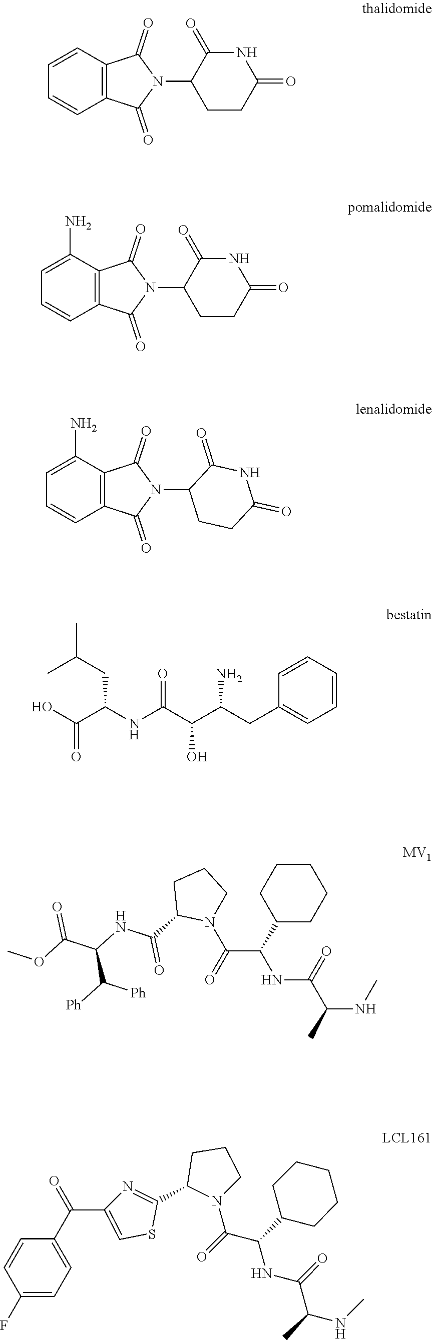

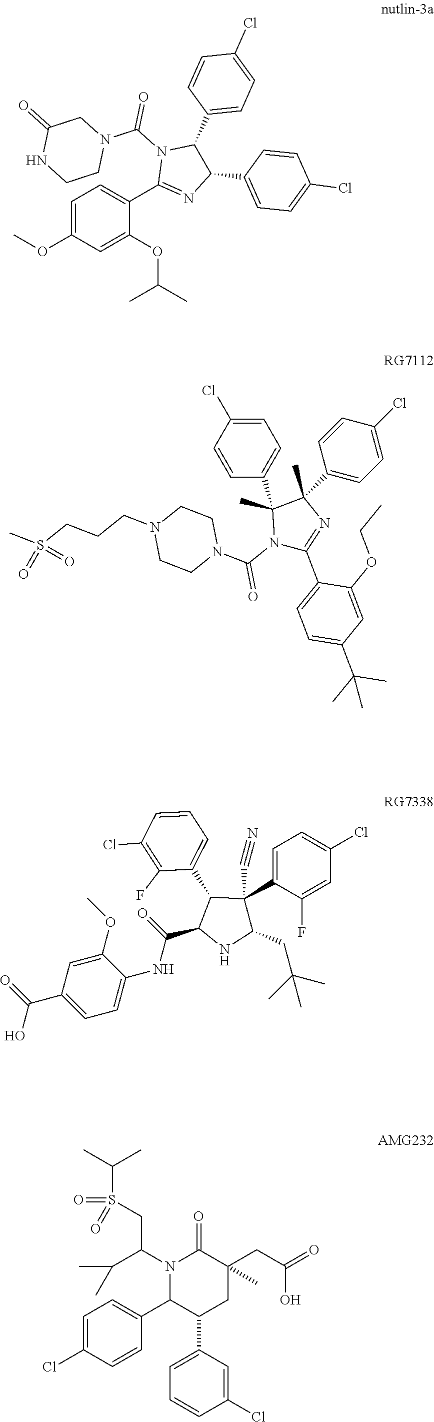

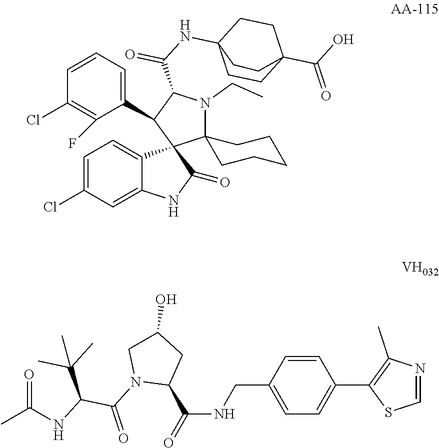 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CC(=O)N[C@H](C(=O)N1C[C@H](O)C[C@H]1C(=O)NCc1ccc(-c2scnc2C)cc1)C(C)(C)C]

In some aspects, the degradation/disruption tag can bind to a ubiquitin ligase (e.g., an E3 ligase such as a cereblon E3 ligase, a VHL E3 ligase, a MDM2 ligase, a TRIM21 ligase, a TRIM24 ligase, and/or an IAP ligase) and/or serve as a hydrophobic group that leads to PRMT5 protein misfolding.

Linkers

In any of the above-described compounds, the PRMT5 ligand can be conjugated to the degradation/disruption tag through a linker. The linker can include, e.g., acyclic or cyclic saturated or unsaturated carbon, ethylene glycol, amide, amino, ether, urea, carbamate, aromatic, heteroaromatic, heterocyclic, and/or carbonyl containing groups with different lengths.

In some aspects, the linker can be a moiety of:

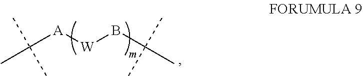

wherein

A, W and B, at each occurrence, are independently selected from null, or bivalent moiety selected from R′—R″, R′COR, R′CO₂R″, R′C(O)NR″R¹, R′C(S)NR″R¹, R′OR″, R′SR″, R′SOR″, R′SO₂R″, R′SO₂NR″R¹, R′NR″R¹, R′NR¹COR″, R′NR¹CONR″R², R′NR¹C(S)R″, R′OCH₂C(O)NR″R¹, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl, or
R′ and R″ together with the atom to which they are connected form a 3-20 membered cycloalkyl or 4-20 membered heterocyclyl ring;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹ and R² together with the atom to which they are connected form a 3-20 membered cycloalkyl or 4-20 membered heterocyclyl ring;

R′ and R¹, R′ and R², R″ and R¹, R″ and R² together with the atom to which they are connected form a 3-20 membered cycloalkyl or 4-20 membered heterocyclyl ring; and

m is 0 to 15.

In some embodiments, with respect for FORMULA 9, A is R′OCH₂C(O)NR″R¹; W is null or optionally substituted C₁-C₈ alkylene; B is null or optionally substituted C₁-C₈ alkylene; R′ is null; R″ is null or optionally substituted C₁-C₈ alkylene; R¹ is hydrogen; m is 0 to 6.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is C₂₋₆ alkylene.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is —(CH₂)₂—.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is —(CH₂)₄—.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is —(CH₂)₆—.

In some aspects, the linker can be a moiety of:

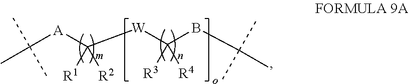

wherein

R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered cycloalkyl, optionally substituted 3-10 membered cycloalkoxy, optionally substituted 3-10 membered cycloalkylamino, optionally substituted 4-10 membered heterocyclyl, optionally substituted aryl, and optionally substituted heteroaryl, or

R¹ and R², R³ and R⁴ together with the atom to which they are connected form a 3-20 membered cycloalkyl or 4-20 membered heterocyclyl ring;

A, W and B, at each occurrence, are independently selected from null, or bivalent moiety selected from R′—R″, R′COR″, R′CO₂R″, R′C(O)NR″R⁵, R′C(S)NR″R⁵, R′OR″, R′SR″, R′SOR″, R′SO₂R″, R′SO₂NR″R⁵, R′NR″R⁵, R′NR⁵COR″, R′NR⁵CONR″R⁶, R′NR⁵C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl;
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;
   m is 0 to 15;
   n, at each occurrence, is 0 to 15; and
   o is 0 to 15.

In some aspects, the linker can be a moiety of:

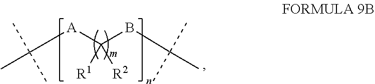

wherein

R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered cycloalkyl, optionally substituted 3-10 membered cycloalkoxy, optionally substituted 3-10 membered cycloalkylamino, optionally substituted 4-10 membered heterocyclyl, optionally substituted aryl, and optionally substituted heteroaryl, or
R¹ and R² together with the atom to which they are connected form a 3-20 membered cycloalkyl or 4-20 membered heterocyclyl ring;

A and B, at each occurrence, are independently selected from null, or bivalent moiety selected from R′—R″, R′COR″, R′CO₂R″, R′C(O)NR″R³, R′C(S)NR″R³, R′OR″, R′SR″, R′SOR″, R′SO₂R″, R′SO₂NR″R³, R′NR″R³, R′NR³COR″, R′NR³CONR″R⁴, R′NR³C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl;
R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;
   each m is 0 to 15; and
   n is 0 to 15.

In some aspects, the linker can be a moiety of:

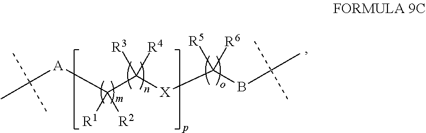

wherein
X is selected from 0, NH, and NR⁷;
R¹, R², R³, R⁴, R⁵, R⁶, and R⁷, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered cycloalkyl, optionally substituted 3-10 membered cycloalkoxy, optionally substituted 4-10 membered heterocyclyl, optionally substituted aryl, and optionally substituted heteroaryl;
   A and B are independently selected from null, or bivalent moiety selected from R′—R″, R′COR″, R′CO₂R″, R′C(O)NR″R⁸, R′C(S)NR″R⁸, R′OR″, R′SR″, R′SOR″, R′SO₂R″, R′SO₂NR″R⁸, R′NR″R⁸, R′NR⁸COR″, R′NR⁸CONR″R⁹, R′NR⁸C(S)R″, R′OCH₂C(O)NR″R¹, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl;
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;
   m, at each occurrence, is 0 to 15;
   n, at each occurrence, is 0 to 15;
   o is 0 to 15; and
   p is 0 to 15.

In some embodiments, with respect to FORMULA 9C, A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂,

In some embodiments, with respect to FORMULA 9C, o is 0 to 5.

In some embodiments, with respect to FORMULA 9C, X is O; R¹, R², R³, R⁴, R⁵, R⁶, and R⁷, at each occurrence, hydrogen; A is R′OCH₂C(O)NR″R¹ (R′=R″=null; R¹=H); B is R′C(O)R″ (R′=R″=null) m=0-2; n=0-2; o=0-10; and p=0-1.

In some embodiments, with respect to FORMULA 9C, X is O; R¹, R², R³, R⁴, R⁵, R⁶, and R⁷, at each occurrence, hydrogen; A is R′OCH₂C(O)NR″R¹ (R′=R″=null; R¹=H); B is R′C(O)R″ (R′=R″=null); m=0-2 and n=0-2; wherein m+n=2; o=1; and p=1.

In some embodiments, with respect to FORMULA 9C, R¹, R², R³, R⁴, R⁵, R⁶, and R⁷, at each occurrence, hydrogen; A is R′OCH₂C(O)NR″R¹ (R′=R″=null; R¹=H); B is R′C(O)R″ (R′=R″=null); o=2-12; and p=0.

In some embodiments, with respect to FORMULA 9C, R¹, R², R³, R⁴, R⁵, R⁶, and R⁷, at each occurrence, hydrogen; A is R′OCH₂C(O)NR″R¹ (R′=R″=null; R¹=H); B is R′ C(O)R″ (R′=R″=null); o=4; and p=0.

In some embodiments, with respect to FORMULA 9C, R¹, R², R³, R⁴, R⁵, R⁶, and R⁷, at each occurrence, hydrogen; A is R′OCH₂C(O)NR″R¹ (R′=R″=null; R¹=H); B is R′ C(O)R″ (R′=R″=null); o=10; and p=0.

In another aspect, the linker moiety comprises a ring selected from the group consisting of a 3 to 13 membered ring, a 3 to 13 membered fused ring, a 3 to 13 membered bridged ring, and a 3 to 13 membered spiro ring.

In some aspects, the linker moiety comprises one or more rings selected from the group consisting of formulae C1, C2, C3, C4 and C5:

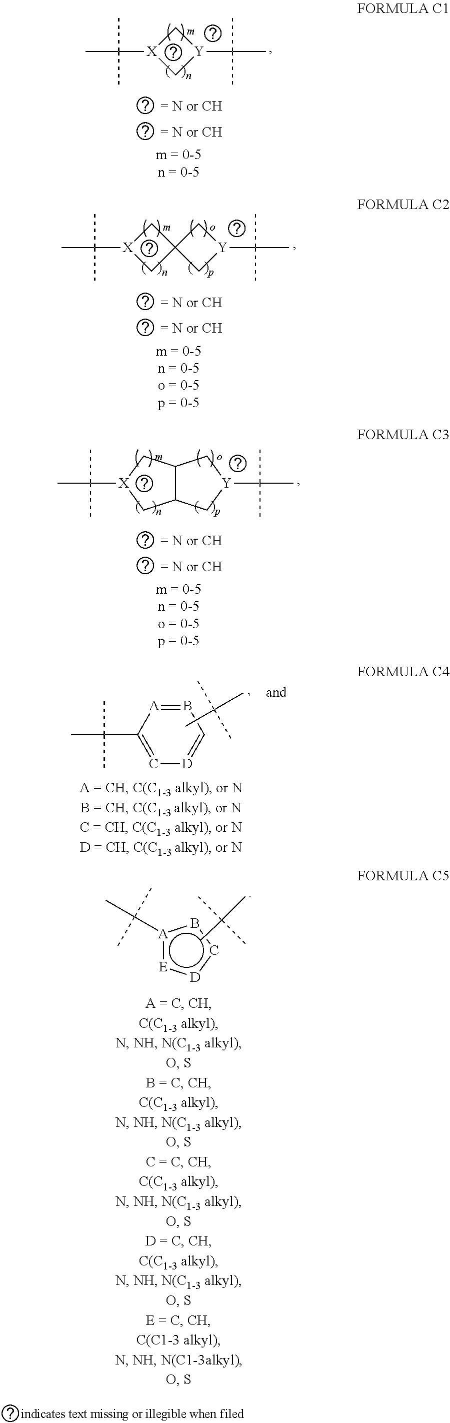

In some aspects, the linker can be a moiety of:

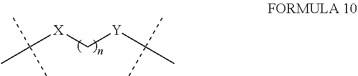

wherein X is CO or CH₂,

### Y is C═O or CH₂, and

n is 0-15;

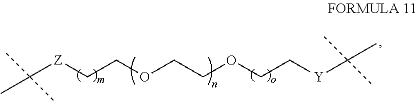

wherein X is C═O or CH₂,

### Y is C═O or CH₂,

m is 0-15,
n is 0-6, and
o is 0-15; or

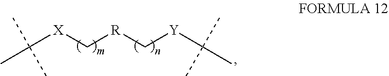

wherein

### X is C═O or CH₂,

### Y is C═O or CH₂,

R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl)-, —C═C—, —O—, —NH—, —N(C₁₋₃ alkyl)-, —C(O)NH—, —C(O)N(C₁₋₃ alkyl)-, a 3-13 membered ring, a 3-13 membered fused ring, a 3-13 membered bridged ring, and/or a 3-13 membered spiro ring,
m is 0-15, and
n is 0-15.

In some aspects of FORMULA 11, X is C═O, Y is C═O, m is 0-4, n is 2-6, and o is 0-4.

In some aspects of FORMULA 11, X is C═O, Y is C═O, m is 0-1, n is 4, and o is 0-1.

In some aspects of FORMULA 11, X is C═O, Y is C═O, m is 0, n is 4, and o is 0.

In some aspects of FORMULA 11, X is C═O, Y is C═O, m is 1, n is 4, and o is 1.

In some aspects of FORMULA 12, X is C═O or CH₂, Y is C═O or CH₂, R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl)-, —C═C—, m is 0-4, and n is 0-4.

In some aspects of FORMULA 12, X is C═O, Y is CH₂, R is —CH₂—, m is 0-4, n is 0-4, and m+n=4.

In some aspects of FORMULA 12, R is a 3-13 membered ring, a 3-13 membered fused ring, a 3-13 membered bridged ring, and/or a 3-13 membered spiro ring, one or more of which can contain one or more heteroatoms.

In some aspects of FORMULA 12, R has a structure of

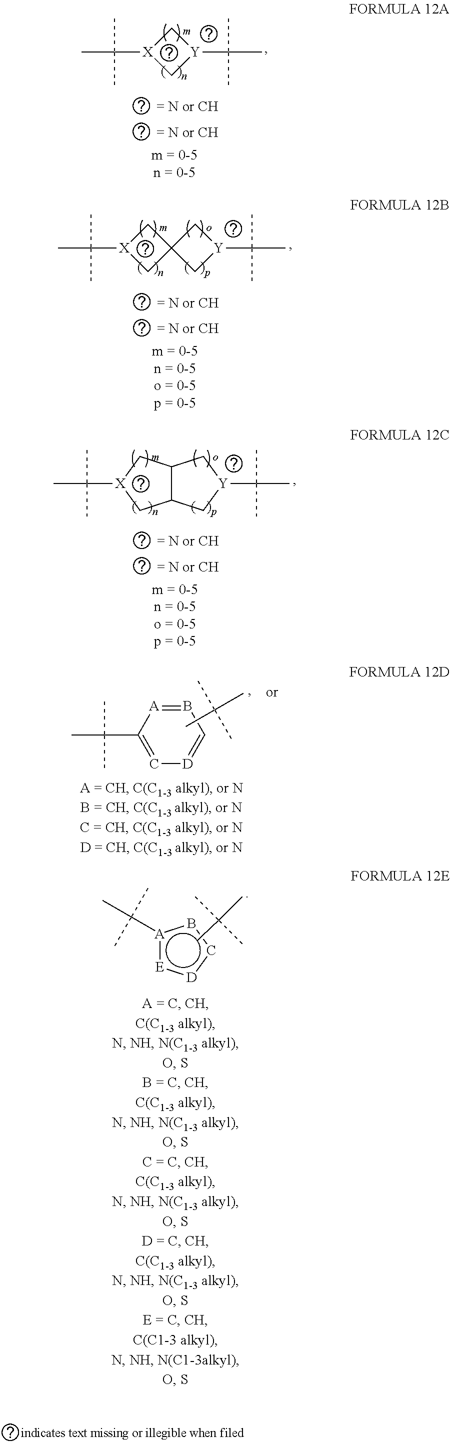

In some aspects, the bivalent compound is a compound selected from those synthesized in the Examples below, including, but not limited to: YS31-58, YS31-59, YS31-60, YS31-61, YS31-62, YS31-63, YS31-64, YS31-65, YS31-66, YS31-67, YS31-68, YS31-69, YS43-6, YS43-7, YS43-8, YS43-9, YS43-10, YS43-11, YS43-12, YS43-13, YS43-14, YS43-15, YS43-16, YS43-17, YS43-18, YS43-19, YS43-20, YS43-21, YS43-22, YS43-25, YS43-26, YS43-27, YS43-28, YS43-29, YS43-30, YS43-31, YS43-32, YS43-33, YS43-34, YS43-35, YS43-36, YS43-37, YS43-38, YS43-39, YS43-40, YS43-41, YS43-42, YS43-43, YS43-44, YS43-45, YS43-46, YS43-47, YS43-48, YS43-49, YS43-50, YS43-51, YS43-52, YS43-53, YS43-54, YS43-88, YS43-89, YS43-90, YS43-91, YS43-92, YS43-93, YS43-94, YS43-95, YS43-96, YS43-97, YS43-98, YS43-99, YS43-100, YS43-101, YS43-102, YS43-103, YS43-104, YS43-105, YS43-106, YS43-107, YS43-108, YS43-109, YS43-110, YS43-111, YS43-112, YS43-113, YS43-114, YS43-115, YS43-116, YS43-117, CPD-90 to CPD-118, or analogs thereof. In some embodiments, the bivalent compound is selected from the group consisting of YS43-93, YS43-95, YS43-97, YS43-100, YS43-111, YS31-60, YS43-8, YS43-16, and YS43-22. In some embodiments, the bivalent compound is selected from the group consisting of YS31-60, YS43-8, YS43-16, and YS43-22. In some embodiments, the bivalent compound is selected from the group consisting of YS43-93, YS43-95, YS43-97, YS43-100, YS43-111 and YS43-117.

In some aspects, this disclosure provides a method of treating the PRMT5-mediated diseases, the method including administering to a subject in need thereof with an PRMT5-mediated disease one or more bivalent compounds including an PRMT5 ligand conjugated to a degradation/disruption tag. The PRMT5-mediated diseases may be a disease resulting from PRMT5 amplification. The PRMT5-mediated diseases can have elevated PRMT5 enzymatic activity relative to a wild-type tissue of the same species and tissue type. Non-limiting examples of PRMT5-mediated diseases include acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, brain cancer (e.g., meningioma; glioma (e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), a hematopoietic cancer (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL), follicular lymphoma, chronic lymphocytic leukemia/small lymphocytic lymphoma (CLL/SLL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (e.g., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MPH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g. seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva). The PRMT5-mediated cancer can be a relapsed cancer. The PRMT5-mediated cancer can have been refractory to one or more previous treatments by different drugs.

In any of the above-described methods, the bivalent compounds can be YS31-58, YS31-59, YS31-60, YS31-61, YS31-62, YS31-63, YS31-64, YS31-65, YS31-66, YS31-67, YS31-68, YS31-69, YS43-6, YS43-7, YS43-8, YS43-9, YS43-10, YS43-11, YS43-12, YS43-13, YS43-14, YS43-15, YS43-16, YS43-17, YS43-18, YS43-19, YS43-20, YS43-21, YS43-22, YS43-25, YS43-26, YS43-27, YS43-28, YS43-29, YS43-30, YS43-31, YS43-32, YS43-33, YS43-34, YS43-35, YS43-36, YS43-37, YS43-38, YS43-39, YS43-40, YS43-41, YS43-42, YS43-43, YS43-44, YS43-45, YS43-46, YS43-47, YS43-48, YS43-49, YS43-50, YS43-51, YS43-52, YS43-53, YS43-54, YS43-88, YS43-89, YS43-90, YS43-91, YS43-92, YS43-93, YS43-94, YS43-95, YS43-96, YS43-97, YS43-98, YS43-99, YS43-100, YS43-101, YS43-102, YS43-103, YS43-104, YS43-105, YS43-106, YS43-107, YS43-108, YS43-109, YS43-110, YS43-111, YS43-112, YS43-113, YS43-114, YS43-115, YS43-116, YS43-117, CPD-90 to CPD-118, or analogs thereof. In some embodiments, the bivalent compound is selected from the group consisting of YS43-93, YS43-95, YS43-97, YS43-100, YS43-111, YS31-60, YS43-8, YS43-16, and YS43-22. In some embodiments, the bivalent compound is selected from the group consisting of YS31-60, YS43-8, YS43-16, and YS43-22. In some embodiments, the bivalent compound is selected from the group consisting of YS43-93, YS43-95, YS43-97, YS43-100, YS43-111 and YS43-117.

In some aspects of the disclosed methods, the bivalent compounds can be administered by any of several routes of administration including, e.g., orally, parenterally, intradermally, subcutaneously, topically, and/or rectally.

Any of the above-described methods can further include treating the subject with one or more additional therapeutic regimens for treating cancer. The one or more additional therapeutic regimens for treating cancer can be, e.g., one or more of surgery, chemotherapy, radiation therapy, hormone therapy, or immunotherapy.

This disclosure additionally provides a method for identifying a bivalent compound which mediates degradation/disruption of PRMT5, the method including providing a heterobifunctional test compound including a PRMT5 ligand conjugated to a degradation/disruption tag, contacting the heterobifunctional test compound with a cell (e.g., a cancer cell such as a PRMT5-mediated cancer cell) including a ubiquitin ligase and PRMT5.

As used herein, the terms “about” and “approximately” are defined as being within plus or minus 10% of a given value or state, preferably within plus or minus 5% of said value or state.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A-1H. Screening of PRMT5 PROTAC Compounds in MCF-7 Cells. FIGS. 1A-1H show a series of Western blots showing the effect of various PRMT5 degraders on reducing PRMT5 protein levels at 5 μM concentration in MCF-7 cells.

FIGS. 2A-2B. PRMT5 PROTACs Concentration- and Time-Dependently down-regulate PRMT5 Protein in MCF-7 Cells. FIGS. 2A-2B show a series of Western blots showing the effects of YS43-8 and YS43-22 on reducing PRMT5 protein levels in MCF-7 cells are concentration- and time-dependent.

FIG. 3. Effect of YS43-22 on Arginine Symmetric Dimethylation in MCF-7 Cells. FIG. 3 shows YS43-22 significantly inhibited the methylation (arginine symmetric demethylation) of PRMT5 substrates.

FIG. 4. PRMT5 PROTACs Downregulate PRMT5 Protein Levels in Different Cell Lines.

FIG. 4 shows YS43-22 significantly reduced of PRMT5 protein levels in different cell lines.

FIG. 5 shows YS43-22 significantly reduced cell proliferation of MCF-7 cells.

FIG. 6 shows YS43-22 is bioavailable in mice.

FIG. 7. Effect of PRMT5 Degraders on Reducing PRMT5 Protein Levels in MDA-MB-231 Cells. FIG. 7 is a series of Western blots showing the effect of selected PRMT5 degraders on reducing PRMT5 protein levels at 2 μM concentration in MDA-MB-231 cells.

FIGS. 8A-8C show that MTAP deletion in MDA-MB-231 cells confers higher susceptibility to PRMT5 degraders. (A) Western blots probing for Vinculin (top, 124 kD) and MTAP (bottom, 29 kD) in lysates from wild type cells or cells expressing RFP (control) or MTAP. (B) FACS analysis of RFP expression of wild type cells or cells expressing RFP or MTAP. (C) Cell viability after 7-day treatment with selected PRMT5 degraders or an inhibitor control (GSK591). Red bars represent MDA-MB-231 cells expressing RFP, and blue bars are cells expressing MTAP. *=p<0.05, **=p<0.005 (paired t-test).

### DEFINITION OF TERMS

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H5 group.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) 7C electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent 0 atoms or two adjacent S atoms. A heterocyclyl group may include fused, bridged or spirocyclic ring systems. In certain embodiments, a hetercyclyl group comprises 3 to 8 ring atoms (C₃-C₈ heterocyclyl; or 3-8 membered heterocyclyl). In certain embodiments, a hetercyclyl group comprises 3 to 10 ring atoms (C₃-C₁₀ heterocyclyl; or 3-10 membered heterocyclyl). In certain embodiments, a hetercyclyl group comprises 4 to 8 ring atoms (C₄-C₈ heterocyclyl; or 4-8 membered heterocyclyl). In certain embodiments, a hetercyclyl group comprises 4 to 10 ring atoms (C₄-C₁₀ heterocyclyl; or 4-10 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3 azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” or “carbocyclyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 6 carbon ring atoms (C₃-C₆ cycloalkyl; 3-6 membered cycloalkyl; or 3-6 membered carbocyclyl). In certain embodiments, a cycloalkyl comprises 3 to 8 carbon ring atoms (C₃-C₈ cycloalkyl; 3-8 membered cycloalkyl; or 3-8 membered carbocyclyl). In certain embodiments, a cycloalkyl comprises 3 to 10 carbon ring atoms (C₃-C₁₀ cycloalkyl; 3-10 membered cycloalkyl; or 3-10 membered carbocyclyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” or “bond” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

As used herein, the same symbol in different FORMULAE refers to a different definition, for example, the definition of R1 in FORMULA 1 is as defined with respect to FORMULA 1 and the definition of R1 in FORMULA 6 is as defined with respect to FORMULA 6.

As used herein, when m (or n or o or p) is definited by a range, for example, “m is 0 to 15” or “m=0-3” mean that m is an integer from 0 to 15 (i.e. m is 0, 1, 2, 3, 4, 5, 6, 7, 8, 9, 10, 11, 12, 13, 14, or 15) or m is an integer from 0 to 3 (i.e. m is 0, 1, 2, or 3) or is any integer in the defined range.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al.

### DETAILED DESCRIPTION

The present disclosure is based, in part, on the discovery that novel heterobifunctional molecules (e.g., small molecules) which degrade PRMT5, PRMT5 fusion proteins, and/or PRMT5 mutant proteins (“PROteolysis TArgeting Chimeras” or “PROTACs”) are useful in the treatment of PRMT5-mediated diseases, particularly lymphomas, melanoma, adenocarcinoma, pancreatic cancer, prostate cancer, lung cancer, breast cancer, colorectal cancer, and ovarian cancer.

Successful strategies for selective degradation/disruption of the target protein induced by a small molecule (e.g., bifunctional molecule) include recruiting an E3 ubiquitin ligase and mimicking protein misfolding with a hydrophobic tag (Buckley and Crews, 2014). PROTACs are bivalent molecubles (e.g., inhibitors) with one moiety that binds an E3 ubiquitin ligase and another moiety that binds the protein target of interest (Buckley and Crews, 2014). The induced proximity leads to (selective) ubiquitination of the target followed by its degradation at the proteasome. Two types of high affinity small-molecule E3 ligase ligands have been identified/developed: immunomodulatory drugs (IMiDs) such as thalidomide and pomalidomide, which bind cereblon (CRBN or CRL4CRBN), a component of a cullin-RING ubiquitin ligase (CRL) complex (Bondeson et al., 2015; Chamberlain et al., 2014; Fischer et al., 2014; Ito et al., 2010; Winter et al., 2015); and VHL-1, a hydroxyproline-containing ligand, which binds van Hippel-Lindau protein (VHL or CRL2VHL), a component of another CRL complex (Bondeson et al., 2015; Buckley et al., 2012a; Buckley et al., 2012b; Galdeano et al., 2014; Zengerle et al., 2015). The PROTAC technology has been successfully applied to degradation of multiple targets (Bondeson et al., 2015; Buckley et al., 2015; Lai et al., 2016; Lu et al., 2015; Winter et al., 2015; Zengerle et al., 2015), but not to degradation of PRMT5. In addition, a hydrophobic tagging approach, which utilizes a bulky and hydrophobic adamantyl group, has been developed to mimic protein misfolding, leading to the degradation of the target protein by proteasome (Buckley and Crews, 2014). This approach has also been successfully applied to selective degradation of the pseudokinase Her3 (Xie et al., 2014), but not to degradation of PRMT5 proteins.

As discussed in the following examples, this disclosure provides specific examples of novel PRMT5 degraders/disruptors, and examined the effect of exemplary degraders/disruptors on inhibiting/disrupting PRMT5 activity, suppressing PRMT5 expression (e.g., reducing PRMT5 protein levels), and inhibiting cancer cell proliferation. The results indicated that these novel small molecules can be beneficial in treating cancer, especially PRMT5-positive lymphomas, melanoma, adenocarcinoma, pancreatic cancer, prostate cancer, lung cancer, breast cancer, colorectal cancer, and ovarian cancer.

A number of selective small-molecule PRMT5 catalytic inhibitors, such as EPZ015666, GSK591, GSK3326595 (EPZ015938), BLL-1, HLCL-61, LLY-283, and PF-06855800 have recently been reported. Several compounds, including GSK3326595, are being investigated in clinical trials for treating patients with solid tumors and non-Hodgkin's lymphoma.

Current drugs (e.g., compounds) targeting PRMT5 generally focus on inhibition of its catalytic function/activity. In the present disclosure a different approach was taken: to develop compounds that directly and selectively target not only the catalytic function of PRMT5, but also its level of expression at the protein level (i.e., protein level in cells). Strategies for inducing protein degradation include recruiting E3 ubiquitin ligases, mimicking protein misfolding with hydrophobic tags, and inhibiting chaperones. For example, a thalidomide-JQ1 bivalent compound has been used to hijack the cereblon E3 ligase, inducing highly selective BET protein degradation in vitro and in vivo and resulting in a demonstrated delay in leukemia progression in mice (Winter et al., 2015). Similarly, BET protein degradation has also been induced via another E3 ligase, VHL (Zengerle et al., 2015). Partial degradation of the Her3 protein has been induced using an adamantane-modified compound (Xie et al., 2014). Such an approach, based on the use of bivalent small molecule compounds, permits more flexible regulation of protein expression in vitro and in vivo compared with techniques such as gene knockout or shRNA (short hairpin RNA) knockdown. Unlike gene knockout or shRNA knockdown, a small molecule approach provides an opportunity to study dose and time dependency in a disease model by varying the concentrations and frequencies of administration of the relevant small molecule.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof.

This disclosure includes pharmaceutically acceptable salts of the structures depicted and compounds named herein.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. In some embodiments, the compound includes at least one fluorine atom. In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

### PROTACs/Degraders

In some aspects, the present disclosure provides bivalent compounds, also referred to herein as PROTACs or degraders, comprising a PRMT5 ligand (or targeting moiety) conjugated to a degradation tag. Linkage of the PRMT5 ligand to the degradation tag can be direct, or indirect via a linker.

As used herein, the terms “protein arginine methyltransferase 5 (PRMT5) ligand” or “PRMT5 ligand” or “PRMT5 targeting moiety” are to be construed broadly, and encompass a wide variety of molecules ranging from small molecules to large proteins that associate with or bind to PRMT5. The PRMT5 ligand or targeting moiety can be, for example, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof.

The PRMT5 ligand or targeting moiety can be derived from a PRMT5 inhibitor (e.g., EPZ015666, GSK591, GSK3326595 (EPZ015938), BLL-1, HLCL-61, LLY-283, PF-06855800, and analogs thereof), which is capable of interfering with the enzymatic activity of PRMT5. As used herein, an “inhibitor” refers to an agent that restrains, retards, or otherwise causes inhibition of a physiological, chemical or enzymatic action or function. As used herein an inhibitor causes a decrease in enzyme activity of at least 5%. An inhibitor can also or alternatively refer to a drug, compound, or agent that prevents or reduces the expression, transcription, or translation of a gene or protein. An inhibitor can reduce or prevent the function of a protein, e.g., by binding to or activating/inactivating another protein or receptor.

Exemplary PRMT5 ligands include, but are not limited to, the compounds listed below:

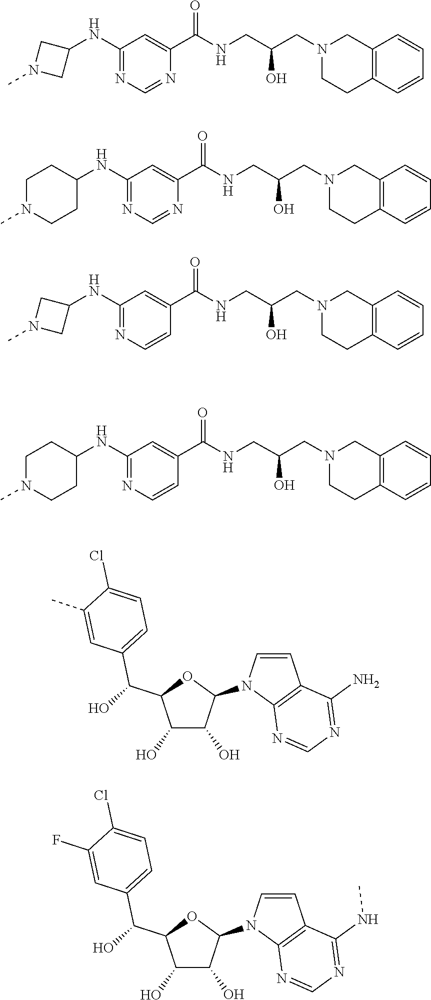

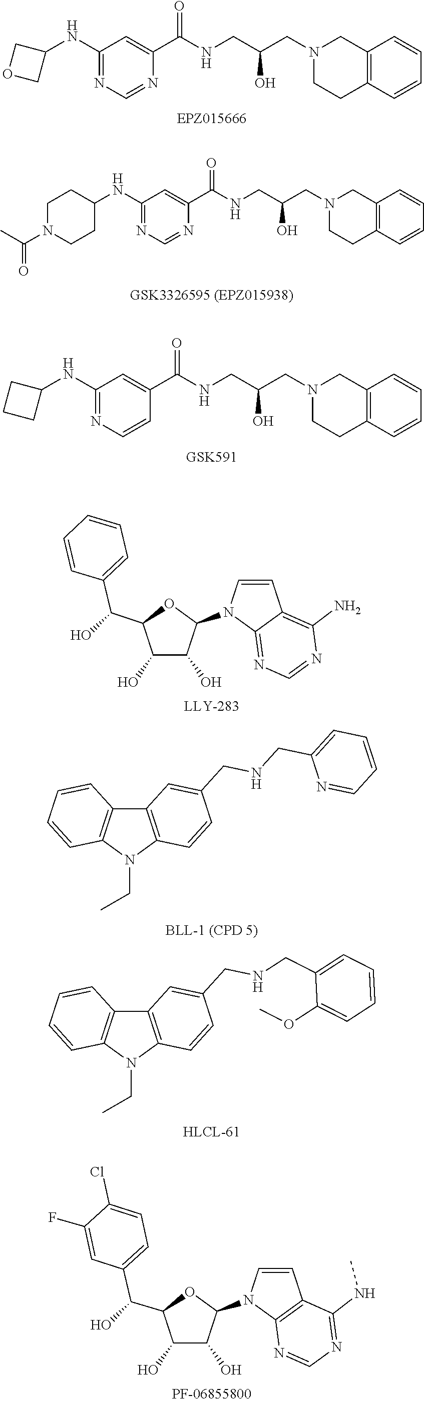

As used herein, the term “degradation/disruption tag” refers to a compound, which associates with or binds to a ubiquitin ligase for recruitment of the corresponding ubiquitination machinery to PRMT5 or induces PRMT5 protein misfolding and subsequent degradation at the proteasome or loss of function.

In some aspects, the degradation/disruption tags of the present disclosure include, e.g., thalidomide, pomalidomide, lenalidomide, VHL-1, adamantane, 1-(4,4,5,5,5-pentafluoropentyl)sulfinyl)nonane, nutlin-3a, RG7112, RG7338, AMG232, AA-115, bestatin, MV-1, LCL161, and/or analogs thereof.

As used herein, a “linker” is a bond, molecule, or group of molecules that binds two separate entities to one another. Linkers can provide for optimal spacing of the two entities. The term “linker” in some aspects refers to any agent or molecule that bridges the PRMT5 ligand to the degradation/disruption tag. One of ordinary skill in the art recognizes that sites on the PRMT5 ligand or the degradation/disruption tag, which are not necessary for the function of the PROTACs of the present disclosure, are ideal sites for attaching a linker, provided that the linker, once attached to the conjugate of the present disclosure, does not interfere with the function of the PROTAC, i.e., its ability to target PRMT5 and its ability to recruit a ubiquitin ligase.

The length of the linker of the bivalent compound can be adjusted to minimize the molecular weight of the disruptors/degraders and avoid any potential clash of the PRMT5 ligand or targeting moiety with either the ubiquitin ligase or the induction of PRMT5 misfolding by the hydrophobic tag at the same time.

In some aspects, the degradation/disruption tags of the present disclosure include, for example, thalidomide, pomalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, and analogs thereof. The degradation/disruption tags can be attached to any portion of the structure of a PRMT5 ligand or targeting moiety (e.g., EPZ015666, GSK591, GSK3326595 (EPZ015938), BLL-1, HLCL-61, LLY-283, and PF-06855800) with linkers of different types and lengths in order to generate effective bivalent compounds. In particular, attaching VHL1, pomalidomide, or LCL161 to any portion of the molecule can recruit the E3 ligase to PRMT5.

The bivalent compounds disclosed herein can selectively affect PRMT5-mediated cancer cells compared to WT (wild-type) cells (i.e., a PRMT5 degrader/disruptor able to kill or inhibit the growth of a PRMT5-mediated cancer cell while also having a relatively low ability to lyse or inhibit the growth of a WT cell), e.g., possess a GI₅₀ for one or more PRMT5-mediated cancer cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the PRMT5-mediated cancer cells.

Additional bivalent compounds (i.e., PRMT5 degraders/disruptors) can be developed using the principles and methods disclosed herein. For example, other linkers, degradation tags, and PRMT5 binding/inhibiting moieties (not limited to EPZ015666, GSK591, GSK3326595 (EPZ015938), BLL-1, HLCL-61, LLY-283 and PF-06855800) can be synthesized and tested. Non-limiting examples of PRMT5 disruptors/degraders (e.g., bivalent compounds) are shown in Table 1 (below). The left portion of each PRMT5 disruptors/degrader compound as shown binds to PRMT5 (as EPZ015666, GSK591, GSK3326595 (EPZ015938), BLL-1, HLCL-61, or LLY-283 and PF-06855800 do), and the right portion of each compound recruits for the ubiquitination machinery to PRMT5, which induces the poly-ubiquitination and degradation of PRMT5 at the proteasome.

In some aspects, the PRMT5 degraders/disruptors have the form “PI-linker-EL”, as shown below:

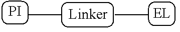

wherein PI (protein of interest) comprises a PRMT5 ligand (e.g., a PRMT5 inhibitor) and EL (E3 ligase) comprises a degradation/disruption tag (e.g., E3 ligase ligand). Exemplary PRMT5 ligands (PI), exemplary degradation/disruption tags (EL), and exemplary linkers (Linker) are illustrated below:

### PRMT5 Ligands

In one aspect, the PRMT5 Ligand (PI) comprises:

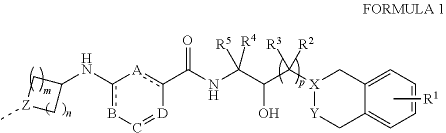

wherein
A, B, C, and D are independently a bond, CR⁶, NR⁷, N, O, or S;
X and Z are independently CR⁷, CR⁸, or N;
Y is a bond, CR⁸, CR⁹, N, or NR¹⁰,
R¹, R², R³, R⁴, R⁵, R⁶, R⁷, R⁸, R⁹, and R¹⁹ are independently hydrogen, halogen, optionally substituted C₁-C₈ alkyl, optionally substituted C₁-C₈ alkoxy, and optionally substituted C₁-C₈ alkoxyalkyl;
m and n are independently 0, 1, 2, 3, or 4; and
p is 0 or 1.

In some embodiments with respect to FORMULA 1,

the “Linker” moiety of the bivalent compound is attached to Z;
A, B, C, and D are independently a bond, CR⁶, NR⁷, N, O, or S;
X and Z are independently CR⁸, or N;
Y is a bond, CR⁹, or NR¹⁰,
R¹, R², R³, R⁴, R⁵, R⁶, R⁷, R⁸, R⁹, and R¹⁹ are independently hydrogen, halogen, optionally substituted C₁-C₈ alkyl, optionally substituted C₁-C₈ alkoxy, and optionally substituted C₁-C₈ alkoxyalkyl;
m and n are independently 0, 1, 2, 3, or 4; and
p is 0 or 1.

In some embodiments with respect to FORMULA 1,

A, B, C, and D are independently a bond, CR⁶, N, O, or S;
X and Z are independently CR⁷ or N;
Y is a bond, CR⁸, N, or NR¹⁰,
R¹, R², R³, R⁴, R⁵, R⁶, R⁷, and R⁸ are independently hydrogen, halogen, C₁-C₈ alkyl, C₁-C₈ alkoxy, and C₁-C₈ alkoxyalkyl;
m and n are independently 0-3; and
p is 0 or 1.

In another embodiment, with respect to FORMULA 1, A and C are CH; B is N; D is optionally selected from CH or N.

In another embodiment, with respect to FORMULA 1, X and Z are N.

In another embodiment, with respect to FORMULA 1, Y is a bond or CH₂.

In another embodiment, with respect to FORMULA 1, R¹, R², R³, R⁴, R⁵, R⁶, R⁷, R⁸, R⁹, and R¹⁰ are independently selected from hydrogen and halogen.

In another embodiment, with respect to FORMULA 1, m and n are independently selected from 1 and 2.

In another embodiment, with respect to FORMULA 1, p is 1.

In another aspect, the PRMT5 Ligand (PI) comprises:

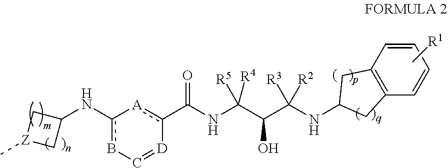

wherein,
A, B, C, and D are independently selected from a bond, CR⁶, NR⁷, N, O, and S;
Z is independently selected from CR⁷, CR⁸ and N;
R¹, R², R³, R⁴, R⁵, R⁶, R⁷, and R⁸ are independently selected from hydrogen, halogen, optionally substituted C₁-C₈ alkyl, optionally substituted C₁-C₈ alkoxy, and optionally substituted C₁-C₈ alkoxyalkyl; and
m, n, p, and q are independently selected from 0, 1, 2, 3, and 4.

In some embodiments, with respect to FORMULA 2,

A, B, C, and D are independently a bond, CR⁶, N, O, or S;
Z is independently CR⁷, or N;
R¹, R², R³, R⁴, R⁵, R⁶, R⁷, and R⁸ are independently hydrogen, halogen, C₁-C₈ alkyl, C₁-C₈ alkoxy, or C₁-C₈ alkoxyalkyl; and
m, n, and p are 0-3.

In some embodiments, with respect to FORMULA 2,

the “Linker” moiety of the bivalent compound is attached to Z;
A, B, C, and D are independently selected from a bond, CR⁶, NR⁷, N, O, and S;
Z is independently selected from CR⁸ and N;
R¹, R², R³, R⁴, R⁵, R⁶, R⁷, and R⁸ are independently selected from hydrogen, halogen, optionally substituted C₁-C₈ alkyl, optionally substituted C₁-C₈ alkoxy, and optionally substituted C₁-C₈ alkoxyalkyl; and
m, n, p, and q are independently selected from 0, 1, 2, 3, and 4.

In some embodiments, with respect to FORMULA 2, A and C are CH; B is N; D is optionally selected from CH and N.

In some embodiments, with respect to FORMULA 2, Z is N.

In some embodiments, with respect to FORMULA 2, R¹, R², R³, R⁴, R⁵, R⁶, R⁷, and R⁸ are independently selected from hydrogen and halogen.

In some embodiments, with respect to FORMULA 2, m, n, p and q are independently selected from 1 and 2.

In another aspect, the PRMT5 Ligand (PI) comprises:

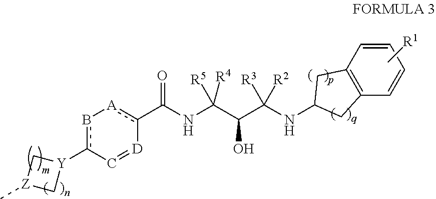

wherein
the “Linker” moiety of the bivalent compound is attached to Z;
A, B, C, and D are independently selected from a bond, CR⁶, NR⁷, N, O, or S;
Y and Z are independently selected from CR⁸ or N;
R¹, R², R³, R⁴, R⁵, R⁶, R⁷, and R⁸ are independently selected from hydrogen, halogen, optionally substituted C₁-C₈ alkyl, optionally substituted C₁-C₈ alkoxy, and optionally substituted C₁-C₈ alkoxyalkyl; and
m, n, p, and q are independently selected from 0, 1, 2, 3, and 4.

In some embodiments, with respect to FORMULA 3, A and C are CH; B is N; D is optionally selected from CH or N.

In some embodiments, with respect to FORMULA 3, Y and Z independently selected from CH and N.

In some embodiments, with respect to FORMULA 3, R¹, R², R³, R⁴, R⁵, R⁶, R⁷, and R⁸ are independently selected from hydrogen and halogen.

In some embodiments, with respect to FORMULA 3, m, n, p and q are independently selected from 1 and 2.

In the formulas above, the reference to a “bond” means that the respective letter A, B, C or D refers to the absence of an atom or moiety, and there is a bond between adjacent atoms in the structure.

In another aspect, the PRMT5 Ligand (PI) comprises:

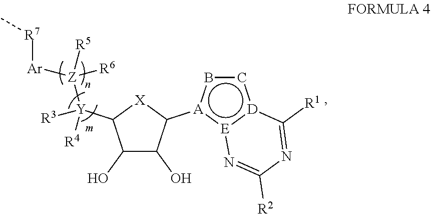

wherein
the “Linker” moiety of the bivalent compound is attached to R⁷;
X is selected from CH₂ and O;
Y and Z are selected from null, C, O, and S;
A, B, C, D, and E are independently selected from null, CR⁸, CR⁸═CR⁹, CNR¹⁰R¹¹, CNR¹⁰C(O)R¹¹, C NR⁸C(O)NR¹⁰R¹¹, CNR⁸SOR¹⁰, CNR⁸SO₂R¹⁰, NR¹⁰, N, N═N, CR⁸═N, O, and S, wherein
R⁸, R⁹, R¹⁰, and R¹¹ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered cycloalkyl, optionally substituted 4-10 membered heterocyclyl;
   R¹ is selected from hydrogen, halogen, cyano, nitro, OR¹², SR₁₂, NR₁₃R¹⁴, COR¹², CO₂R¹², C(O)NR¹³R¹⁴, SOR¹², SO₂R¹², SO₂NR¹³R¹⁴, NR¹²C(O)R¹³, NR¹²C(O)NR¹³R¹⁴, NR¹²SOR¹³, NR¹²SO₂R¹³, optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl, optionally substituted aryl, and optionally substituted heteroaryl, wherein
R¹², R¹³, and R¹⁴ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹² and R¹³, R¹³ and R¹⁴ together with the atom to which they are connected form an optionally substituted 4-10 membered heterocyclyl ring;
   R², R³, R⁴, R⁵ and R⁶ are independently selected from null, hydrogen, halogen, OR¹⁵, NR¹⁶R¹⁷, optionally substituted C₁-C₈ alkyl, optionally substituted C₂-C₈ alkenyl, optionally substituted C₂-C₈ alkynyl, optionally substituted 3-10 membered cycloalkyl, and optionally substituted 4-10 membered heterocyclyl, wherein
R¹⁵, R¹⁶, and R¹⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted C₂-C₈ alkenyl, optionally substituted C₂-C₈ alkynyl, or
R¹⁶ and R¹⁷ together with the atom to which they are connected form an optionally substituted 4-10 membered heterocyclyl ring;
   R⁷ is selected from null, OR¹⁸, SR¹⁸, NR¹⁸R¹⁹, OC(O)R¹⁸, OC(O)OR¹⁸, OCONR¹⁸R¹⁹, C(O)R¹⁸, C(O)OR¹⁸, CONR¹⁸R¹⁹, S(O)R¹⁸, S(O)₂R¹⁸, SO₂NR¹⁸R¹⁹, NR²⁰C(O)OR¹⁸, NR²⁰C(O)R¹⁸, NR²⁰C(O)NR¹⁸R¹⁹, NR²⁰S(O)R¹⁸, NR²⁰S(O)₂R¹⁸, NR²⁰S(O)₂NR¹⁸R¹⁹, optionally substituted C₁-C₈ alkylene, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹⁸ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹⁹ and R²⁰ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl; or
R¹⁸ and R¹⁹, R¹⁸ and R²⁰, R¹⁹ and R²⁰ together with the atom to which they are connected form a 4-20 membered heterocyclyl ring;
   Ar is selected from null, aryl and heteroaryl, each of which is substituted with R⁷ and optionally substituted with one or more substituents independently selected from hydrogen, halogen, oxo, CN, NO₂, OR²¹, SR²¹, NR²¹R²², OCOR²¹, OCO₂R²¹, OCONR²¹R²², COR²¹, CO₂R²¹, CONR²¹R²², SOR²¹, SO₂R²¹, SO₂NR²¹R²², NR²³CO₂R²¹, NR²³COR²¹, NR²³C(O)NR²¹R²², NR²³SOR²¹, NR²³SO₂R²¹, NR²³SO₂NR²¹R²², optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R²¹, R²² and R²³ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R²¹ and R²², R²¹ and R²³ together with the atom to which they are connected form a 4-20 membered heterocyclyl ring; and
   m and n are independently selected from 0 and 1.

In some embodiments, FORMULA 4 is FORMULA 4A:

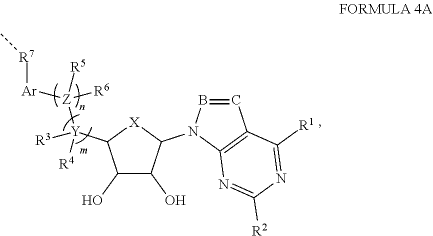

wherein
the definitions of X, Y, Z, B, C, R¹, R², R³, R⁴, R⁵, R⁶, R⁷ and Ar are the same as FORMULA 4.

In some embodiments, FORMULA 4 is FORMULA 4B:

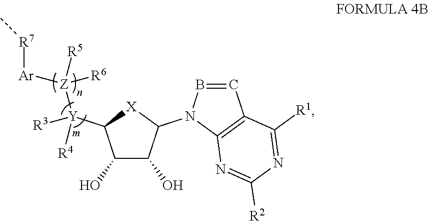

wherein
the definitions of X, Y, Z, B, C, R¹, R², R³, R⁴, R⁵, R⁶, R⁷ and Ar are the same as FORMULA 4.

In some embodiments, FORMULA 4 is FORMULAE 4C, 4D and 4E:

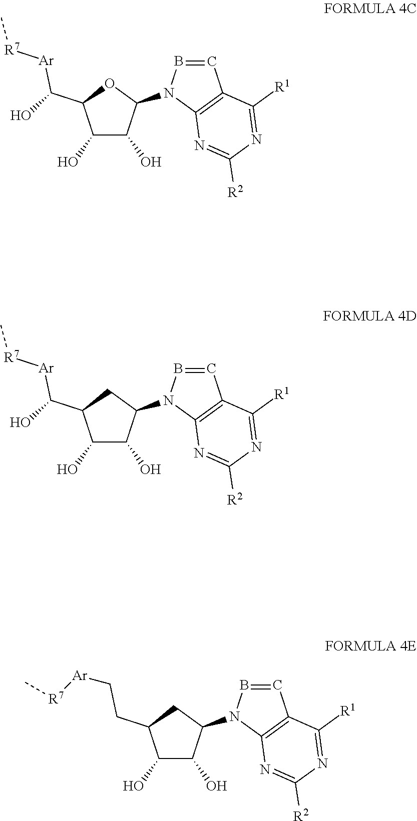

wherein
the definitions of B, C, R¹, R², R⁷ and Ar are the same as FORMULA 4.

In some embodiments, with respect to FORMULA 4 and FORMULAS 4A-4E,

B is selected from CH and N;
C is selected from CR⁸, CNR¹⁰R¹¹, CNR¹⁰C(O)R¹¹, C NR⁸C(O)NR¹⁰R¹¹, CNR⁸SOR¹⁰, CNR⁸SO₂R¹⁰, and N, wherein
R⁸, R¹⁰, and R¹¹ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkylamino, optionally substituted 3-10 membered cycloalkyl, optionally substituted 4-10 membered heterocyclyl;
   R¹ is selected from NR¹³R¹⁴, NR¹²C(O)R¹³, NR¹²C(O)NR¹³R¹⁴, NR¹²SOR¹³, NR¹²SO₂R¹³ optionally substituted C₁-C₈ alkyl, optionally substituted 3-10 membered cycloalkyl, optionally substituted 4-10 membered heterocyclyl, wherein

R¹², R¹³ and R¹⁴ are independently selected from hydrogen, optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl, optionally substituted aryl, and optionally substituted heteroaryl, or

R¹³ and R¹⁴ together with the atom to which they are connected form an optionally substituted 4-10 membered heterocyclyl ring;

R² is selected from hydrogen, methyl, and NH₂;
R⁷ is selected from null, OR¹⁸, SR¹⁸, NR¹⁸R¹⁹, C(O)R¹⁸, C(O)OR¹⁸, CONR¹⁸R¹⁹, S(O)R¹⁸, S(O)₂R¹⁸, SO₂NR¹⁸R¹⁹, NR²⁰C(O)OR¹⁸, NR²⁰C(O)R¹⁸, NR²⁰C(O)NR¹⁸R¹⁹, NR²⁰S(O)R¹⁸, NR²⁰S(O)₂R¹⁸, NR²⁰S(O)₂NR¹⁸R¹⁹, optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹⁸ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹⁹ and R²⁰ are independently selected from optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl, optionally substituted aryl, and optionally substituted heteroaryl; or
R¹⁸ and R¹⁹, R¹⁸ and R²⁰, R¹⁹ and R²⁰ together with the atom to which they are connected form a 4-20 membered heterocyclyl ring;
   Ar is selected from null, aryl and heteroaryl, each of which is substituted with R⁷ and optionally substituted with one or more substituents independently selected from hydrogen, halogen, oxo, CN, NO₂, OR²¹, SR²¹, NR²¹R²², OCOR²¹, OCO₂R²¹, OCONR²¹R²², COR²¹, CO₂R²¹, CONR²¹R²², SOR²¹, SO₂R²¹, SO₂NR²¹R²², NR²³CO₂R²¹, NR²³COR²¹, NR²³C(O)NR²¹R²², NR²³SOR²¹, NR²³SO₂R²¹, NR²³SO₂NR²¹R²², optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R²¹, R²² and R²³ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R²¹ and R²², R²¹ and R²³ together with the atom to which they are connected form a 4-20 membered heterocyclyl ring.

In some embodiments, FORMULA 4 is FORMULA 4F:

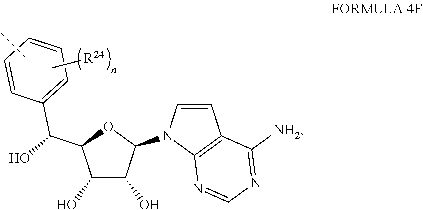

wherein
each R²⁴ is independently selected from null, hydrogen, halogen, oxo, CN, NO₂, OR²⁵, SR²⁵, NR²⁵R²⁶, OCOR²⁵, OCO₂R²⁵, OCONR²⁵R²⁶, COR²⁵, CO₂R²⁵, CONR²⁵R²⁶, SOR²⁵, SO₂R²⁵, SO₂NR²⁵R²⁶, NR²⁷CO₂R²⁵, NR²⁷COR²⁵, NR²⁷C(O)NR²⁵R²⁶, NR²⁷SOR²⁵, NR²⁷SO₂R²⁵, NR²⁷SO₂NR²⁵R²⁶, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R²⁵, R²⁶ and R²⁷ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted C₃-C₈ cycloalkoxy, optionally substituted 4-10 membered heterocyclyl, optionally substituted aryl, and optionally substituted heteroaryl, or
R²⁵ and R²⁶, R²⁵ and R²⁷ together with the atom to which they are connected form a 4-20 membered heterocyclyl ring; and
   n is independently selected from 0, 1, 2, 3, and 4.

In some embodiments, FORMULA 4 is FORMULA 4G:

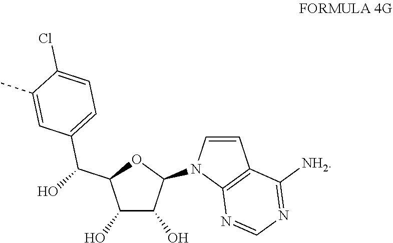

In another aspect, the PRMT5 Ligand (PI) comprises:

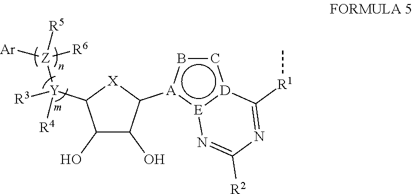

wherein
the “Linker” moiety of the bivalent compound is attached to R¹;
X is selected from CH₂ and O;
Y and Z are selected from null, C, O, and S;
A, B, C, D, and E are independently selected from null, CR⁷, CR⁷═CR⁸, CNR⁹R¹⁰, CNR⁹C(O)R¹⁰, CNR⁸C(O)NR⁹R¹⁰, CNR⁷SOR⁹, CNR⁷SO₂R⁹, NR⁹, N, N═N, CR⁷═N, O, and S, wherein
R⁷, R⁸, R⁹ and R¹⁰ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈alkoxy, optionally substituted C₁-C₈alkoxyC₁-C₈alkyl, optionally substituted C₁-C₈alkylamino, optionally substituted C₁-C₈alkylaminoC₁-C₈alkyl, optionally substituted 3-10 membered cycloalkyl, optionally substituted 4-10 membered heterocyclyl;
   R¹ is selected from null, OR¹¹, SR¹¹, NR¹¹R¹², OC(O)R¹¹, OC(O)OR¹¹, OCONR¹¹R¹², C(O)R¹¹, C(O)OR¹¹, CONR¹¹R¹², S(O)R¹¹, S(O)₂R¹¹, SO₂NR¹¹R¹², NR¹³C(O)OR¹¹, NR¹³C(O)R¹¹, NR¹³C(O)NR¹¹R¹², NR¹³S(O)R¹¹, NR¹³S(O)₂R¹¹, NR¹³S(O)₂NR¹¹R¹², optionally substituted C₁-C₈ alkylene, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹¹ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹² and R¹³ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl; or
R¹¹ and R¹², R¹¹ and R¹³, R¹² and R¹³ together with the atom to which they are connected form a 4-20 membered heterocyclyl ring;
   R², R³, R⁴, R⁵ and R⁶ are independently selected from hydrogen, halogen, OR¹⁴, NR¹⁵R¹⁶, optionally substituted C₁-C₈ alkyl, optionally substituted C₂-C₈ alkenyl, optionally substituted C₂-C₈ alkynyl, optionally substituted 3-10 membered cycloalkyl, and optionally substituted 4-10 membered heterocyclyl, wherein
R¹⁴, R¹⁵ and R¹⁶ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl; or
R¹⁵ and R¹⁶ together with the atom to which they are connected form an optionally substituted 4-10 membered heterocyclyl ring;
   Ar is selected from aryl and heteroaryl, each of which is optionally substituted with one or more substituents independently selected from hydrogen, halogen, oxo, CN, NO₂, OR¹⁷, SR¹⁷, NR¹⁷R¹⁸, OCOR¹⁷, OCO₂R¹⁷, OCONR¹⁷R¹⁸, COR^{D}, CO₂R¹⁷, CONR¹⁷R¹⁸, SOR¹⁷, SO₂R¹⁷, SO₂NR¹⁷R¹⁸, NR¹⁹CO₂R¹⁷, NR¹⁹COR¹⁷, NR¹⁹C(O)NR¹⁷R¹⁸, NR¹⁹SOR¹⁷, NR¹⁹SO₂R¹⁷, NR¹⁹SO₂NR¹⁷R¹⁸, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R¹⁷, R¹⁸ and R¹⁹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹⁷ and R¹⁸, R¹⁷ and R¹⁹ together with the atom to which they are connected form a 4-20 membered heterocyclyl ring; and
   m and n are independently selected from 0 and 1.

In some embodiments, the FORMULA 5 is FORMULA 5A:

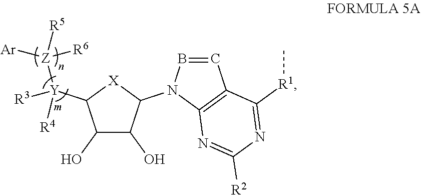

wherein
the definitions of X, Y, Z, B, C, R¹, R², R³, R⁴, R⁵, R⁶ and Ar are the same as FORMULA 5.

In some embodiments, the FORMULA 5 is FORMULA 5B:

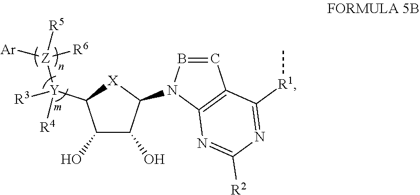

wherein
the definitions of X, Y, Z, B, C, R¹, R², R³, R⁴, R⁵, R⁶ and Ar are the same as FORMULA 5.

In some embodiments, the FORMULA 5 is FORMULAE 5C, 5D, and 5E:

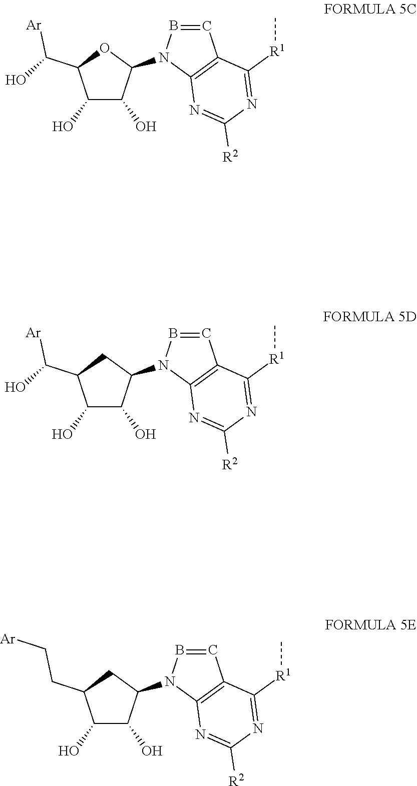

wherein
the definitions of B, C, R¹, R² and Ar are the same as FORMULA 5.

In some embodiments, with respect to FORMULAS 5C-5E,

B is selected from CH and N;
C is selected from CR⁸, CNR¹⁰R¹¹, CNR¹⁰C(O)R¹¹, C NR⁸C(O)NR¹⁰R¹¹, CNR⁸SOR¹⁰, CNR⁸SO₂R¹⁰, and N, wherein
R⁸, R¹⁰, and R¹¹ are independently selected from hydrogen, optionally substituted C₁-C₈ alkyl, optionally substituted C₂-C₈ alkenyl, optionally substituted C₂-C₈ alkynyl, optionally substituted 3-10 membered cycloalkyl, optionally substituted 4-10 membered heterocyclyl;
   R¹ is selected from null, OR¹¹, SR¹¹, NR¹¹R¹², OC(O)R¹¹, OC(O)OR¹¹, OCONR¹¹R¹², C(O)R¹¹, C(O)OR¹¹, CONR¹¹R¹², S(O)R¹¹, S(O)₂R¹¹, SO₂NR¹¹R¹², NR¹³C(O)OR¹¹, NR¹³C(O)R¹¹, NR¹³C(O)NR¹¹R¹², NR¹³S(O)R¹¹, NR¹³S(O)₂R¹¹, NR₁₃S(O)₂NR¹¹R¹², optionally substituted C₁-C₈ alkylene, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl, wherein
R¹¹ is null, or a bivalent moiety selected from optionally substituted C₁-C₈ alkylenyl, optionally substituted C₂-C₈ alkenylene, optionally substituted C₂-C₈ alkynylene, optionally substituted 3-10 membered cycloalkyl, optionally substituted 4-10 membered heterocyclyl, optionally substituted aryl, and optionally substituted heteroaryl;
R¹² and R¹³ are independently selected from optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl; or
R¹¹ and R¹², R¹¹ and R¹³, R¹² and R¹³ together with the atom to which they are connected form a 4-20 membered heterocyclyl ring;
   R² is selected from hydrogen, methyl, and NH₂; and
   Ar is selected from aryl and heteroaryl, each of which is optionally substituted with one or more substituents independently selected from hydrogen, halogen, oxo, CN, NO₂, OR¹⁷, SR¹⁷, NR¹⁷R¹⁸, OCOR¹⁷, OCO₂R¹⁷, OCONR¹⁷R¹⁸, COR¹⁷, CO₂R¹⁷, CONR¹⁷R¹⁸, SOR¹⁷, SO₂R¹⁷, SO₂NR¹⁷R¹⁸, NR¹⁹CO₂R¹⁷, NR¹⁹COR¹⁷, NR¹⁹C(O)NR¹⁷R¹⁸, NR¹⁹SOR¹⁷, NR¹⁹SO₂R¹⁷, NR¹⁹SO₂NR¹⁷R¹⁸, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, wherein
R¹⁷, R¹⁸ and R¹⁹ are independently selected from hydrogen, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹⁷ and R¹⁸, R¹⁷ and R¹⁹ together with the atom to which they are connected form a 4-20 membered heterocyclyl ring.

In some embodiments, the FORMULA 5 is FORMULA 5F:

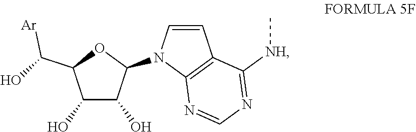

wherein
the definitions of Ar is the same as FORMULA 5.

In addition, the PRMT5 ligand can be a PRMT5 inhibitor, such as, EPZ015666 (Chan-Penebre et al., 2015), GSK591 (Kaniskan et al., 2017), GSK3326595 (EPZ015938) (Kaniskan et al., 2017), BLL-1 (CPD 5) (Alinari et al., 2015), HLCL-61 (Tarighat et al., 2016), LLY-283 (Kaniskan et al., 2017), PF-06855800 (Mcalpine et al., 2018) and/or analogs thereof.

In some aspects, the PRMT5 ligand can be, e.g.,

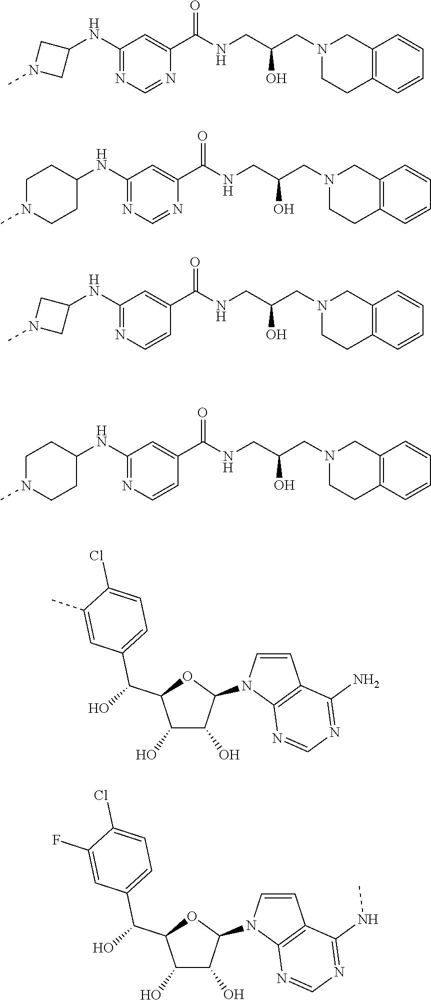

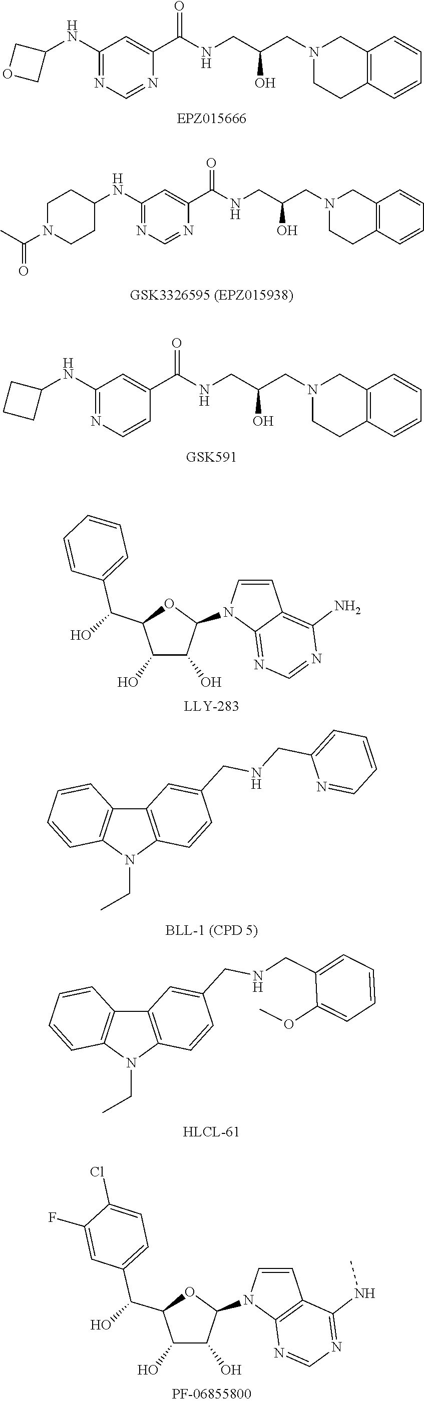

### Degradation/Disruption Tags

In some aspects, the Degradation/Disruption tag (EL) comprises any one of FORMULA 6A-6D:

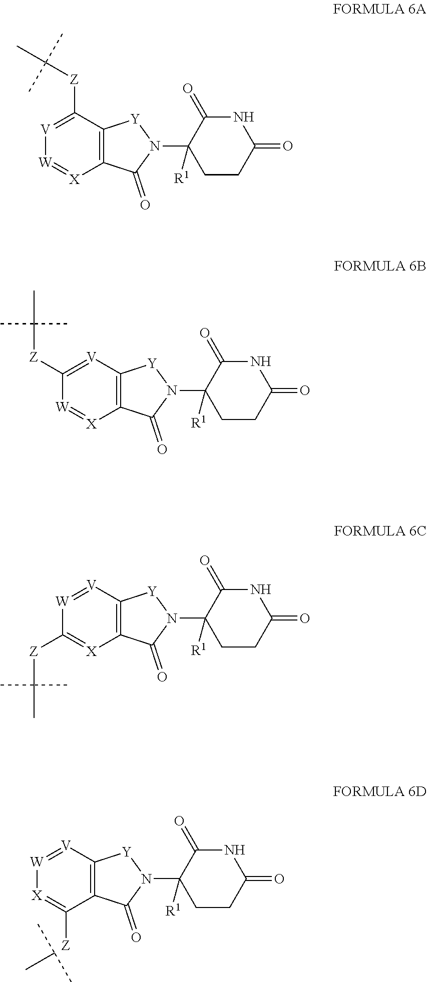

wherein
V, W, and X are independently selected from CR² and N;
Y is selected from CO, CH₂, and N═N;
Z is selected from CH₂, NH, and O;
R¹ is selected from hydrogen, methyl, fluoro, C₁-C₅ alkyl, and halogen; and
R² is hydrogen, halogen, or C₁-C₅ alkyl.

In certain embodiments, with respect to FORMULAS 6A-6D,

V, W, and X are independently selected from CR² and N;
Y is selected from CO and CH₂;
Z is selected from CH₂, NH, and O;
R¹ is selected from hydrogen, methyl, and fluoro; and
R² is hydrogen, halogen, or C₁-C₅ alkyl.

In certain embodiments, with respect to FORMULAS 6A-6D,

V, W, and X are independently selected from CR² or N;
Y is selected from CO, CH₂, N═N;
Z is selected from CH₂, NH, or O;
R¹ is selected from hydrogen, C₁-C₅ alkyl and halogen; and
R² is hydrogen, halogen, or C₁-C₅ alkyl;

In some aspects, the Degradation/Disruption tag (EL) comprises:

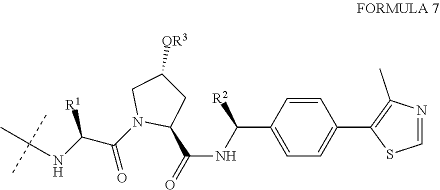

wherein
R¹ and R² are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-8 membered cycloalkyl, optionally substituted 4-8 membered heterocyclyl, optionally substituted C₂-C₈ alkenyl, and optionally substituted C₂-C₈ alkynyl;
R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)C₃-C₈ cycloalkyl, optionally substituted C(O)(4-8 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)OC₃-C₈ cycloalkyl, optionally substituted C(O)O(4-8 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)NC₃-C₈ cycloalkyl, optionally substituted C(O)N(4-8 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In some aspects, the Degradation/Disruption tags (EL) comprises:

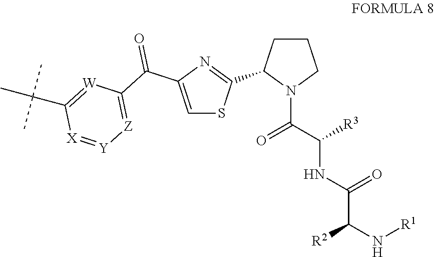

wherein
V, W, X, and Z are independently selected from CR⁴ and N; and
R¹, R², R³, and R⁴ are independently selected from hydrogen, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted 3-8 membered cycloalkyl, optionally substituted 4-8 membered heterocyclyl, optionally substituted C₂-C₈ alkenyl, and optionally substituted C₂-C₈ alkynyl.

In some aspects, the degradation/disruption tag can be, e.g., pomalidomide (Fischer et al., 2014), thalidomide (Fischer et al., 2014), lenalidomide (Fischer et al., 2014), VH032 (Galdeano et al., 2014; Maniaci et al., 2017), adamantine (Xie et al., 2014), 1-((4,4,5,5,5-pentafluoropentyl)sulfinyl)nonane (E. Wakeling, 1995), nutlin-3a (Vassilev et al., 2004), RG7112 (Vu et al., 2013), RG7338, AMG 232 (Sun et al., 2014), AA-115 (Aguilar et al., 2017), bestatin (Hiroyuki Suda et al., 1976), MV1 (Varfolomeev et al., 2007), LCL161 (Weisberg et al., 2010), and/or analogs thereof.

In some aspects, the degradation/disruption tag can be, e.g., one of the following structures:

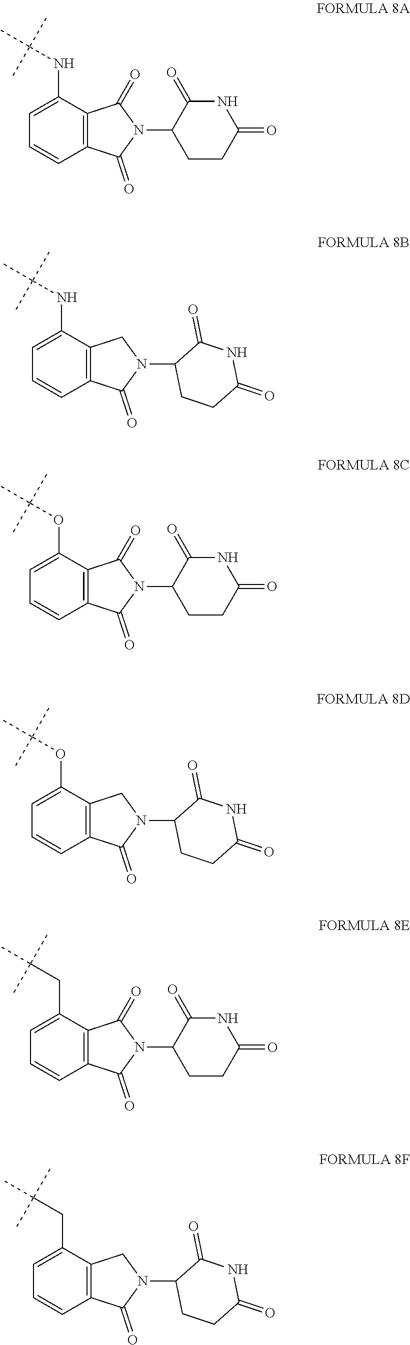

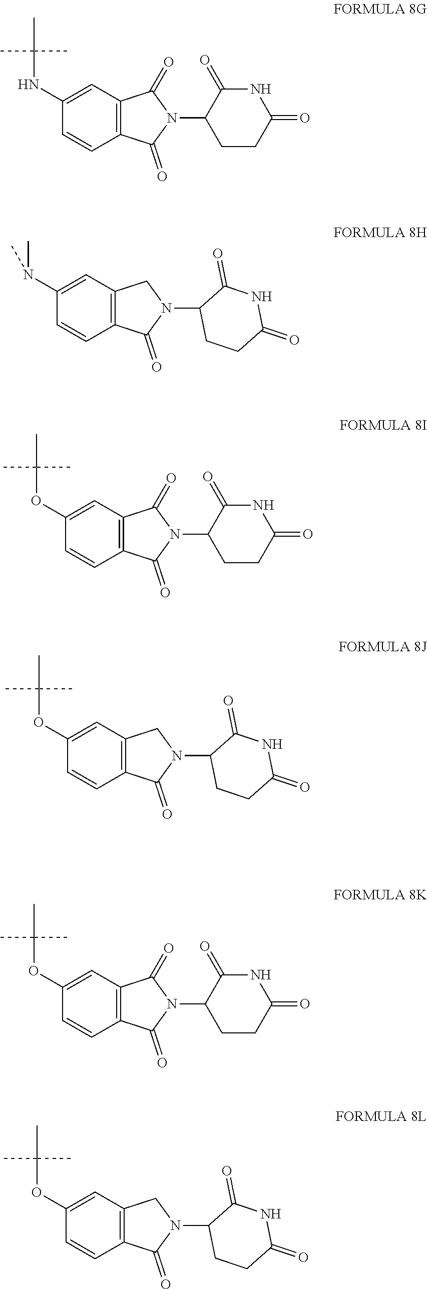

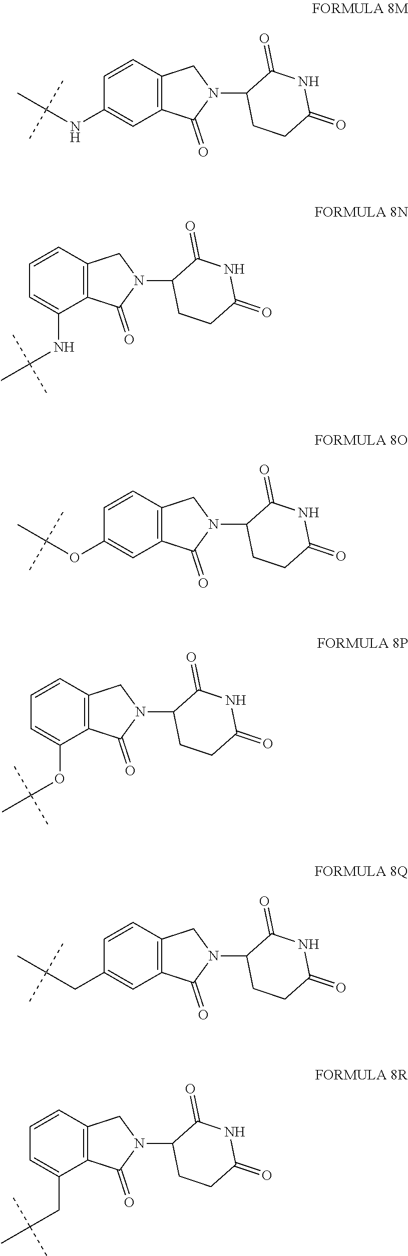

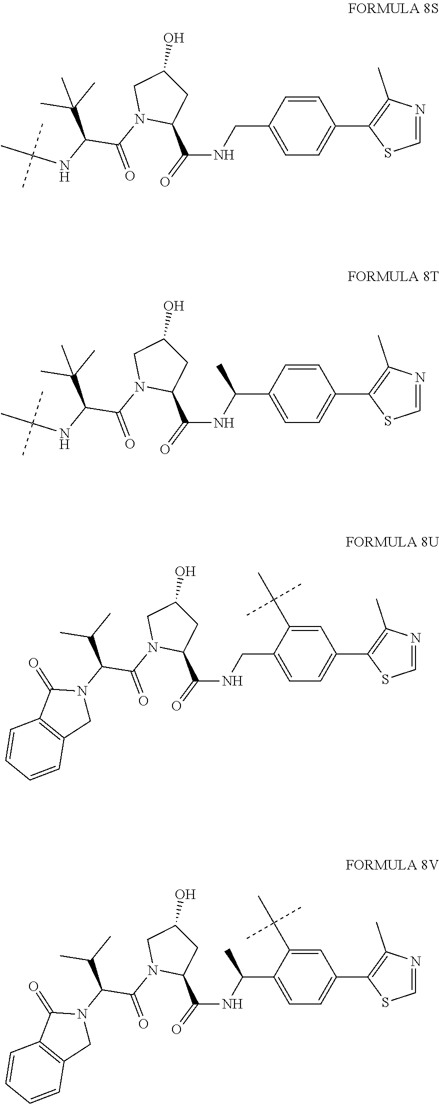

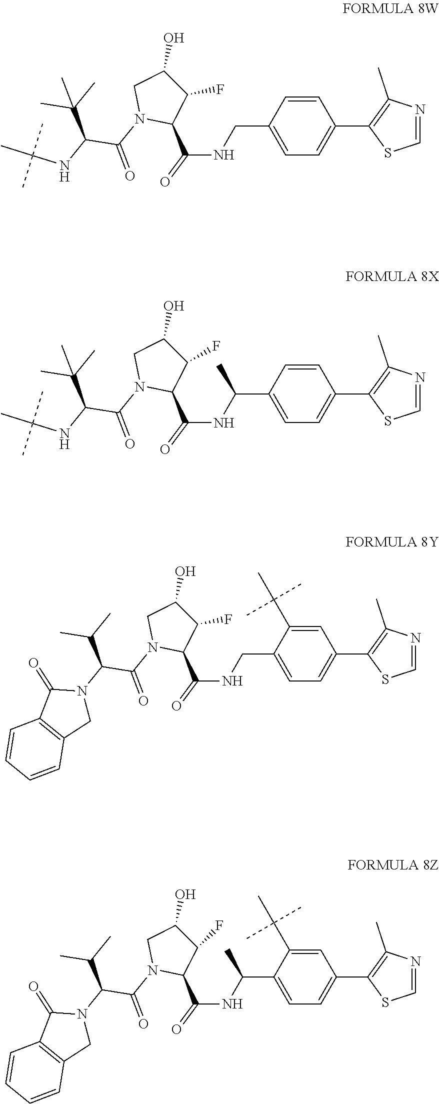

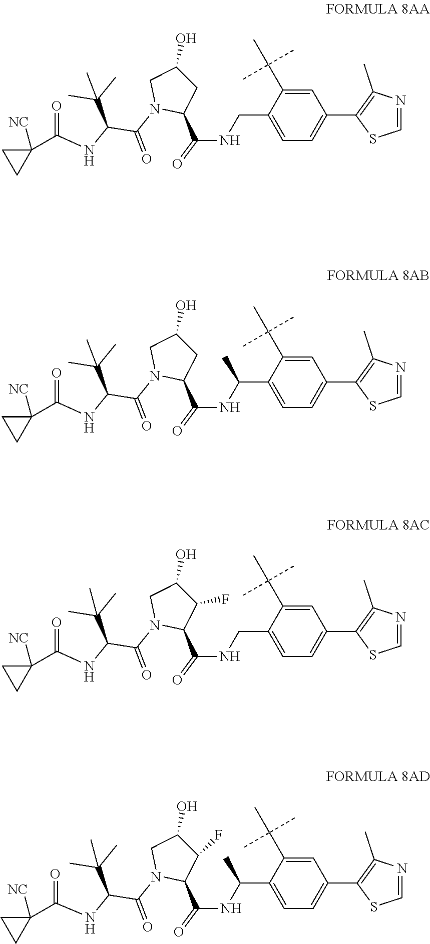

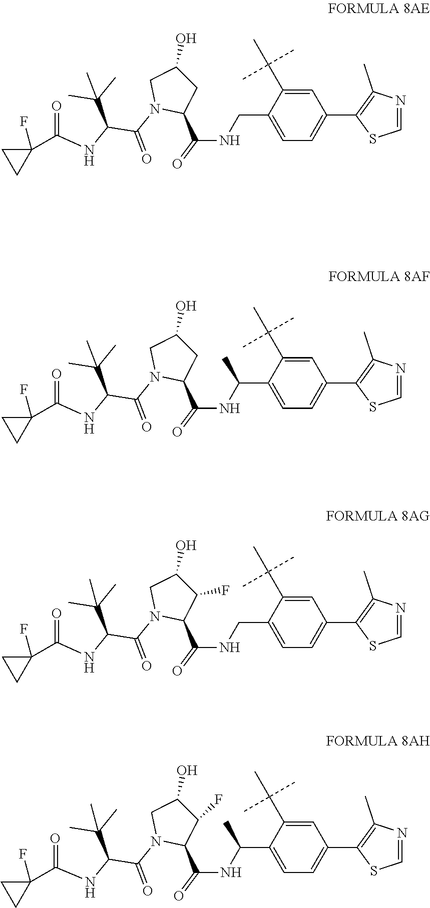

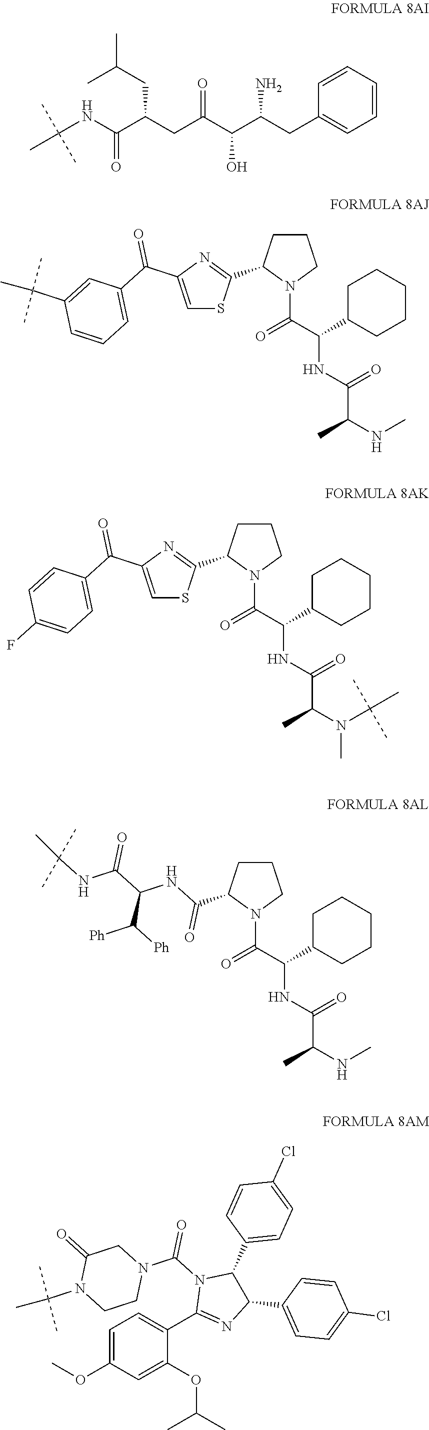

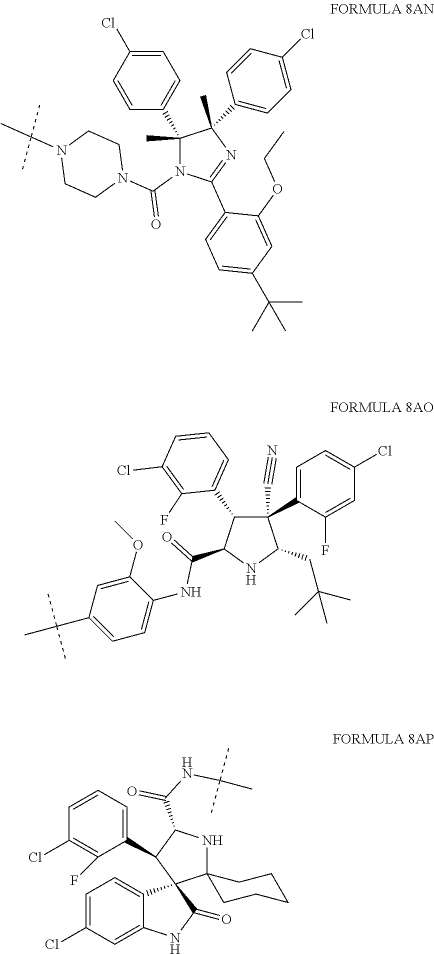

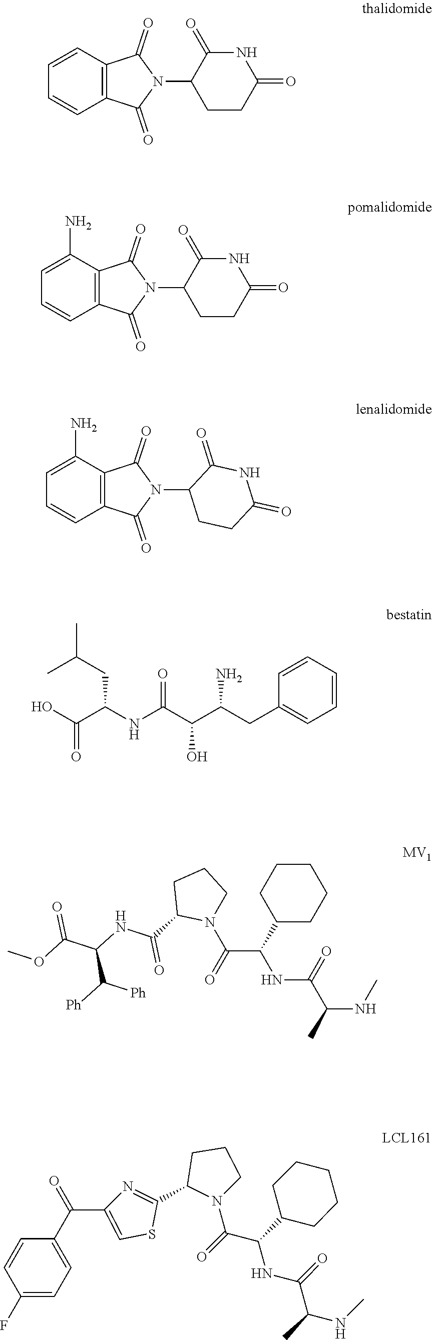

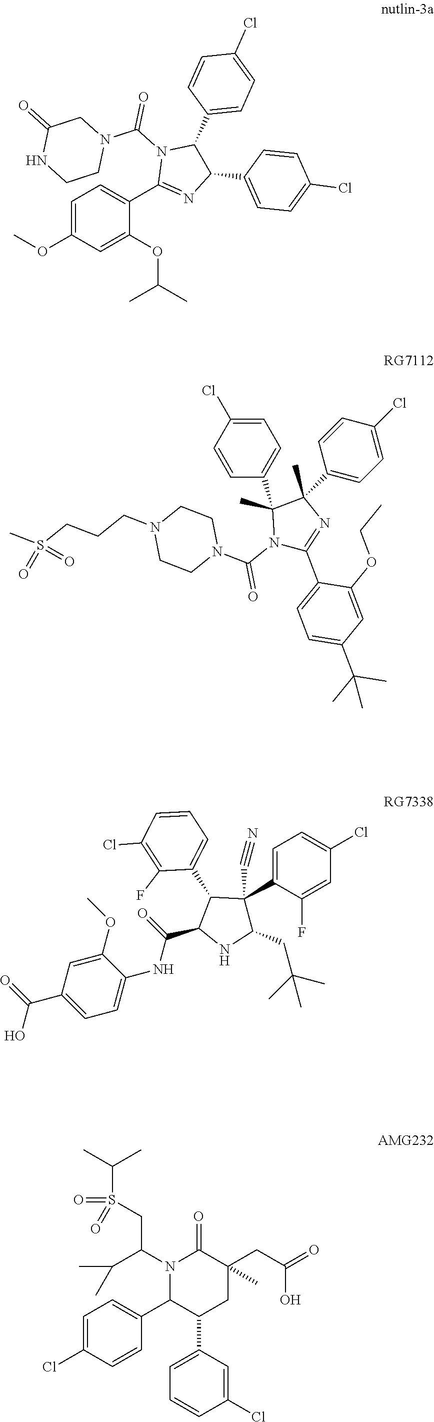

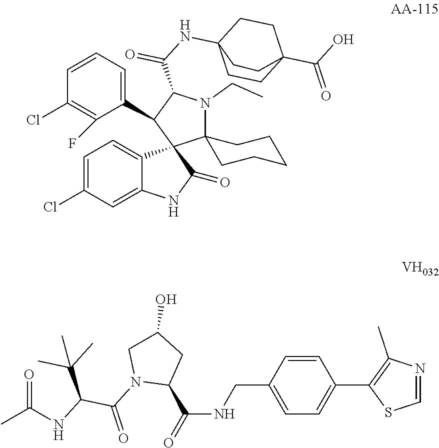 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CC(=O)N[C@H](C(=O)N1C[C@H](O)C[C@H]1C(=O)NCc1ccc(-c2scnc2C)cc1)C(C)(C)C]

In some aspects, the degradation/disruption tag can bind to a ubiquitin ligase (e.g., an E3 ligase such as a cereblon E3 ligase, a VHL E3 ligase, a MDM2 ligase, a TRIM21 ligase, a TRIM24 ligase, and/or an IAP ligase) and/or serve as a hydrophobic group that leads to PRMT5 protein misfolding.

Linkers

In any of the above-described compounds, the PRMT5 ligand can be conjugated to the degradation/disruption tag through a linker. The linker can include, e.g., acyclic or cyclic saturated or unsaturated carbon, ethylene glycol, amide, amino, ether, urea, carbamate, aromatic, heteroaromatic, heterocyclic, and/or carbonyl containing groups with different lengths.

In some aspects, the linker can be a moiety of:

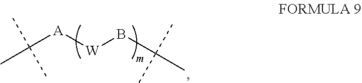

wherein

A, W and B, at each occurrence, are independently selected from null, or bivalent moiety selected from R′—R″, R′COR″, R′CO₂R″, R′C(O)NR″R′, R′C(S)NR″R¹, R′OR″, R′SR″, R′SOR″, R′SO₂R″, R′SO₂NR″R¹, R′NR″R¹, R′NR¹COR″, R′NR¹CONR″R², R′NR¹C(S)R″, R′OCH₂C(O)NR″R¹, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl, or
R′ and R″ together with the atom to which they are connected form a 3-20 membered cycloalkyl or 4-20 membered heterocyclyl ring;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl, or
R¹ and R² together with the atom to which they are connected form a 3-20 membered cycloalkyl or 4-20 membered heterocyclyl ring;
R′ and R¹, R′ and R², R″ and R¹, R″ and R² together with the atom to which they are connected form a 3-20 membered cycloalkyl or 4-20 membered heterocyclyl ring; and
   m is 0 to 15.

In some embodiments, with respect for FORMULA 9, A is R′OCH₂C(O)NR″R¹; W is null or optionally substituted C₁-C₈ alkylene; B is null or optionally substituted C₁-C₈ alkylene; R′ is null; R″ is null or optionally substituted C₁-C₈ alkylene; R¹ is hydrogen; m is 0 to 6.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is C₂₋₆ alkylene.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is —(CH₂)₂—.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is —(CH₂)₄—.

In some embodiments, with respect to FORMULA 9, A is R′OCH₂C(O)NR″R¹; W is null or optionally substituted C₁-C₈ alkylene; B is null or optionally substituted C₁-C₈ alkylene; R′ is null; R″ is null; R¹ is hydrogen; m is 0 to 6; wherein (W-B)ₘ is —(CH₂)₆—.

In some aspects, the linker can be a moiety of:

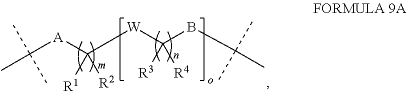

wherein

R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered cycloalkyl, optionally substituted 3-10 membered cycloalkoxy, optionally substituted 3-10 membered cycloalkylamino, optionally substituted 4-10 membered heterocyclyl, optionally substituted aryl, and optionally substituted heteroaryl, or

R¹ and R², R³ and R⁴ together with the atom to which they are connected form a 3-20 membered cycloalkyl or 4-20 membered heterocyclyl ring;

A, W and B, at each occurrence, are independently selected from null, or bivalent moiety selected from R′—R″, R′COR″, R′CO₂R″, R′C(O)NR″R⁵, R′C(S)NR″R⁵, R′OR″, R′SR″, R′SOR″, R′SO₂R″, R′SO₂NR″R⁵, R′NR″R⁵, R′NR⁵COR″, R′NR⁵CONR″R⁶, R′NR⁵C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl;
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;
   m is 0 to 15;
   n, at each occurrence, is 0 to 15; and
   o is 0 to 15.

In some aspects, the linker can be a moiety of:

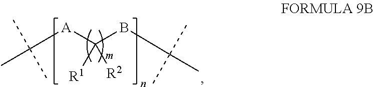

wherein

R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered cycloalkyl, optionally substituted 3-10 membered cycloalkoxy, optionally substituted 3-10 membered cycloalkylamino, optionally substituted 4-10 membered heterocyclyl, optionally substituted aryl, and optionally substituted heteroaryl, or
R¹ and R² together with the atom to which they are connected form a 3-20 membered cycloalkyl or 4-20 membered heterocyclyl ring;

A and B, at each occurrence, are independently selected from null, or bivalent moiety selected from R′—R″, R′COR″, R′CO₂R″, R′C(O)NR″R³, R′C(S)NR″R³, R′OR″, R′SR″, R′SOR″, R′SO₂R″, R′SO₂NR″R³, R′NR″R³, R′NR³COR″, R′NR³CONR″R⁴, R′NR³C(S)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl;
R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;
   each m is 0 to 15; and
   n is 0 to 15.

In some aspects, the linker can be a moiety of:

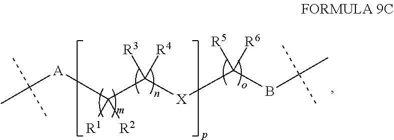

wherein
X is selected from 0, NH, and NR⁷;

R¹, R², R³, R⁴, R⁵, R⁶, and R⁷, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered cycloalkyl, optionally substituted 3-10 membered cycloalkoxy, optionally substituted 4-10 membered heterocyclyl, optionally substituted aryl, and optionally substituted heteroaryl;

A and B are independently selected from null, or bivalent moiety selected from R′—R″, R′COR″, R′CO₂R″, R′C(O)NR″R⁸, R′C(S)NR″R⁸, R′OR″, R′SR″, R′SOR″, R′SO₂R″, R′SO₂NR″R⁸, R′NR″R⁸, R′NR⁸COR″, R′NR⁸CONR″R⁹, R′NR⁸C(S)R″, R′OCH₂C(O)NR″R¹, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered cycloalkyl, optionally substituted 4-10 membered heterocyclyl, optionally substituted aryl, and optionally substituted heteroaryl, wherein
R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered cycloalkyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered cycloalkyl, optionally substituted 4-10 membered heterocyclyl, optionally substituted aryl, and optionally substituted heteroaryl;
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;
   m, at each occurrence, is 0 to 15;
   n, at each occurrence, is 0 to 15;
   o is 0 to 15; and
   p is 0 to 15.

In some embodiments, with respect to FORMULA 9C, A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH—CH₂—NH—CH₂, CH₂—NH—CH₂,

In some embodiments, with respect to FORMULA 9C, o is 0 to 5. In another refinement, the linker moiety comprises a ring selected from the group consisting of a 3 to 13 membered ring, a 3 to 13 membered fused ring, a 3 to 13 membered bridged ring, and a 3 to 13 membered spiro ring.

In some embodiments, with respect to FORMULA 9C, X is O; R¹, R², R³, R⁴, R⁵, R⁶, and R⁷, at each occurrence, hydrogen; A is R′OCH₂C(O)NR″R¹ (R′=R″=null; R¹=H); B is R′C(O)R″ (R′=R″=null) m=0-2; n=0-2; o=0-10; and p=0-1.

In some embodiments, with respect to FORMULA 9C, X is O; R¹, R², R³, R⁴, R⁵, R⁶, and R⁷, at each occurrence, hydrogen; A is R′OCH₂C(O)NR″R¹ (R′=R″=null; R¹=H); B is R′ C(O)R″ (R′=R″=null); m=0-2 and n=0-2; wherein m+n=2; o=1; and p=1.

In some embodiments, with respect to FORMULA 9C, R¹, R², R³, R⁴, R⁵, R⁶, and R⁷, at each occurrence, hydrogen; A is R′OCH₂C(O)NR″R¹ (R′=R″=null; R¹=H); B is R′C(O)R″ (R′=R″=null); o=2-12; and p=0.

In some embodiments, with respect to FORMULA 9C, R¹, R², R³, R⁴, R⁵, R⁶, and R⁷, at each occurrence, hydrogen; A is R′OCH₂C(O)NR″R¹ (R′=R″=null; R¹=H); B is R′ C(O)R″ (R′=R″=null); o=4; and p=0.

In some embodiments, with respect to FORMULA 9C, R¹, R², R³, R⁴, R⁵, R⁶, and R⁷, at each occurrence, hydrogen; A is R′OCH₂C(O)NR″R¹ (R′=R″=null; R¹=H); B is R′C(O)R″ (R′=R″=null); o=10; and p=0.

In some aspects, the linker moiety comprises a ring selected from the group consisting of formulae C1, C2, C3, C4 and C5:

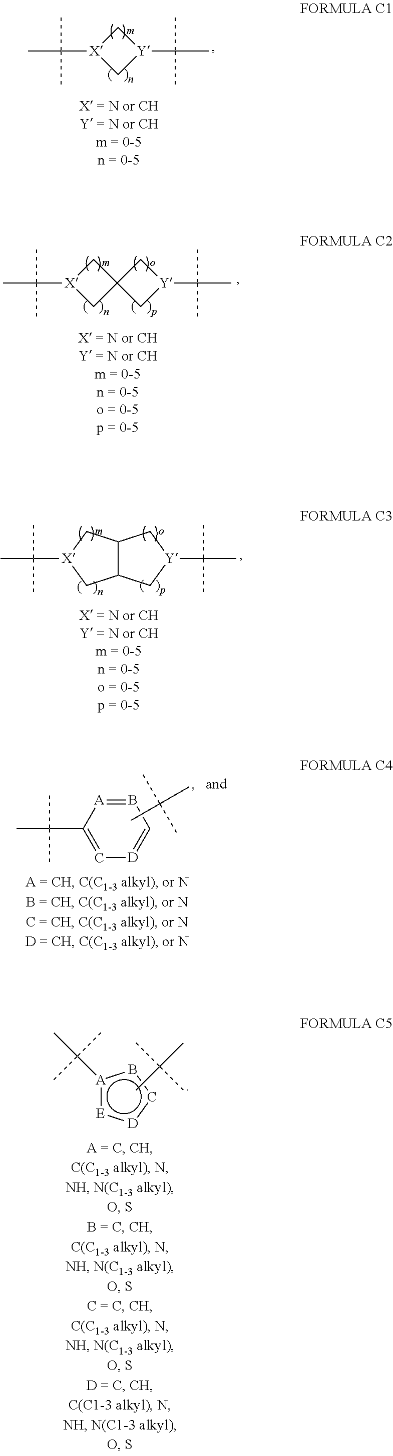

In some aspects, the linker can be a moiety of:

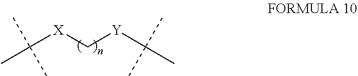

wherein X is C═O or CH₂,

### Y is C═O or CH₂, and

n is 0-15;

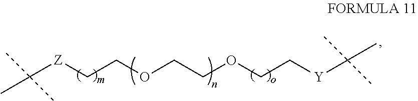

wherein X is C═O or CH₂,

### Y is C═O or CH₂,

m is 0-15,
n is 0-6, and
o is 0-15; or

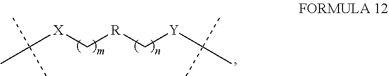

wherein

### X is C═O or CH₂,

### Y is C═O or CH₂,

R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl)-, —C═C—, —O—, —NH—, —N(C₁₋₃ alkyl)-, —C(O)NH—C(O)N(C₁₋₃ alkyl)-, a 3-13 membered ring, a 3-13 membered fused ring, a 3-13 membered bridged ring, and/or a 3-13 membered spiro ring,
m is 0-15, and
n is 0-15.

In some aspects of FORMULA 11, X is C═O, Y is C═O, m is 0-4, n is 2-6, and o is 0-4.

In some aspects of FORMULA 11, X is C═O, Y is C═O, m is 0-1, n is 4, and o is 0-1.

In some aspects of FORMULA 11, X is C═O, Y is C═O, m is 0, n is 4, and o is 0.

In some aspects of FORMULA 11, X is C═O, Y is C═O, m is 1, n is 4, and o is 1.

In some aspects of FORMULA 12, X is C═O or CH₂, Y is C═O or CH₂, R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl)-, —C═C—, m is 0-4, and n is 0-4.

In some aspects of FORMULA 12, X is C═O, Y is CH₂, R is —CH₂—, m is 0-4, n is 0-4, and m+n=4.

In some aspects of FORMULA 12, R is a 3-13 membered ring, a 3-13 membered fused ring, a 3-13 membered bridged ring, and/or a 3-13 membered spiro ring, one or more of which can contain one or more heteroatoms.

In some aspects of FORMULA 12, R has a structure of

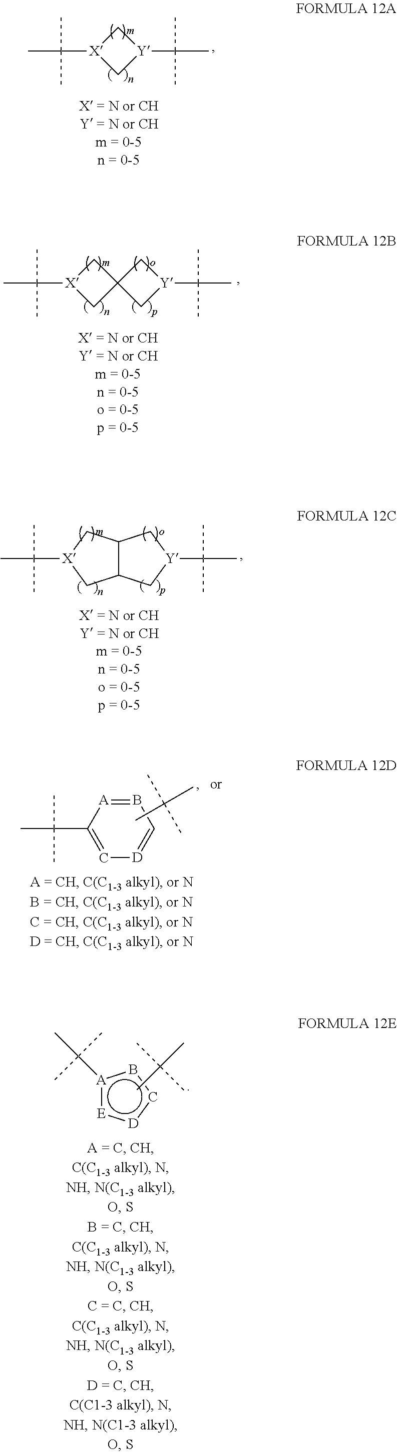

In some aspects, the bivalent compound is a compound selected from those synthesized in the Examples below, including, but not limited to: YS31-58, YS31-59, YS31-60, YS31-61, YS31-62, YS31-63, YS31-64, YS31-65, YS31-66, YS31-67, YS31-68, YS31-69, YS43-6, YS43-7, YS43-8, YS43-9, YS43-10, YS43-11, YS43-12, YS43-13, YS43-14, YS43-15, YS43-16, YS43-17, YS43-18, YS43-19, YS43-20, YS43-21, YS43-22, YS43-25, YS43-26, YS43-27, YS43-28, YS43-29, YS43-30, YS43-31, YS43-32, YS43-33, YS43-34, YS43-35, YS43-36, YS43-37, YS43-38, YS43-39, YS43-40, YS43-41, YS43-42, YS43-43, YS43-44, YS43-45, YS43-46, YS43-47, YS43-48, YS43-49, YS43-50, YS43-51, YS43-52, YS43-53, YS43-54, YS43-88, YS43-89, YS43-90, YS43-91, YS43-92, YS43-93, YS43-94, YS43-95, YS43-96, YS43-97, YS43-98, YS43-99, YS43-100, YS43-101, YS43-102, YS43-103, YS43-104, YS43-105, YS43-106, YS43-107, YS43-108, YS43-109, YS43-110, YS43-111, YS43-112, YS43-113, YS43-114, YS43-115, YS43-116, YS43-117, CPD-90 to CPD-118, or analogs thereof. In some embodiments, the bivalent compound is selected from the group consisting of YS43-93, YS43-95, YS43-97, YS43-100, YS43-111, YS31-60, YS43-8, YS43-16, and YS43-22. In some embodiments, the bivalent compound is selected from the group consisting of YS31-60, YS43-8, YS43-16, and YS43-22. In some embodiments, the bivalent compound is selected from the group consisting of YS43-93, YS43-95, YS43-97, YS43-100, YS43-111 and YS43-117.

### Synthesis and Testing of Bivalent Compounds

The binding affinity of novel synthesized bivalent compounds (i.e., PRMT5 degraders/disruptors) can be assessed using standard biophysical assays known in the art (e.g., isothermal titration calorimetry (ITC)). Cellular assays can then be used to assess the bivalent compound's ability to induce PRMT5 degradation and inhibit cancer cell proliferation. Besides evaluating bivalent compound's-induced changes in the protein expression of PRMT5, enzymatic activity can also be assessed. Assays suitable for use in any or all of these steps are known in the art, and include, e.g., Western blotting, quantitative mass spectrometry (MS) analysis, flow cytometry, enzymatic inhibition, ITC, SPR, cell growth inhibition and xenograft and PDX models. Suitable cell lines for use in any or all of these steps are known in the art and include, e.g., AML cells: MV4-11 (FLT3-ITD) and THP-1 (FLT3-WT) cell lines and patient blasts (FLT3-ITD or FLT3-WT); MCF-7 breast cancer cells, A375 melanoma cells, A549 lung carcinoma cells, Hela cervical cancer cells, Jurkat acute T cell leukemia cells, HCT116 colorectal carcinoma cells, 293T human embryonic kidney cells, H2171 small cell lung carcinoma cells, and NCI-H1048 lung cancer cells.

By way of non-limiting example, detailed synthesis protocols are described in the Examples for specific exemplary PRMT5 degraders/disruptors.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

### Characterization of Exemplary PRMT5 Degraders/Disruptors

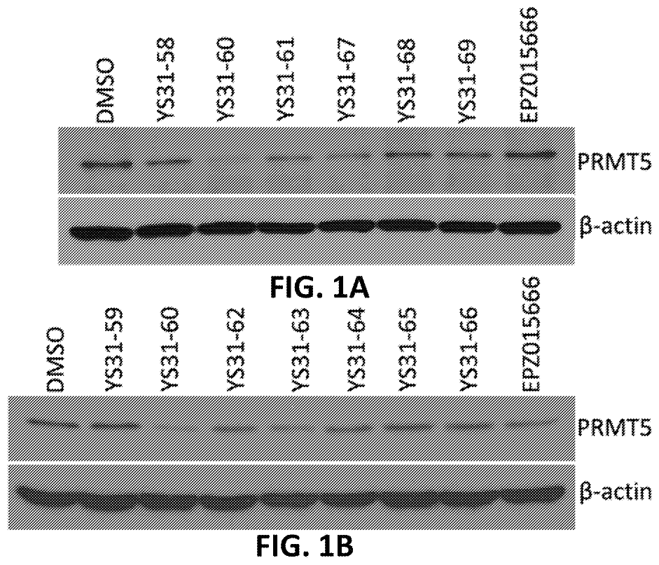

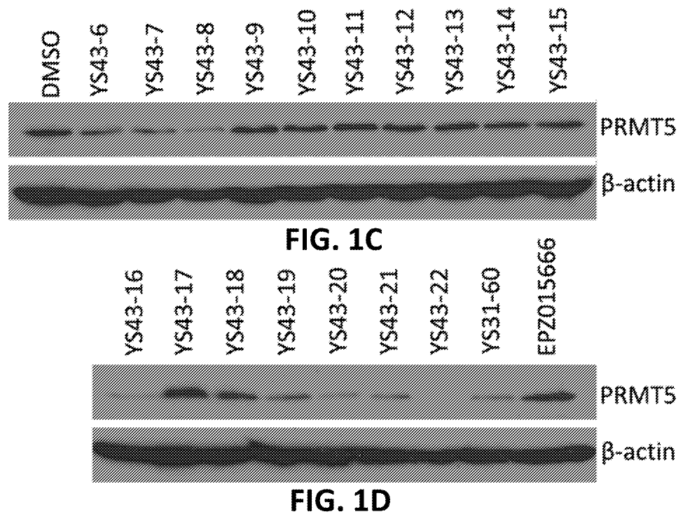

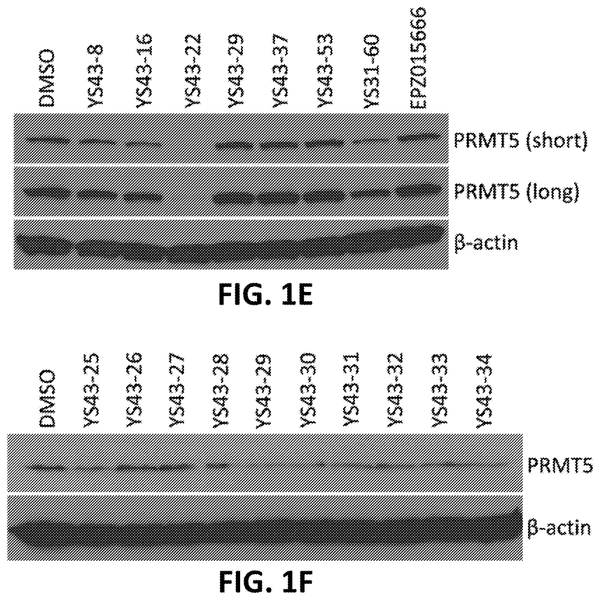

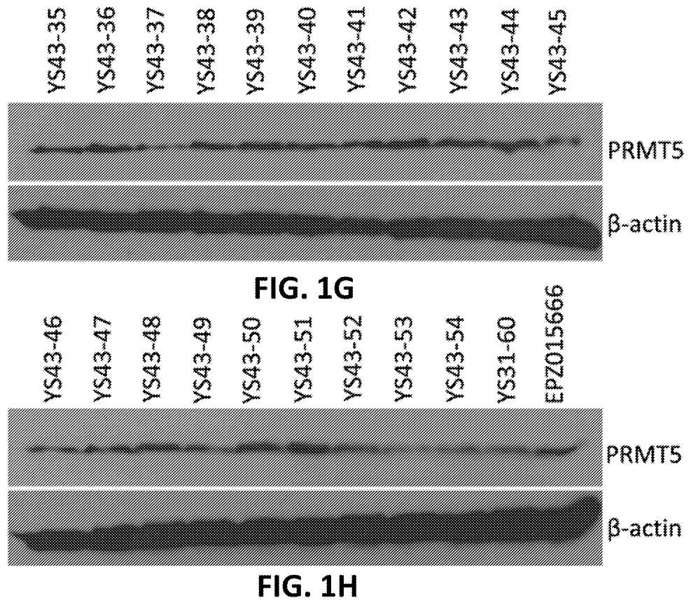

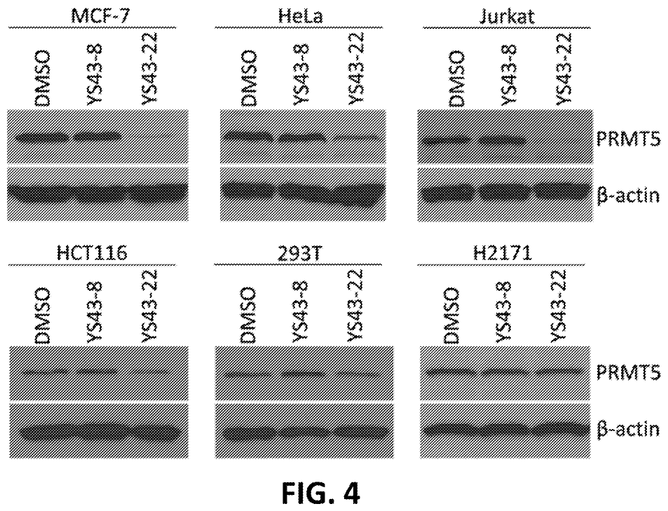

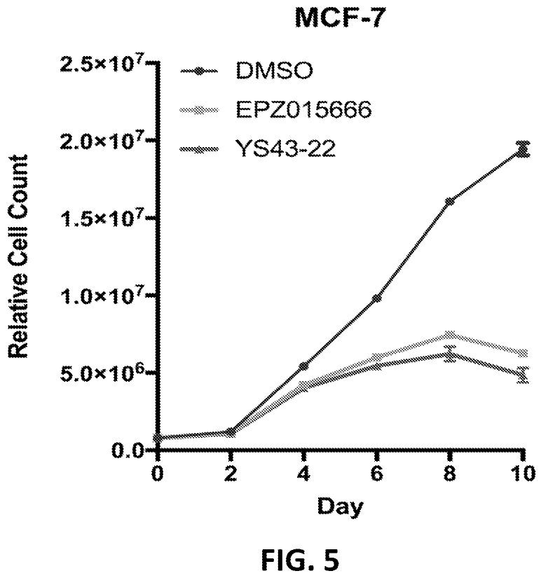

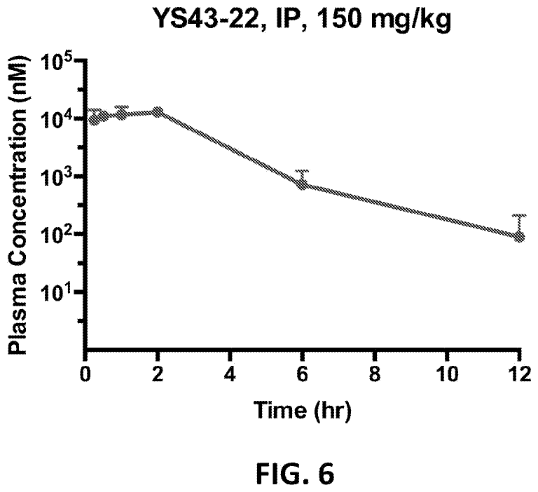

Specific exemplary PRMT5 degraders/disruptors were characterized using MCF-7 cells (Examples 90-92, FIGS. 1-3). YS31-60, YS43-8, YS43-16, and YS43-22 in particular were found to be especially effective in suppressing both PRMT5 expression and PRMT5 activity. This efficacy in suppressing PRMT5 expression and PRMT5 activity correlated with efficacy in inhibiting cancer cell proliferation (Example 94, FIG. 5). In particular, the effect of YS43-22 on reducing PRMT5 protein levels and inhibiting the PRMT5 catalytic activity correlated with efficacy in lowering cancer cell proliferation (FIG. 5). Similar to EPZ015666, YS43-22 showed significant potency on the inhibition of MCF-7 cell growth (Example 94, FIG. 5). YS43-8 and YS43-22 were further tested in five additional cell lines, Hela, Jurkat, HCT116, 293T, and H2171 cells (Example 93, FIG. 4). YS43-22 reduced PRMT5 protein levels very well in MCF-7 cells and Jurkat cells; moderately in HeLa cells; slightly in HCT116 and 293T cells; not obviously in H2171 cells. In mice, YS43-22 is bioavailable at 150 mg/kg via IP administration (Example 95, FIG. 6).

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents known in the art. When co-administered, PRMT5 degraders/disruptors disclosed herein can operate in conjunction with conventional chemotherapeutic agents to produce mechanistically additive or synergistic therapeutic effects.

In some aspects, the pH of the compositions disclosed herein can be adjusted with pharmaceutically acceptable acids, bases, or buffers to enhance the stability of the PRMT5 degraders/disruptor or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the PRMT5 degraders/disruptors disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5th Edition, Vol. 1: Principles and Practice, which is incorporated herein by reference to the extent of teaching such derivatives.

The PRMT5 degraders/disruptors disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the PRMT5 degraders/disruptors disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., PRMT5 alkali metal (e.g., sodium), PRMT5 alkaline earth metal (e.g., magnesium), ammonium and N-(PRMT5yl)4+ salts. The invention also envisions the quaternization of any basic nitrogen-containing groups of the PRMT5 degraders/disruptors disclosed herein. Water or oil-soluble or dispersible products can be obtained by such quaternization.

In some aspects, the pharmaceutical compositions disclosed herein can include an effective amount of one or more PRMT5 degraders/disruptors. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein (e.g., a PRMT5 degraders/disruptors) and one or more additional compounds (e.g., one or more additional compounds, drugs, or agents used for the treatment of cancer or any other condition or disease, including conditions or diseases known to be associated with or caused by cancer), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising a PRMT5 degrader/disruptor, including pharmaceutical compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering (e.g., a PRMT5-mediated cancer). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some aspects, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

As used herein, the term “treating cancer” means causing a partial or complete decrease in the rate of growth of a tumor, and/or in the size of the tumor and/or in the rate of local or distant tumor metastasis, and/or the overall tumor burden in a subject, and/or any decrease in tumor survival, in the presence of a degrader/disruptor (e.g., a PRMT5 degrader/disruptor) described herein.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. Exemplary PRMT5-mediated cancers that can be treated with PRMT5 degraders/disruptors include, for example, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL), follicular lymphoma, chronic lymphocytic leukemia/small lymphocytic lymphoma (CLL/SLL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (e.g., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MPH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

As used herein, the term “preventing a disease” (e.g., preventing cancer) in a subject means for example, to stop the development of one or more symptoms of a disease in a subject before they occur or are detectable, e.g., by the patient or the patient's doctor. Preferably, the disease (e.g., cancer) does not develop at all, i.e., no symptoms of the disease are detectable. However, it can also mean delaying or slowing of the development of one or more symptoms of the disease. Alternatively, or in addition, it can mean decreasing the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

### EXAMPLES

The following Examples describe the synthesis of exemplary PRMT5 degrader/disrupter compounds according to the present invention.

### General Chemistry Methods

For the synthesis of intermediates and examples (1-89) below, HPLC spectra for all compounds were acquired using an Agilent 1200 Series system with DAD detector. Chromatography was performed on a 2.1×150 mm Zorbax 300SB-C 18 5 μm column with water containing 0.1% formic acid as solvent A and acetonitrile containing 0.1% formic acid as solvent B at a flow rate of 0.4 ml/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Nuclear Magnetic Resonance (NMR) spectra were acquired on a Bruker DRX-600 spectrometer with 600 MHz for proton OH NMR) and 150 MHz for carbon (¹³C NMR); chemical shifts are reported in (8). Preparative HPLC was performed on Agilent Prep 1200 series with UV detector set to 254 nm. Samples were injected onto a Phenomenex Luna 250×30 mm, 5 μm, C18 column at room temperature. The flow rate was 40 ml/min. A linear gradient was used with 10% (or 50%) of MeOH (A) in H₂O (with 0.1% TFA) (B) to 100% of MeOH (A). HPLC was used to establish the purity of target compounds. All final compounds had >95% purity using the HPLC methods described above.

### Synthesis of Intermediates

### 1. Synthesis of Intermediates 1 and 31.

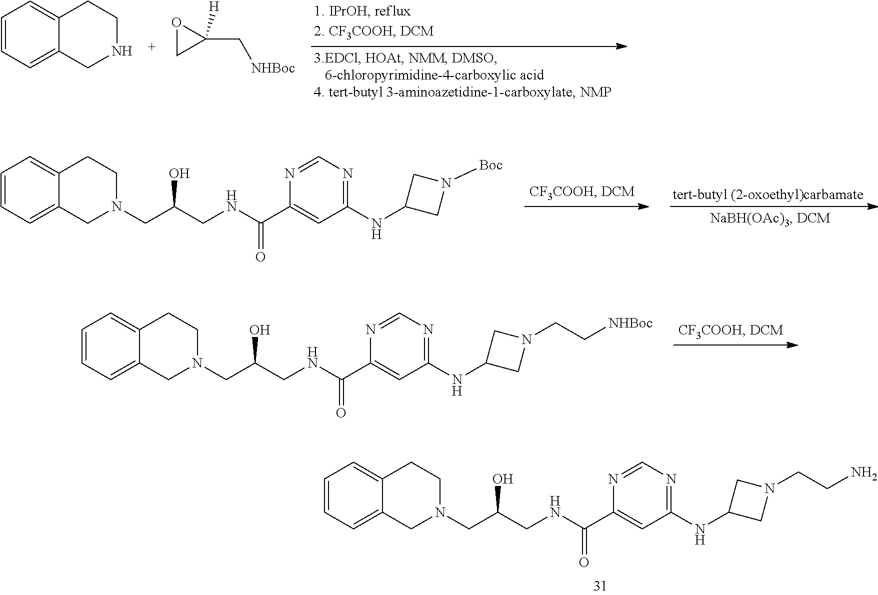 [SMILES: c1ccc2c(c1)CCNC2 ; CC(C)(C)OC(=O)NC[C@H]1CO1 ; CC(C)(C)OC(=O)N1CC(Nc2cc(C(=O)NC[C@H](O)CN3CCc4ccccc4C3)ncn2)C1 ; CC(C)(C)OC(=O)NCCN1CC(Nc2cc(C(=O)NC[C@H](O)CN3CCc4ccccc4C3)ncn2)C1 ; NCCN1CC(Nc2cc(C(=O)NC[C@H](O)CN3CCc4ccccc4C3)ncn2)C1]

To the solution of tert-butyl (oxiran-2-ylmethyl)carbamate (1 g, 5.77 mmol) in 10 mL of isopropanol, was added 1,2,3,4-tetrahydroisoquinoline (770 mg, 5.77 mmol). The solution was heated to reflux for 6 h and the volatile was removed under reduced pressure. The resulting residue was treated for 1 h with 5 mL of trifluoroacetic acid in 5 mL of dichloromethane. The solution was evaporated into dryness under reduced pressure. The resulting brown oil was used for next step without purification. The residue was added to the solution of 6-chloropyrimidine-4-carboxylic acid (951 mg, 6 mmol), EDCI (1-ethyl-3-(3-dimethylaminopropyl)carbodiimide) (1.66 g, 8.67 mmol), HOAt (1-hydroxy-7-azabenzo-triazole) (1.9 g, 14.45 mmol) and NMM (N-Methylmorpholine) (1.12 g, 11.12 mmol) in 30 mL of DMSO and the solution was stirred for 6 h. Then 100 mL of water was added and the mixture was extracted with ethyl acetate (3×100 mL). The organic phase was washed with another 100 mL of water, 50 mL of brine successively, dried over anhydrous sodium sulfate and evaporated into dryness under reduced pressure. The resulting residue was dissolved in 10 mL of NMP (N-Methyl-2-Pyrrolidone), and tert-butyl 3-aminoazetidine-1-carboxylate (1 g, 5.81 mmol) was added. The resulting mixture was stirred overnight and followed by addition of 50 mL of water. The mixture was extracted with ethyl acetate (3×50 mL) and concentrated under reduced pressure. The residue was purified by flash chromatography on silica gel with eluent (Methanol/DCM, 0-10%) to afford tert-butyl (S)-3-((6-((3-(3,4-dihydroisoquinolin-2(1H)-yl)-2-hydroxypropyl)carbamoyl)pyrimidin-4-yl)amino)azetidine-1-carboxylate (445 mg, yield 16% over 4 steps). ¹H NMR (600 MHz, Methanol-d₄) δ 8.25 (s, 1H), 7.14-7.02 (m, 4H), 6.99 (d, J=7.3 Hz, 1H), 4.71-4.62 (m, 1H), 4.27 (s, 2H), 4.06 (q, J=6.0 Hz, 1H), 3.81 (dd, J=9.2, 5.2 Hz, 2H), 3.71 (s, 2H), 3.51 (qd, J=13.6, 5.9 Hz, 2H), 2.92 (t, J=5.9 Hz, 2H), 2.83 (dq, J=11.4, 5.5 Hz, 2H), 2.65 (d, J=6.1 Hz, 2H), 1.44 (s, 9H). MS (ESI) m/z 483.2 [M+H]⁺.

tert-butyl (5)-3-((6-((3-(3,4-dihydroisoquinolin-2(1H)-yl)-2-hydroxypropyl)carbamoyl)pyrimidin-4-yl)amino)azetidine-1-carboxylate was treated with 5 mL of trifluoroacetic acid and 5 mL of dichloromethane to give intermediate 1 in TFA salt form (560 mg, yield 100%). MS (ESI) m/z 383.2 [M+H]⁺.

To a solution of intermediate 1 (180 mg, 0.3 mmol) and tert-butyl (2-oxoethyl)carbamate (72 mg, 0.45 mmol) in 15 mL of dichloromethane, was added NaBH(OAc)₃ (126 mg, 0.6 mmol). The resulting mixture was stirred overnight, followed by adding aqueous sodium bicarbonate to quench the reaction. The mixture was extracted with dichloromethane (3×10 mL). The organic phase was dried over anhydrous sodium sulfate and concentrated under reduced pressure. The residue was purified by flash chromatography on silica gel with eluent (Methanol/DCM, 0-10%) to afford tert-butyl (S)-(2-(3-((6-((3-(3,4-dihydroisoquinolin-2(1H)-yl)-2-hydroxypropyl)carbamoyl)pyrimidin-4-yl)amino)azetidin-1-yl)ethyl)carbamate (81 mg, yield 52%). MS (ESI) m/z 526.3 [M+H]⁺.

tert-butyl (5)-(2-(3-((6-((3-(3,4-dihydroisoquinolin-2(1H)-yl)-2-hydroxypropyl)carbamoyl)pyrimidin-4-yl)amino)azetidin-1-yl)ethyl)carbamate was treated with 3 mL of trifluoroacedtic acid in 3 mL of dichloromethane for 0.5 h to give intermediate 31 in TFA salt form after removal of the volatile (126 mg, 100%). MS (ESI) m/z 426.2 [M+H]⁺.

### 2. Synthesis of VHL-1 Alkyl Linkers

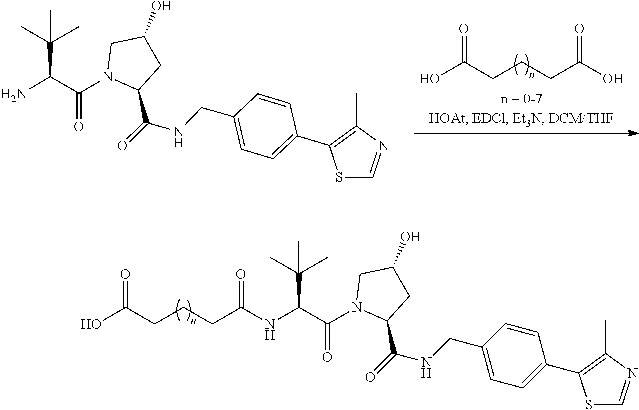

To a solution of diacid (10 mmol) in DCM/THF (1:1, 200 ml) was added VHL-1 (2 mmol), triethylamine (1 ml, 7.1 mmol), HOAt (300 mg, 2.2 mmol), and EDCI (420 mg, 2.2 mmol) sequentially at 0° C. The resulting solution was stirred for 2 h at 0° C., before being warmed to room temperature (RT). After stirring overnight at RT, the reaction was quenched with water. After concentration under reduced pressure, the resulting residue was purified by reverse-phase chromatography to yield the desired product.

### Synthesis of Intermediate 25.

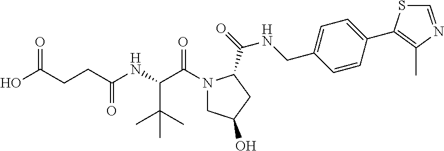 [SMILES: Cc1ncsc1-c1ccc(CNC(=O)[C@@H]2C[C@@H](O)CN2C(=O)[C@@H](NC(=O)CCC(=O)O)C(C)(C)C)cc1]

4-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-4-oxobutanoic acid (810 mg, 85%) as white solid. ¹H NMR (600 MHz CD₃OD) δ 9.10 (s, 1H), 7.51 (d, J=7.8 Hz, 2H), 7.44 (d, J=8.4 Hz, 2H), 4.64 (s, 1H), 4.60-4.49 (m, 3H), 4.39 (d, J=15.6 Hz, 1H), 3.91 (d, J=10.8 Hz, 1H), 3.82 (dd, J=9.6, 3.6 Hz, 1H), 2.67-2.55 (m, 4H), 2.52 (s, 3H), 2.25-2.22 (m, 1H), 2.12-2.07 (m, 1H), 1.06 (s, 9H). HRMS (ESI-TOF) m/z: [M+H]⁺ calculated for C₂₆H₃₅N₄O₆S, 531.2272, found 531.2280.

### Synthesis of Intermediate 26.

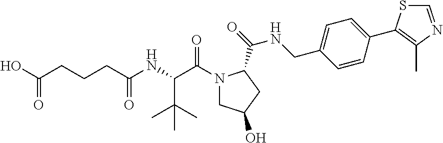 [SMILES: Cc1ncsc1-c1ccc(CNC(=O)[C@@H]2C[C@@H](O)CN2C(=O)[C@@H](NC(=O)CCCC(=O)O)C(C)(C)C)cc1]

5-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-5-oxopentanoic acid (230 mg, 43%) as white solid. ¹H NMR (600 MHz CD₃OD) δ 9.14 (s, 1H), 7.51 (d, J=9.0 Hz, 2H), 7.46 (d, J=8.4 Hz, 2H), 4.65 (s, 1H), 4.60-4.57 (m, 1H), 4.56 (d, J=15.6 Hz, 1H), 4.53-4.50 (m, 1H), 4.38 (d, J=15.6 Hz, 1H), 3.94 (d, J=11.4 Hz, 1H), 3.82 (dd, J=11.4, 3.6 Hz, 1H), 2.52 (s, 3H), 2.40-2.30 (m, 4H), 2.26-2.22 (m, 1H), 2.12-2.08 (m, 1H), 1.91 (t, J=7.8 Hz, 2H), 1.06 (s, 9H). HRMS (ESI-TOF) m/z: [M+H]⁺ calculated for C₂₇H₃₇N₄O₆S, 545.2428, found 545.2432.

### Synthesis of Intermediate 11.

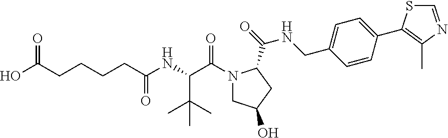 [SMILES: Cc1ncsc1-c1ccc(CNC(=O)[C@@H]2C[C@@H](O)CN2C(=O)[C@@H](NC(=O)CCCCC(=O)O)C(C)(C)C)cc1]

6-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-6-oxohexanoic acid (700 mg, 63%) as white solid. ¹H NMR (600 MHz CD₃OD) δ 9.12 (s, 1H), 7.51 (d, J=9.0 Hz, 2H), 7.46 (d, J=8.4 Hz, 2H), 4.65 (s, 1H), 4.60-4.55 (m, 2H), 4.53-4.50 (m, 1H), 4.38 (d, J=16.8 Hz, 1H), 3.93 (d, J=10.8 Hz, 1H), 3.82 (dd, J=11.4, 3.6 Hz, 1H), 2.52 (s, 3H), 2.38-2.21 (m, 5H), 2.12-2.08 (m, 1H), 1.71-1.62 (m, 4H), 1.06 (s, 9H). HRMS (ESI-TOF) m/z: [M+H]⁺ calculated for C₂₈H₃₉N₄O₆S, 559.2585, found 559.2605.

### Synthesis of Intermediate 12.

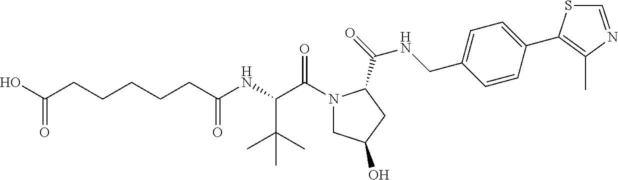 [SMILES: Cc1ncsc1-c1ccc(CNC(=O)[C@@H]2C[C@@H](O)CN2C(=O)[C@@H](NC(=O)CCCCCC(=O)O)C(C)(C)C)cc1]

7-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-7-oxoheptanoic acid (810 mg, 79%) as white solid. ¹H NMR (600 MHz CD₃OD) δ 8.98 (s, 1H), 7.50 (d, J=8.4 Hz, 2H), 7.44 (d, J=9.0 Hz, 2H), 4.65 (s, 1H), 4.60-4.49 (m, 3H), 4.38 (d, J=15.6 Hz, 1H), 3.93 (d, J=10.8 Hz, 1H), 3.82 (dd, J=11.4, 3.6 Hz, 1H), 2.51 (s, 3H), 2.35-2.22 (m, 5H), 2.13-2.08 (m, 1H), 1.68-1.59 (m, 4H), 1.42-1.34 (m, 2H), 1.06 (s, 9H). HRMS (ESI-TOF) m/z: [M+H]⁺ calculated for C₂₉H₄₁N₄O₆S, 573.2741, found 573.2754.

### Synthesis of Intermediate 27.

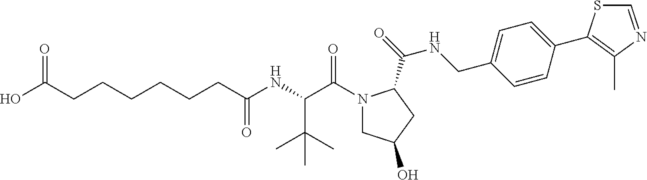 [SMILES: Cc1ncsc1-c1ccc(CNC(=O)[C@@H]2C[C@@H](O)CN2C(=O)[C@@H](NC(=O)CCCCCCC(=O)O)C(C)(C)C)cc1]

8-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-8-oxooctanoic acid (980 mg, 78%) as white solid. ¹H NMR (600 MHz, CD₃OD) δ 8.94 (s, 1H), 7.47 (d, J=8.1 Hz, 2H), 7.42 (d, J=8.2 Hz, 2H), 4.63 (s, 1H), 4.59-4.47 (m, 3H), 4.35 (d, J=15.4 Hz, 1H), 3.90 (d, J=11.0 Hz, 1H), 3.80 (dd, J=10.9, 3.9 Hz, 1H), 2.48 (s, 3H), 2.32-2.17 (m, 5H), 2.08 (ddd, J=13.3, 9.1, 4.5 Hz, 1H), 1.67-1.55 (m, 4H), 1.40-1.28 (m, 4H), 1.03 (s, 9H). HRMS (ESI-TOF) m/z: [M+H]⁺ calculated for C₃₀H₄₃N₄O₆S, 587.2898; found: 587.2903.

### Synthesis of Intermediate 27.

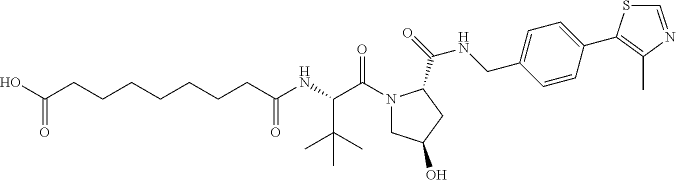 [SMILES: Cc1ncsc1-c1ccc(CNC(=O)[C@@H]2C[C@@H](O)CN2C(=O)[C@@H](NC(=O)CCCCCCCC(=O)O)C(C)(C)C)cc1]

9-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-9-oxononanoic acid (750 mg, 66%) as white solid. ¹H NMR (600 MHz CD₃OD) δ 9.09 (s, 1H), 7.51 (d, J=9.0 Hz, 2H), 7.46 (d, J=8.4 Hz, 2H), 4.66 (s, 1H), 4.61-4.50 (m, 3H), 4.38 (d, J=15.6 Hz, 11H), 3.93 (d, J=10.8 Hz, 1H), 3.82 (dd, J=11.4, 3.6 Hz, 1H), 2.52 (s, 3H), 2.36-2.22 (m, 5H), 2.12-2.07 (m, 1H), 1.68-1.59 (m, 4H), 1.40-1.34 (m, 8H), 1.06 (s, 9H); HPLC 98%; t_{R}=4.24 min; HRMS(TOF) calculated for C₃₁H₄₅N₄O₆S [M+H]⁺, 601.3054, found 601.3064.

### Synthesis of Intermediate 29.

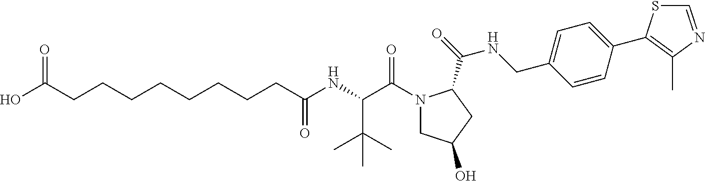 [SMILES: Cc1ncsc1-c1ccc(CNC(=O)[C@@H]2C[C@@H](O)CN2C(=O)[C@@H](NC(=O)CCCCCCCCC(=O)O)C(C)(C)C)cc1]

10-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-10-oxodecanoic acid (900 mg, 73%) as white solid. ¹H NMR (600 MHz CD₃OD) δ 8.98 (s, 1H), 7.50 (d, J=8.4 Hz, 2H), 7.45 (d, J=9.0 Hz, 2H), 4.66 (s, 1H), 4.61-4.50 (m, 3H), 4.38 (d, J=14.4 Hz, 11H), 3.93 (d, J=10.8 Hz, 1H), 3.83 (dd, J=11.4, 3.6 Hz, 1H), 2.51 (s, 3H), 2.35-2.22 (m, 5H), 2.13-2.08 (m, 1H), 1.66-1.58 (m, 4H), 1.38-1.32 (m, 10H), 1.06 (s, 9H). HRMS(TOF) calculated for C₃₂H₄₇N₄O₆S [M+H]⁺, 615.3211, found 615.3224.

### Synthesis of Intermediate 13.

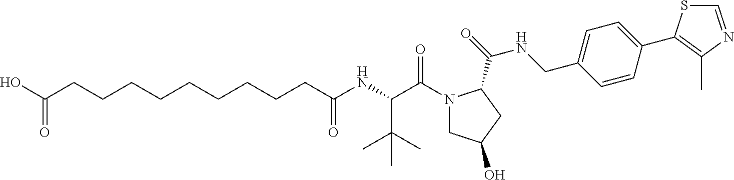 [SMILES: Cc1ncsc1-c1ccc(CNC(=O)[C@@H]2C[C@@H](O)CN2C(=O)[C@@H](NC(=O)CCCCCCCCCC(=O)O)C(C)(C)C)cc1]

11-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-11-oxoundecanoic acid (930 mg, 78%) as white solid. ¹H NMR (600 MHz CD₃OD) δ 8.95 (s, 1H), 7.49 (d, J=8.4 Hz, 2H), 7.44 (d, J=7.8 Hz, 2H), 4.66 (s, 1H), 4.61-4.50 (m, 3H), 4.38 (d, J=15.6 Hz, 1H), 3.93 (d, J=9.6 Hz, 1H), 3.82 (dd, J=11.4, 3.6 Hz, 1H), 2.50 (s, 3H), 2.35-2.21 (m, 5H), 2.12-2.07 (m, 1H), 1.66-1.57 (m, 4H), 1.37-1.29 (m, 12H), 1.06 (s, 9H). HRMS (ESI-TOF) calculated for C₃₃H₄₉N₄O₆S, 629.3367, found 629.3368.

### 3. Procedures for the Synthesis of VHL-1 PEG Linkers

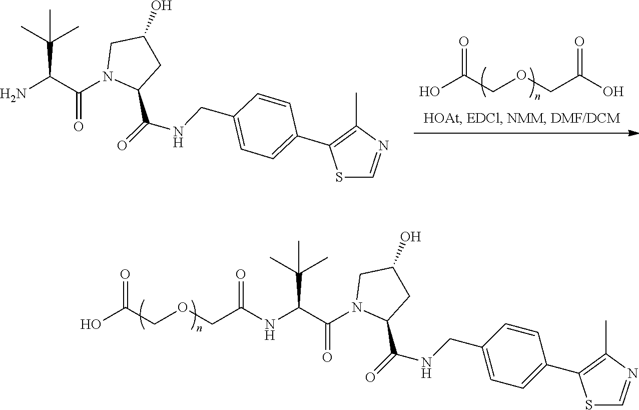

To a solution of diacid (4 mmol) in DMF (10 ml) and DCM (250 ml) was added NMM (10 mmol), VHL-1 (2 mmol), HOAt (2.4 mmol), and EDCI (2.4 mmol) at 0° C. The resulting reaction solution was stirred at 0° C. for 6 h and then at RT overnight. The progress of the reaction was monitored by LC/MS. After VHL-1 was totally consumed, the reaction was concentrated and the resulting residue was purified by reverse-phase chromatography to yield the product.

### Synthesis of Intermediate 19.

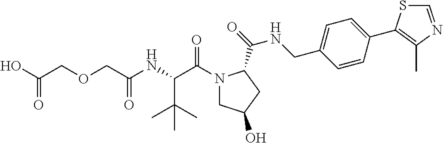 [SMILES: Cc1ncsc1-c1ccc(CNC(=O)[C@@H]2C[C@@H](O)CN2C(=O)[C@@H](NC(=O)COCC(=O)O)C(C)(C)C)cc1]

2-(2-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-2-oxoethoxy)acetic acid (810 mg, 69%) as white solid. ¹H NMR (600 MHz, CD₃OD) δ 8.97 (s, 1H), 7.47 (d, J=8.2 Hz, 2H), 7.43 (d, J=8.1 Hz, 2H), 4.69 (s, 1H), 4.60-4.47 (m, 3H), 4.36 (d, J=15.5 Hz, 1H), 4.27-4.17 (m, 2H), 4.16-4.07 (m, 2H), 3.89 (d, J=11.0 Hz, 1H), 3.81 (dd, J=11.0, 3.8 Hz, 1H), 2.48 (s, 3H), 2.22 (dd, J=13.1, 7.6 Hz, 1H), 2.08 (ddd, J=13.3, 9.2, 4.5 Hz, 1H), 1.05 (s, 9H). HRMS (ESI-TOF) m/z: [M+H]⁺ calculated for C₂₆H₃₅N₄O₇S, 547.2221; found: 547.2230.

### Synthesis of Intermediate 2.

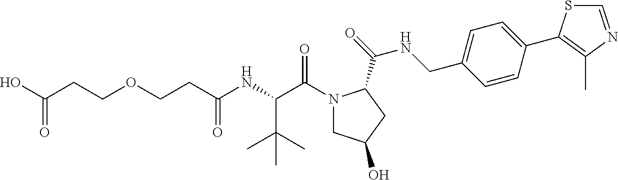 [SMILES: Cc1ncsc1-c1ccc(CNC(=O)[C@@H]2C[C@@H](O)CN2C(=O)[C@@H](NC(=O)CCOCCC(=O)O)C(C)(C)C)cc1]

3-(3-(((S)-1-((2S,4R)-4-Hydroxy-2-((4-(4-methylthiazol-5-yl)benzyl)carbamoyl) pyrrolidin-1-yl)-3,3-dimethyl-1-oxobutan-2-yl)amino)-3-oxopropoxy)propanoic acid (450 mg, 63%) as white solid. ¹H NMR (600 MHz, CD₃OD) δ 9.00 (s, 1H), 7.45 (d, J=22.1 Hz, 4H), 4.64 (s, 1H), 4.61-4.44 (m, 3H), 4.36 (d, J=15.4 Hz, 1H), 3.84 (dd, J=57.3, 10.5 Hz, 2H), 3.75-3.56 (m, 4H), 2.60-2.39 (m, 7H), 2.24-2.17 (m, 1H), 2.11-2.03 (m, 1H), 1.03 (s, 9H). HRMS (ESI-TOF) m/z: [M+H]⁺ calculated for C₂₈H₃₉N₄O₇S, 575.2534; found: 575.2543.

### Synthesis of Intermediate 20.

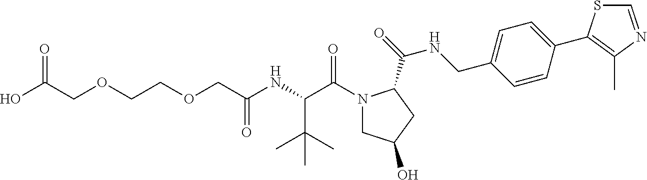 [SMILES: Cc1ncsc1-c1ccc(CNC(=O)[C@@H]2C[C@@H](O)CN2C(=O)[C@@H](NC(=O)COCCOCC(=O)O)C(C)(C)C)cc1]

2-(2-(2-(((S)-1-((2S,4R)-4-Hydroxy-2-((4-(4-methylthiazol-5-yl)benzyl) carbamoyl)pyrrolidin-1-yl)-3,3-dimethyl-1-oxobutan-2-yl)amino)-2-oxoethoxy)ethoxy)acetic acid (680 mg, 54%) as white solid. ¹H NMR (600 MHz, CD₃OD) δ 9.05 (s, 1H), 7.48 (d, J=8.1 Hz, 2H), 7.44 (d, J=8.2 Hz, 2H), 4.69 (s, 1H), 4.56 (dd, J=18.6, 12.1 Hz, 2H), 4.50 (s, 1H), 4.36 (d, J=15.5 Hz, 1H), 4.21 (d, J=16.8 Hz, 1H), 4.13 (d, J=16.9 Hz, 1H), 4.08 (d, J=15.6 Hz, 1H), 4.04 (d, J=15.7 Hz, 1H), 3.88 (d, J=11.0 Hz, 1H), 3.83-3.69 (m, 5H), 2.49 (s, 3H), 2.25-2.19 (m, 1H), 2.08 (ddd, J=13.3, 9.2, 4.4 Hz, 1H), 1.04 (s, 9H). HRMS (ESI-TOF) m/z: [M+H]⁺ calculated for C₂₈H₃₉N₄O₈S, 591.2483; found: 591.2477.

### Synthesis of Intermediate 21.

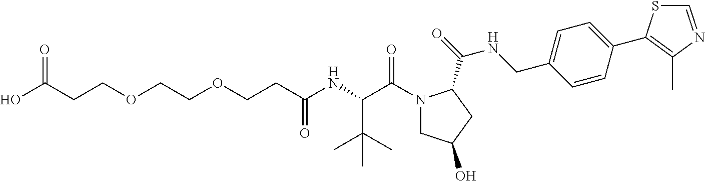 [SMILES: Cc1ncsc1-c1ccc(CNC(=O)[C@@H]2C[C@@H](O)CN2C(=O)[C@@H](NC(=O)CCOCCOCCC(=O)O)C(C)(C)C)cc1]

3-(2-(3-(((S)-1-((2S,4R)-4-Hydroxy-2-((4-(4-methylthiazol-5-yl)benzyl) carbamoyl)pyrrolidin-1-yl)-3,3-dimethyl-1-oxobutan-2-yl)amino)-3-oxopropoxy)ethoxy) propanoic acid (680 mg, 64%) as white solid. ¹H NMR (600 MHz, CD₃OD) δ 8.98 (d, J=20.1 Hz, 1H), 7.48 (d, J=8.0 Hz, 2H), 7.43 (d, J=8.1 Hz, 2H), 4.64 (s, 1H), 4.59-4.51 (m, 2H), 4.49 (s, 1H), 4.35 (d, J=15.5 Hz, 1H), 3.89 (d, J=11.0 Hz, 1H), 3.80 (dd, J=10.9, 3.8 Hz, 1H), 3.76-3.67 (m, 4H), 3.63-3.55 (m, 4H), 2.60-2.43 (m, 7H), 2.21 (dd, J=13.1, 7.6 Hz, 1H), 2.08 (ddd, J=13.2, 9.1, 4.5 Hz, 1H), 1.04 (s, 9H). HRMS (ESI-TOF) m/z: [M+H]⁺ calculated for C₃₀H₄₃N₄O₈S, 619.2796; found: 619.2800.

### Synthesis of Intermediate 3.

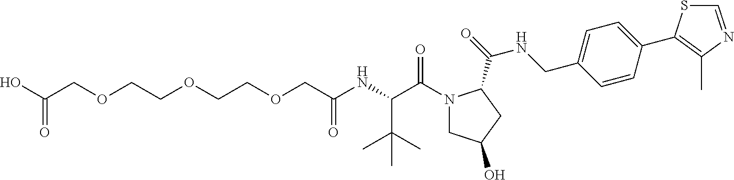 [SMILES: Cc1ncsc1-c1ccc(CNC(=O)[C@@H]2C[C@@H](O)CN2C(=O)[C@@H](NC(=O)COCCOCCOCC(=O)O)C(C)(C)C)cc1]

(S)-13-((2S,4R)-4-Hydroxy-2-((4-(4-methylthiazol-5-yl)benzyl)carbamoyl) pyrrolidine-1-carbonyl)-14,14-dimethyl-11-oxo-3,6,9-trioxa-12-azapentadecanoic acid (880 mg, 54%) as white solid. ¹H NMR (600 MHz, CD₃OD) δ 9.05 (s, 1H), 7.48 (d, J=8.2 Hz, 2H), 7.44 (d, J=8.3 Hz, 2H), 4.69 (s, 1H), 4.60-4.51 (m, 2H), 4.50 (s, 1H), 4.36 (d, J=15.5 Hz, 1H), 4.10 (s, 1H), 4.07 (d, J=15.6 Hz, 1H), 4.03 (d, J=15.6 Hz, 1H), 3.87 (d, J=11.0 Hz, 1H), 3.80 (dd, J=11.0, 3.8 Hz, 1H), 3.76-3.64 (m, 9H), 2.50 (s, 3H), 2.22 (dd, J=13.1, 7.6 Hz, 1H), 2.08 (ddd, J=13.3, 9.2, 4.4 Hz, 1H), 1.04 (s, 9H). HRMS (ESI-TOF) m/z: [M+H]⁺ calculated for C₃₀H₄₃N₄O₉S, 635.2745; found: 635.2751.

### Synthesis of Intermediate 9.

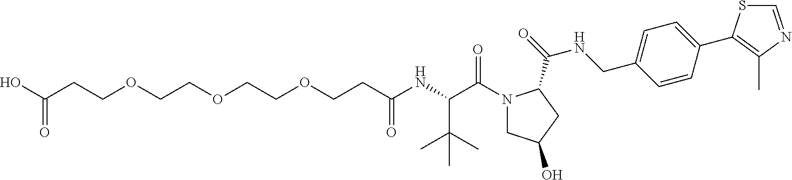 [SMILES: Cc1ncsc1-c1ccc(CNC(=O)[C@@H]2C[C@@H](O)CN2C(=O)[C@@H](NC(=O)CCOCCOCCOCCC(=O)O)C(C)(C)C)cc1]

(S)-15-((2S,4R)-4-Hydroxy-2-((4-(4-methylthiazol-5-yl)benzyl)carbamoyl) pyrrolidine-1-carbonyl)-16,16-dimethyl-13-oxo-4,7,10-trioxa-14-azaheptadecanoic acid (677 mg, 57%) as white solid. ¹H NMR (600 MHz, CD₃OD) δ 8.95 (s, 1H), 7.47 (d, J=8.1 Hz, 2H), 7.42 (d, J=8.1 Hz, 2H), 4.65 (s, 1H), 4.59-4.51 (m, 2H), 4.49 (s, 1H), 4.35 (d, J=15.5 Hz, 1H), 3.89 (d, J=11.1 Hz, 1H), 3.80 (dd, J=10.9, 3.9 Hz, 1H), 3.76-3.67 (m, 4H), 3.66-3.54 (m, 8H), 2.60-2.50 (m, 3H), 2.50-2.43 (m, 4H), 2.21 (dd, J=13.1, 7.6 Hz, 1H), 2.08 (ddd, J=13.3, 9.1, 4.5 Hz, 1H), 1.04 (s, 9H). HRMS (ESI-TOF) in z: [M+H]⁺ calculated for C₃₂H₄₇N₄O₉S, 663.3058; found: 663.3059.

### Synthesis of Intermediate 18.

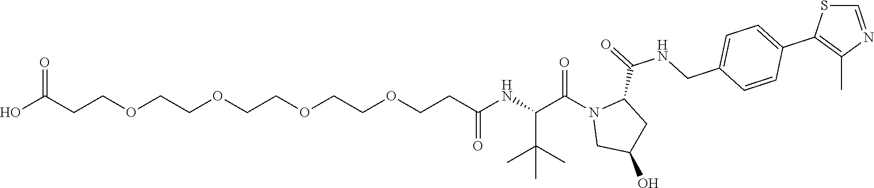 [SMILES: Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1]

(S)-18-((2S,4R)-4-Hydroxy-2-((4-(4-methylthiazol-5-yl)benzyl)carbamoyl) pyrrolidine-1-carbonyl)-19,19-dimethyl-16-oxo-4,7,10,13-tetraoxa-17-azaicosanoic acid (590 mg, 65%) as white solid. ¹H NMR (600 MHz, CD₃OD) δ 8.99 (s, 1H), 7.48 (d, J=8.1 Hz, 2H), 7.42 (d, J=8.2 Hz, 2H), 4.65 (s, 1H), 4.59-4.51 (m, 2H), 4.49 (s, 1H), 4.35 (d, J=15.5 Hz, 1H), 3.89 (d, J=11.0 Hz, 1H), 3.80 (dd, J=10.9, 3.8 Hz, 1H), 3.77-3.67 (m, 4H), 3.67-3.54 (m, 12H), 2.61-2.43 (m, 7H), 2.21 (dd, J=13.0, 7.6 Hz, 1H), 2.08 (ddd, J=13.2, 9.1, 4.4 Hz, 1H), 1.04 (s, 9H). HRMS (ESI-TOF) m/z: [M+H]⁺ calculated for C₃₄H₅₁N₄O₁₀S, 707.3320; found: 707.3321.

### Synthesis of Intermediate 4.

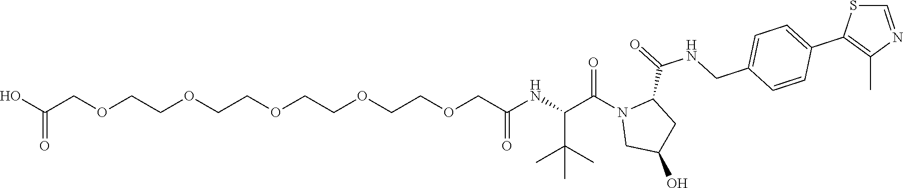 [SMILES: Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1]

(S)-19-((2S,4R)-4-Hydroxy-2-((4-(4-methylthiazol-5-yl)benzyl)carbamoyl) pyrrolidine-1-carbonyl)-20,20-dimethyl-17-oxo-3,6,9,12,15-pentaoxa-18-azahenicosanoic acid (496 mg, 54%) as white solid. ¹H NMR (600 MHz, CD₃OD) δ 8.89 (s, 1H), 7.47 (d, J=8.1 Hz, 2H), 7.42 (d, J=8.1 Hz, 2H), 4.69 (s, 1H), 4.59-4.46 (m, 3H), 4.36 (d, J=15.5 Hz, 1H), 4.16-4.00 (m, 4H), 3.87 (d, J=11.0 Hz, 1H), 3.80 (dd, J=11.0, 3.7 Hz, 1H), 3.76-3.53 (m, 16H), 2.48 (s, 3H), 2.22 (dd, J=13.1, 7.6 Hz, 1H), 2.08 (ddd, J=13.3, 9.2, 4.4 Hz, 1H), 1.04 (s, 7H). HRMS (ESI-TOF) m/z: [M+H]⁺ calculated for C₃₄H₅₁N₄O₁₁S, 723.3270; found: 723.3269.

### Synthesis of Intermediate 10.

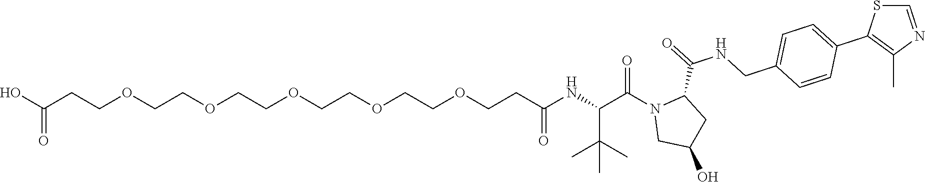 [SMILES: Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1]

(S)-21-((2S,4R)-4-Hydroxy-2-((4-(4-methylthiazol-5-yl)benzyl)carbamoyl) pyrrolidine-1-carbonyl)-22,22-dimethyl-19-oxo-4,7,10,13,16-pentaoxa-20-azatricosanoic acid (420 mg, 42%) as white solid. ¹H NMR (600 MHz, CD₃OD) δ 8.89 (s, 1H), 7.47 (d, J=8.0 Hz, 2H), 7.42 (d, J=8.1 Hz, 2H), 4.65 (s, 1H), 4.59-4.51 (m, 2H), 4.49 (s, 1H), 4.35 (d, J=15.5 Hz, 1H), 3.89 (d, J=11.0 Hz, 1H), 3.80 (dd, J=10.9, 3.8 Hz, 1H), 3.77-3.67 (m, 4H), 3.67-3.51 (m, 16H), 2.61-2.42 (m, 7H), 2.24-2.18 (m, 1H), 2.08 (ddd, J=13.2, 9.1, 4.4 Hz, 1H), 1.02 (d, J=14.3 Hz, 9H). HRMS (ESI-TOF) m/z: [M+H]⁺ calculated for C₃₆H₅₅N₄O₁₁S, 751.3583; found: 751.3589.

### Synthesis of Intermediate 30.

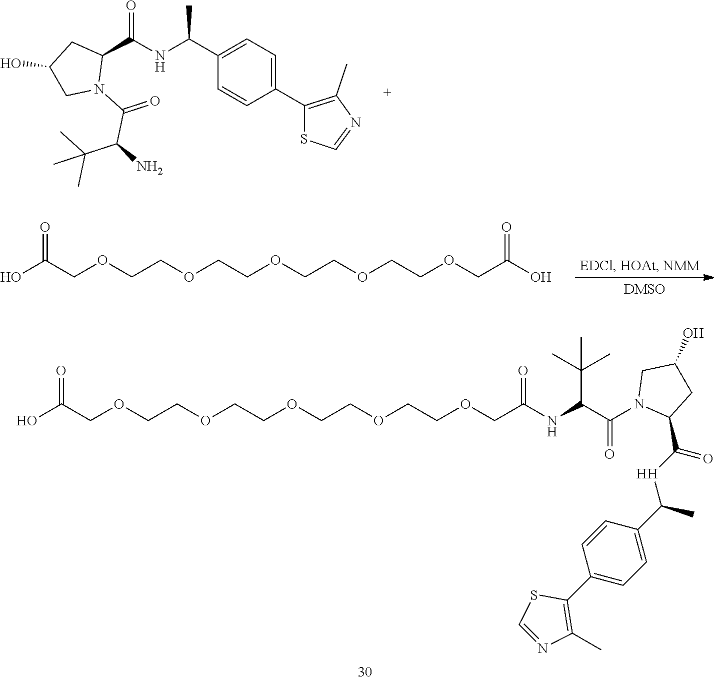

(S)-19-((2S,4R)-4-hydroxy-2-(((S)-1-(4-(4-methylthiazol-5-yl)phenyl)ethyl)carbamoyl)pyrrolidine-1-carbonyl)-20,20-dimethyl-17-oxo-3,6,9,12,15-pentaoxa-18-azahenicosanoic acid To a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (Raina et al., 2016) (33.6 mg, 0.05 mmol), 3,6,9,12,15-pentaoxaheptadecanedioic acid (31.03 mg, 0.1 mmol), EDCI (1-ethyl-3-(3-dimethylaminopropyl)carbodiimide) (12 mg, 0.06 mmol), and HOAt (1-hydroxy-7-azabenzo-triazole) (8 mg, 0.06 mmol) in 2 mL of DMSO, was added NMM (N-Methylmorpholine) (30 mg, 0.3 mmol). After being stirring overnight at room temperature, the resulting mixture was purified by preparative HPLC (10%-100% methanol/0.1% TFA in H₂O) to afford Intermediate 30 as oil in TFA salt form (22 mg, yield 52%). ¹H NMR (600 MHz, Methanol-d₄) δ 9.25 (s, 1H), 7.51-7.38 (m, 4H), 5.04-4.97 (m, 2H), 4.68 (s, 1H), 4.60-4.54 (m, 1H), 4.44 (s, 1H), 4.13 (s, 1H), 4.06-4.04 (m, 1H), 3.85 (d, J=11.1 Hz, 1H), 3.76-3.61 (m, 17H), 3.31 (dq, J=3.0, 1.3 Hz, 1H), 2.52 (s, 3H), 2.20 (dd, J=13.2, 7.7 Hz, 1H), 1.95 (ddd, J=13.3, 9.2, 4.4 Hz, 1H), 1.51 (d, J=7.0 Hz, 3H), 1.05 (s, 9H). MS (ESI) m/z 737.3 [M+H]⁺.

### 4. Procedures for the Synthesis of Pomalidomide Linkers

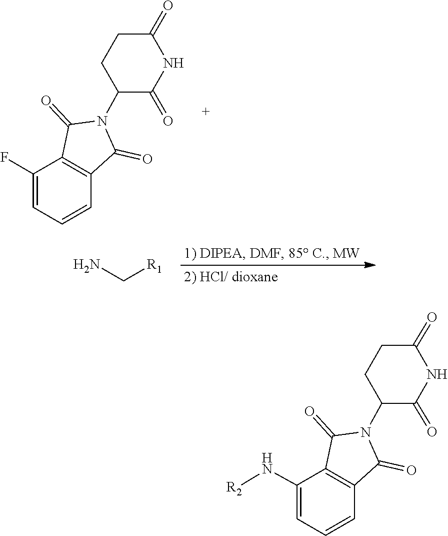

A solution of pomalidomide analogue (1 eq.), amine (1 eq.), and N,N-diisopropylethylamine (1.5 eq.) in DMF (2.0 ml per mmol of pomalidamide) was heated to 85° C. in a microwave reactor for 40 min. After cooling to RT, the reaction was quenched with water and extracted with ethyl acetate (3×). The combined organic phase was dried over anhydrous sodium sulfate and concentrated under reduced pressure. The resulting residue was purified by silica gel chromatography (eluted with hexanes/EtOAc: 0-100%) to give the desired t-Bu ester intermediate as oil. This intermediate was treated with a solution of hydrogen chloride in dioxane (4 M, 5 ml per mmol of pomalidamide) for overnight. After concentration under reduced pressure, the desired acid product was obtained as yellow oil.

### Synthesis of Intermediate 8.

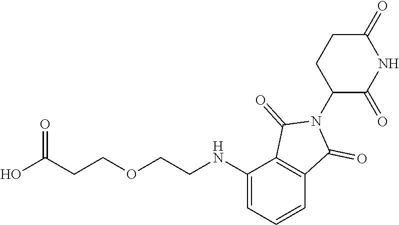 [SMILES: O=C(O)CCOCCNc1cccc2c1C(=O)N(C1CCC(=O)NC1=O)C2=O]

3-(2-((2-(2,6-Dioxopiperidin-3-yl)-1,3-dioxoisoindolin-4-yl)amino)ethoxy) propanoic acid. tert-Butyl 3-(2-aminoethoxy)propanoate (1.0 g, 5.3 mmol) was used to prepare the title compound (500 mg, 24%) according to the above procedures. ¹H NMR (600 MHz, CD₃OD) δ 7.54 (dd, J=8.3, 7.0, 1.2 Hz, 1H), 7.09 (d, 1H), 7.04 (d, J=7.0, 1.1 Hz, 1H), 5.05 (dd, J=12.5, 5.4, 1.2 Hz, 1H), 3.75 (t, J=6.2, 1.2 Hz, 2H), 3.65-3.69 (m, 2H), 3.45-3.49 (m, 2H), 2.88-2.82 (m, 1H), 2.76-2.70 (m, 2H), 2.56 (t, J=6.2, 1.2 Hz, 2H), 2.10 (ddt, J=14.9, 7.6, 3.7, 1.6 Hz, 1H). MS (ESI) m/z 390.2 [M+H]⁺.

### Synthesis of Intermediate 17.

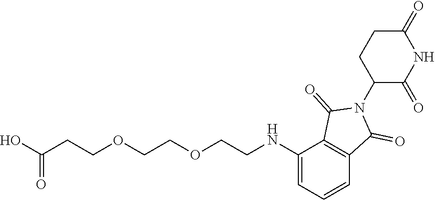 [SMILES: O=C(O)CCOCCOCCNc1cccc2c1C(=O)N(C1CCC(=O)NC1=O)C2=O]

3-(2-(2-((2-(2,6-Dioxopiperidin-3-yl)-1,3-dioxoisoindolin-4-yl)amino)ethoxy) ethoxy)-propanoic acid. tert-Butyl 3-(2-(2-aminoethoxy)ethoxy)propanoate (0.70 g, 3.0 mmol) was used to prepare tert-butyl 3-(2-(2-((2-(2,6-dioxopiperidin-3-yl)-1,3-dioxoisoindolin-4-yl)amino)-ethoxy)ethoxy)propanoate (575 mg, 39%) according to the above procedures. 1H NMR (600 MHz, CDCl₃) δ 8.13 (s, 1H), 7.53-7.45 (m, 1H), 7.10 (d, J=7.1 Hz, 1H), 6.92 (d, J=8.5 Hz, 1H), 6.49 (t, J=5.6 Hz, 1H), 4.91 (dd, J=12.4, 5.3 Hz, 1H), 3.76-3.69 (m, 4H), 3.67-3.60 (m, 4H), 3.46 (q, J=5.5 Hz, 2H), 2.89 (dt, J=16.8, 3.2 Hz, 1H), 2.84-2.69 (m, 2H), 2.51 (t, J=6.6 Hz, 2H), 2.16-2.08 (m, 1H), 1.44 (s, 9H). MS (ESI) m/z 490.2 [M+H]⁺. The t-Bu ester intermediate was dissolved in formic acid (10 ml) and the resulting solution was stirred at RT overnight. After removal of the solvent under reduced pressure, the tittle compound (512 mg, 100%) was obtained and used for the following reactions without further purification.

### Synthesis of Intermediate 9.

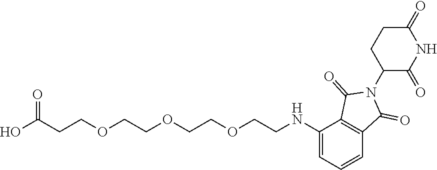 [SMILES: O=C(O)CCOCCOCCOCCNc1cccc2c1C(=O)N(C1CCC(=O)NC1=O)C2=O]

3-(2-(2-(2-((2-(2,6-Dioxopiperidin-3-yl)-1,3-dioxoisoindolin-4-yl)amino)ethoxy) ethoxy)ethoxy)propanoic acid. tert-Butyl 3-(2-(2-(2-aminoethoxy)ethoxy)ethoxy)propanoate (1.0 g, 3.6 mmol) was used to prepare the title compound (240 mg, 10%) according to the above procedures. ¹H NMR (600 MHz, CD₃OD) δ 7.55 (dd, J=8.4, 7.2 Hz, 1H), 7.10 (d, J=8.6 Hz, 1H), 7.05 (d, J=7.1 Hz, 1H), 5.05 (dd, J=12.4, 5.4 Hz, 1H), 3.71 (dt, J=9.4, 5.7 Hz, 4H), 3.66-3.63 (m, 4H), 3.62 (dd, J=6.0, 3.5 Hz, 2H), 3.58 (dd, J=6.1, 3.5 Hz, 2H), 3.50 (t, J=5.3 Hz, 2H), 2.86 (ddd, J=19.1, 14.1, 5.3 Hz, 1H), 2.77-2.66 (m, 2H), 2.52 (t, J=6.3 Hz, 2H), 2.11 (ddt, J=10.3, 5.0 Hz, 1H). MS (ESI) m/z 478.3 [M+H]⁺.

### Synthesis of Intermediate 18.

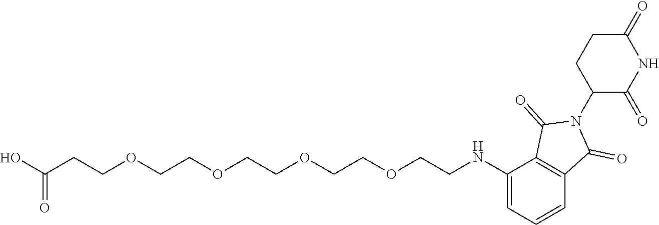 [SMILES: O=C(O)CCOCCOCCOCCOCCNc1cccc2c1C(=O)N(C1CCC(=O)NC1=O)C2=O]

1-((2-(2,6-Dioxopiperidin-3-yl)-1,3-dioxoisoindolin-4-yl)amino)-3,6,9,12-tetraoxapentadecan-15-oic acid. tert-Butyl 1-amino-3,6,9,12-tetraoxapentadecan-15-oate (0.96 g, 3.0 mmol) was used to prepare the t-Bu ester intermediate according to the general procedures. The t-Bu ester intermediate was dissolved in formic acid (10 ml) and the resulting solution was stirred at RT overnight. After removal of the solvent under reduced pressure, the title compound (950 mg, 61%) was obtained and used for the following reactions without further purification. ¹H NMR (600 MHz, CD₃OD) δ 7.55 (t, J=7.8 Hz, 1H), 7.10 (d, J=8.5 Hz, 1H), 7.06 (d, J=7.0 Hz, 1H), 5.05 (dd, J=12.6, 5.3 Hz, 1H), 3.75-3.68 (m, 4H), 3.68-3.55 (m, 12H), 3.50 (t, J=4.9 Hz, 2H), 2.90-2.81 (m, 1H), 2.78-2.66 (m, 2H), 2.52 (t, J=6.0 Hz, 2H), 2.14-2.07 (m, 1H). MS (ESI) m/z 522.2 [M+H]⁺.

### Synthesis of Intermediate 10.

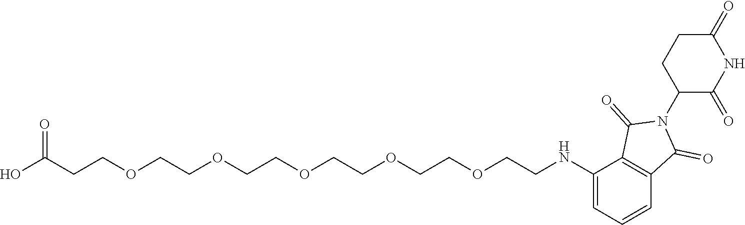 [SMILES: O=C(O)CCOCCOCCOCCOCCOCCNc1cccc2c1C(=O)N(C1CCC(=O)NC1=O)C2=O]

1-((2-(2,6-Dioxopiperidin-3-yl)-1,3-dioxoisoindolin-4-yl)amino)-3,6,9,12,15-pentaoxaoctadecan-18-oic acid. tert-Butyl 1-amino-3,6,9,12,15-pentaoxaoctadecan-18-oate (1.10 g, 3.0 mmol) was used to prepare tert-butyl 1-((2-(2,6-dioxopiperidin-3-yl)-1,3-dioxoisoindolin-4-yl)amino)-3,6,9,12,15-pentaoxaoctadecan-18-oate (1.35 g, 72%) according to the above procedures. ¹H NMR (600 MHz, CDCl₃) δ 8.32 (s, 1H), 7.48 (dd, J=8.5, 7.1 Hz, 1H), 7.10 (d, J=7.1 Hz, 1H), 6.91 (d, J=8.6 Hz, 1H), 6.49 (t, J=5.7 Hz, 1H), 4.91 (dd, J=12.4, 5.3 Hz, 1H), 3.74-3.68 (m, 4H), 3.68-3.63 (m, 12H), 3.63-3.58 (m, 4H), 3.46 (q, J=5.6 Hz, 2H), 2.92-2.85 (m, 1H), 2.83-2.68 (m, 2H), 2.49 (t, J=6.6 Hz, 2H), 2.15-2.08 (m, 1H), 1.43 (s, 9H). MS (ESI) m/z 622.2 [M+H]⁺. The t-Bu ester intermediate was dissolved in formic acid (10 ml) and the resulting solution was stirred at RT overnight. After removal of the solvent under reduced pressure, the tittle compound (1.23 g, 100%) was obtained and used for the following reactions without further purification.

### Synthesis of Intermediate 5.

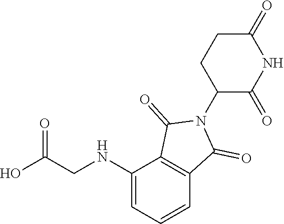 [SMILES: O=C(O)CNc1cccc2c1C(=O)N(C1CCC(=O)NC1=O)C2=O]

(2-(2,6-Dioxopiperidin-3-yl)-1,3-dioxoisoindolin-4-yl)glycine. tert-Butyl glycinate (838 mg, 5.0 mmol) was used to prepare the title compound (240 mg, 14%) according to the general procedures. ¹H NMR (600 MHz, CD₃OD) δ 7.57 (dd, J=8.5, 7.1 Hz, 1H), 7.11 (d, J=7.1 Hz, 1H), 6.95 (d, J=8.5 Hz, 1H), 5.07 (dd, J=12.6, 5.5 Hz, 1H), 4.12 (s, 2H), 2.86 (ddd, J=18.0, 14.4, 5.4 Hz, 1H), 2.74-2.67 (m, 2H), 2.15-2.08 (m, 1H). MS (ESI) m/z 332.1 [M+H]⁺.

### Synthesis of Intermediate 14.

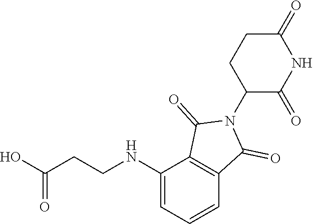 [SMILES: O=C(O)CCNc1cccc2c1C(=O)N(C1CCC(=O)NC1=O)C2=O]

3-((2-(2,6-Dioxopiperidin-3-yl)-1,3-dioxoisoindolin-4-yl)amino)propanoic acid. tert-Butyl 3-aminopropanoate HCl salt (1.0 g, 5.97 mmol) was used to prepare the title compound (700 mg, 34%) according to the above procedures. ¹H NMR (600 MHz, CD₃OD) δ 7.57 (dd, J=8.6, 7.1 Hz, 1H), 7.11 (d, J=8.6 Hz, 1H), 7.06 (d, J=7.1 Hz, 1H), 5.05 (dd, J=12.6, 5.5 Hz, 1H), 3.62 (t, J=6.5 Hz, 2H), 2.88-2.82 (m, 1H), 2.76-2.69 (m, 2H), 2.64 (t, J=6.5 Hz, 2H), 2.13-2.07 (m, 1H). MS (ESI) m/z 346.2 [M+H]⁺.

### Synthesis of Intermediate 15.

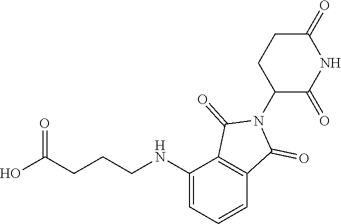 [SMILES: O=C(O)CCCNc1cccc2c1C(=O)N(C1CCC(=O)NC1=O)C2=O]

4-((2-(2,6-Dioxopiperidin-3-yl)-1,3-dioxoisoindolin-4-yl)amino)butanoic acid. tert-Butyl 4-aminobutanoate (1.0 g, 6.2 mmol) was used to prepare the title compound (550 mg, 25%) according to the above procedures. ¹H NMR (600 MHz, CD₃OD) δ 7.55 (dd, J=8.6, 7.1 Hz, 1H), 7.10 (d, J=8.5 Hz, 1H), 7.04 (d, J=7.1 Hz, 1H), 5.05 (dd, J=12.4, 5.5 Hz, 1H), 3.39 (t, J=7.2 Hz, 2H), 2.85-2.82 (m, 1H), 2.76-2.69 (m, 2H), 2.42 (t, J=7.1 Hz, 2H), 2.10 (tq, J=8.0, 3.8 Hz, 1H), 1.94 (dp, J=14.3, 7.0 Hz, 2H). MS (ESI) m/z 360.1 [M+H]⁺.

### Synthesis of Intermediate 6.

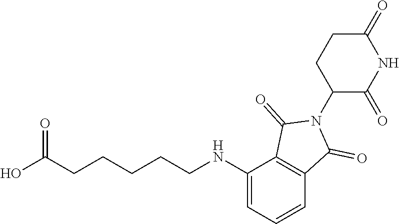 [SMILES: O=C(O)CCCCCNc1cccc2c1C(=O)N(C1CCC(=O)NC1=O)C2=O]

6-((2-(2,6-Dioxopiperidin-3-yl)-1,3-dioxoisoindolin-4-yl)amino)hexanoic acid. tert-Butyl 6-aminohexanoate (1.0 g, 4.47 mmol) was used to prepare the title compound (460 mg, 27%) according to the above procedures. ¹H NMR (600 MHz, CD₃OD) δ 7.54 (dd, J=8.6, 7.1 Hz, 1H), 7.03 (dd, J=7.8, 3.8 Hz, 2H), 5.05 (dd, J=12.5, 5.4 Hz, 1H), 3.33 (t, J=7.1 Hz, 2H), 2.88-2.82 (m, 1H), 2.75-2.67 (m, 2H), 2.31 (t, J=7.4 Hz, 2H), 2.10 (tdd, J=10.1, 5.3, 3.1 Hz, 1H), 1.70-1.64 (m, 4H), 1.46 (dddd, J=13.0, 8.9, 7.1, 4.2 Hz, 2H). MS (ESI) m/z 388.1 [M+H]⁺.

### Synthesis of Intermediate 16.

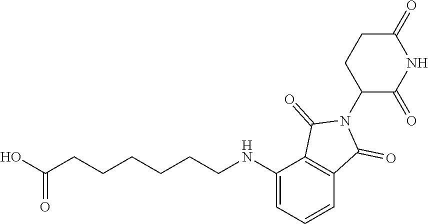 [SMILES: O=C(O)CCCCCCNc1cccc2c1C(=O)N(C1CCC(=O)NC1=O)C2=O]

7-((2-(2,6-Dioxopiperidin-3-yl)-1,3-dioxoisoindolin-4-yl)amino)heptanoic acid. tert-Butyl 7-aminoheptanoate (1.0 g, 4.96 mmol) was used to prepare the title compound (500 mg, 25%) according to the above procedures. ¹H NMR (600 MHz, CD₃OD) δ 7.54 (dd, 1H), 7.03 (dd, J=7.8, 3.7 Hz, 2H), 5.05 (dd, J=12.5, 5.5 Hz, 1H), 3.30-3.33 (m, 2H), 2.90-2.79 (m, 1H), 2.77-2.68 (m, 2H), 2.29 (t, J=7.4 Hz, 2H), 2.13-2.07 (m, 1H), 1.68-1.61 (m, 4H), 1.46-1.40 (m, 4H). MS (ESI) m/z 402.3 [M+H]⁺.

### Synthesis of Intermediate 7.

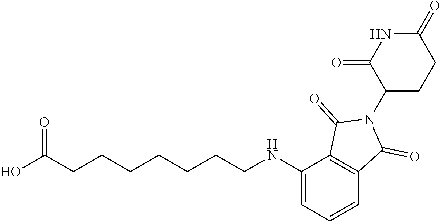 [SMILES: O=C(O)CCCCCCCNc1cccc2c1C(=O)N(C1CCC(=O)NC1=O)C2=O]

8-((2-(2,6-Dioxopiperidin-3-yl)-1,3-dioxoisoindolin-4-yl)amino)octanoic acid. tert-Butyl 8-aminooctanoate (1.0 g, 4.6 mmol) was used to prepare the title compound (620 mg, 32%) according to the above procedures. ¹H NMR (600 MHz, CD₃OD) δ 7.53 (dd, J=8.6, 7.0, 1.5 Hz, 1H), 7.08-6.93 (m, 2H), 5.05 (dd, J=12.5, 5.5, 1.5 Hz, 1H), 3.31 (t, 2H), 2.90-2.79 (m, 1H), 2.75-2.66 (m, 2H), 2.28 (t, J=7.5, 1.5 Hz, 2H), 2.13-2.07 (m, 1H), 1.66-1.51 (m, 4H), 1.43-1.33 (m, 6H). MS (ESI) m/z 416.4[M+H]⁺.

### Synthesis of Intermediate 32.

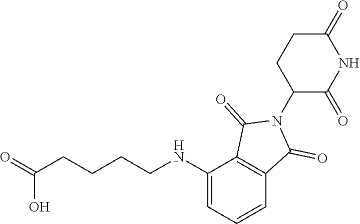 [SMILES: O=C(O)CCCCNc1cccc2c1C(=O)N(C1CCC(=O)NC1=O)C2=O]

5-((2-(2,6-dioxopiperidin-3-yl)-1,3-dioxoisoindolin-4-yl)amino)pentanoic acid. tert-butyl 5-aminopentanoate (1 g, 5.7 mmol) was used to prepare the title compound (638 mg, 30%) according to the above procedures. ¹H NMR (600 MHz, Methanol-d₄) δ 7.54 (dd, J=8.6, 7.1 Hz, 1H), 7.10-6.96 (m, 2H), 5.05 (dd, J=12.6, 5.5 Hz, 1H), 3.34 (t, J=6.5 Hz, 2H), 2.89-2.80 (m, 1H), 2.77-2.66 (m, 2H), 2.38-2.33 (m, 2H), 2.13-2.07 (m, 1H), 1.71 (qd, J=4.7, 1.9 Hz, 4H). MS (ESI) m/z 374.1 [M+H]⁺.

### Synthesis of Intermediate 36.

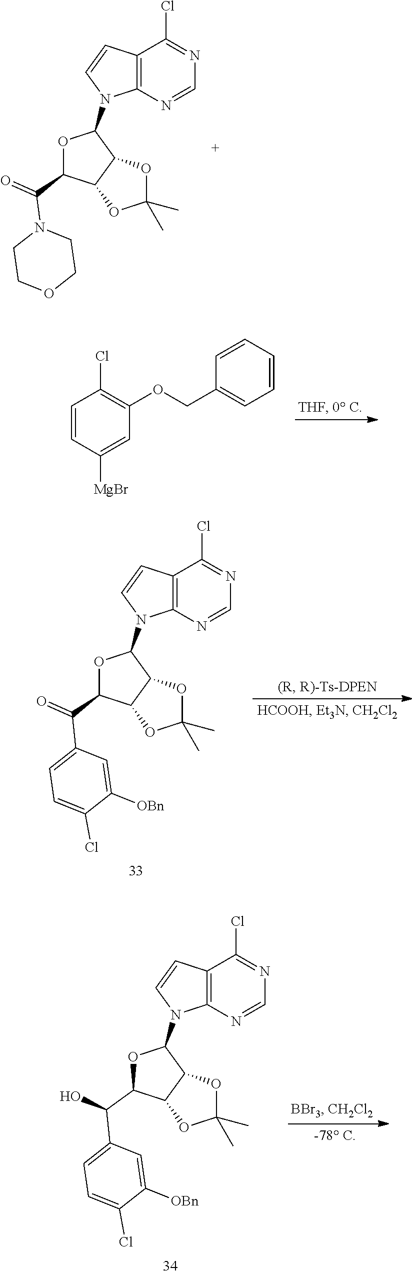

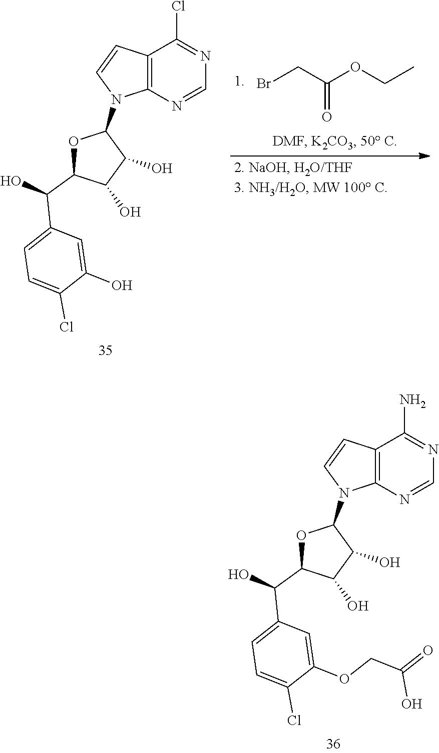

To the solution of ((3aS,4S,6R,6aR)-6-(4-chloro-7H-pyrrolo[2,3-d]pyrimidin-7-yl)-2,2-dimethyltetrahydrofuro[3,4-d][1,3]dioxol-4-yl)(morpholino)methanone (prepared according to ACS Med. Chem. Lett. 2018, 9, 612-617) (818 mg, 2 mmol) in THF (8 mL) at 0° C., was added (3-(benzyloxy)-4-chlorophenyl)magnesium bromide (4.5 mL, 1 M in THF, 4.5 mmol) dropwise during 10 min. The resulting mixture was stirred for 30 min at 0° C. before saturated NH₄Cl aqueous solution (2 mL) was added to quench the reaction. The mixture was extracted with ethyl acetate three times. The organic phase was dried over sodium sulfate, filtered and concentrated. The resulting residue was purified by flash chromatography on silica gel column with eluent (EtOAt/Hexanes, 0-50%) to afford compound 33 (900 mg, yield 83%) as colorless oil. MS (ESI) m/z 540.1 [M+H]⁺.

The stirring solution of compound 33 (900 mg, 1.67 mmol) in dichloromethane (10 mL) at −78° C. was degassed with nitrogen under reduced pressure for 30 min before HCOOH (92 mg, 2 mmol) and trimethylamine (202 mg, 2 mmol) were added. After the resulting solution was degassed for another 30 min, a solution of (R,R)-Ts-DPEN (53 mg, 0.08 mmol) in dichloromethane (1 mL) was added. After the resulting solution was degassed for another 10 min, the reaction was warmed to room temperature slowly and stirred overnight. The solution was concentrated and purified by flash chromatography on silica gel column with eluent (EtOAt/C₆H₁₄, 0-50%) to afford compound 34 (890 mg, yield 99%) as colorless oil. MS (ESI) m/z 542.2 [M+H]⁺.

To a solution of compound 34 (890 mg, 1.64 mmol) in dichloromethane (10 mL) was added BBr₃ (1.22 g, 4.9 mmol) dropwise at −78° C. The mixture was stirred at the temperature for 2 h, before saturated NH₄Cl aqueous solution (0.5 mL) was added to quench the reaction. The resulting mixture was concentrated and purified by reverse-phase flash chromatography on C18 column with eluent (CH₃CN/0.1% CF₃COOH in water, 5-100%) to afford compound 35 (667 mg, yield 92%) colorless oil. MS (ESI) m/z 412.2 [M+H]⁺.

To a mixture of compound 35 (667 mg, 1.61 mmol) and potassium carbonate (444 mg, 3.22 mmol) in dimethylformamide (8 mL) was added ethyl 2-bromoacetate (323 mg, 1.93 mmol). After the mixture was heated to 50° C. for 1 h, the reaction mixture was extracted with ethyl acetate three times. The organic phase was concentrated and the resulting residue was dissolved in THE (10 mL). To the solution was added a solution of sodium hydroxide (193 mg, 4.8 mmol) in water (2 mL). The resulting suspension was heated to 60° C. for 1 h before the mixture was concentrated. The resulting residue was dissolved into water (5 mL) and ammonia (1 mL, 28-30% aq. solution). The reaction solution was stirred in microwave reactor at 105° C. for 3 h. After cooling down to room temperature, the solution was concentrated and purified by reverse-phase flash chromatography on C18 column with eluent (CH₃CN/H₂O-0.1% CF₃COOH, 5-100%) to afford compound 36 (392 mg, yield 54%) as white solid. ¹H NMR (800 MHz, Methanol-d₄) δ 8.26 (s, 1H), 7.64 (d, J=3.9 Hz, 1H), 7.35 (d, J=8.1 Hz, 1H), 7.08 (s, 1H), 7.05 (d, J=8.2 Hz, 1H), 6.94 (d, J=3.9 Hz, 1H), 6.24 (d, J=6.6 Hz, 1H), 4.94-4.93 (m, 1H), 4.72 (s, 2H), 4.62 (t, J=5.8 Hz, 1H), 4.32-4.29 (m, 1H), 4.26 (d, J=3.5 Hz, 1H). MS (ESI) m/z 451.3 [M+H]⁺.

The following Examples 1-89 are directed to the synthesis of representative compounds according to the present disclosure:

### Example 1—Synthesis of YS31-58

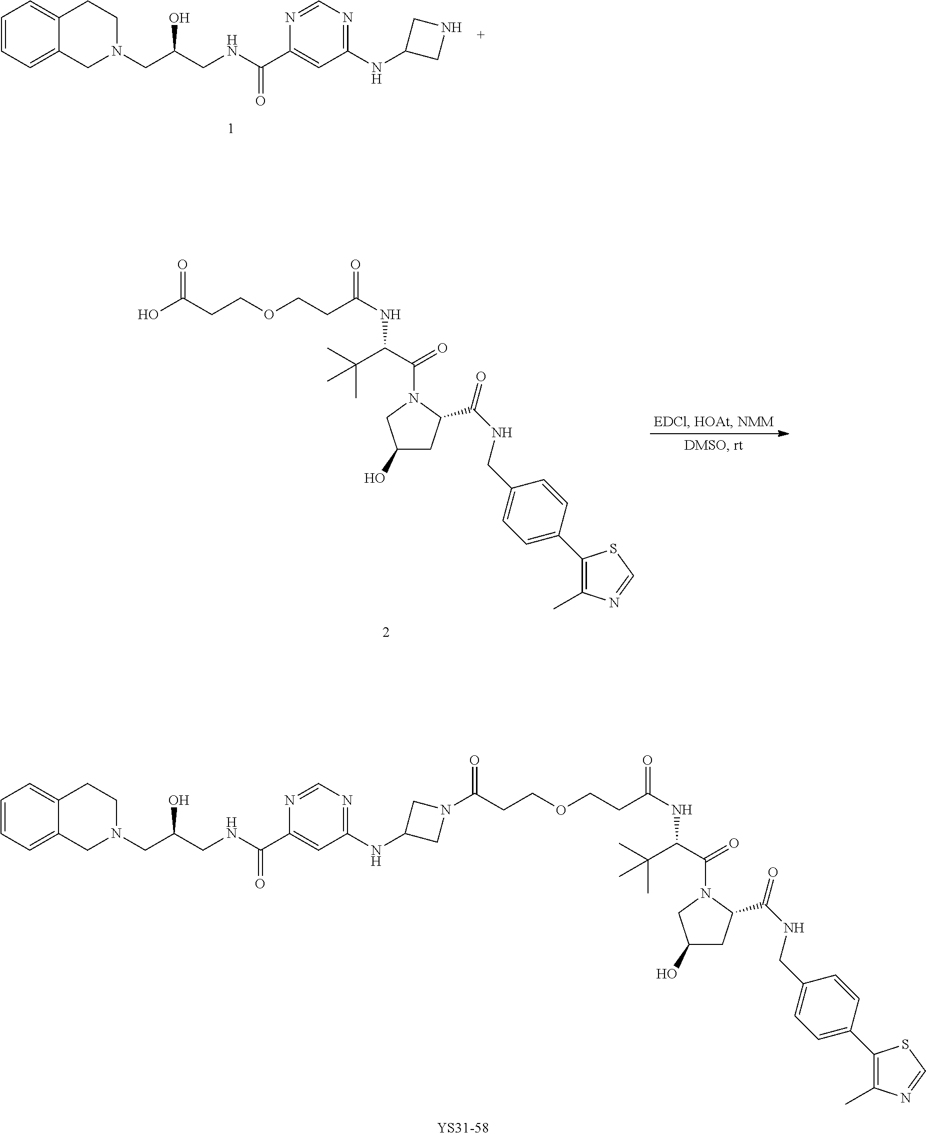 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (20 mg, 0.02 mmol), intermediate 2 (12 mg, 0.02 mmol, 1.0 equiv), EDCI (1-ethyl-3-(3- dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-58 as white solid in TFA salt form (8 mg, yield [?] 43%). ¹H NMR (600 MHz, Methanol-d₄) δ 8.92 (s, 1H), 8.51 (s, 1H), 7.54-7.39 (m, 4H), 7.32-7.06 (m, 5H), 4.79-4.73 (m, 1H), 4.68-4.55 (m, 5H), 4.52-4.46 (m, 2H), 4.44-4.27 (m, 5H), 4.18-4.09 (m, 1H), 3.90-3.79 (m, 4H), 3.71 (s, 5H), 3.55-3.49 (m, 2H), 3.40-3.35 (m, 2H), 2.46 (dt, J=5.7, 2.9 Hz, 7H), 2.25-2.21 (m, 1H), 2.09-2.04 (m, 1H), 1.03 (s, 9H). HRMS (m/z) for C₄₈H₆₃N₁₀O₈S⁺ [M+H]⁺: molecular weight calculated 939.4546, found 939.4532.

### Example 2—Synthesis of YS31-59

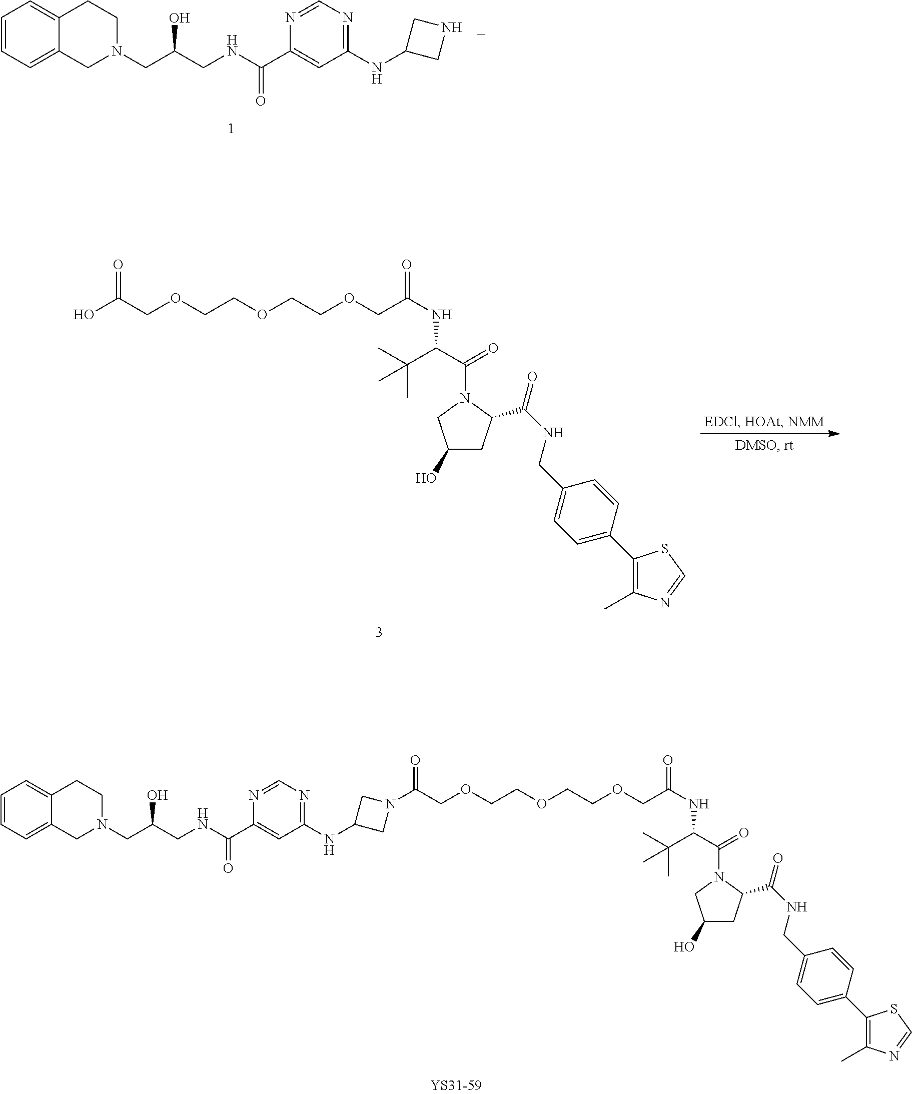 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)COCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (20 mg, 0.02 mmol), intermediate 3 (14 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-59 as white solid in TFA salt form (7 mg, yield 35%). ¹H NMR (600 MHz, Methanol-d₄) δ 8.91 (s, 1H), 8.52 (s, 1H), 7.48-7.42 (m, 4H), 7.34-7.18 (m, 5H), 4.81-4.66 (m, 5H), 4.65-4.49 (m, 7H), 4.41-4.32 (m, 5H), 4.22-4.21 (m, 1H), 4.06-3.86 (m, 2H), 3.97-3.63 (m, 13H), 3.55-3.50 (m, 2H), 2.48 (s, 3H), 2.26-2.23 (m, 1H), 2.10-2.07 (m, 1H), 1.04 (s, 9H). HRMS (m/z) for C₅₀H₆₇N₁₀O₁₀S⁺ [M+H]⁺: molecular weight calculated 999.4757, found 999.4763.

### Example 3—Synthesis of YS31-60

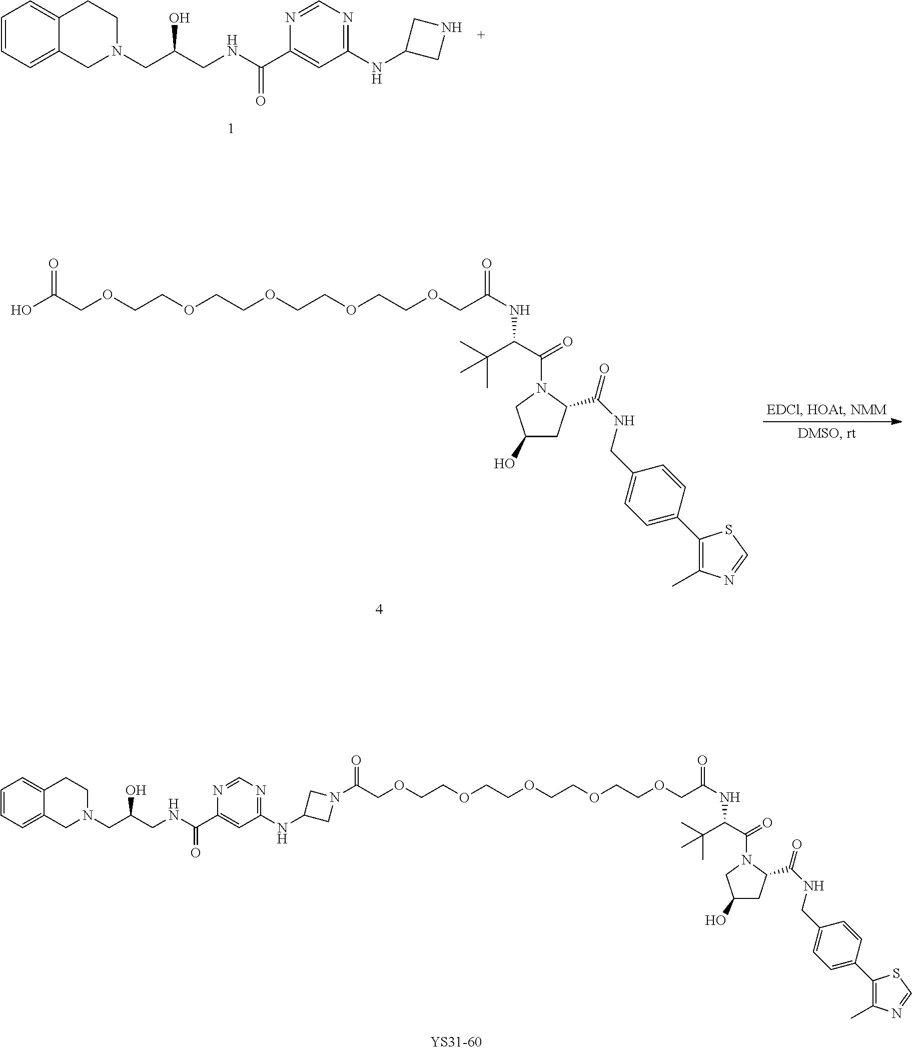 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCCOCCOCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (20 mg, 0.02 mmol), intermediate 4 (16 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-60 as white solid in TFA salt form (10 mg, yield 46%). ¹H NMR (600 MHz, Methanol-d₄) δ 8.90 (s, 1H), 8.52 (s, 1H), 7.48-7.41 (m, 4H), 7.35-7.16 (m, 5H), 4.81-4.66 (m, 6H), 4.62-4.46 (m, 6H), 4.42-4.32 (m, 5H), 4.25-4.21 (m, 1H), 4.10-4.02 (m, 4H), 3.89-3.67 (m, 4H), 3.70-3.43 (m, 17H), 2.47 (s, 3H), 2.26-2.21 (m, 1H), 2.08 (t, J=11.2 Hz, 1H), 1.03 (s, 9H). HRMS (m/z) for C₅₄H₇₅N₁₀O₁₂S⁺ [M+H]⁺: molecular weight calculated 1087.5281, found 1087.5261.

### Example 4—Synthesis of YS31-61

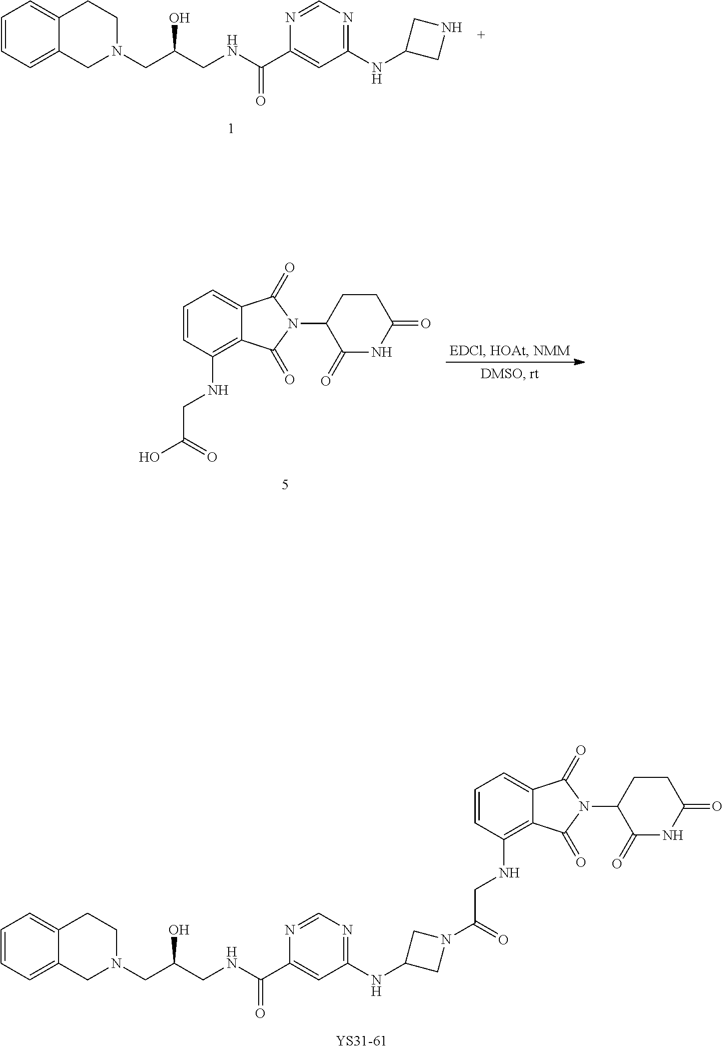 [SMILES: O=C(NC[C@H](O)CN1CCc2ccccc2C1)c1cc(NC2CNC2)ncn1 ; O=C(O)CNc1cccc2c1C(=O)N(C1CCC(=O)NC1=O)C2=O ; O=C1CCC(N2C(=O)c3cccc(NCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (20 mg, 0.02 mmol), intermediate 5 (7 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-61 as yellow solid in TFA salt form (9 mg, yield 64%). ¹H NMR (600 MHz, Methanol-d₄) δ 8.53 (s, 1H), 7.58 (t, J=7.8 Hz, 1H), 7.29 (dt, J=25.6, 7.8 Hz, 3H), 7.21-7.15 (m, 2H), 7.12 (d, J=7.0 Hz, 1H), 6.97 (d, J=8.6 Hz, 1H), 5.06 (dd, J=12.4, 5.6 Hz, 1H), 4.82-4.74 (m, 1H), 4.72-4.57 (m, 2H), 4.46-4.31 (m, 3H), 4.22-4.16 (m, 1H), 4.06 (s, 2H), 3.97 (dd, J=10.4, 5.2 Hz, 1H), 3.87-3.79 (m, 1H), 3.57-3.48 (m, 2H), 3.46-3.16 (m, 4H), 2.89-2.69 (m, 4H), 2.15-2.05 (m, 1H). HRMS (m/z) for C₃₅H₃₈N₉O₇⁺ [M+H]⁺: molecular weight calculated 696.2889, found 696.2887.

### Example 5—Synthesis of YS31-62

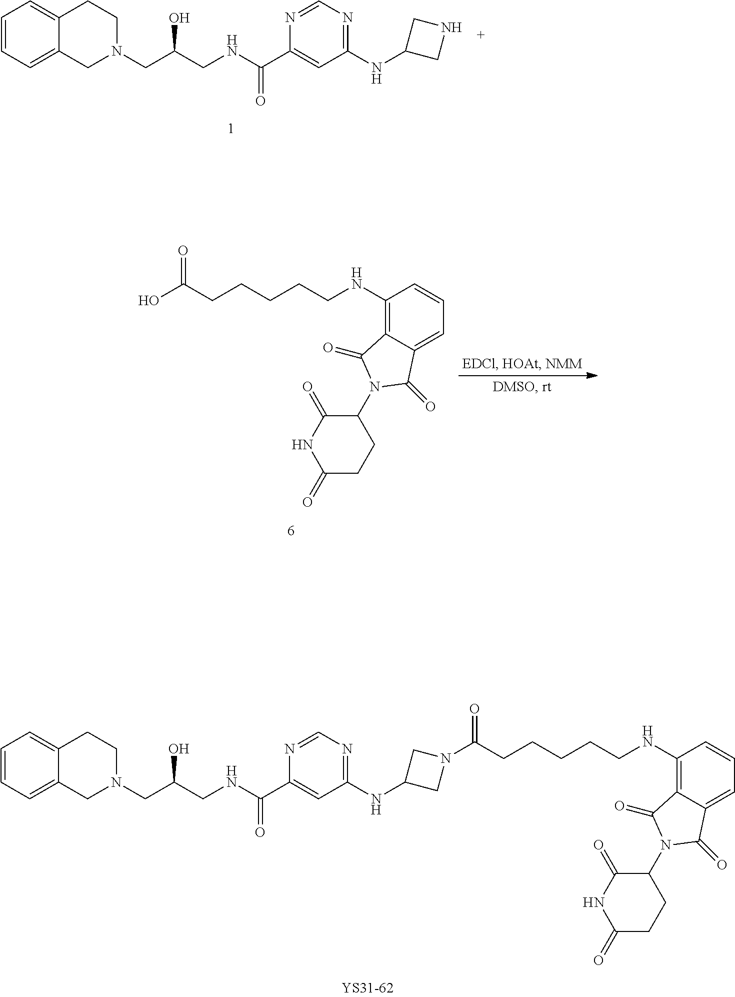 [SMILES: O=C(NC[C@H](O)CN1CCc2ccccc2C1)c1cc(NC2CNC2)ncn1 ; O=C(O)CCCCCNc1cccc2c1C(=O)N(C1CCC(=O)NC1=O)C2=O ; O=C1CCC(N2C(=O)c3cccc(NCCCCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (20 mg, 0.02 mmol), intermediate 6 (8 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-62 as yellow solid in TFA salt form (5 mg, yield 33%). ¹H NMR (600 MHz, Methanol-d₄) δ 8.52 (s, 1H), 7.55 (t, J=7.8 Hz, 1H), 7.34-7.27 (m, 3H), 7.19 (d, J=7.8 Hz, 2H), 7.04 (dd, J=17.4, 7.8 Hz, 2H), 5.04 (dd, J=12.4, 4.9, 2.4, 1.2 Hz, 1H), 4.79-4.70 (m, 1H), 4.64-4.52 (m, 2H), 4.45-4.33 (m, 3H), 4.07 (s, 1H), 3.88 (dd, J=10.4, 5.2 Hz, 2H), 3.53 (q, J=6.8, 6.4 Hz, 2H), 3.31 (dq, J=3.3, 1.6 Hz, 6H), 2.87-2.63 (m, 4H), 2.19 (t, J=7.3 Hz, 2H), 2.11-2.05 (m, 1H), 1.67 (dt, J=15.0, 9.0 Hz, 4H), 1.47 (q, J=8.0 Hz, 2H). HRMS (m/z) for C₃₉H₄₆N₉O₇⁺ [M+H]⁺: molecular weight calculated 752.3515, found 752.3515.

### Example 6—Synthesis of YS31-63

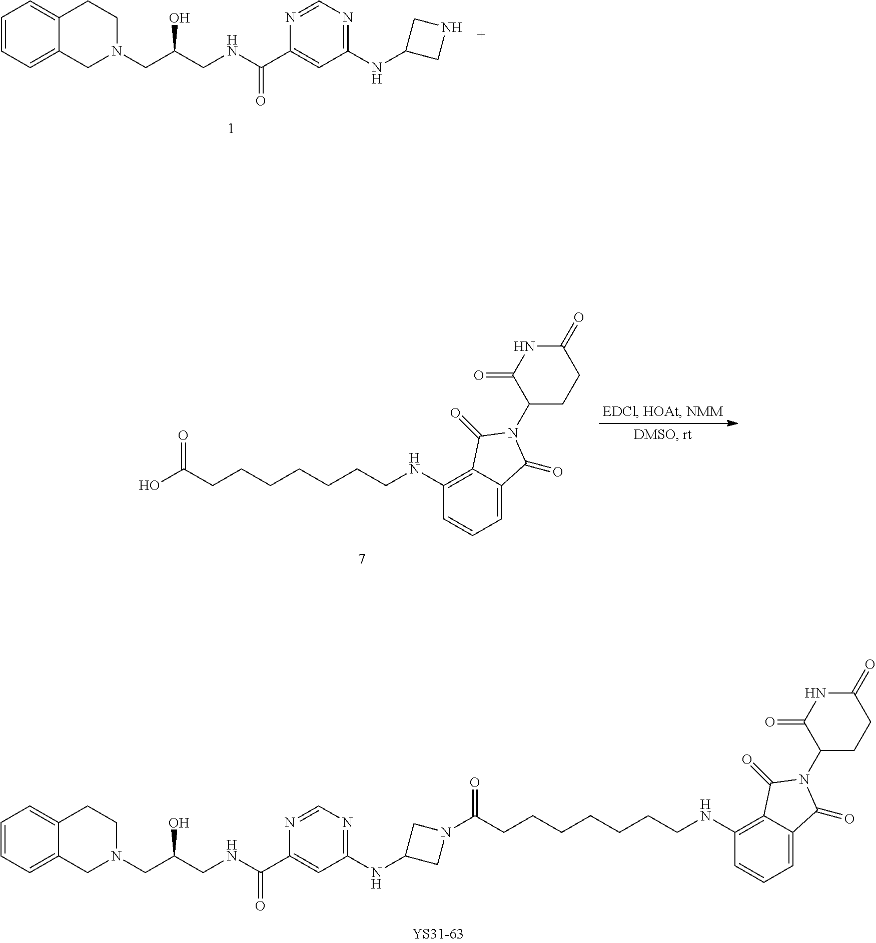 [SMILES: O=C(NC[C@H](O)CN1CCc2ccccc2C1)c1cc(NC2CNC2)ncn1 ; O=C(O)CCCCCCCNc1cccc2c1C(=O)N(C1CCC(=O)NC1=O)C2=O ; O=C1CCC(N2C(=O)c3cccc(NCCCCCCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (20 mg, 0.02 mmol), intermediate 7 (10 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-63 as yellow solid in TFA salt form (7 mg, yield 44%). ¹H NMR (600 MHz, Methanol-d₄) δ 8.52 (s, 1H), 7.54 (t, J=7.8 Hz, 1H), 7.33-7.25 (m, 3H), 7.23-7.15 (m, 2H), 7.03 (t, J=8.1 Hz, 2H), 5.04 (dd, J=12.6, 5.5 Hz, 1H), 4.79-4.70 (m, 1H), 4.66-4.52 (m, 2H), 4.45-4.29 (m, 3H), 4.10-4.02 (m, 1H), 3.89-3.81 (m, 2H), 3.57-3.47 (m, 2H), 3.43-3.14 (m, 6H), 2.88-2.64 (m, 4H), 2.20-2.07 (m, 3H), 1.64 (dp, J=39.4, 7.1 Hz, 4H), 1.49-1.33 (m, 6H). HRMS (m/z) for C₄₁H₅₀N₉O₇⁺ [M+H]⁺: molecular weight calculated 780.3828, found 780.3831.

### Example 7—Synthesis of YS31-64

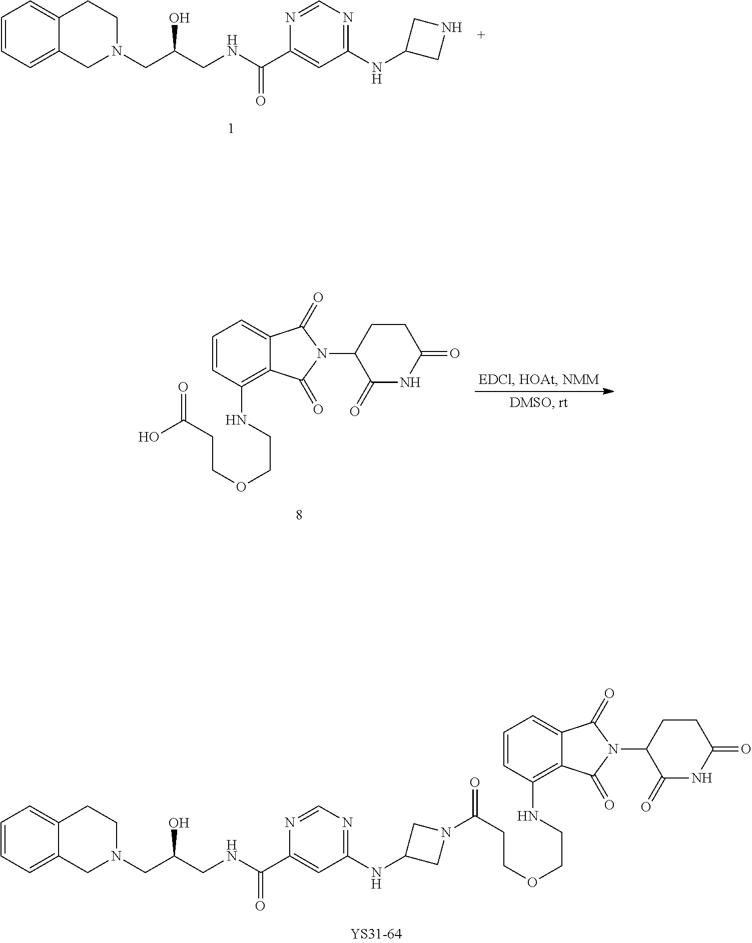 [SMILES: O=C(NC[C@H](O)CN1CCc2ccccc2C1)c1cc(NC2CNC2)ncn1 ; O=C(O)CCOCCNc1cccc2c1C(=O)N(C1CCC(=O)NC1=O)C2=O ; O=C1CCC(N2C(=O)c3cccc(NCCOCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (20 mg, 0.02 mmol), intermediate 8 (8 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-64 as yellow solid in TFA salt form (7 mg, yield 47%). ¹H NMR (600 MHz, Methanol-d₄) δ 8.49 (s, 1H), 7.59-7.47 (m, 1H), 7.35-7.16 (m, 4H), 7.14-6.99 (m, 3H), 5.11-5.00 (m, 1H), 4.77-4.68 (m, 1H), 4.68-4.58 (m, 2H), 4.38 (d, J=37.8 Hz, 3H), 4.17-4.10 (m, 1H), 3.92-3.82 (m, 2H), 3.76 (dt, J=12.0, 5.9 Hz, 3H), 3.69 (q, J=4.7 Hz, 3H), 3.55-3.52 (m, 2H), 3.50 (t, J=5.2 Hz, 2H), 3.42-3.33 (m, 2H), 2.90-2.56 (m, 4H), 2.46-2.37 (m, 2H), 2.13-2.02 (m, 1H). HRMS (m/z) for C₃₈H₄₄N₉O₈⁺ [M+H]⁺: molecular weight calculated 754.3307, found 754.3324.

### Example 8—Synthesis of YS31-65

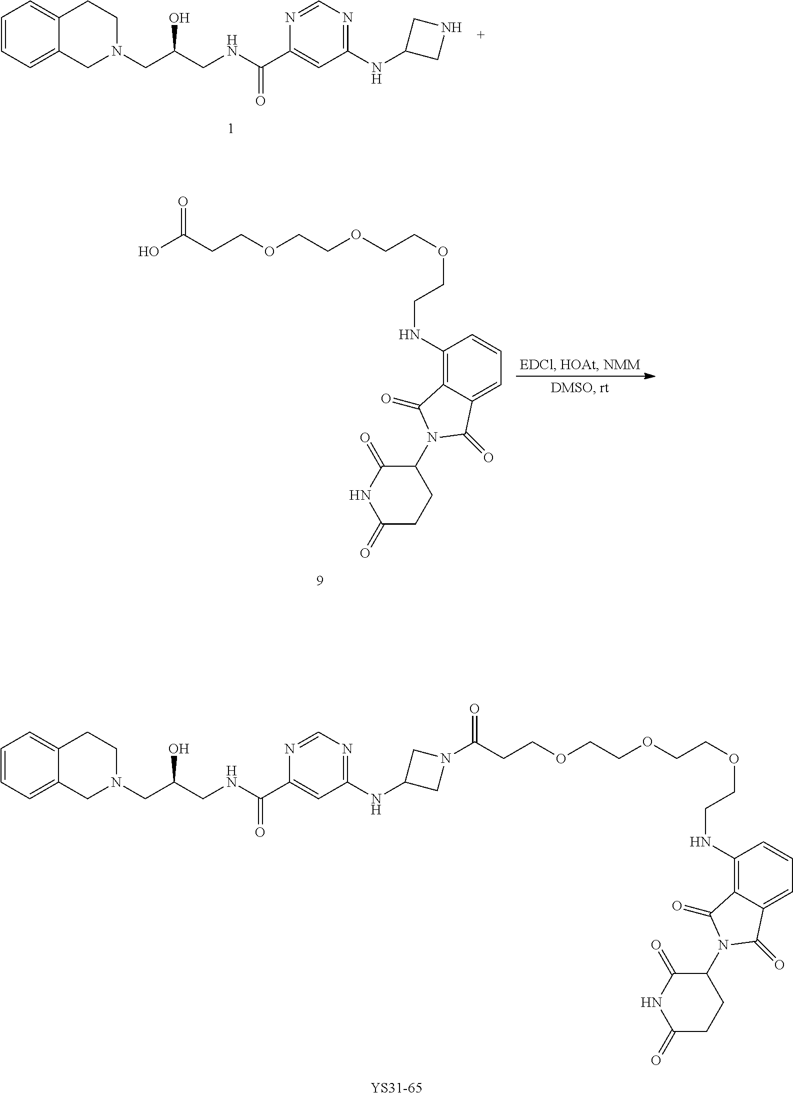 [SMILES: O=C(NC[C@H](O)CN1CCc2ccccc2C1)c1cc(NC2CNC2)ncn1 ; O=C(O)CCOCCOCCOCCNc1cccc2c1C(=O)N(C1CCC(=O)NC1=O)C2=O ; O=C1CCC(N2C(=O)c3cccc(NCCOCCOCCOCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (20 mg, 0.02 mmol), intermediate 9 (10 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-65 as yellow solid in TFA salt form (9 mg, yield 56%). ¹H NMR (600 MHz, Methanol-d₄) δ 8.48 (s, 1H), 7.51 (t, J=7.8 Hz, 1H), 7.29 (dt, J=25.8, 7.6 Hz, 3H), 7.19 (d, J=7.6 Hz, 1H), 7.10 (d, J=9.1 Hz, 1H), 7.03 (dd, J=26.4, 7.8 Hz, 2H), 5.04 (dd, J=12.8, 5.4 Hz, 1H), 4.73-4.70 (m, 1H), 4.64-4.58 (m, 2H), 4.45-4.30 (m, 3H), 4.14-4.09 (m, 1H), 3.88-3.82 (m, 2H), 3.71 (t, J=4.8 Hz, 6H), 3.67-3.64 (m, 4H), 3.64-3.61 (m, 2H), 3.58 (dd, J=5.4, 3.1 Hz, 2H), 3.52 (q, J=6.7, 6.1 Hz, 2H), 3.48 (t, J=5.2 Hz, 2H), 3.40-3.33 (m, 2H), 2.89-2.80 (m, 1H), 2.77-2.65 (m, 3H), 2.37 (q, J=5.8 Hz, 2H), 2.13-2.08 (m, 1H). HRMS (m/z) for C₄₂H₅₂N₉O₁₀⁺ [M+H]⁺: molecular weight calculated 842.3832, found 842.3831.

### Example 9—Synthesis of YS31-66

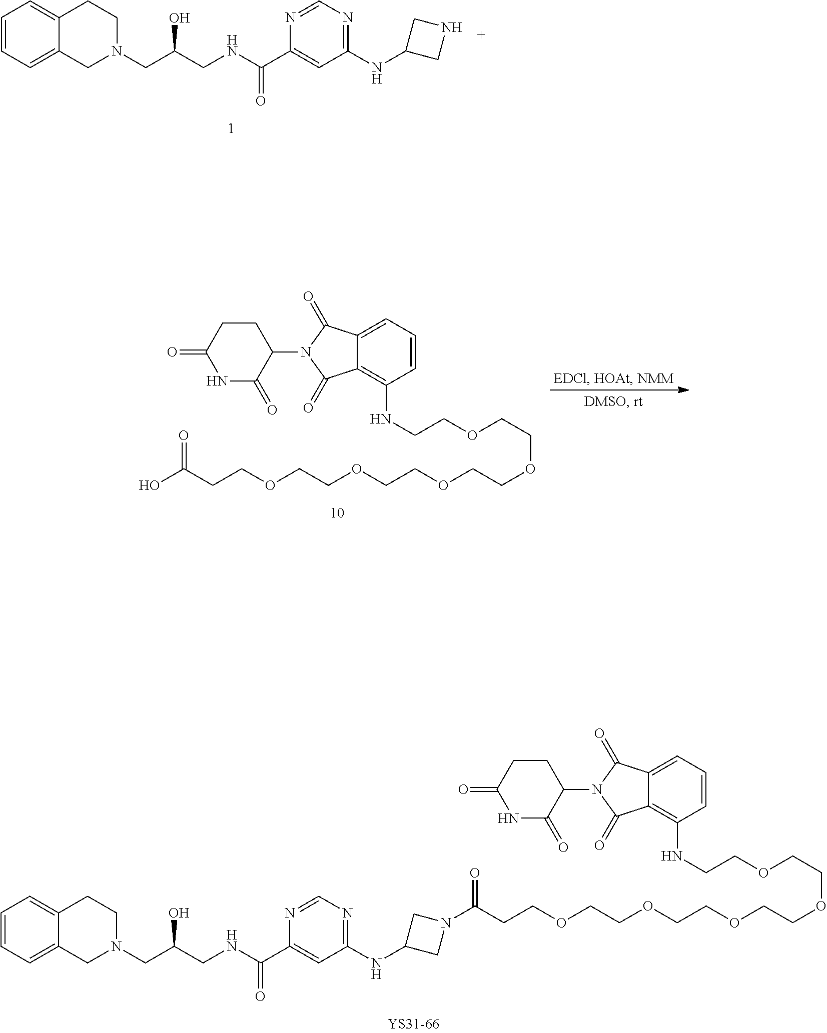 [SMILES: O=C(NC[C@H](O)CN1CCc2ccccc2C1)c1cc(NC2CNC2)ncn1 ; O=C(O)CCOCCOCCOCCOCCOCCNc1cccc2c1C(=O)N(C1CCC(=O)NC1=O)C2=O ; O=C1CCC(N2C(=O)c3cccc(NCCOCCOCCOCCOCCOCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (20 mg, 0.02 mmol), intermediate 10 (12 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-66 as yellow solid in TFA salt form (8 mg, yield 44%). HRMS (m/z) for C₄₆H₆₀N₉O₁₂⁺ [M+H]⁺: molecular weight calculated 930.4356, found 930.4361.

### Example 10—Synthesis of YS31-67

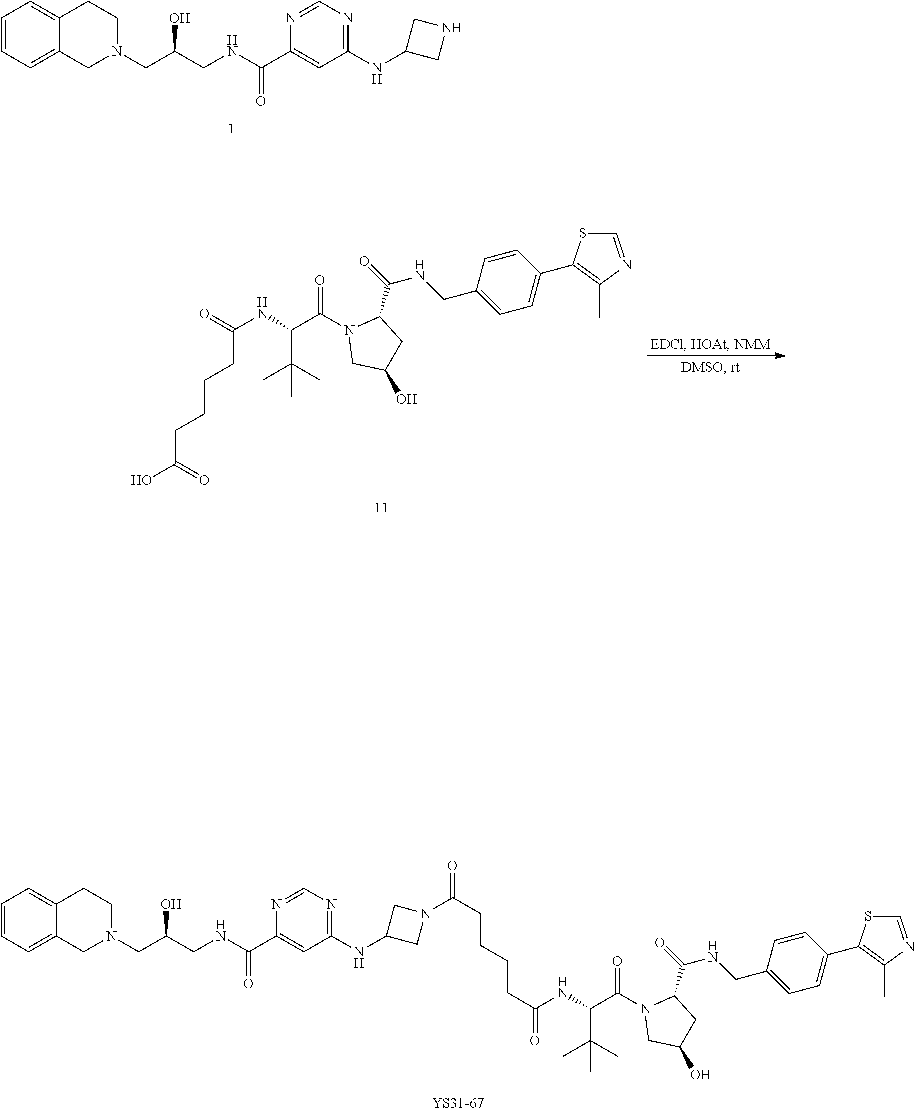 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCCCC(=O)O)C(C)(C)C)cc1 ; Cc1ncsc1-c1ccc(CNC(=O)[C@@H]2C[C@@H](O)CN2C(=O)[C@@H](NC(=O)CCCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (20 mg, 0.02 mmol), intermediate 11 (12 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-67 as white solid in TFA salt form (10 mg, yield 56%). ¹H NMR (600 MHz, Methanol-d₄) δ 8.91 (s, 1H), 8.52 (s, 1H), 7.58-7.38 (m, 4H), 7.40-7.24 (m, 3H), 7.19 (d, J=9.3 Hz, 2H), 4.76 (dddt, J=7.4, 3.7, 2.5, 1.2 Hz, 1H), 4.65-4.47 (m, 7H), 4.44-4.28 (m, 5H), 4.09 (s, 1H), 3.92-3.77 (m, 5H), 3.53 (dt, J=13.8, 6.3 Hz, 2H), 3.38-3.32 (m, 2H), 2.49-2.40 (m, 3H), 2.31 (dd, J=13.9, 6.6 Hz, 2H), 2.24-2.16 (m, 3H), 2.09 (d, J=13.5 Hz, 1H), 1.62 (s, 4H), 1.03 (d, J=2.5 Hz, 9H). HRMS (m/z) for C₄₆H₆₃N₁₀O₇S⁺ [M+H]⁺: molecular weight calculated 923.4596, found 923.4596.

### Example 11—Synthesis of YS31-68

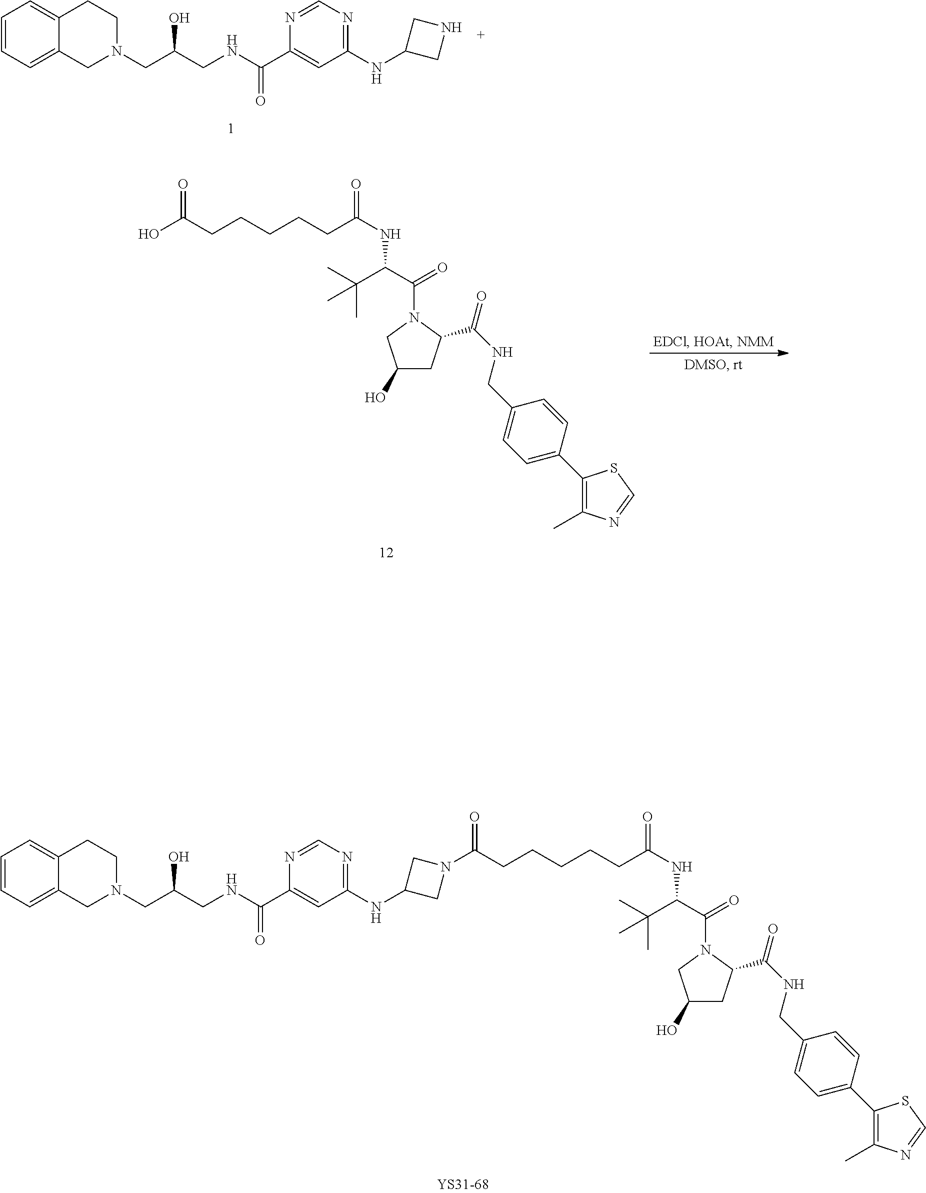 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (20 mg, 0.02 mmol), intermediate 12 (14 mg, 0.02 mmol, 1.0 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-68 as white solid in TFA salt form (9 mg, yield 48%). ¹H NMR (600 MHz, Methanol-d₄) δ 8.93 (s, 1H), 8.53 (s, 1H), 7.48-7.40 (m, 4H), 7.33-7.17 (m, 5H), 4.79-4.74 (m, 1H), 4.64-4.50 (m, 7H), 4.35 (d, J=15.4 Hz, 5H), 4.09 (s, 1H), 3.91-3.79 (m, 5H), 3.54-3.50 (m, 2H), 3.40-3.33 (m, 2H), 2.47 (d, J=1.4 Hz, 3H), 2.28-2.10 (m, 6H), 1.63-1.59 (m, 4H), 1.37-1.33 (m, 2H), 1.03 (s, 9H). HRMS (m/z) for C₄₉H₆₅N₁₀O₇S⁺ [M+H]⁺: molecular weight calculated 937.4753, found 937.4770.

### Example 12—Synthesis of YS31-69

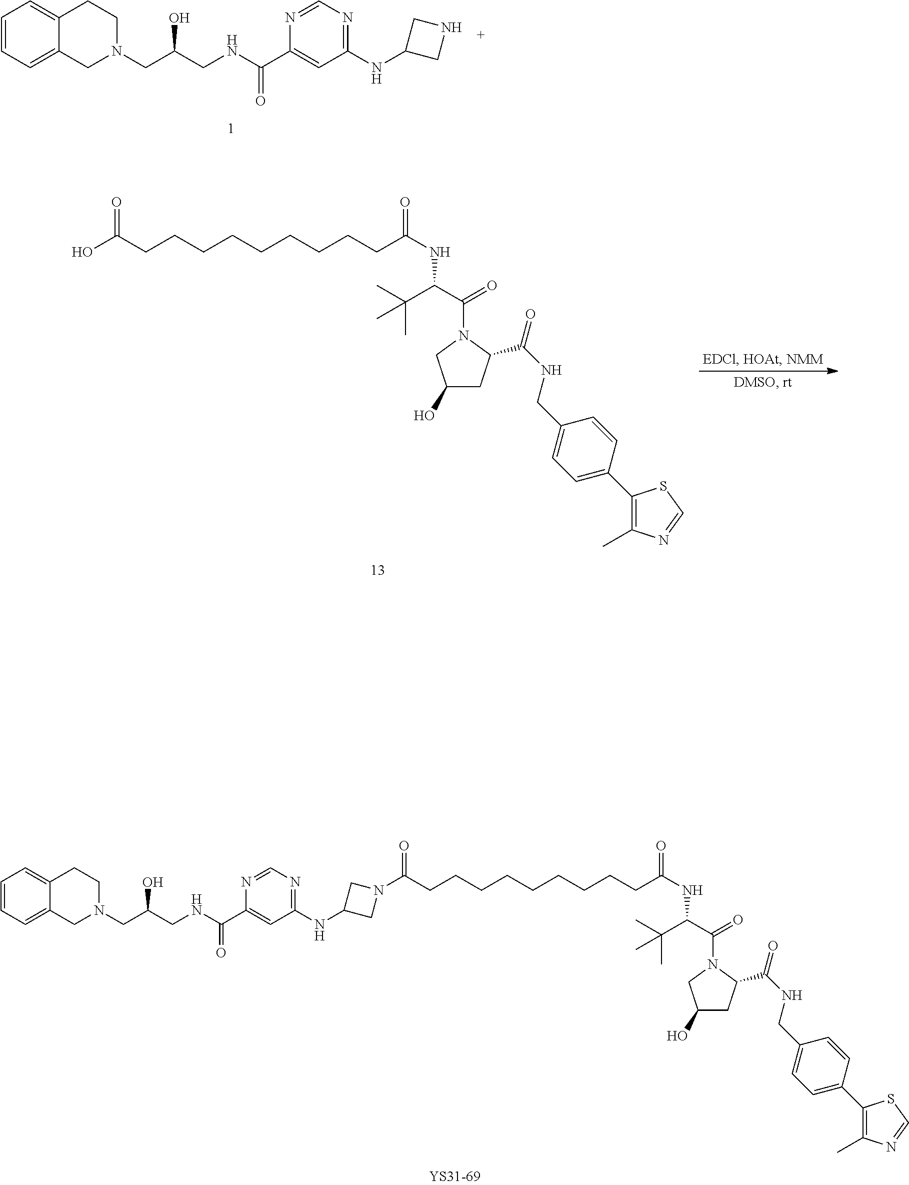 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (20 mg, 0.02 mmol), intermediate 13 (16 mg, 0.03 mmol, 1.5 equiv), EDCI (1-ethyl-3-(3-dimethylaminopropyl)carbodiimide) (6 mg, 0.03 mmol, 1.5 equiv), and HOAt (1-hydroxy-7-azabenzo-triazole) (4 mg, 0.03 mmol, 1.5 equiv) in 1 mL of DMSO, was added NMM (N-Methylmorpholine) (30 mg, 0.30 mmol, 15 equiv). After stirring overnight at room temperature, the resulting mixture was purified by preparative HPLC (10%-100% methanol/0.1% TFA in H₂O) to afford YS31-69 as white solid in TFA salt form (7 mg, yield 35%). ¹H NMR (600 MHz, Methanol-d₄) δ 8.92 (s, 1H), 8.53 (s, 1H), 7.49-7.39 (m, 4H), 7.34-7.18 (m, 5H), 4.79-4.73 (m, 1H), 4.65-4.48 (m, 7H), 4.43-4.32 (m, 5H), 4.09-4.05 (m, 1H), 3.92-3.79 (m, 5H), 3.55-3.50 (m, 2H), 3.42-3.37 (m, 2H), 2.47 (s, 3H), 2.26-2.08 (m, 6H), 1.59 (s, 4H), 1.32 (s, 10H), 1.03 (s, 9H). HRMS (m/z) for C₅₃H₇₃N₁₀O₇S⁺ [M+H]⁺: molecular weight calculated 993.5379, found 993.5378.

### Example 13—Synthesis of YS43-6

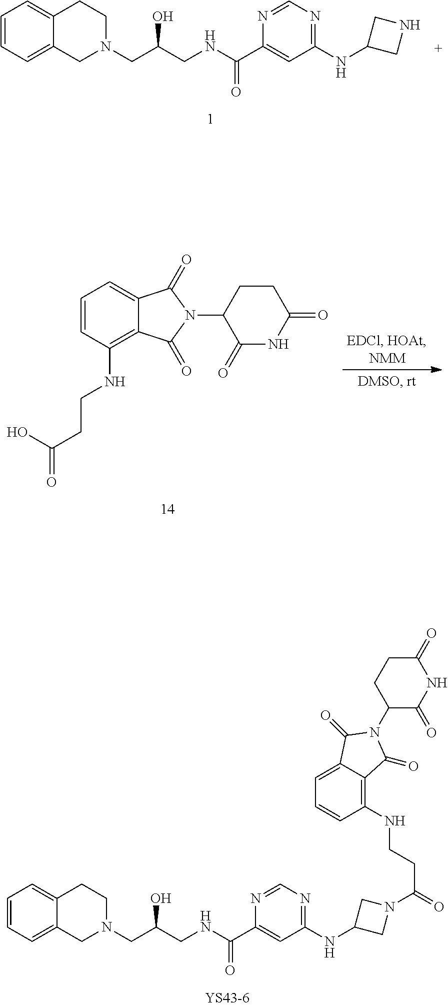 [SMILES: O=C(NC[C@H](O)CN1CCc2ccccc2C1)c1cc(NC2CNC2)ncn1 ; O=C(O)CCNc1cccc2c1C(=O)N(C1CCC(=O)NC1=O)C2=O ; O=C1CCC(N2C(=O)c3cccc(NCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (10 mg, 0.01 mmol), intermediate 14 (10 mg, 0.03 mmol, 3 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-6 as yellow solid in TFA salt form (6 mg, yield 86%). ¹H NMR (600 MHz, Methanol-d₄) δ 8.49 (d, J=1.6 Hz, 1H), 7.56 (dtd, J=8.5, 4.6, 2.3 Hz, 1H), 7.28 (dt, J=24.4, 7.6 Hz, 3H), 7.19 (d, J=7.6 Hz, 1H), 7.16-7.08 (m, 2H), 7.07-6.96 (m, 1H), 5.08-5.01 (m, 1H), 4.62 (d, J=18.1 Hz, 2H), 4.52 (q, J=7.6, 7.0 Hz, 1H), 4.42-4.29 (m, 3H), 3.98-3.81 (m, 3H), 3.63 (dt, J=25.5, 6.0 Hz, 3H), 3.59-3.47 (m, 3H), 3.44-3.30 (m, 2H), 2.90-2.83 (m, 1H), 2.78-2.68 (m, 3H), 2.49 (d, J=53.0 Hz, 2H), 2.10 (ddd, J=9.7, 5.1, 2.7 Hz, 1H). HRMS (m/z) for C₃₆H₄₀N₉O₇⁺ [M+H]⁺: molecular weight calculated 710.3045, found 710.3050.

### Example 14—Synthesis of YS43-7

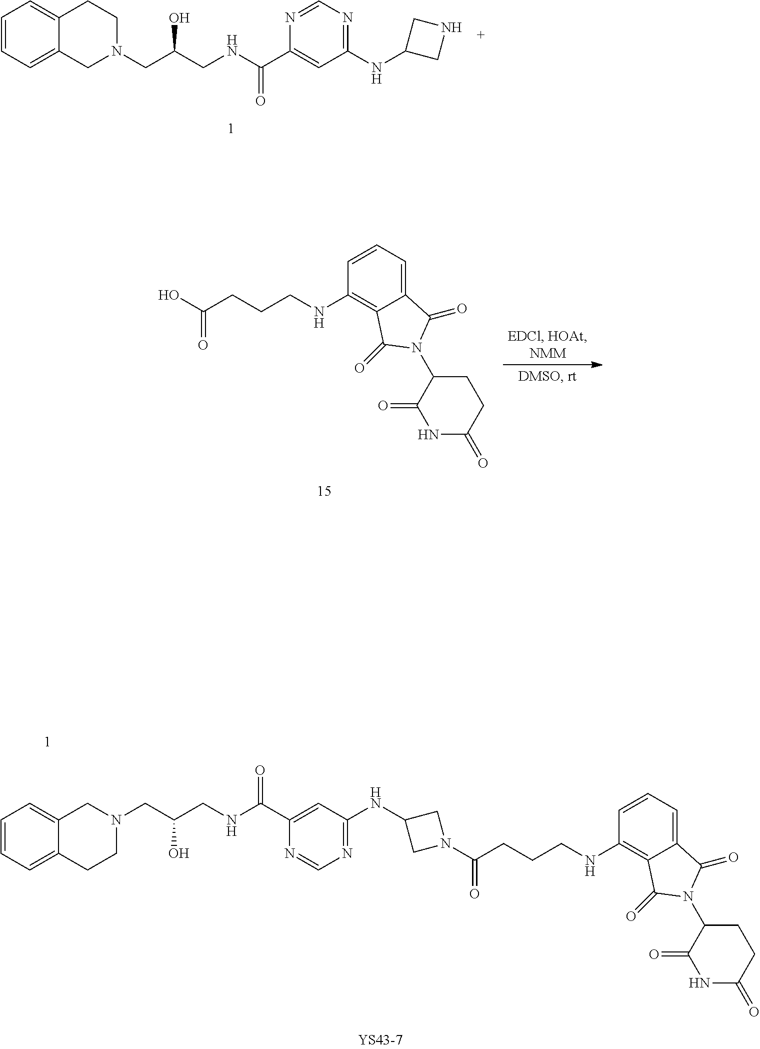 [SMILES: O=C(NC[C@H](O)CN1CCc2ccccc2C1)c1cc(NC2CNC2)ncn1 ; O=C(O)CCCNc1cccc2c1C(=O)N(C1CCC(=O)NC1=O)C2=O ; O=C1CCC(N2C(=O)c3cccc(NCCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (10 mg, 0.01 mmol), intermediate 15 (10 mg, 0.03 mmol, 3 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-7 as yellow solid in TFA salt form (5 mg, yield 71%). ¹H NMR (600 MHz, Methanol-d₄) δ 8.51 (s, 1H), 7.54 (ddd, J=8.6, 7.0, 1.7 Hz, 1H), 7.32-7.25 (m, 3H), 7.21-7.13 (m, 2H), 7.08 (d, J=8.6 Hz, 1H), 7.03-7.00 (m, 1H), 5.06-5.01 (m, 1H), 4.72-4.50 (m, 3H), 4.48-4.29 (m, 3H), 4.05 (s, 1H), 3.86 (td, J=9.9, 5.0 Hz, 2H), 3.59-3.49 (m, 3H), 3.40 (d, J=5.6 Hz, 3H), 3.29-3.11 (m, 2H), 2.84 (ddd, J=18.6, 13.9, 5.4 Hz, 1H), 2.77-2.67 (m, 3H), 2.31-2.21 (m, 2H), 2.11-2.06 (m, 1H), 2.02-1.93 (m, 2H). HRMS (m/z) for C₃₇H₄₂N₉O₇⁺ [M+H]⁺: molecular weight calculated 724.3202, found 724.3201.

### Example 15—Synthesis of YS43-8

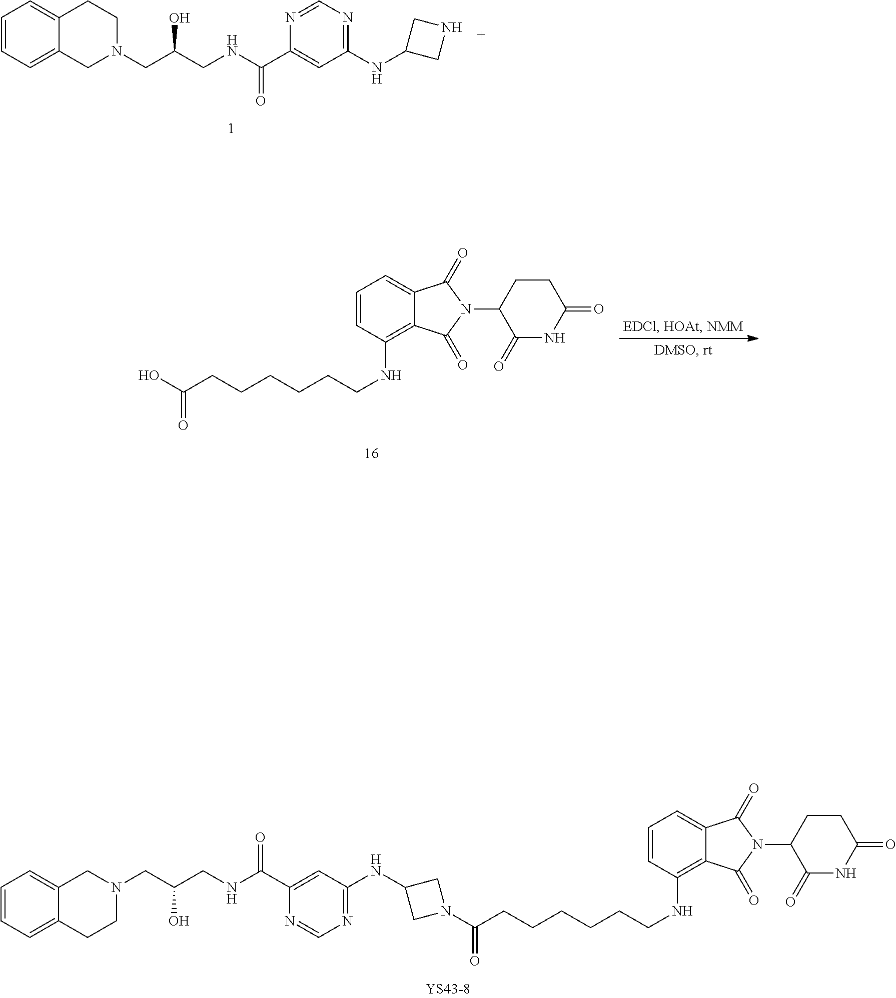 [SMILES: O=C(NC[C@H](O)CN1CCc2ccccc2C1)c1cc(NC2CNC2)ncn1 ; O=C(O)CCCCCCNc1cccc2c1C(=O)N(C1CCC(=O)NC1=O)C2=O ; O=C1CCC(N2C(=O)c3cccc(NCCCCCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (10 mg, 0.01 mmol), intermediate 16 (10 mg, 0.03 mmol, 3 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-8 as yellow solid in TFA salt form (4 mg, yield 53%). ¹H NMR (600 MHz, Methanol-d₄) δ 8.53 (s, 1H), 7.54 (dd, J=8.6, 7.1 Hz, 1H), 7.40-7.14 (m, 5H), 7.03 (dd, J=17.0, 7.8 Hz, 2H), 5.03 (dd, J=11.0, 4.9, 2.5, 1.2 Hz, 1H), 4.80-4.74 (m, 1H), 4.65-4.52 (m, 2H), 4.45-4.29 (m, 3H), 4.09-4.04 (m, 1H), 3.89-3.79 (m, 2H), 3.56-3.48 (m, 2H), 3.42-3.12 (m, 6H), 2.88-2.66 (m, 4H), 2.20-2.05 (m, 3H), 1.65 (dp, J=29.7, 7.1 Hz, 4H), 1.44 (dq, J=22.4, 7.8, 7.2 Hz, 4H). HRMS (m/z) for C₄₀H₄₈N₉O₇⁺ [M+H]⁺: molecular weight calculated 766.3671, found 766.3681.

### Example 16—Synthesis of YS43-9

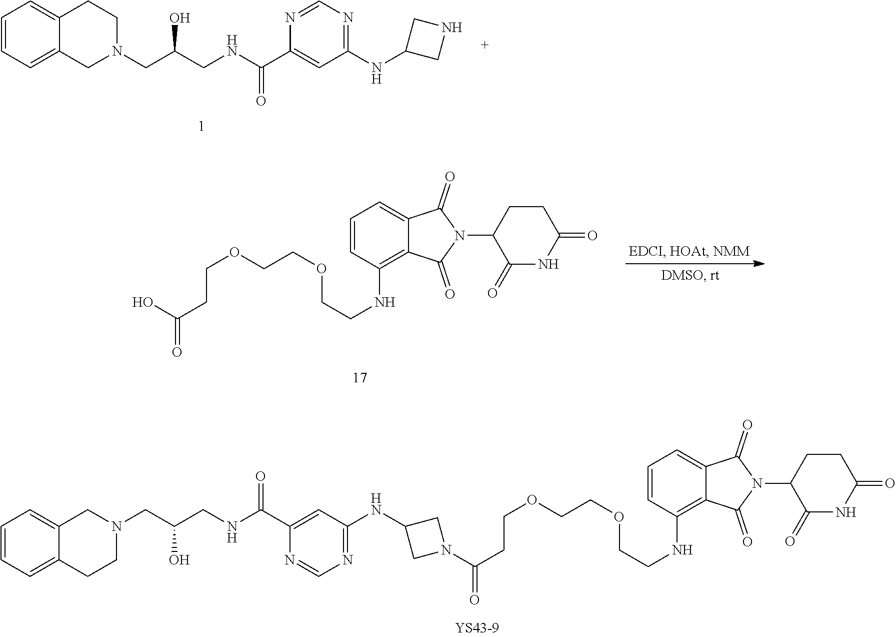 [SMILES: O=C(NC[C@H](O)CN1CCc2ccccc2C1)c1cc(NC2CNC2)ncn1 ; O=C(O)CCOCCOCCNc1cccc2c1C(=O)N(C1CCC(=O)NC1=O)C2=O ; O=C1CCC(N2C(=O)c3cccc(NCCOCCOCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (10 mg, 0.01 mmol), intermediate 17 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-9 as yellow solid in TFA salt form (4 mg, yield 50%). ¹H NMR (600 MHz, Methanol-d₄) δ 8.45 (s, 1H), 7.58-7.46 (m, 1H), 7.32-7.17 (m, 4H), 7.10-6.94 (m, 3H), 5.06-5.01 (m, 1H), 4.72-4.56 (m, 3H), 4.47-4.26 (m, 3H), 4.09 (s, 1H), 3.87-3.81 (m, 2H), 3.75-3.69 (m, 5H), 3.67-3.61 (m, 5H), 3.54-3.46 (m, 4H), 3.42-3.33 (m, 2H), 2.89-2.79 (m, 1H), 2.78-2.64 (m, 3H), 2.54 (t, J=6.3 Hz, 1H), 2.45-2.30 (m, 1H), 2.13-2.07 (m, 1H). HRMS (m/z) for C₄₀H₄₈N₉O₉⁺ [M+H]⁺: molecular weight calculated 798.3570, found 798.3551.

### Example 17—Synthesis of YS43-10

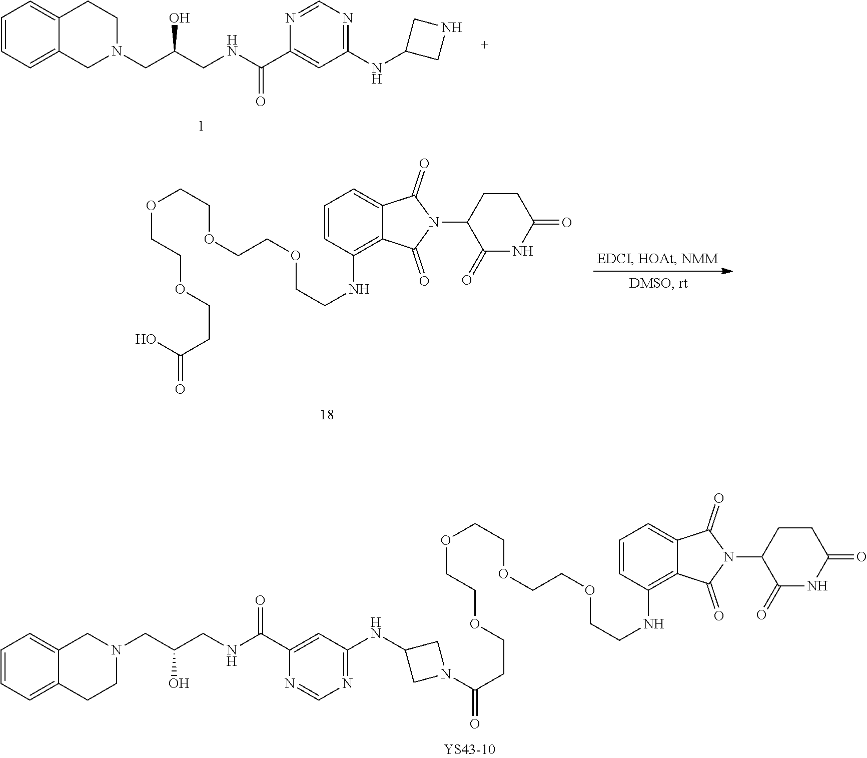 [SMILES: O=C(NC[C@H](O)CN1CCc2ccccc2C1)c1cc(NC2CNC2)ncn1 ; O=C(O)CCOCCOCCOCCOCCNc1cccc2c1C(=O)N(C1CCC(=O)NC1=O)C2=O ; O=C1CCC(N2C(=O)c3cccc(NCCOCCOCCOCCOCCC(=O)N4CC(Nc5cc(C(=O)NC[C@H](O)CN6CCc7ccccc7C6)ncn5)C4)c3C2=O)C(=O)N1]

To a solution of Intermediate 1 in TFA salt form (10 mg, 0.01 mmol), intermediate 18 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-10 as yellow solid in TFA salt form (8 mg, yield 91%). ¹H NMR (600 MHz, Methanol-d₄) δ 8.52 (s, 1H), 7.54-7.51 (m, 1H), 7.31-7.24 (m, 3H), 7.18 (d, J=7.7 Hz, 1H), 7.07-7.01 (m, 3H), 5.04 (dd, J=12.9, 5.5 Hz, 1H), 4.78-4.70 (m, 1H), 4.62 (t, J=8.5 Hz, 2H), 4.45-4.31 (m, 3H), 4.18-4.12 (m, 1H), 3.91-3.81 (m, 2H), 3.72-3.70 (m, 4H), 3.64 (d, J=16.1 Hz, 7H), 3.61-3.58 (m, 5H), 3.57-3.55 (m, 2H), 3.54-3.50 (m, 2H), 3.48 (q, J=5.3 Hz, 2H), 3.41-3.31 (m, 2H), 2.85 (ddd, J=17.5, 14.0, 5.3 Hz, 1H), 2.76-2.59 (m, 3H), 2.40-2.35 (m, 2H), 2.13-2.07 (m, 1H). HRMS (m/z) for C₄₄H₅₆N₉O₁₁⁺ [M+H]⁺: molecular weight calculated 886.4094, found 886.4088.

### Example 18—Synthesis of YS43-11

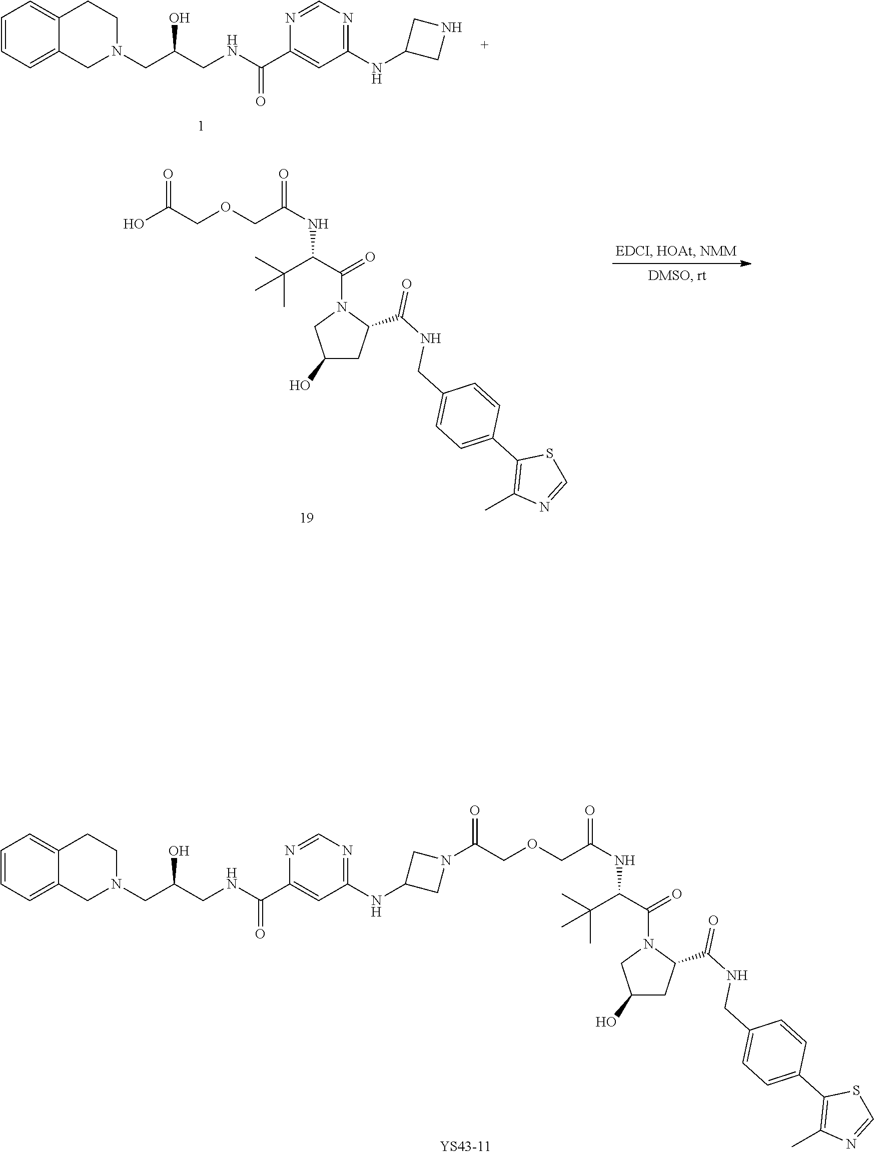 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)COCC(=O)O)C(C)(C)C)cc1 ; Cc1ncsc1-c1ccc(CNC(=O)[C@@H]2C[C@@H](O)CN2C(=O)[C@@H](NC(=O)COCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 19 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-11 as white solid in TFA salt form (6 mg, yield 67%). ¹H NMR (600 MHz, Methanol-d₄) δ 8.96 (s, 1H), 8.52 (s, 1H), 7.48-7.41 (m, 4H), 7.34-7.16 (m, 5H), 4.71-4.47 (m, 4H), 4.44-4.33 (m, 4H), 4.25-4.05 (m, 9H), 3.98-3.95 (m, 1H), 3.91-3.76 (m, 5H), 3.57-3.49 (m, 2H), 3.44-3.35 (m, 2H), 2.47 (s, 3H), 2.26-2.21 (m, 1H), 2.12-2.06 (m, 1H), 1.04 (s, 9H). HRMS (m/z) for C₄₆H₅₉N₁₀O₈S⁺ [M+H]⁺: molecular weight calculated 911.4233, found 911.4233.

### Example 19—Synthesis of YS43-12

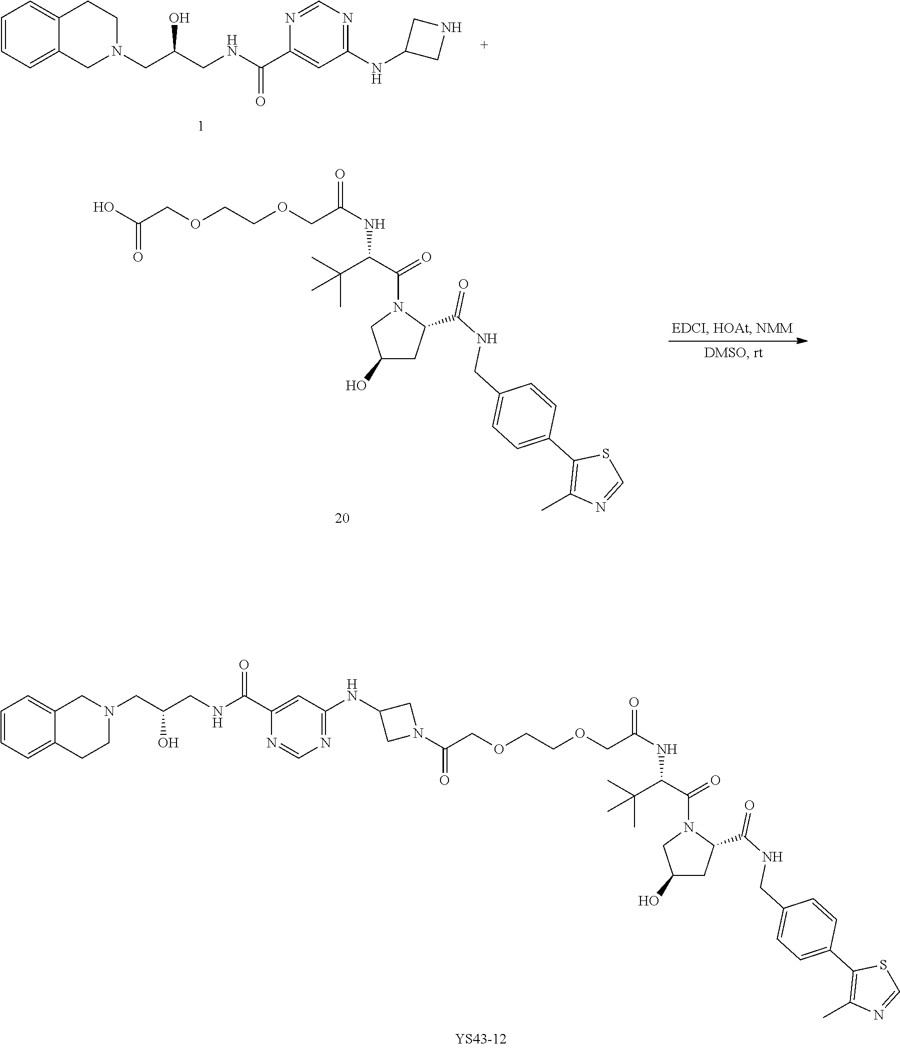 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)COCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 20 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-12 as white solid in TFA salt form (7 mg, yield 73%). ¹H NMR (600 MHz, Methanol-d₄) δ 8.93 (s, 1H), 8.50 (s, 1H), 7.47-7.37 (m, 4H), 7.32-7.17 (m, 5H), 4.72-4.69 (m, 2H), 4.63-4.49 (m, 5H), 4.42-4.31 (m, 5H), 4.25-4.12 (m, 4H), 4.08-4.02 (m, 2H), 3.92-3.80 (m, 5H), 3.75-3.69 (m, 4H), 3.53-3.48 (m, 2H), 3.42-3.33 (m, 2H), 2.47 (s, 3H), 2.26-2.21 (m, 1H), 2.13-2.08 (m, 1H), 1.04 (s, 9H). HRMS (m/z) for C₄₈H₆₃N₁₀O₉S⁺ [M+H]⁺: molecular weight calculated 955.4495, found 955.4511.

### Example 20—Synthesis of YS43-13

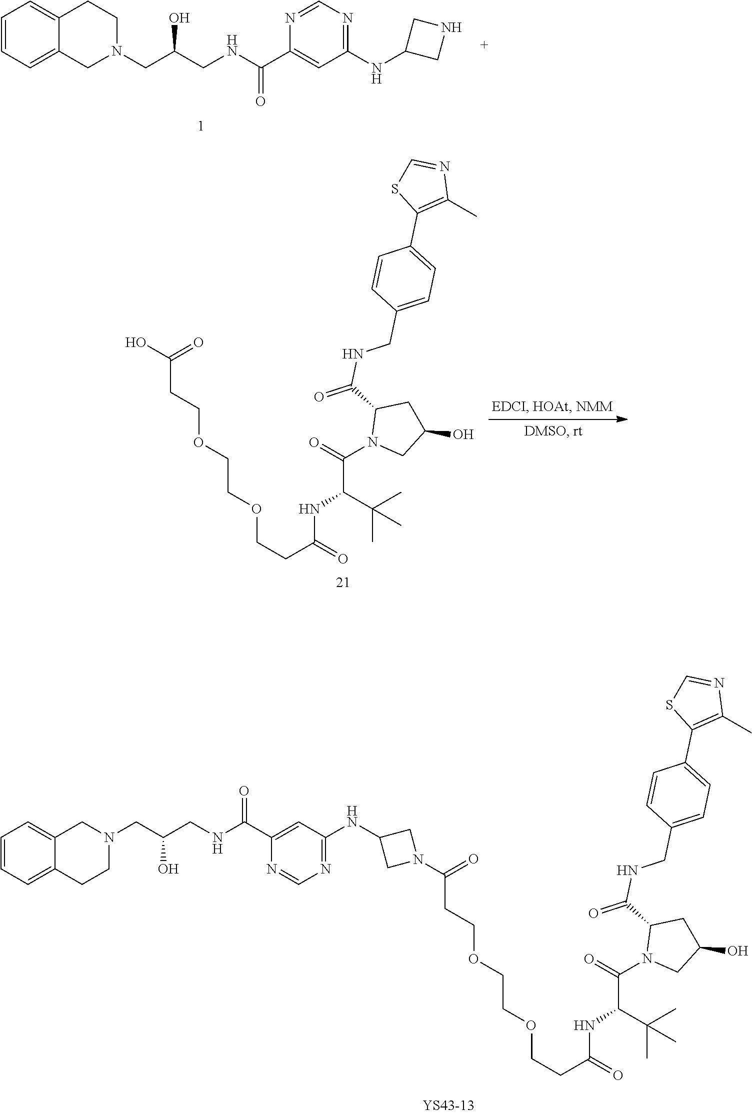 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 21 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-13 as white solid in TFA salt form (8 mg, yield 81%). ¹H NMR (600 MHz, Methanol-d₄) δ 8.95 (s, 1H), 8.54 (s, 1H), 7.49-7.34 (m, 4H), 7.33-7.14 (m, 5H), 4.79-4.74 (m, 1H), 4.66-4.44 (m, 7H), 4.44-4.28 (m, 5H), 4.16-4.10 (m, 1H), 3.90-3.77 (m, 5H), 3.75-3.67 (m, 4H), 3.61-3.49 (m, 6H), 3.44-3.37 (m, 2H), 2.56-2.38 (m, 7H), 2.25-2.20 (m, 1H), 2.11-2.03 (m, 1H), 1.03 (s, 9H). HRMS (m/z) for C₅₀H₆₇N₁₀O₉S⁺ [M+H]⁺: molecular weight calculated 983.4808, found 983.4795.

### Example 21—Synthesis of YS43-14

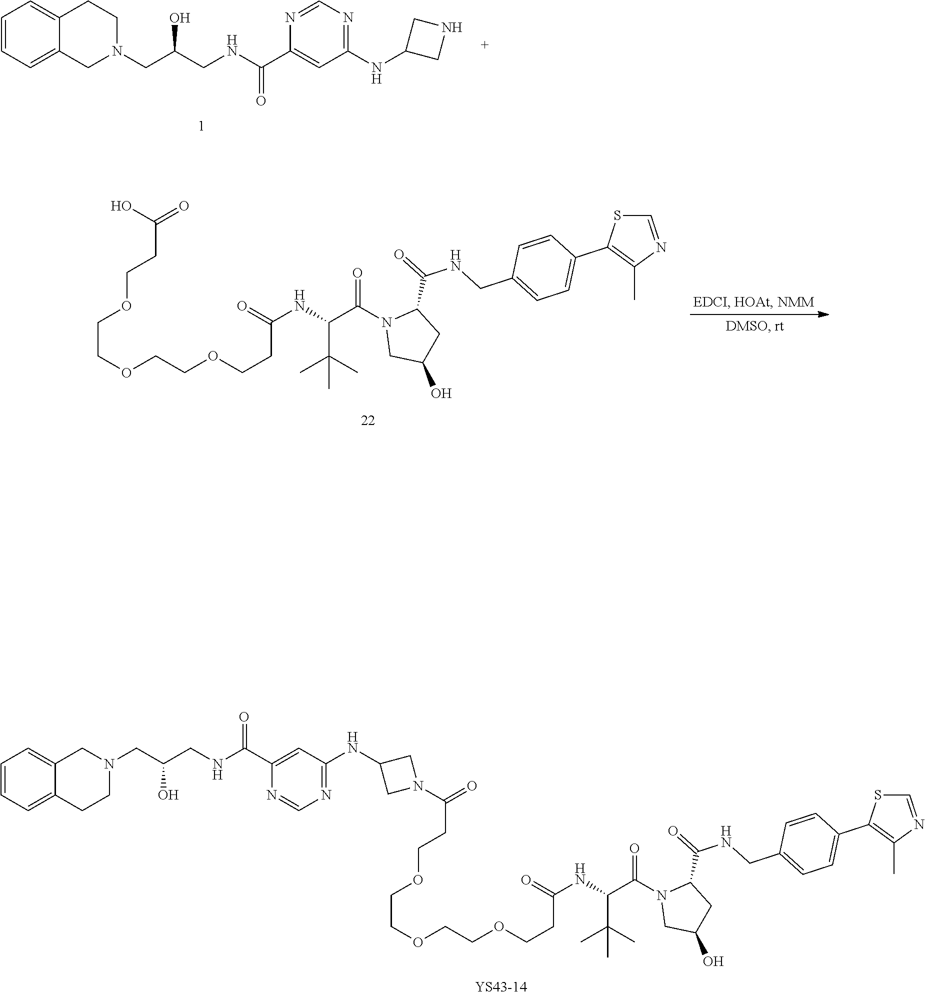 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 22 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-14 as white solid in TFA salt form (10 mg, yield 97%). ¹H NMR (600 MHz, Methanol-d₄) δ 8.96 (s, 1H), 8.55 (s, 1H), 7.50-7.39 (m, 4H), 7.33-7.15 (m, 5H), 4.81-4.76 (m, 1H), 4.66-4.48 (m, 7H), 4.44-4.31 (m, 5H), 4.17-4.14 (m, 1H), 3.91-3.78 (m, 5H), 3.73-3.69 (m, 4H), 3.64-3.48 (m, 10H), 3.41-3.35 (m, 2H), 2.58-2.38 (m, 7H), 2.25-2.20 (m, 1H), 2.11-2.07 (m, 1H), 1.03 (s, 9H). HRMS (m/z) for C₅₂H₇₁N₁₀O₁₀S⁺ [M+H]⁺: molecular weight calculated 1027.5070, found 127.5066.

### Example 22—Synthesis of YS43-15

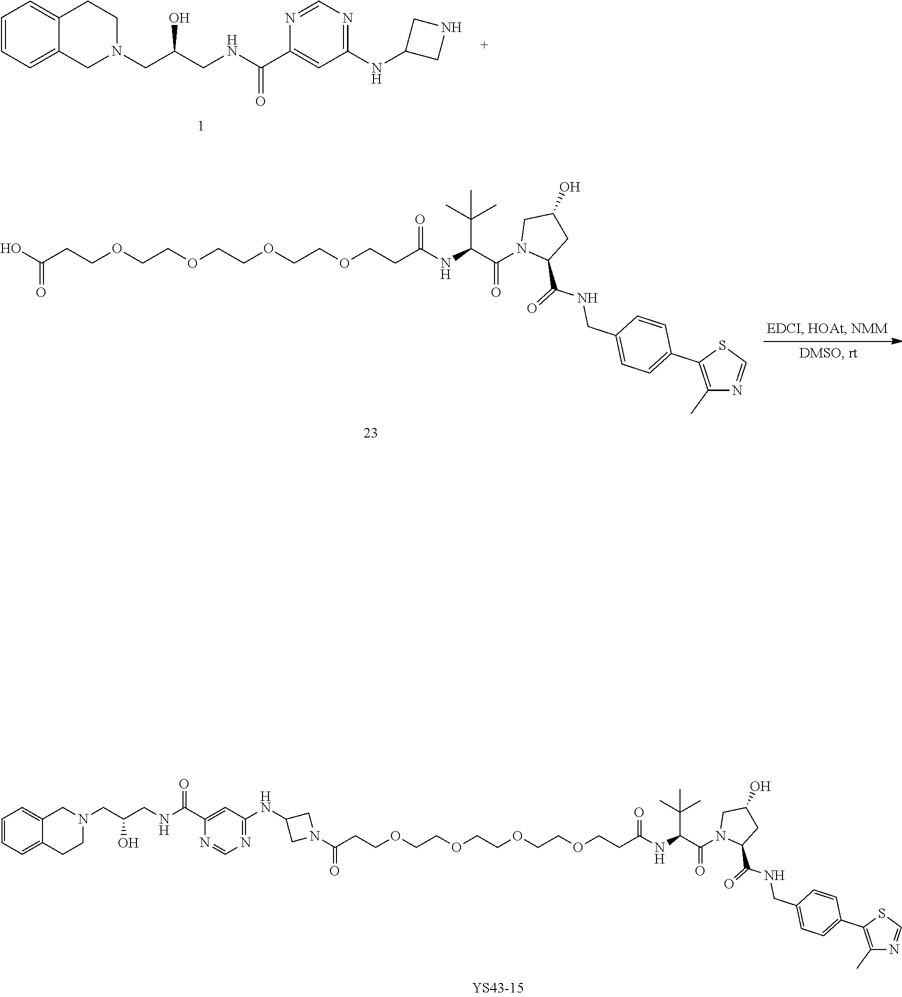 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 23 (10 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-15 as white solid in TFA salt form (11 mg, yield 100% [??]). ¹H NMR (600 MHz, Methanol-d₄) δ 8.98 (s, 1H), 8.55 (s, 1H), 7.49-7.41 (m, 4H), 7.32-7.18 (m, 5H), 4.80-4.76 (m, 1H), 4.64-4.47 (m, 7H), 4.45-4.33 (m, 5H), 4.15 (s, 1H), 3.97-3.79 (m, 5H), 3.74-3.71 (m, 4H), 3.65-3.47 (m, 14H), 3.41-3.34 (m, 2H), 2.58-2.39 (m, 7H), 2.24-2.20 (m, 1H), 2.10-2.06 (m, 1H), 1.03 (s, 9H). HRMS (m/z) for C₅₄H₇₅N₁₀O₁₁S⁺ [M+1-1]⁺: molecular weight calculated 1071.5332, found 1071.5354.

### Example 23—Synthesis of YS43-16

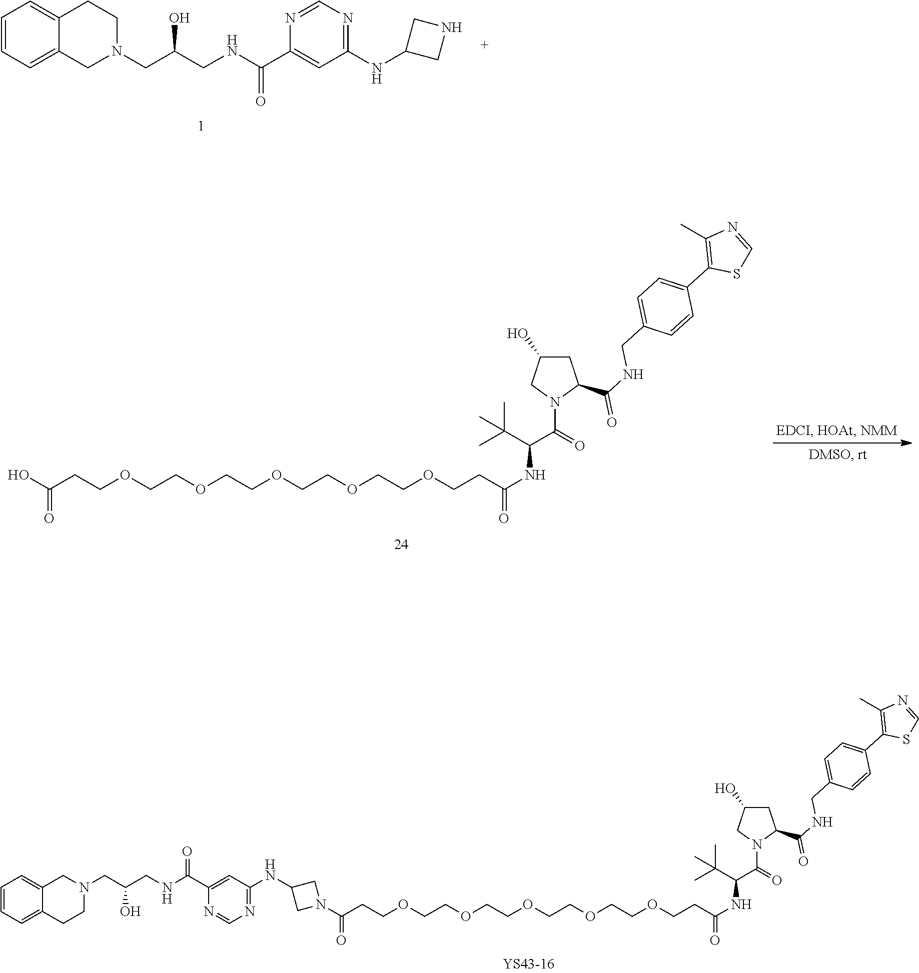 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 24 (10 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-16 as white solid in TFA salt form (6 mg, yield 49%). ¹H NMR (600 MHz, Methanol-d₄) δ 9.05 (s, 1H), 8.58 (s, 1H), 7.47-7.38 (m, 4H), 7.32-7.19 (m, 5H), 4.82-4.77 (m, 1H), 4.66-4.47 (m, 7H), 4.47-4.29 (m, 5H), 4.21-4.16 (m, 1H), 3.95-3.79 (m, 5H), 3.76-3.69 (m, 4H), 3.65-3.46 (m, 18H), 3.41-3.33 (m, 2H), 2.58-2.36 (m, 7H), 2.26-2.19 (m, 1H), 2.10-2.05 (m, 1H), 1.03 (s, 9H). HRMS (m/z) for C₅₆H₇₉N₁₀O₁₂S⁺ [M+H]⁺: molecular weight calculated 1115.55594, found 1115.5610.

### Example 24—Synthesis of YS43-17

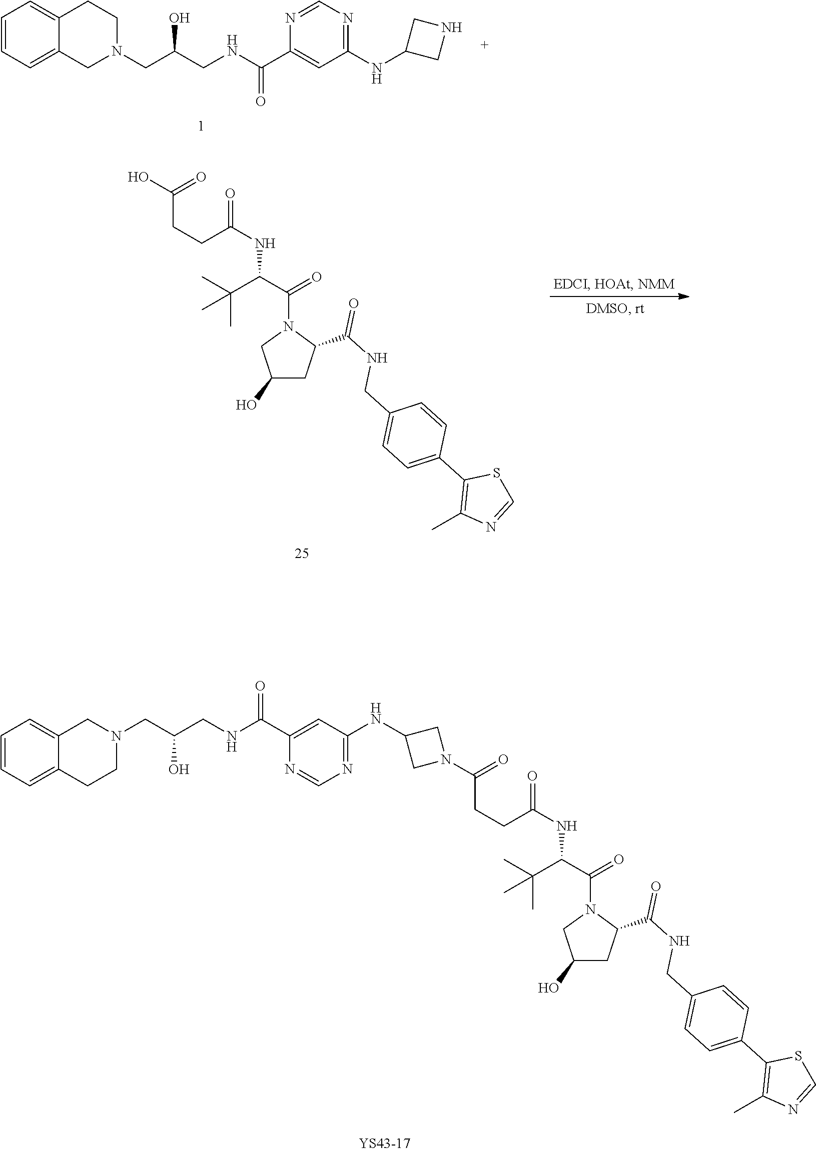 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCC(=O)O)C(C)(C)C)cc1 ; Cc1ncsc1-c1ccc(CNC(=O)[C@@H]2C[C@@H](O)CN2C(=O)[C@@H](NC(=O)CCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 25 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-17 as white solid in TFA salt form (7 mg, yield 78%). ¹H NMR (600 MHz, Methanol-d₄) δ 9.00 (s, 1H), 8.55 (s, 1H), 7.48-7.40 (m, 4H), 7.34-7.17 (m, 5H), 4.82-4.74 (m, 1H), 4.65-4.47 (m, 7H), 4.36 (dd, J=15.5, 5.3 Hz, 5H), 4.16-4.11 (m, 1H), 3.92-3.80 (m, 5H), 3.56-3.49 (m, 2H), 3.43-3.33 (m, 2H), 2.63-2.36 (m, 7H), 2.26-2.21 (m, 1H), 2.12-2.07 (m, 1H), 1.03 (s, 9H). HRMS (m/z) for C₄₆H₅₉N₁₀O₇S⁺ [M+H]⁺: molecular weight calculated 895.4283, found 895.4264.

### Example 25—Synthesis of YS43-18

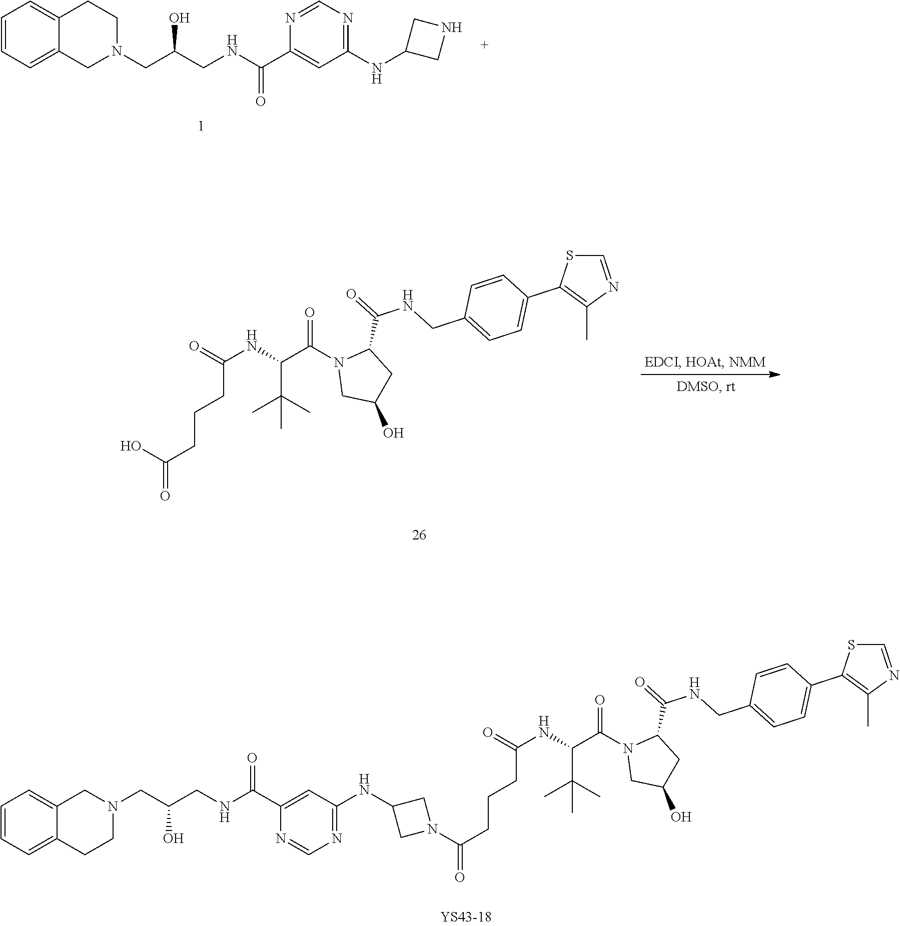 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCCC(=O)O)C(C)(C)C)cc1 ; Cc1ncsc1-c1ccc(CNC(=O)[C@@H]2C[C@@H](O)CN2C(=O)[C@@H](NC(=O)CCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 26 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-18 as white solid in TFA salt form (9 mg, yield 99%). ¹H NMR (600 MHz, Methanol-d₄) δ 8.96 (s, 1H), 8.53 (s, 1H), 7.48-7.39 (m, 4H), 7.33-7.16 (m, 5H), 4.80-4.75 (m, 1H), 4.63-4.48 (m, 7H), 4.44-4.31 (m, 5H), 4.10-4.05 (m, 1H), 3.94-3.77 (m, 5H), 3.56-3.49 (m, 2H), 3.44-3.33 (m, 2H), 2.47 (s, 3H), 2.35-2.28 (m, 2H), 2.22-2.17 (m, 3H), 2.11-2.06 (m, 1H), 1.92-1.85 (m, 2H), 1.04 (s, 9H). HRMS (m/z) for C₄₇H₆₁N₁₀O₇S⁺ [M+H]⁺: molecular weight calculated 909.4440, found 909.4462.

### Example 26—Synthesis of YS43-19

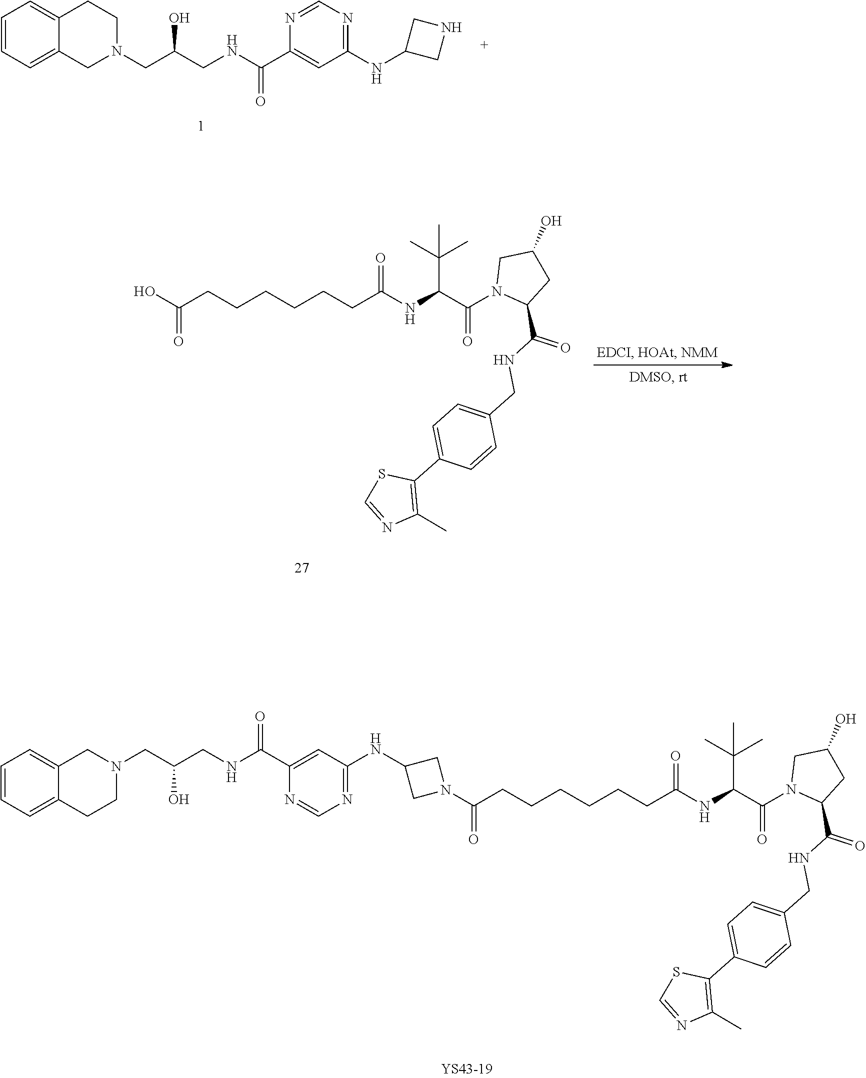 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 27 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-19 as white solid in TFA salt form (6 mg, yield 63%). ¹H NMR (600 MHz, Methanol-d₄) δ 9.00 (s, 1H), 8.55 (s, 1H), 7.51-7.40 (m, 4H), 7.33-7.18 (m, 5H), 4.84-4.77 (m, 1H), 4.64-4.47 (m, 7H), 4.45-4.28 (m, 5H), 4.14-4.07 (m, 1H), 3.93-3.77 (m, 5H), 3.58-3.48 (m, 2H), 3.41-3.32 (m, 2H), 2.48 (s, 3H), 2.32-2.06 (m, 6H), 1.64-1.58 (m, 4H), 1.38-1.31 (m, 4H), 1.03 (s, 9H). HRMS (m/z) for C₅₀H67N₁₀O₇S⁺ [M+H]⁺: molecular weight calculated 951.4909, found 951.4887.

### Example 27—Synthesis of YS43-20

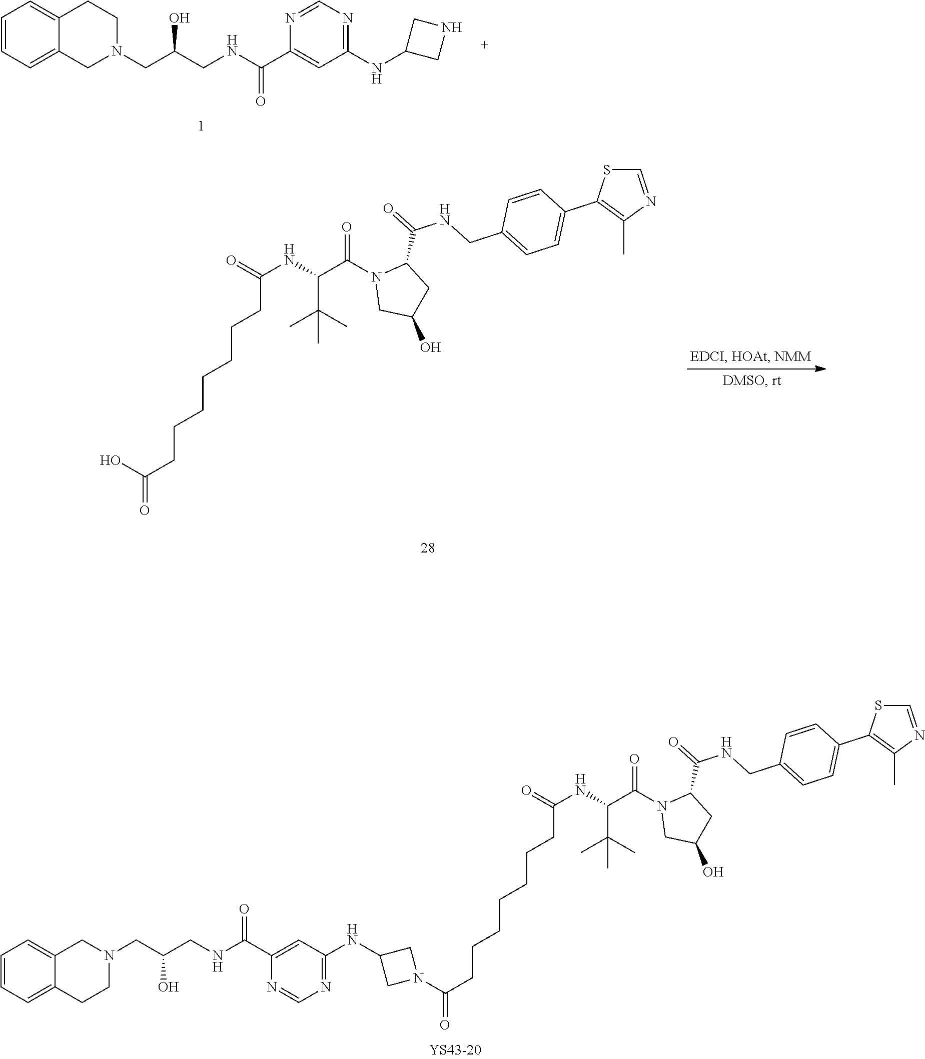 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 28 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-20 as white solid in TFA salt form (4 mg, yield 41%). ¹H NMR (600 MHz, Methanol-d₄) δ 9.00 (s, 1H), 8.56 (s, 1H), 7.48-7.41 (m, 4H), 7.33-7.17 (m, 5H), 4.83-4.74 (m, 1H), 4.66-4.50 (m, 7H), 4.42-4.30 (m, 5H), 4.13-4.09 (m, 1H), 3.93-3.77 (m, 5H), 3.55-3.52 (m, 2H), 3.40-3.31 (m, 2H), 2.48 (s, 3H), 2.27-2.09 (m, 6H), 1.62-1.58 (m, 4H), 1.36-1.28 (m, 6H), 1.03 (s, 9H). HRMS (m/z) for C₅₁H₆₉N₁₀O₇S⁺ [M+H]⁺: molecular weight calculated 965.5066, found 965.5077.

### Example 28—Synthesis of YS43-21

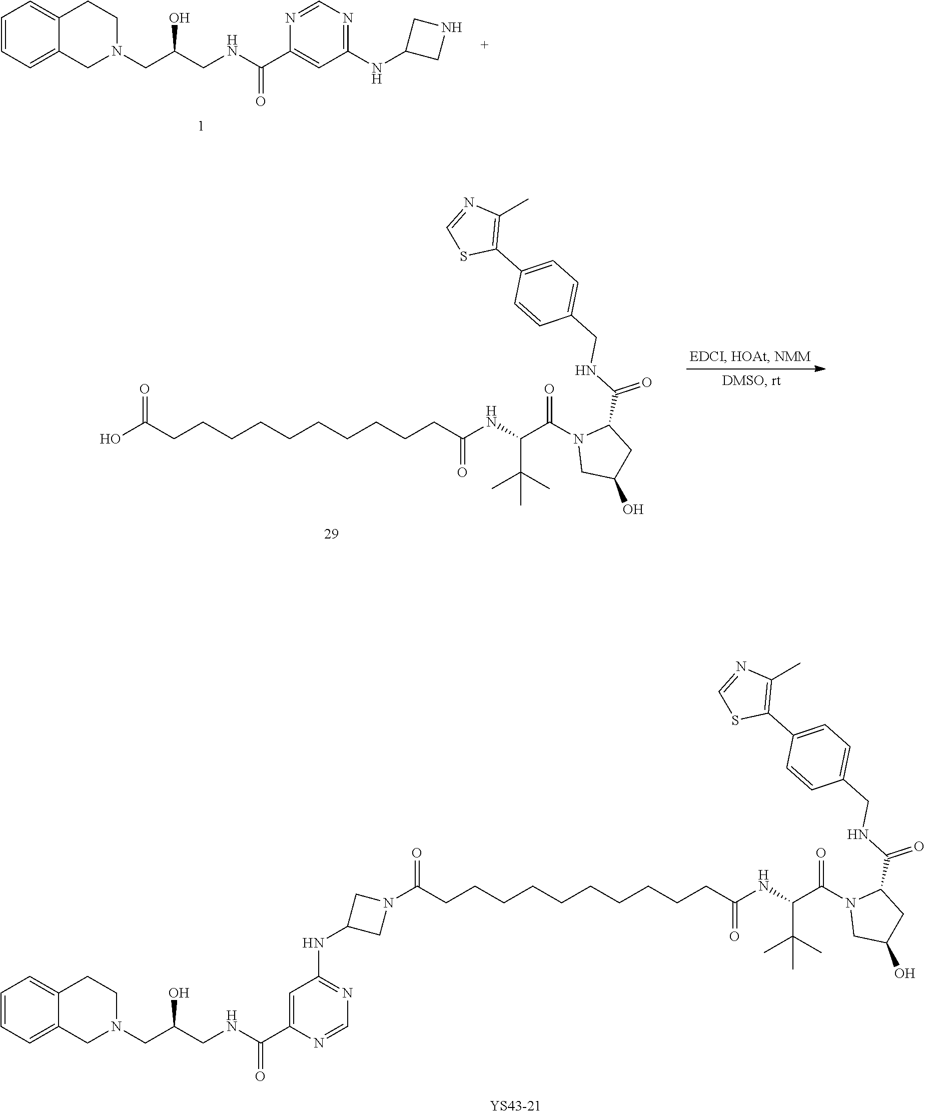 [SMILES: O=C(NC[C@H](O)CN1CCc2ccccc2C1)c1cc(NC2CNC2)ncn1 ; Cc1ncsc1-c1ccc(CNC(=O)[C@@H]2C[C@@H](O)CN2C(=O)[C@@H](NC(=O)CCCCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCCCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 29 (10 mg, 0.02 mmol, 2 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-21 as white solid in TFA salt form (6 mg, yield 61%). ¹H NMR (600 MHz, Methanol-d₄) δ 8.97 (s, 1H), 8.55 (s, 1H), 7.49-7.41 (m, 4H), 7.33-7.18 (m, 5H), 4.80-4.74 (m, 1H), 4.65-4.48 (m, 7H), 4.43-4.30 (m, 5H), 4.11-4.06 (m, 1H), 3.92-3.76 (m, 5H), 3.57-3.50 (m, 2H), 3.40-3.32 (m, 2H), 2.48 (s, 3H), 2.33-2.06 (m, 6H), 1.61-1.58 (m, 4H), 1.36-1.28 (m, 8H), 1.03 (s, 9H). HRMS (m/z) for C₅₂H₇₁N₁₀O₇S⁺ [M+H]⁺: molecular weight calculated 979.5222, found 979.5250.

### Example 29—Synthesis of YS43-22

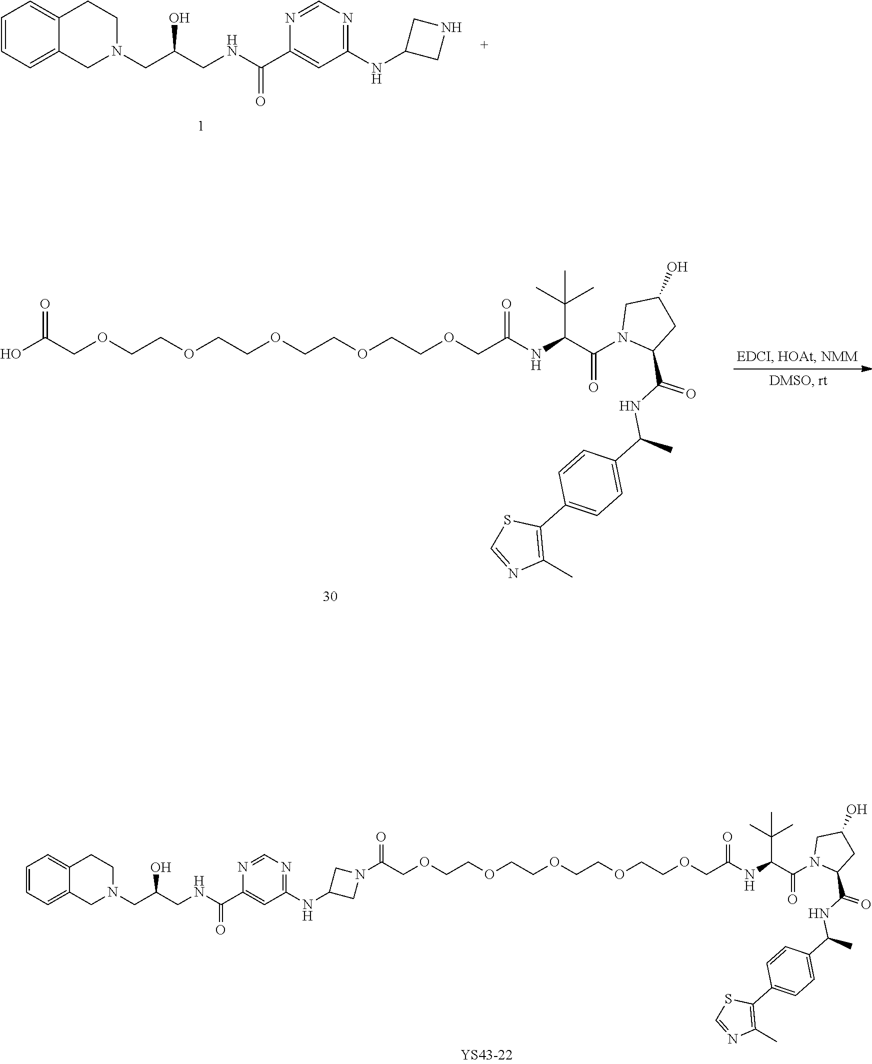 [SMILES: O=C(NC[C@H](O)CN1CCc2ccccc2C1)c1cc(NC2CNC2)ncn1 ; Cc1ncsc1-c1ccc([C@H](C)NC(=O)[C@@H]2C[C@@H](O)CN2C(=O)[C@@H](NC(=O)COCCOCCOCCOCCOCC(=O)O)C(C)(C)C)cc1 ; Cc1ncsc1-c1ccc([C@H](C)NC(=O)[C@@H]2C[C@@H](O)CN2C(=O)[C@@H](NC(=O)COCCOCCOCCOCCOCC(=O)N2CC(Nc3cc(C(=O)NC[C@H](O)CN4CCc5ccccc5C4)ncn3)C2)C(C)(C)C)cc1]

To a solution of Intermediate 1 in TFA salt form (10 mg, 0.01 mmol), intermediate 30 (10 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-22 as white solid in TFA salt form (6 mg, yield 73%). ¹H NMR (600 MHz, Methanol-d₄) δ 9.15 (s, 1H), 8.91 (s, 1H), 7.48-7.43 (m, 4H), 7.31-7.18 (m, 5H), 4.90-4.86 (m, 1H), 4.74-4.53 (m, 8H), 4.40 (d, J=43.9 Hz, 5H), 4.06-3.92 (m, 8H), 3.86-3.48 (m, 18H), 3.42-3.33 (m, 2H), 2.50 (s, 3H), 2.27-2.23 (m, 1H), 1.98-1.94 (m, 1H), 1.53-1.49 (m, 3H), 1.03 (s, 9H). HRMS (m/z) for C₅₅H₇₇N₁₀O₁₂S⁺ [M+H]⁺: molecular weight calculated 1101.5438, found 1101.5427.

### Example 30—Synthesis of YS43-25

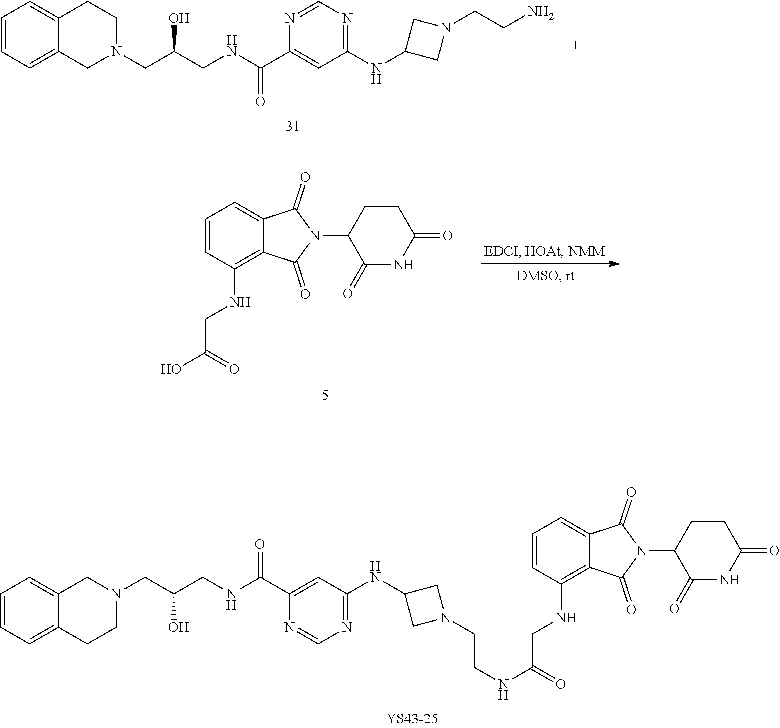 [SMILES: NCCN1CC(Nc2cc(C(=O)NC[C@H](O)CN3CCc4ccccc4C3)ncn2)C1 ; O=C(O)CNc1cccc2c1C(=O)N(C1CCC(=O)NC1=O)C2=O ; O=C(CNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 5 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-25 as yellow solid in TFA salt form (6 mg, yield 81%). ¹H NMR (600 MHz, Methanol-d₄) δ 8.88 (s, 1H), 7.91-7.54 (m, 3H), 7.38-7.11 (m, 4H), 6.96 (d, J=6.8 Hz, 1H), 5.38-5.09 (m, 3H), 4.68 (d, J=76.4 Hz, 3H), 4.39 (d, J=42.9 Hz, 3H), 4.08 (s, 2H), 3.88-3.81 (m, 1H), 3.68-3.15 (m, 10H), 2.97-2.61 (m, 4H), 2.17-2.10 (m, 1H). HRMS (m/z) for C₃₇H₄₃N₁₀O₇⁺ [M+H]⁺: molecular weight calculated 739.3311, found 739.3337.

### Example 31—Synthesis of YS43-26

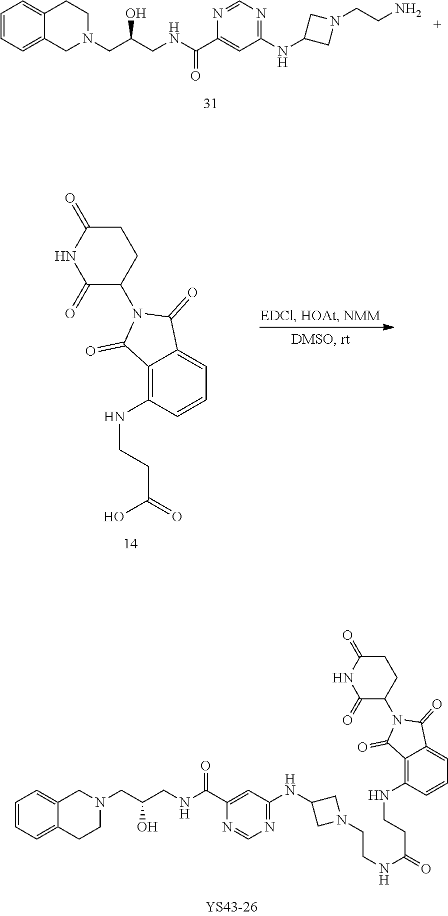 [SMILES: NCCN1CC(Nc2cc(C(=O)NC[C@H](O)CN3CCc4ccccc4C3)ncn2)C1 ; O=C(O)CCNc1cccc2c1C(=O)N(C1CCC(=O)NC1=O)C2=O ; O=C(CCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 14 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-26 as yellow solid in TFA salt form (7 mg, yield 93%). ¹H NMR (600 MHz, Methanol-d₄) δ 8.89 (s, 1H), 7.68-7.53 (m, 2H), 7.32-7.24 (m, 3H), 7.20-7.04 (m, 3H), 5.07-5.01 (m, 1H), 4.96 (q, J=11.4 Hz, 2H), 4.76-4.72 (m, 1H), 4.67-4.53 (m, 2H), 4.36 (s, 3H), 3.91-3.76 (m, 1H), 3.65 (t, J=6.1 Hz, 2H), 3.59-3.43 (m, 4H), 3.35-3.32 (m, 2H), 3.26-3.14 (m, 4H), 2.91-2.51 (m, 6H), 2.19-2.06 (m, 1H). HRMS (m/z) for C₃₈H₄₅N₁₀O₇⁺ [M+H]⁺: molecular weight calculated 753.3467, found 753.3476.

### Example 32—Synthesis of YS43-27

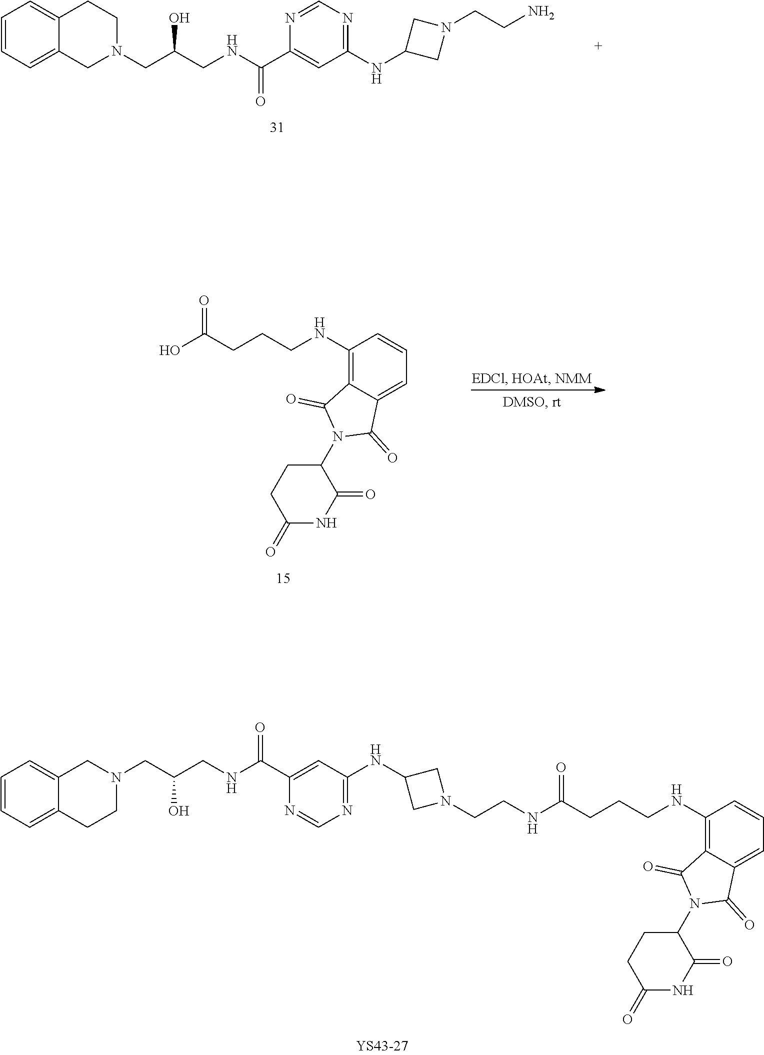 [SMILES: NCCN1CC(Nc2cc(C(=O)NC[C@H](O)CN3CCc4ccccc4C3)ncn2)C1 ; O=C(O)CCCNc1cccc2c1C(=O)N(C1CCC(=O)NC1=O)C2=O ; O=C(CCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 15 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-27 as yellow solid in TFA salt form (4 mg, yield 54%). ¹H NMR (600 MHz, Methanol-d₄) δ 8.85 (s, 1H), 7.63-7.53 (m, 2H), 7.29 (dt, J=26.7, 7.9 Hz, 3H), 7.18 (d, J=7.6 Hz, 1H), 7.05 (dd, J=17.2, 7.8 Hz, 2H), 5.05 (dd, J=12.7, 5.5 Hz, 1H), 4.94 (t, J=11.7 Hz, 2H), 4.72-4.67 (m, 1H), 4.60 (s, 2H), 4.47-4.31 (m, 3H), 3.89-3.78 (m, 1H), 3.56-3.45 (m, 5H), 3.42-3.36 (m, 3H), 3.25-3.12 (m, 4H), 2.89-2.81 (m, 1H), 2.78-2.64 (m, 3H), 2.43-2.34 (m, 2H), 2.13-2.07 (m, 1H), 1.98 (hept, J=6.6 Hz, 2H). HRMS (m/z) for C₃₉H₄₇N₁₀O₇⁺ [M+H]⁺: molecular weight calculated 767.3624, found 767.3647.

### Example 33—Synthesis of YS43-28

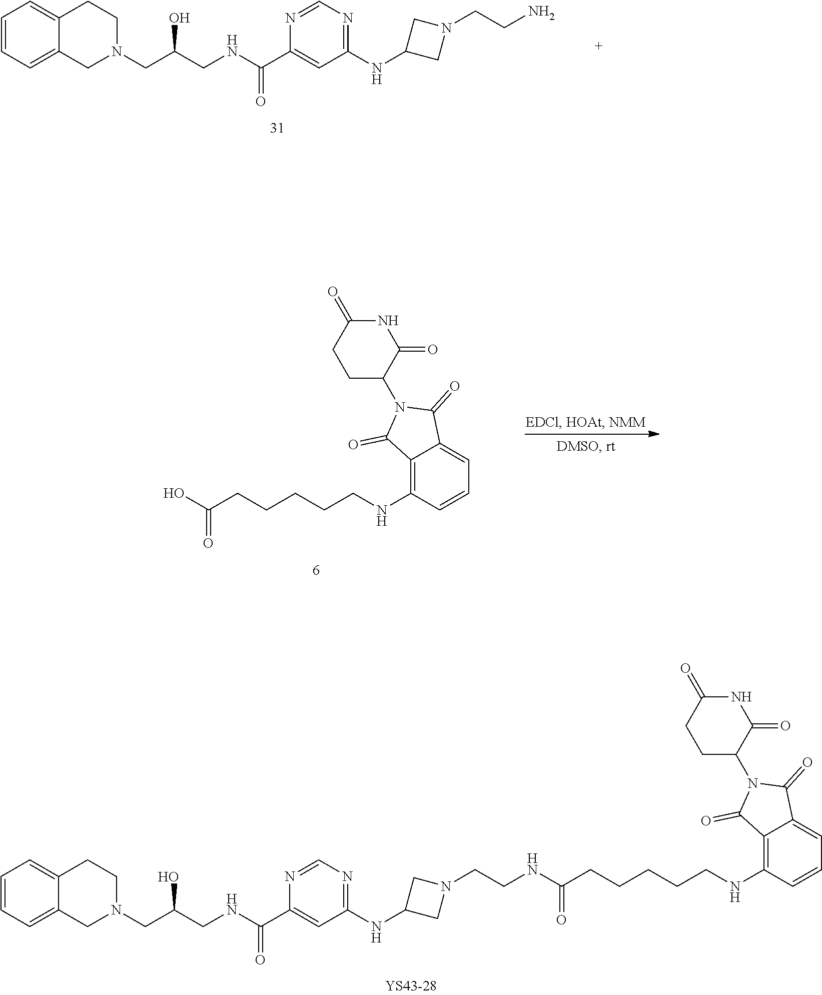 [SMILES: NCCN1CC(Nc2cc(C(=O)NC[C@H](O)CN3CCc4ccccc4C3)ncn2)C1 ; O=C(O)CCCCCNc1cccc2c1C(=O)N(C1CCC(=O)NC1=O)C2=O ; O=C(CCCCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 6 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-28 as yellow solid in TFA salt form (3 mg, yield 38%). ¹H NMR (600 MHz, Methanol-d₄) δ 8.88 (s, 1H), 7.67-7.49 (m, 2H), 7.34-7.17 (m, 4H), 7.04 (dd, J=7.7, 4.3 Hz, 2H), 5.07-5.02 (m, 1H), 4.96 (t, J=11.9 Hz, 2H), 4.72-4.56 (m, 3H), 4.50-4.33 (m, 3H), 3.86-3.80 (m, 1H), 3.60-3.45 (m, 6H), 3.42-3.10 (m, 6H), 2.98-2.57 (m, 4H), 2.27 (t, J=7.4 Hz, 2H), 2.14-2.07 (m, 1H), 1.78-1.63 (m, 4H), 1.48-1.42 (m, 2H). HRMS (m/z) for C₄₁H₅₁N₁₀O₇⁺ [M+H]⁺: molecular weight calculated 795.3937, found 795.3915.

### Example 34—Synthesis of YS43-29

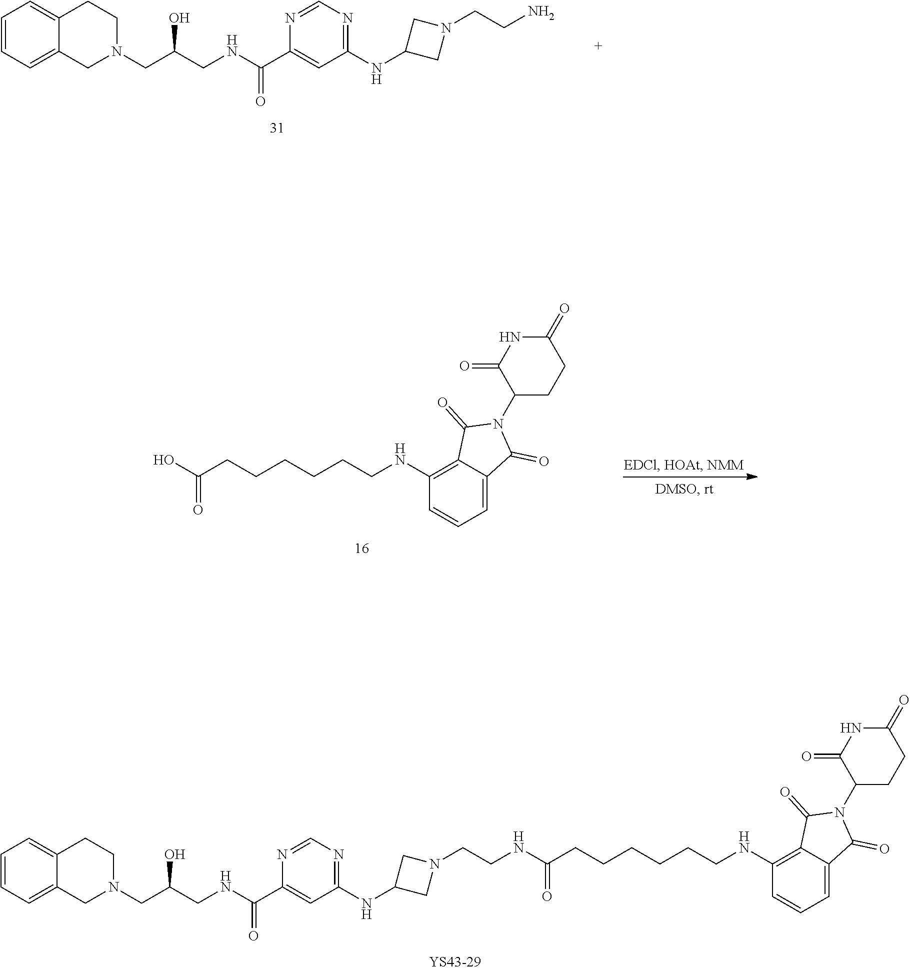 [SMILES: NCCN1CC(Nc2cc(C(=O)NC[C@H](O)CN3CCc4ccccc4C3)ncn2)C1 ; O=C(O)CCCCCCNc1cccc2c1C(=O)N(C1CCC(=O)NC1=O)C2=O ; O=C(CCCCCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 16 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-29 as yellow solid in TFA salt form (3 mg, yield 37%). ¹H NMR (600 MHz, Methanol-d₄) δ 8.85 (s, 1H), 7.65-7.48 (m, 2H), 7.34-7.16 (m, 4H), 7.04 (dd, J=7.8, 4.8 Hz, 2H), 5.08-5.03 (m, 1H), 4.94 (p, J=11.8 Hz, 2H), 4.77-4.50 (m, 3H), 4.47-4.21 (m, 3H), 3.83 (s, 1H), 3.57-3.43 (m, 6H), 3.40-3.18 (m, 6H), 2.90-2.60 (m, 4H), 2.28-2.19 (m, 2H), 2.10 (ddt, J=13.1, 5.5, 2.8 Hz, 1H), 1.65 (dp, J=22.8, 7.3 Hz, 4H), 1.42 (ddt, J=38.7, 14.8, 7.4 Hz, 4H). HRMS (m/z) for C₄₂H₅₃N₁₀O₇⁺ [M+H]⁺: molecular weight calculated 809.4093, found 809.4071.

### Example 35—Synthesis of YS43-30

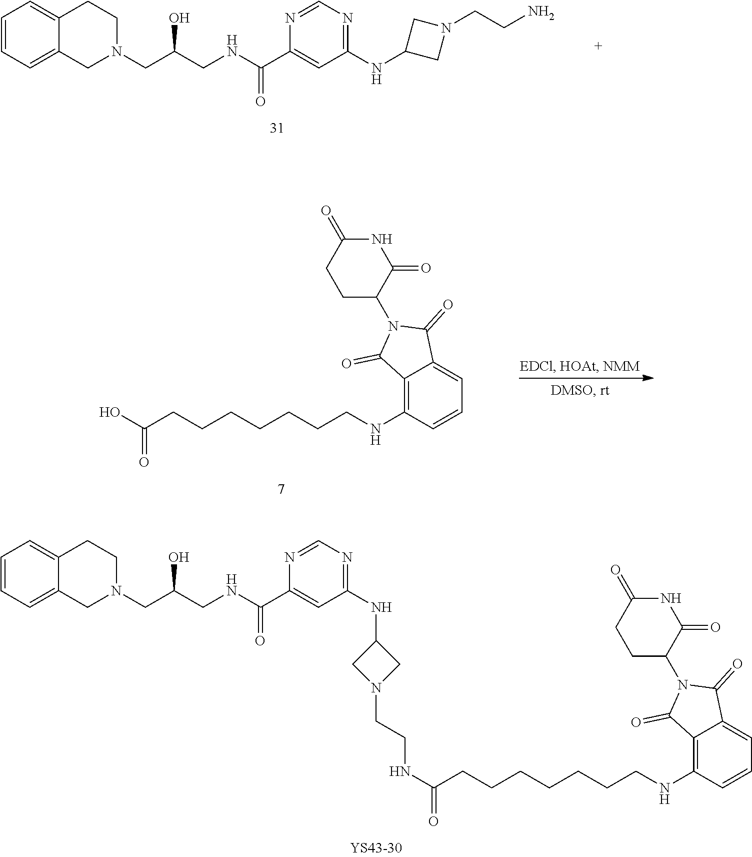 [SMILES: NCCN1CC(Nc2cc(C(=O)NC[C@H](O)CN3CCc4ccccc4C3)ncn2)C1 ; O=C(O)CCCCCCCNc1cccc2c1C(=O)N(C1CCC(=O)NC1=O)C2=O ; O=C(CCCCCCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 7 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-30 as yellow solid in TFA salt form (6 mg, yield 81%). ¹H NMR (600 MHz, Methanol-d₄) δ 8.87 (s, 1H), 7.63-7.48 (m, 2H), 7.33-7.16 (m, 4H), 7.09-7.00 (m, 2H), 5.04 (dd, J=12.8, 5.5 Hz, 1H), 5.00-4.84 (m, 2H), 4.70-4.51 (m, 3H), 4.37 (t, J=22.4 Hz, 3H), 3.87-3.80 (m, 1H), 3.52 (ddd, J=22.4, 11.3, 5.5 Hz, 6H), 3.40-3.15 (m, 6H), 2.89-2.57 (m, 4H), 2.23 (q, J=10.5, 9.0 Hz, 2H), 2.13-2.06 (m, 1H), 1.64 (dp, J=34.7, 7.3 Hz, 4H), 1.48-1.28 (m, 6H). HRMS (m/z) for C₄₃H₅₅N₁₀O₇⁺ [M+H]⁺: molecular weight calculated 823.4250, found 823.4205.

### Example 36—Synthesis of YS43-31

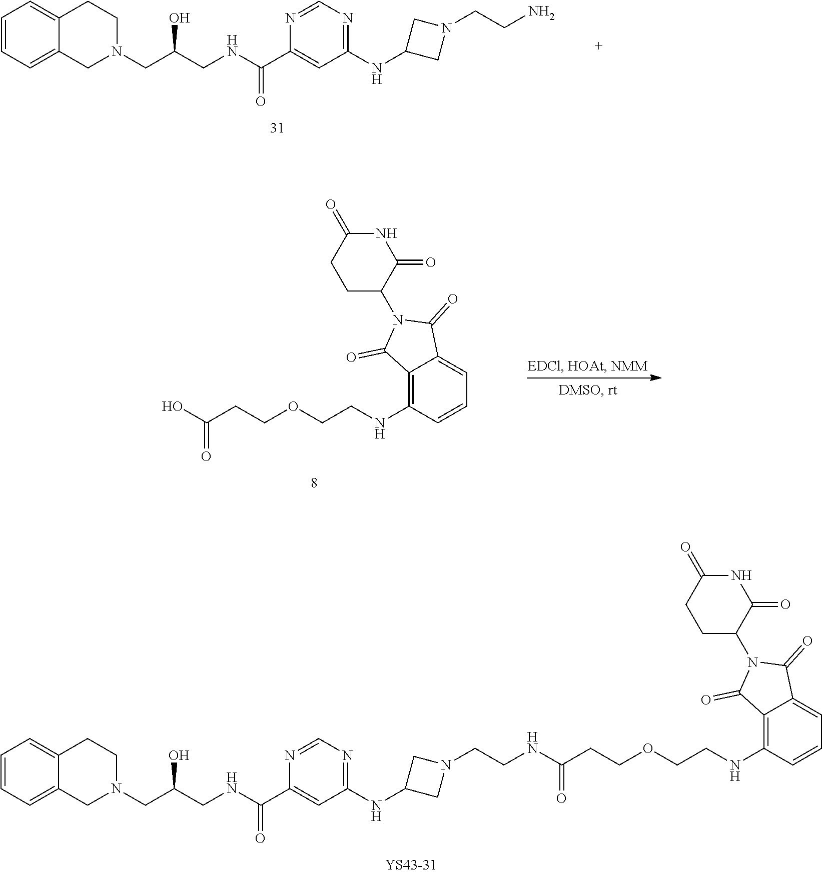 [SMILES: NCCN1CC(Nc2cc(C(=O)NC[C@H](O)CN3CCc4ccccc4C3)ncn2)C1 ; O=C(O)CCOCCNc1cccc2c1C(=O)N(C1CCC(=O)NC1=O)C2=O ; O=C(CCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 8 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-31 as yellow solid in TFA salt form (4 mg, yield 50%). ¹H NMR (600 MHz, Methanol-d₄) δ 8.85 (s, 1H), 7.63-7.51 (m, 2H), 7.34-7.13 (m, 4H), 7.07 (dd, J=15.2, 7.7 Hz, 2H), 5.05 (dd, J=12.7, 5.6 Hz, 1H), 4.93 (q, J=11.9 Hz, 2H), 4.75-4.53 (m, 3H), 4.52-4.26 (m, 3H), 3.77 (p, J=4.9, 4.4 Hz, 3H), 3.70 (t, J=5.1 Hz, 2H), 3.56-3.42 (m, 8H), 3.41-3.14 (m, 4H), 2.90-2.64 (m, 4H), 2.52 (t, J=5.8 Hz, 2H), 2.12-2.08 (m, 1H). HRMS (m/z) for C₄₀H₄₉N₁₀O₈⁺ [M+H]⁺: molecular weight calculated 797.3729, found 797.3733.

### Example 37—Synthesis of YS43-32

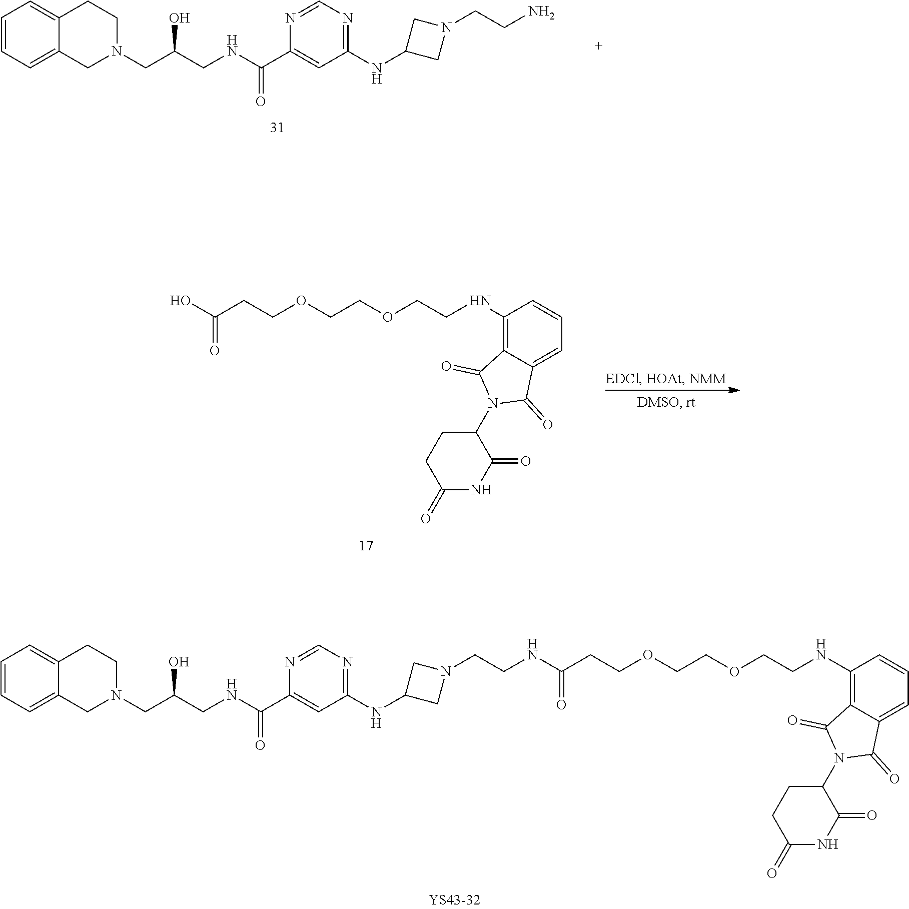 [SMILES: NCCN1CC(Nc2cc(C(=O)NC[C@H](O)CN3CCc4ccccc4C3)ncn2)C1 ; O=C(O)CCOCCOCCNc1cccc2c1C(=O)N(C1CCC(=O)NC1=O)C2=O ; O=C(CCOCCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 17 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-32 as yellow solid in TFA salt form (6 mg, yield 71%). ¹H NMR (600 MHz, Methanol-d₄) δ 8.82 (s, 1H), 7.63-7.50 (m, 2H), 7.34-7.17 (m, 4H), 7.14-7.01 (m, 2H), 5.05 (dd, J=12.6, 5.5, 1.3 Hz, 1H), 4.93-4.82 (m, 2H), 4.64 (dd, J=12.1, 6.1 Hz, 3H), 4.51-4.29 (m, 3H), 3.74 (dt, J=13.4, 5.5 Hz, 5H), 3.69-3.61 (m, 4H), 3.56-3.43 (m, 8H), 3.41-3.16 (m, 4H), 2.90-2.62 (m, 4H), 2.49 (t, J=5.9 Hz, 2H), 2.11 (ddq, J=11.0, 5.4, 2.8 Hz, 1H). HRMS (m/z) for C₄₂H₅₃N₁₀O₉⁺[M+H]⁺: molecular weight calculated 841.3991, found 841.3973.

### Example 38—Synthesis of YS43-33

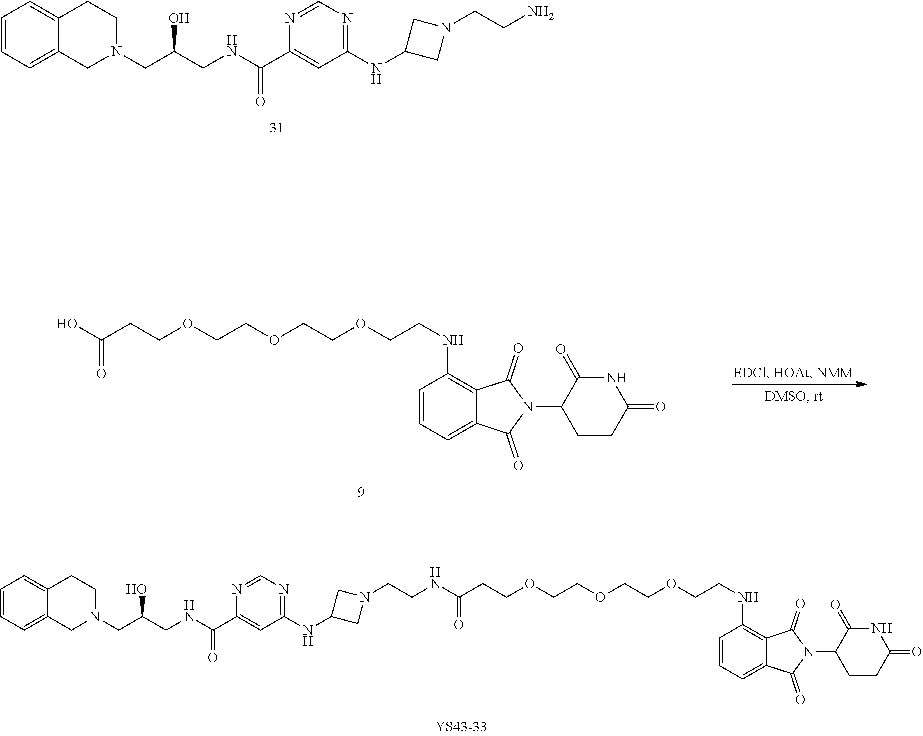 [SMILES: NCCN1CC(Nc2cc(C(=O)NC[C@H](O)CN3CCc4ccccc4C3)ncn2)C1 ; O=C(O)CCOCCOCCOCCNc1cccc2c1C(=O)N(C1CCC(=O)NC1=O)C2=O ; O=C(CCOCCOCCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 9 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-33 as yellow solid in TFA salt form (7 mg, yield 79%). ¹H NMR (600 MHz, Methanol-d₄) δ 8.86 (s, 1H), 7.59-7.51 (m, 2H), 7.39-7.15 (m, 4H), 7.12-7.01 (m, 2H), 5.06 (dd, J=12.7, 5.5 Hz, 1H), 4.93 (q, J=10.5, 9.4 Hz, 2H), 4.78-4.58 (m, 3H), 4.51-4.25 (m, 3H), 3.91-3.70 (m, 5H), 3.70-3.58 (m, 8H), 3.58-3.43 (m, 8H), 3.41-3.16 (m, 4H), 2.90-2.64 (m, 4H), 2.48 (t, J=5.9 Hz, 2H), 2.15-2.08 (m, 1H). HRMS (m/z) for C₄₄H₅₇N₁₀O₁₀⁺ [M+H]⁺: molecular weight calculated 885.4254, found 885.4241.

### Example 39—Synthesis of YS43-34

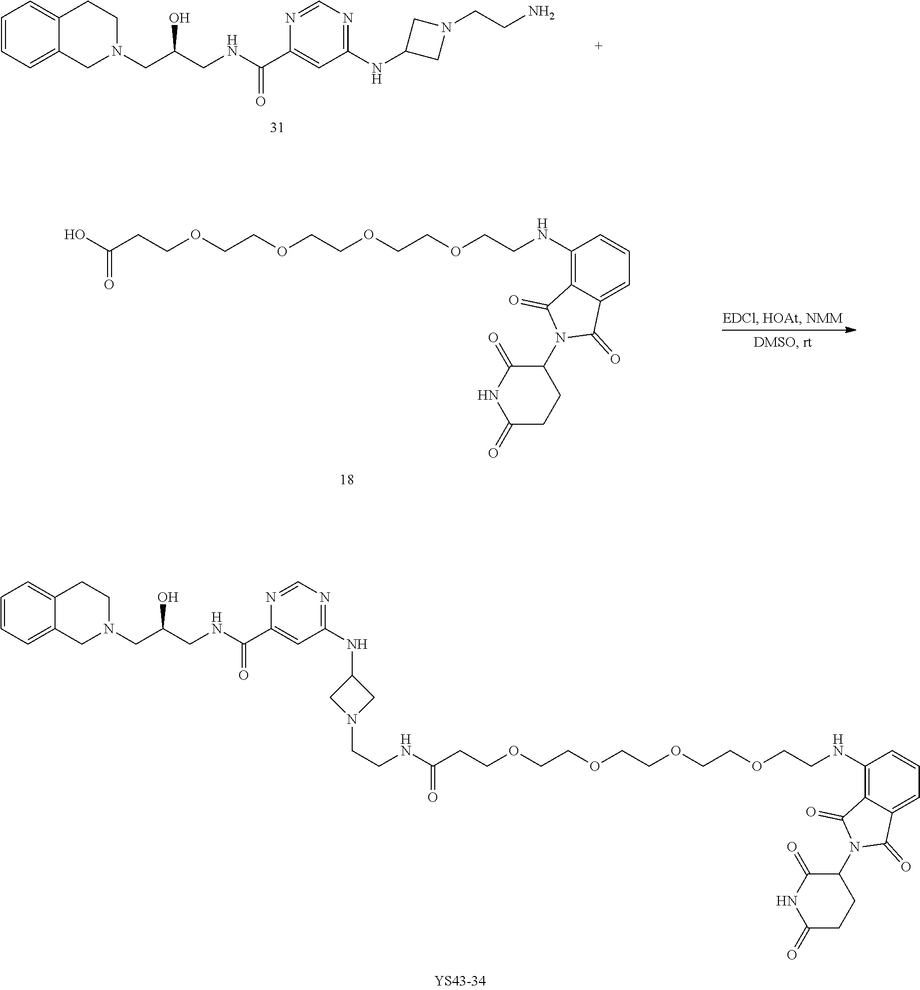 [SMILES: NCCN1CC(Nc2cc(C(=O)NC[C@H](O)CN3CCc4ccccc4C3)ncn2)C1 ; O=C(O)CCOCCOCCOCCOCCNc1cccc2c1C(=O)N(C1CCC(=O)NC1=O)C2=O ; O=C(CCOCCOCCOCCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 18 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-34 as yellow solid in TFA salt form (6 mg, yield 65%). ¹H NMR (600 MHz, Methanol-d₄) δ 8.88 (s, 1H), 7.65-7.50 (m, 2H), 7.35-7.22 (m, 3H), 7.18 (d, J=7.5 Hz, 1H), 7.13-7.00 (m, 2H), 5.05 (dd, J=12.7, 5.5 Hz, 1H), 5.00-4.88 (m, 2H), 4.72-4.50 (m, 3H), 4.50-4.27 (m, 3H), 3.87-3.79 (m, 1H), 3.72 (q, J=6.2, 5.7 Hz, 4H), 3.65 (d, J=16.3 Hz, 6H), 3.63-3.56 (m, 6H), 3.55-3.46 (m, 8H), 3.41-3.16 (m, 4H), 2.90-2.64 (m, 4H), 2.48 (q, J=5.4 Hz, 2H), 2.11 (ddd, J=11.2, 6.2, 3.6 Hz, 1H). HRMS (m/z) for C₄₆H₆₁N₁₀O₁₁⁺ [M+H]⁺: molecular weight calculated 929.4516, found 929.4510.

### Example 40—Synthesis of YS43-35

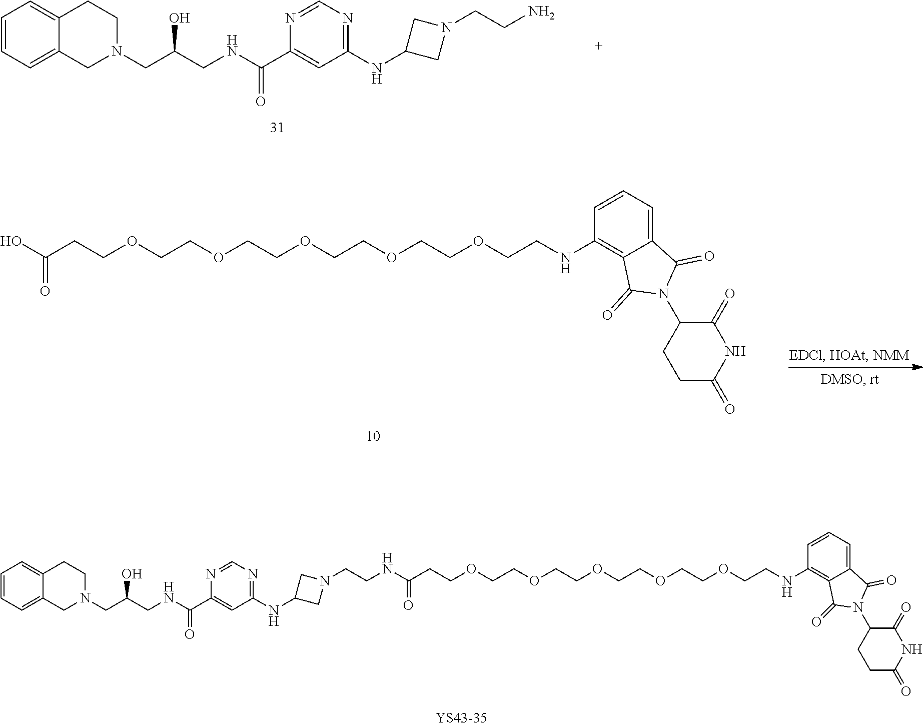 [SMILES: NCCN1CC(Nc2cc(C(=O)NC[C@H](O)CN3CCc4ccccc4C3)ncn2)C1 ; O=C(O)CCOCCOCCOCCOCCOCCNc1cccc2c1C(=O)N(C1CCC(=O)NC1=O)C2=O ; O=C(CCOCCOCCOCCOCCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1]

To a solution of Intermediate 31 in TFA salt form (5 mg, 0.01 mmol), intermediate 10 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-35 as yellow solid in TFA salt form (2 mg, yield 21%). ¹H NMR (600 MHz, Methanol-d₄) δ 8.91 (s, 1H), 7.64-7.61 (m, 1H), 7.59-7.53 (m, 1H), 7.33-7.24 (m, 3H), 7.18 (d, J=7.5 Hz, 1H), 7.11-7.03 (m, 2H), 5.06 (dd, J=12.7, 5.6 Hz, 1H), 5.02-4.91 (m, 2H), 4.74-4.67 (m, 1H), 4.66-4.48 (m, 2H), 4.48-4.26 (m, 3H), 3.87-3.71 (m, 5H), 3.67-3.57 (m, 16H), 3.56-3.47 (m, 8H), 3.42-3.23 (m, 4H), 2.90-2.65 (m, 4H), 2.51-2.47 (m, 2H), 2.14-2.08 (m, 1H). HRMS (m/z) for C₄₈H₆₅N₁₀O₁₂⁺ [M+H]⁺: molecular weight calculated 973.4778, found 973.4766.

### Example 41—Synthesis of YS43-36

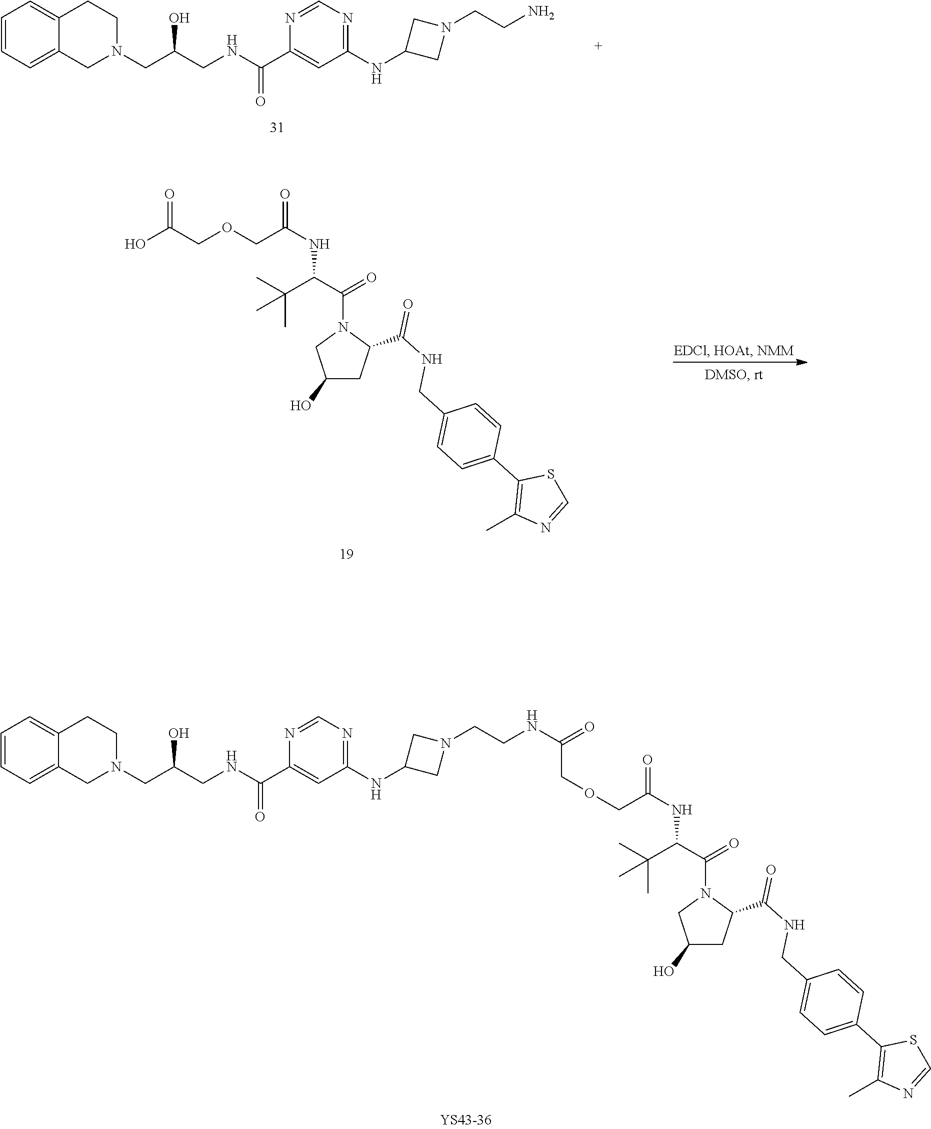 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)COCC(=O)O)C(C)(C)C)cc1 ; Cc1ncsc1-c1ccc(CNC(=O)[C@@H]2C[C@@H](O)CN2C(=O)[C@@H](NC(=O)COCC(=O)NCCN2CC(Nc3cc(C(=O)NC[C@H](O)CN4CCc5ccccc5C4)ncn3)C2)C(C)(C)C)cc1]

To a solution of Intermediate 31 in TFA salt form (5 mg, 0.01 mmol), intermediate 19 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-36 as white solid in TFA salt form (4 mg, yield 42%). ¹H NMR (600 MHz, Methanol-d₄) δ 8.91-8.85 (m, 2H), 7.62 (s, 1H), 7.48-7.37 (m, 4H), 7.33-7.22 (m, 3H), 7.19 (d, J=7.5 Hz, 1H), 5.00-4.87 (m, 2H), 4.77 (s, 1H), 4.73-4.47 (m, 7H), 4.37 (s, 5H), 4.22-4.01 (m, 5H), 3.95-3.71 (m, 5H), 3.65-3.48 (m, 4H), 3.41-3.25 (m, 2H), 2.47 (s, 3H), 2.28-2.19 (m, 1H), 2.14-2.03 (m, 1H), 1.05 (s, 9H). HRMS (m/z) for C₄₈H₆₄N₁₁O₈S⁺ [M+H]⁺: molecular weight calculated 954.4655, found 954.4626.

### Example 42—Synthesis of YS43-37

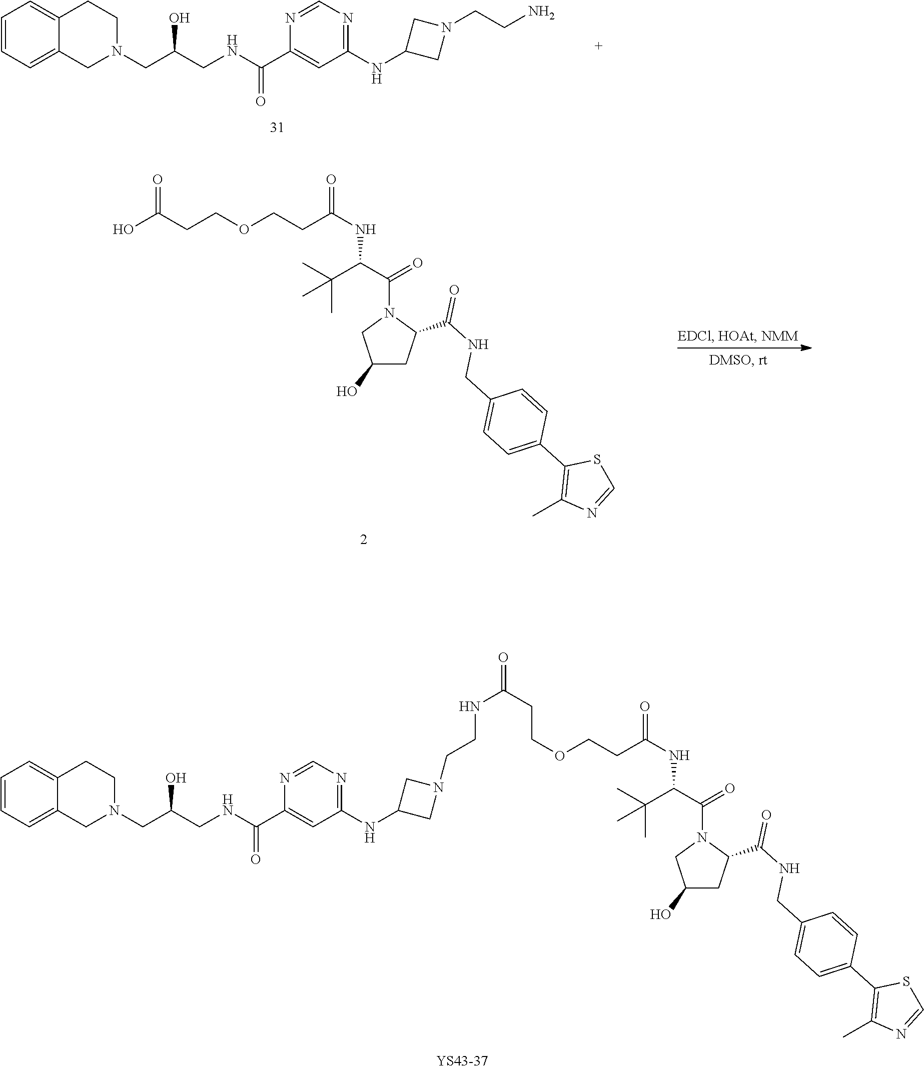 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCC(=O)NCCN2CC(Nc3cc(C(=O)NC[C@H](O)CN4CCc5ccccc5C4)ncn3)C2)C(C)(C)C)cc1]

To a solution of Intermediate 31 in TFA salt form (5 mg, 0.01 mmol), intermediate 2 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-37 as white solid in TFA salt form (4 mg, yield 41%). ¹H NMR (600 MHz, Methanol-d₄) δ 8.89 (s, 2H), 7.64 (s, 1H), 7.49-7.36 (m, 4H), 7.35-7.24 (m, 3H), 7.19 (d, J=7.6 Hz, 1H), 5.03-4.88 (m, 2H), 4.69 (dd, J=12.4, 6.2 Hz, 1H), 4.64-4.46 (m, 7H), 4.46-4.32 (m, 5H), 4.23-4.11 (m, 1H), 3.92-3.77 (m, 5H), 3.75-3.61 (m, 4H), 3.54 (dt, J=22.1, 7.3 Hz, 4H), 3.31-3.23 (m, 2H), 2.59-2.42 (m, 7H), 2.28-2.20 (m, 1H), 2.09 (ddd, J=13.4, 9.3, 4.4 Hz, 1H), 1.03 (s, 9H). HRMS (m/z) for C₅₀H₆₈N₁₁O₈S⁺ [M+H]⁺: molecular weight calculated 982.4968, found 982.4978.

### Example 43—Synthesis of YS43-38

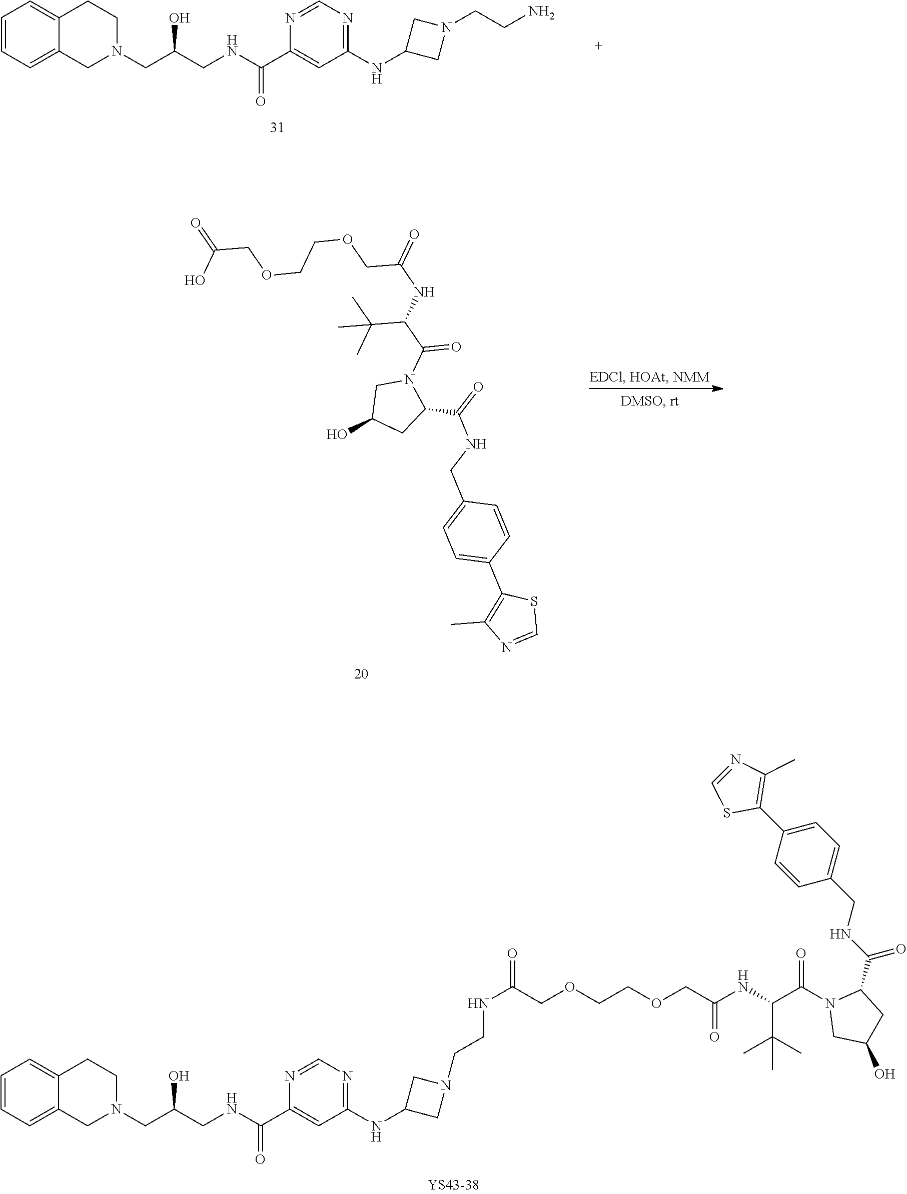 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)COCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCC(=O)NCCN2CC(Nc3cc(C(=O)NC[C@H](O)CN4CCc5ccccc5C4)ncn3)C2)C(C)(C)C)cc1]

To a solution of Intermediate 31 in TFA salt form (5 mg, 0.01 mmol), intermediate 20 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-38 as white solid in TFA salt form (3 mg, yield 30%). ¹H NMR (600 MHz, Methanol-d₄) δ 8.92-8.80 (m, 2H), 7.60 (s, 1H), 7.47-7.38 (m, 4H), 7.32-7.25 (m, 3H), 7.19 (d, J=7.5 Hz, 1H), 4.95-4.84 (m, 2H), 4.81-4.75 (m, 1H), 4.73-4.29 (m, 12H), 4.14-4.02 (m, 5H), 3.91-3.71 (m, 9H), 3.61-3.44 (m, 4H), 3.31 (s, 2H), 2.47 (d, J=5.8 Hz, 3H), 2.31-2.22 (m, 1H), 2.12-2.03 (m, 1H), 1.04 (s, 9H). HRMS (m/z) for C₅₀H₆₈N₁₁O₉S⁺ [M+H]⁺: molecular weight calculated 998.4917, found 998.4913.

### Example 44—Synthesis of YS43-39

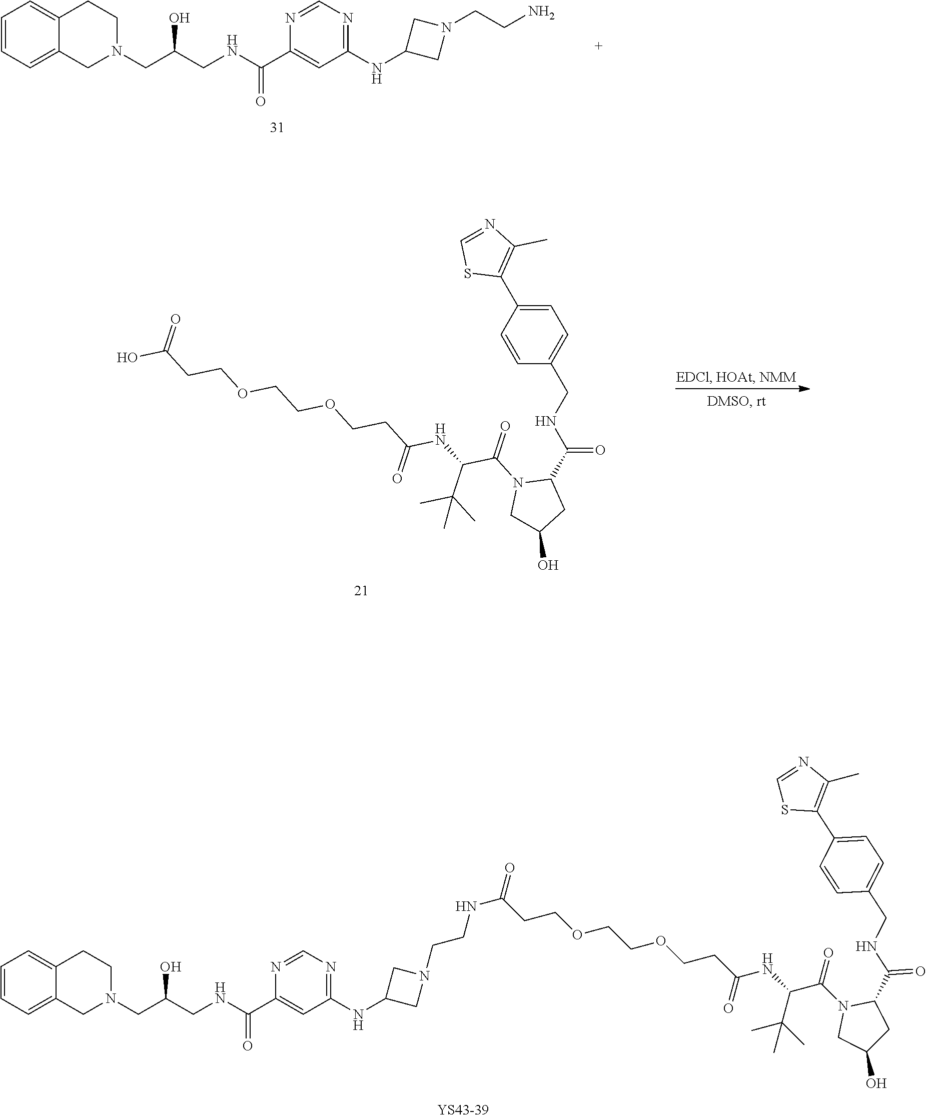

To a solution of Intermediate 31 in TFA salt form (5 mg, 0.01 mmol), intermediate 21 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-39 as white solid in TFA salt form (6 mg, yield 58%). ¹H NMR (600 MHz, Methanol-d₄) δ 9.00-8.80 (m, 2H), 7.71-7.66 (m, 1H), 7.48-7.37 (m, 4H), 7.35-7.24 (m, 3H), 7.19 (d, J=7.6 Hz, 1H), 5.03-4.85 (m, 2H), 4.72 (dd, J=12.7, 6.4 Hz, 1H), 4.65-4.47 (m, 7H), 4.48-4.32 (m, 5H), 4.22-4.13 (m, 1H), 3.93-3.79 (m, 5H), 3.78-3.70 (m, 4H), 3.65-3.50 (m, 8H), 3.44-3.32 (m, 2H), 2.60-2.42 (m, 7H), 2.23 (dd, J=13.2, 7.6 Hz, 1H), 2.08 (ddd, J=13.3, 9.3, 4.4 Hz, 1H), 1.04 (s, 9H). HRMS (m/z) for C₅₂H₇₂N₁₁O₉S⁺ [M+H]⁺: molecular weight calculated 1026.5230, found 1026.5208.

### Example 45—Synthesis of YS43-40

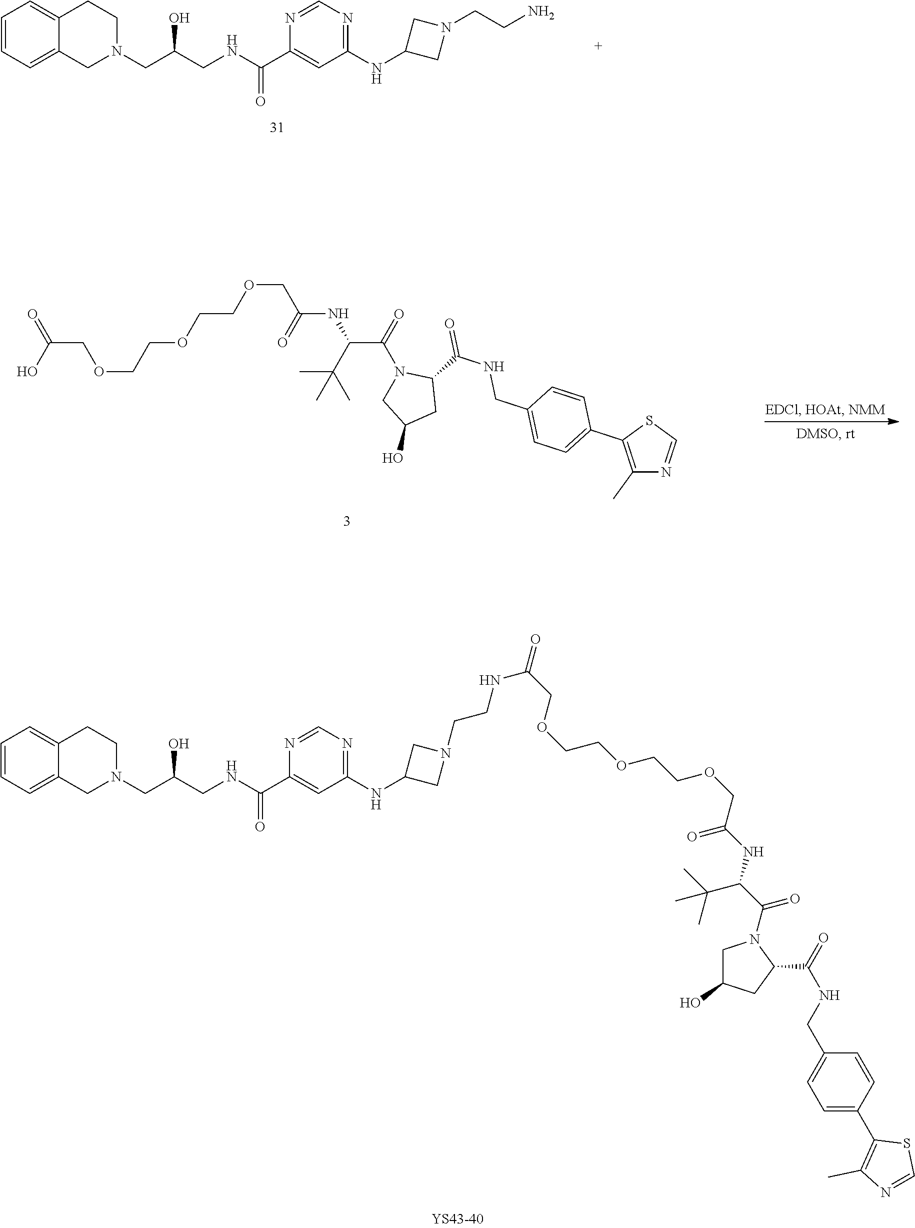 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)COCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCC(=O)NCCN2CC(Nc3cc(C(=O)NC[C@H](O)CN4CCc5ccccc5C4)ncn3)C2)C(C)(C)C)cc1]

To a solution of Intermediate 31 in TFA salt form (5 mg, 0.01 mmol), intermediate 3 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-40 as white solid in TFA salt form (4 mg, yield 38%). ¹H NMR (600 MHz, Methanol-d₄) δ 8.87 (s, 2H), 7.61 (s, 1H), 7.49-7.36 (m, 4H), 7.34-7.24 (m, 3H), 7.19 (d, J=7.6 Hz, 1H), 5.00-4.87 (m, 2H), 4.79-4.73 (m, 1H), 4.72-4.46 (m, 7H), 4.45-4.27 (m, 5H), 4.14-3.93 (m, 5H), 3.90-3.76 (m, 5H), 3.75-3.66 (m, 8H), 3.59-3.47 (m, 4H), 3.41-3.29 (m, 2H), 2.47 (s, 3H), 2.25-2.19 (m, 1H), 2.08 (td, J=9.4, 4.7 Hz, 1H), 1.04 (s, 9H). HRMS (m/z) for C₅₂H₇₂N₁₁O₁₀S⁺ [M+H]⁺: molecular weight calculated 1042.5179, found 1042.5165.

### Example 46—Synthesis of YS43-41

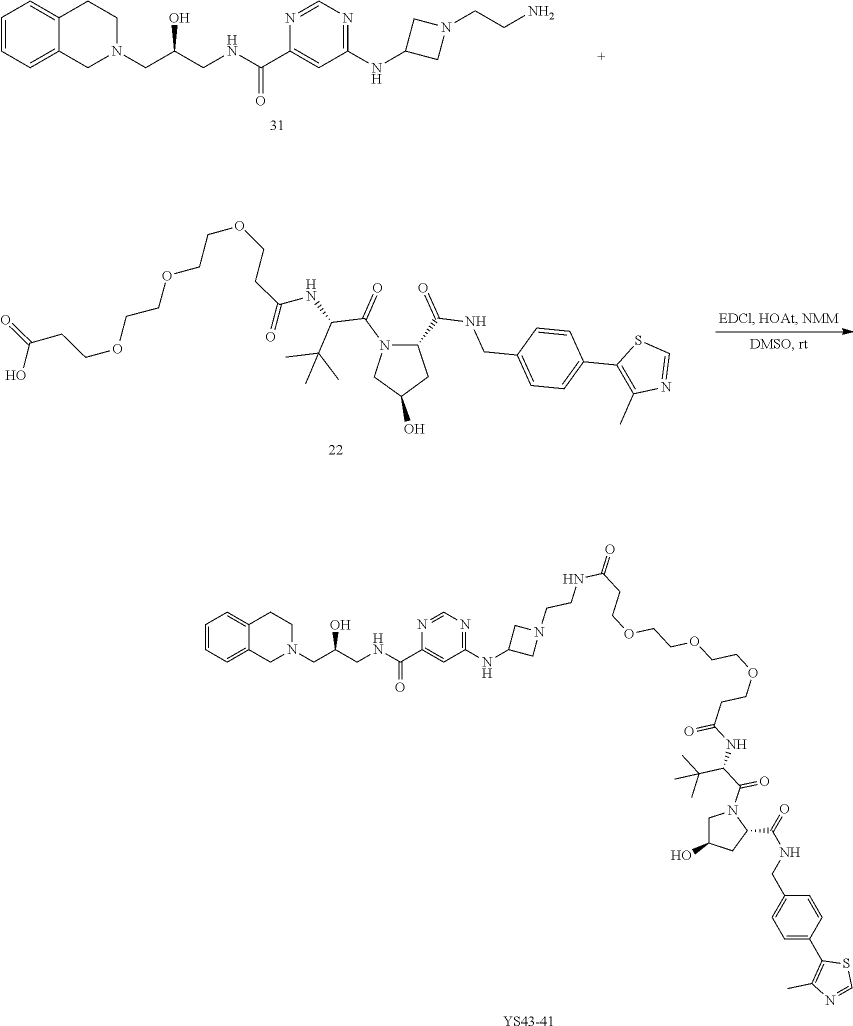 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCOCCOCCOCCC(=O)O)C(C)(C)C)cc1 ; "22" ; Cc1ncsc1-c1ccc(CNC(=O)[C@@H]2C[C@@H](O)CN2C(=O)[C@@H](NC(=O)CCOCCOCCOCCC(=O)NCCN2CC(Nc3cc(C(=O)NC[C@H](O)CN4CCc5ccccc5C4)ncn3)C2)C(C)(C)C)cc1 ; "YS43-41"]

To a solution of Intermediate 31 in TFA salt form (5 mg, 0.01 mmol), intermediate 22 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-41 as white solid in TFA salt form (3 mg, yield 28%). ¹H NMR (600 MHz, Methanol-d₄) δ 8.95-8.84 (m, 2H), 7.66 (s, 1H), 7.49-7.36 (m, 4H), 7.36-7.23 (m, 3H), 7.19 (d, J=7.5 Hz, 1H), 5.01-4.91 (m, 2H), 4.77-4.71 (m, 1H), 4.68-4.47 (m, 7H), 4.47-4.31 (m, 5H), 4.25-4.15 (m, 1H), 3.92-3.77 (m, 5H), 3.73 (s, 4H), 3.67-3.44 (m, 12H), 3.41-3.28 (m, 2H), 2.60-2.41 (m, 7H), 2.23 (dd, J=13.3, 7.6 Hz, 1H), 2.08 (ddd, J=13.4, 9.3, 4.4 Hz, 1H), 1.03 (d, J=3.0 Hz, 9H). HRMS (m/z) for C₅₄H₇₆N₁₁O₁₀S⁺ [M+H]⁺: molecular weight calculated 1070.5492, found 1070.5462.

### Example 47—Synthesis of YS43-42

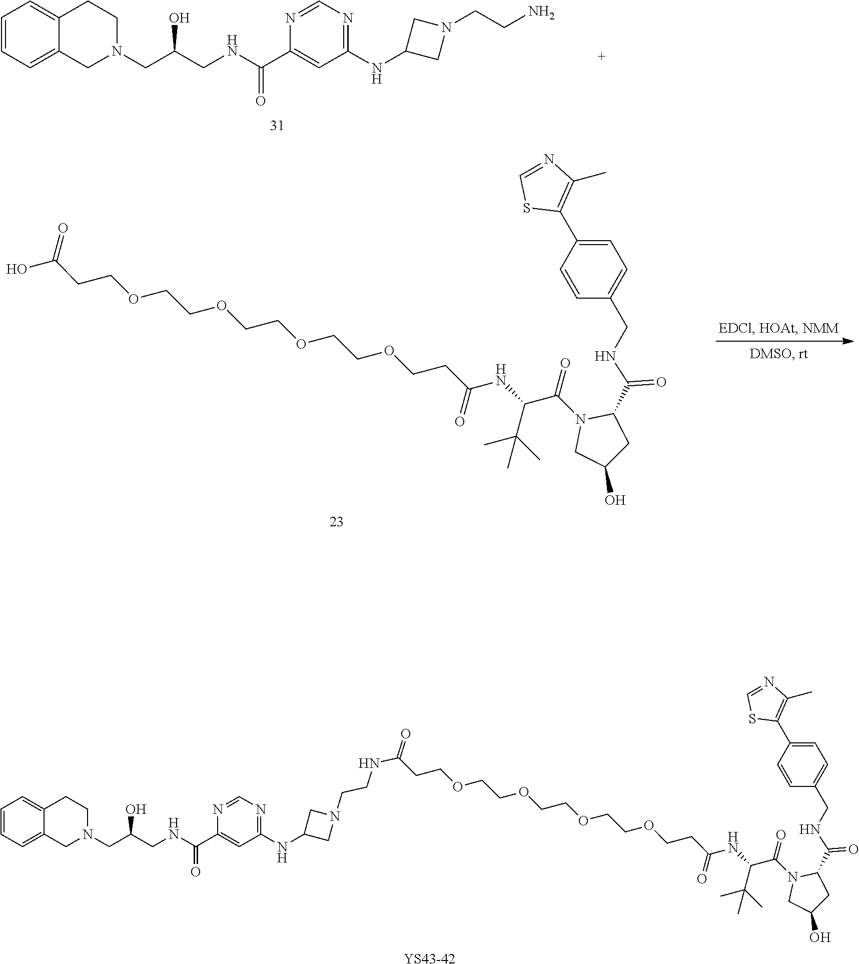 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; "23" ; Cc1ncsc1-c1ccc(CNC(=O)[C@@H]2C[C@@H](O)CN2C(=O)[C@@H](NC(=O)CCOCCOCCOCCOCCC(=O)NCCN2CC(Nc3cc(C(=O)NC[C@H](O)CN4CCc5ccccc5C4)ncn3)C2)C(C)(C)C)cc1 ; "YS43-42"]

To a solution of Intermediate 31 in TFA salt form (5 mg, 0.01 mmol), intermediate 23 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-42 as white solid in TFA salt form (4 mg, yield 36%). ¹H NMR (600 MHz, Methanol-d₄) δ 8.93-8.86 (m, 2H), 7.65 (s, 1H), 7.48-7.39 (m, 4H), 7.33-7.24 (m, 3H), 7.19 (d, J=7.6 Hz, 1H), 5.01-4.89 (m, 2H), 4.71 (dd, J=12.7, 6.4 Hz, 1H), 4.66-4.46 (m, 7H), 4.46-4.31 (m, 5H), 4.21-4.14 (m, 1H), 3.91-3.76 (m, 5H), 3.75-3.69 (m, 4H), 3.65-3.50 (m, 16H), 3.41-3.29 (m, 2H), 2.61-2.40 (m, 7H), 2.23 (dd, J=13.2, 7.6 Hz, 1H), 2.08 (ddd, J=13.3, 9.3, 4.4 Hz, 1H), 1.04 (s, 9H). HRMS (m/z) for C₅₆H₈₀N₁₁O₁₁S⁺ [M+H]⁺: molecular weight calculated 1114.5754, found 1114.5745.

### Example 48—Synthesis of YS43-43

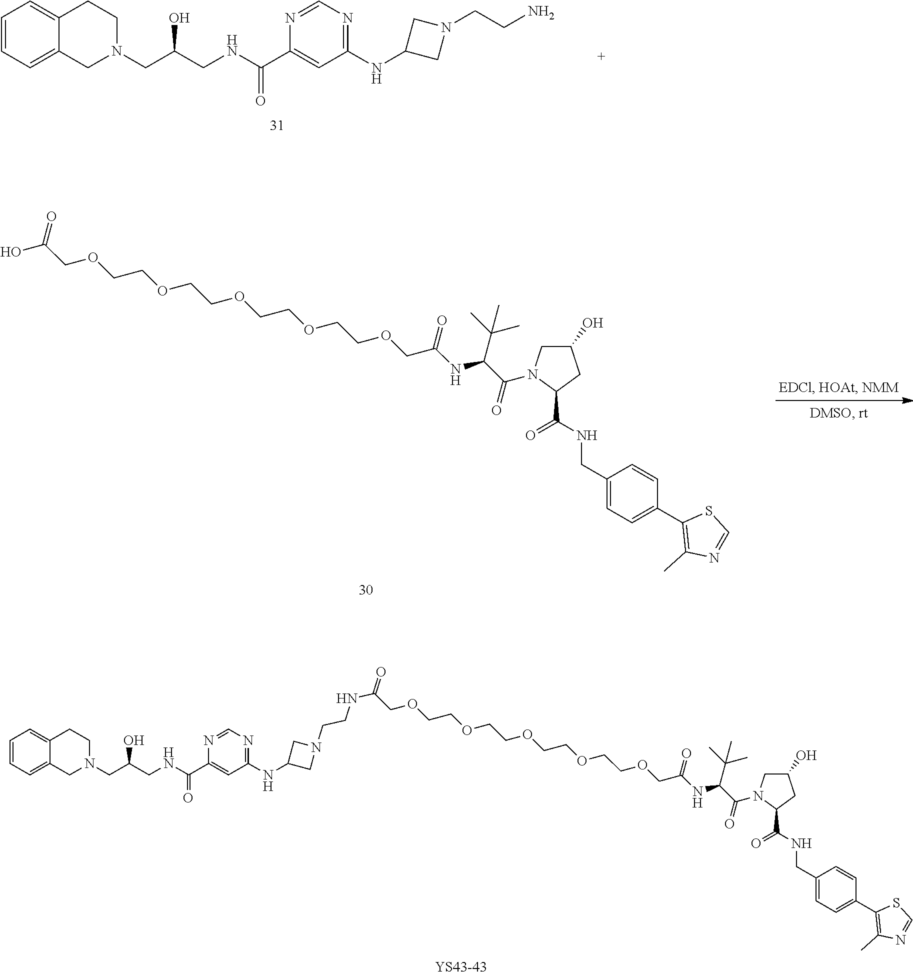 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; "30" ; Cc1ncsc1-c1ccc(CNC(=O)[C@@H]2C[C@@H](O)CN2C(=O)[C@@H](NC(=O)COCCOCCOCCOCCOCC(=O)NCCN2CC(Nc3cc(C(=O)NC[C@H](O)CN4CCc5ccccc5C4)ncn3)C2)C(C)(C)C)cc1 ; "YS43-43"]

To a solution of Intermediate 31 in TFA salt form (5 mg, 0.01 mmol), intermediate 30 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-43 as white solid in TFA salt form (3 mg, yield 27%). ¹H NMR (600 MHz, Methanol-d₄) δ 8.91-8.84 (m, 2H), 7.63 (s, 1H), 7.47-7.39 (m, 4H), 7.34-7.22 (m, 3H), 7.19 (d, J=7.6 Hz, 1H), 4.86-4.77 (m, 2H), 4.78-4.72 (m, 1H), 4.69-4.47 (m, 7H), 4.48-4.28 (m, 5H), 4.20-3.95 (m, 5H), 3.92-3.77 (m, 5H), 3.73-3.47 (m, 20H), 3.39-3.30 (m, 2H), 2.47 (s, 3H), 2.29-2.19 (m, 1H), 2.15-2.02 (m, 1H), 1.05 (s, 9H). HRMS (m/z) for C₅₆H₈₀N₁₁O₁₂S⁺ [M+H]⁺: molecular weight calculated 1130.5703, found 1130.5676.

### Example 49—Synthesis of YS43-44

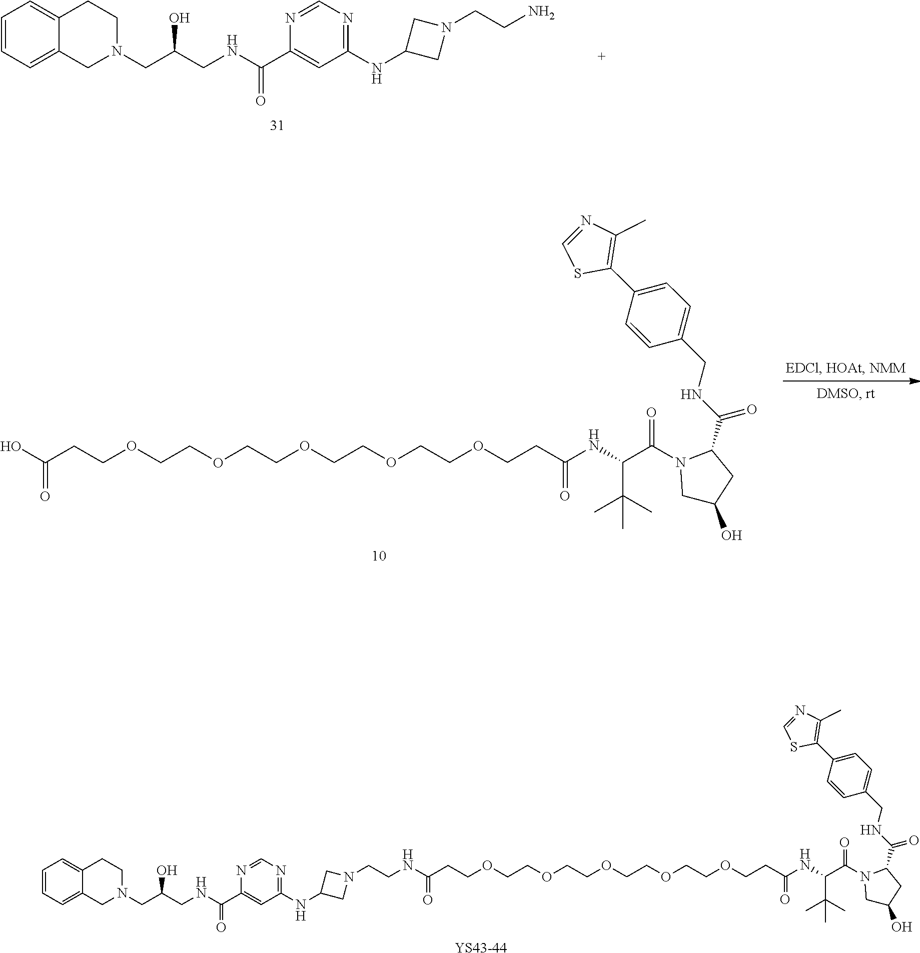 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; "10" ; Cc1ncsc1-c1ccc(CNC(=O)[C@@H]2C[C@@H](O)CN2C(=O)[C@@H](NC(=O)CCOCCOCCOCCOCCOCCC(=O)NCCN2CC(Nc3cc(C(=O)NC[C@H](O)CN4CCc5ccccc5C4)ncn3)C2)C(C)(C)C)cc1 ; "YS43-44"]

To a solution of Intermediate 31 in TFA salt form (5 mg, 0.01 mmol), intermediate 10 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-44 as white solid in TFA salt form (7 mg, yield 60%). ¹H NMR (600 MHz, Methanol-d₄) δ 8.89 (s, 2H), 7.63 (s, 1H), 7.48-7.37 (m, 4H), 7.34-7.24 (m, 3H), 7.19 (d, J=7.5 Hz, 1H), 4.98-4.90 (m, 2H), 4.68 (dd, J=12.4, 6.2 Hz, 1H), 4.65-4.46 (m, 7H), 4.46-4.29 (m, 5H), 4.20-4.11 (m, 1H), 3.90-3.78 (m, 5H), 3.76-3.69 (m, 4H), 3.66-3.47 (m, 20H), 3.41-3.31 (m, 2H), 2.62-2.41 (m, 7H), 2.22 (dd, J=13.2, 7.6 Hz, 1H), 2.08 (ddd, J=13.3, 9.3, 4.4 Hz, 1H), 1.04 (s, 9H). HRMS (m/z) for C₅₈H₈₄N₁₁O₁₂S⁺ [M+H]⁺: molecular weight calculated 1158.6016, found 1158.5996.

### Example 50—Synthesis of YS43-45

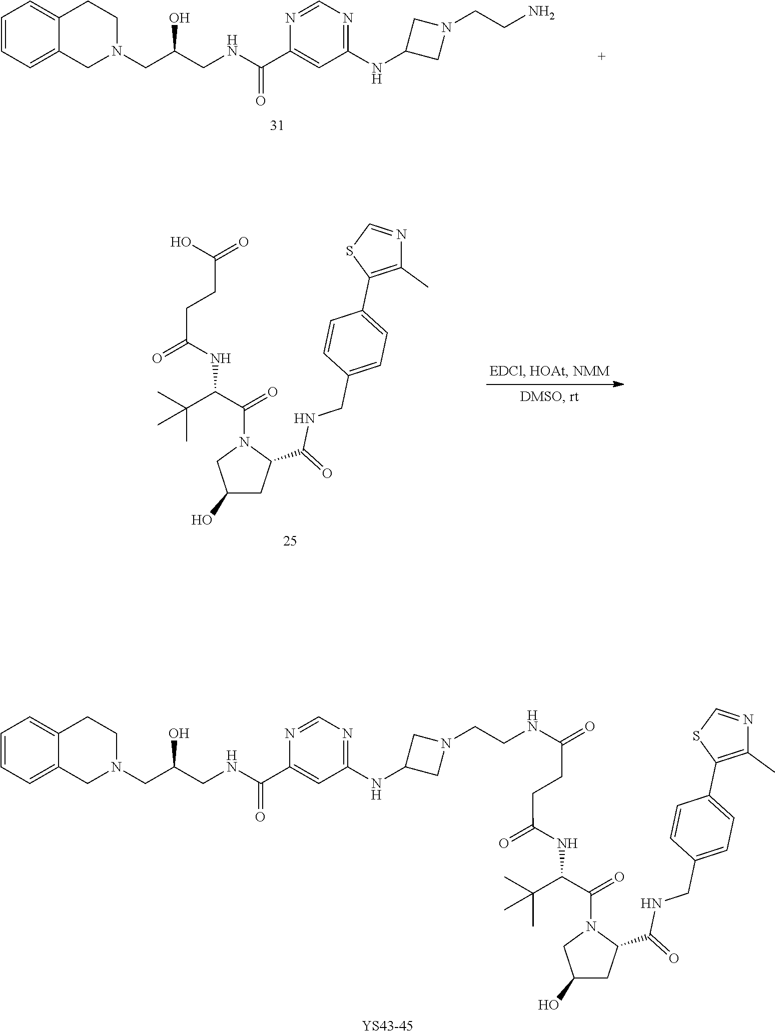 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCC(=O)O)C(C)(C)C)cc1 ; "25" ; Cc1ncsc1-c1ccc(CNC(=O)[C@@H]2C[C@@H](O)CN2C(=O)[C@@H](NC(=O)CCC(=O)NCCN2CC(Nc3cc(C(=O)NC[C@H](O)CN4CCc5ccccc5C4)ncn3)C2)C(C)(C)C)cc1 ; "YS43-45"]

To a solution of Intermediate 31 in TFA salt form (5 mg, 0.01 mmol), intermediate 25 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-45 as white solid in TFA salt form (6 mg, yield 64%). ¹H NMR (600 MHz, Methanol-d₄) δ 8.93-8.80 (m, 2H), 7.61 (s, 1H), 7.47-7.36 (m, 4H), 7.35-7.23 (m, 3H), 7.19 (d, J=7.5 Hz, 1H), 4.80-4.25 (m, 15H), 3.90-3.72 (m, 5H), 3.61-3.45 (m, 5H), 3.41-3.29 (m, 2H), 2.70-2.59 (m, 2H), 2.51-2.37 (m, 5H), 2.28-2.24 (m, 1H), 2.11-2.06 (m, 1H), 1.05 (s, 9H). HRMS (m/z) for C₄₈H₆₄N₁₁O₇S⁺ [M+H]⁺: molecular weight calculated 938.4705, found 938.4695.

### Example 51—Synthesis of YS43-46

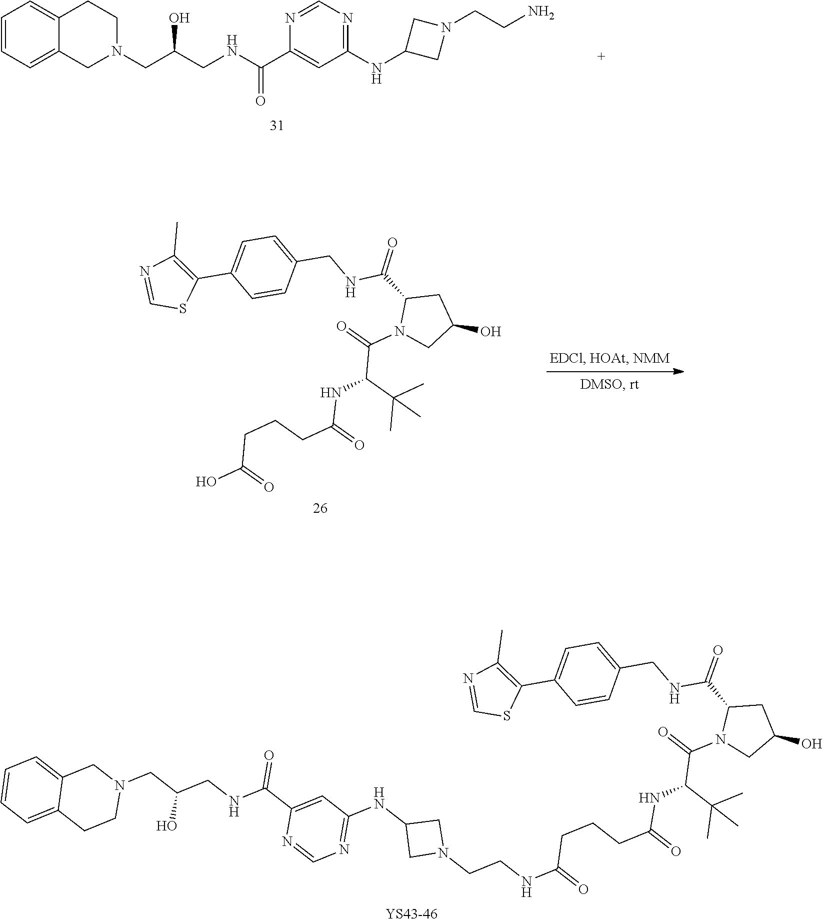 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCCC(=O)O)C(C)(C)C)cc1 ; "26" ; Cc1ncsc1-c1ccc(CNC(=O)[C@@H]2C[C@@H](O)CN2C(=O)[C@@H](NC(=O)CCCC(=O)NCCN2CC(Nc3cc(C(=O)NC[C@H](O)CN4CCc5ccccc5C4)ncn3)C2)C(C)(C)C)cc1 ; "YS43-46"]

To a solution of Intermediate 31 in TFA salt form (5 mg, 0.01 mmol), intermediate 26 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-46 as white solid in TFA salt form (5 mg, yield 52%). ¹H NMR (600 MHz, Methanol-d₄) δ 8.96-8.86 (m, 2H), 7.64 (s, 1H), 7.43 (s, 4H), 7.35-7.25 (m, 3H), 7.19 (s, 1H), 4.76-4.64 (m, 1H), 4.68-4.45 (m, 9H), 4.46-4.26 (m, 5H), 3.97-3.70 (m, 5H), 3.54 (d, J=17.9 Hz, 5H), 3.38-3.32 (m, 2H), 2.47 (s, 3H), 2.39-2.20 (m, 5H), 2.12-2.02 (m, 1H), 1.96-1.85 (m, 2H), 1.04 (s, 9H). HRMS (m/z) for C₄₉H₆₆N₁₁O₇S⁺ [M+H]⁺: molecular weight calculated 952.4862, found 952.4871.

### Example 52—Synthesis of YS43-47

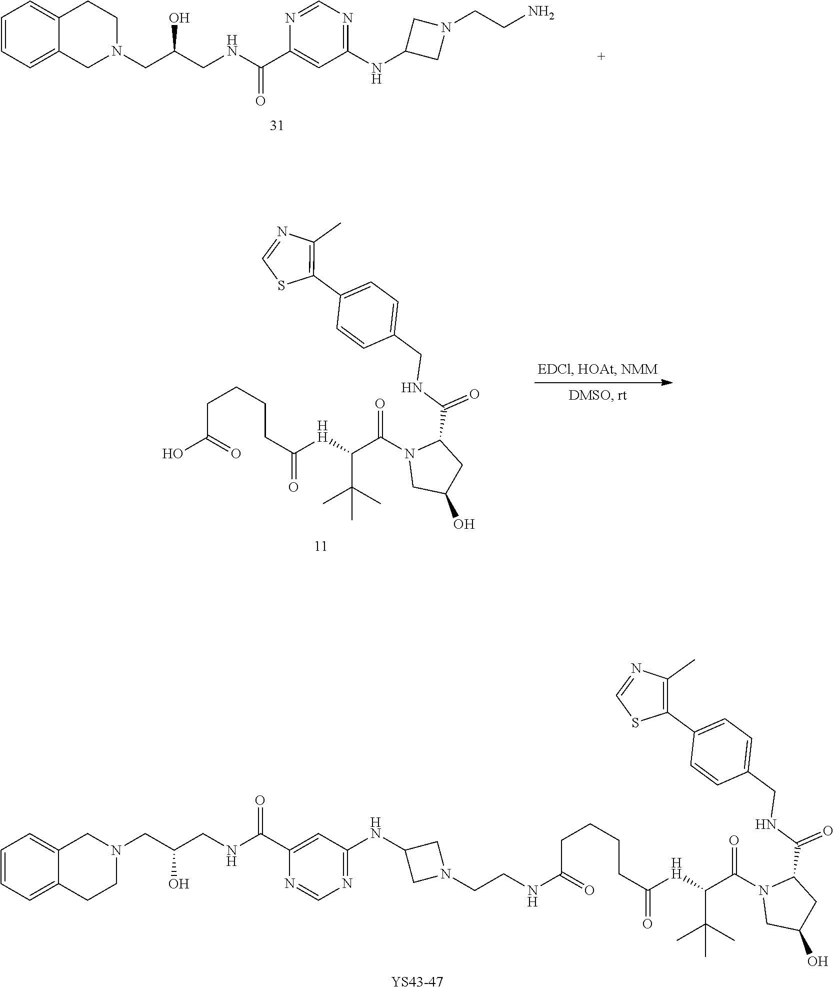

To a solution of Intermediate 31 in TFA salt form (5 mg, 0.01 mmol), intermediate 11 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-47 as white solid in TFA salt form (6 mg, yield 62%). ¹H NMR (600 MHz, Methanol-d₄) δ 9.00-8.71 (m, 2H), 7.67 (s, 1H), 7.50-7.39 (m, 4H), 7.34-7.24 (m, 3H), 7.19 (s, 1H), 4.84-4.24 (m, 15H), 4.01-3.71 (m, 5H), 3.60-3.44 (m, 5H), 3.30-3.13 (m, 2H), 2.47 (s, 3H), 2.34-2.18 (m, 5H), 2.14-2.05 (m, 1H), 1.66-1.56 (m, 4H), 1.03 (s, 9H). HRMS (m/z) for C₅₀H₆₈N₁₁O₇S⁺ [M+H]⁺: molecular weight calculated 966.5018, found 966.5021.

### Example 53—Synthesis of YS43-48

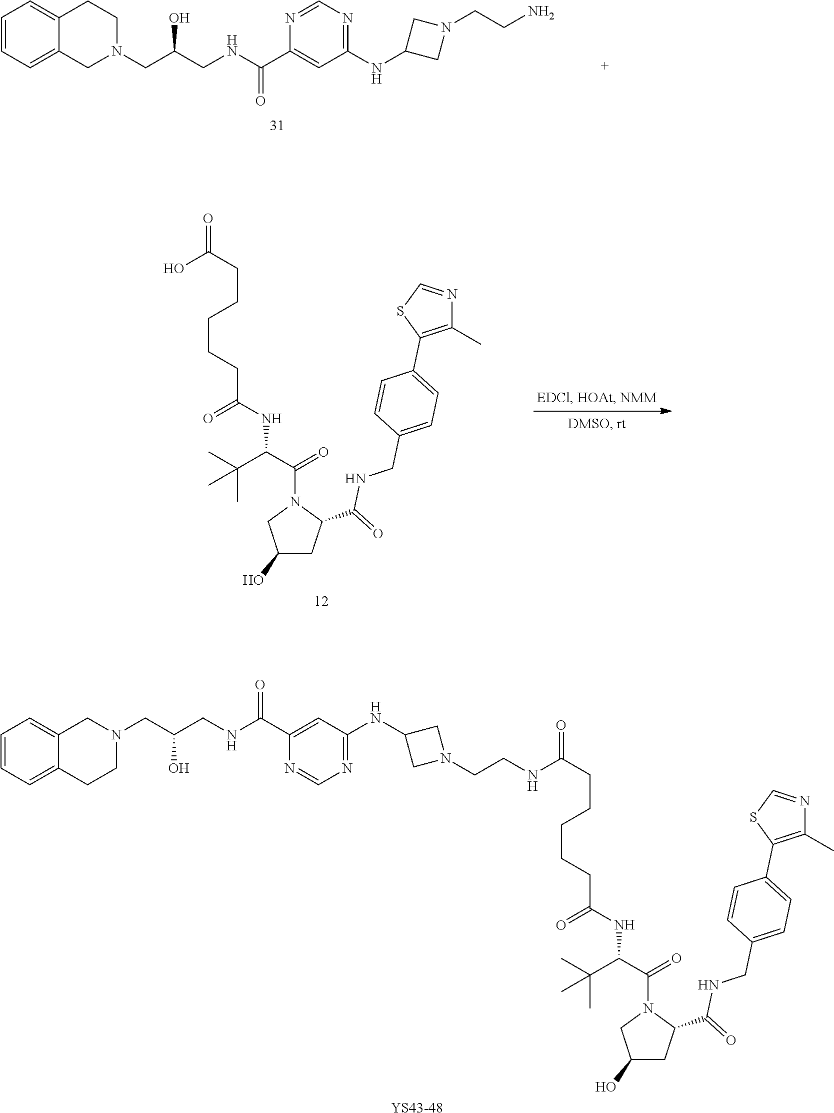 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCCCCC(=O)O)C(C)(C)C)cc1 ; "12" ; Cc1ncsc1-c1ccc(CNC(=O)[C@@H]2C[C@@H](O)CN2C(=O)[C@@H](NC(=O)CCCCCC(=O)NCCN2CC(Nc3cc(C(=O)NC[C@H](O)CN4CCc5ccccc5C4)ncn3)C2)C(C)(C)C)cc1 ; "YS43-48"]

To a solution of Intermediate 31 in TFA salt form (5 mg, 0.01 mmol), intermediate 12 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-48 as white solid in TFA salt form (8 mg, yield 82%). ¹H NMR (600 MHz, Methanol-d₄) δ 9.13-8.80 (m, 2H), 7.66 (s, 1H), 7.61-7.38 (m, 4H), 7.35-7.25 (m, 3H), 7.23-7.15 (m, 1H), 4.91-4.46 (m, 10H), 4.47-4.24 (m, 5H), 3.99-3.77 (m, 5H), 3.64-3.45 (m, 5H), 3.42-3.30 (m, 2H), 2.47 (s, 3H), 2.33-2.01 (m, 6H), 1.66-1.59 (m, 4H), 1.41-1.26 (m, 2H), 1.02 (s, 9H). HRMS (m/z) for C₅₁H₇₀N₁₁O₇S⁺ [M+H]⁺: molecular weight calculated 980.5175, found 980.5156.

### Example 54—Synthesis of YS43-49

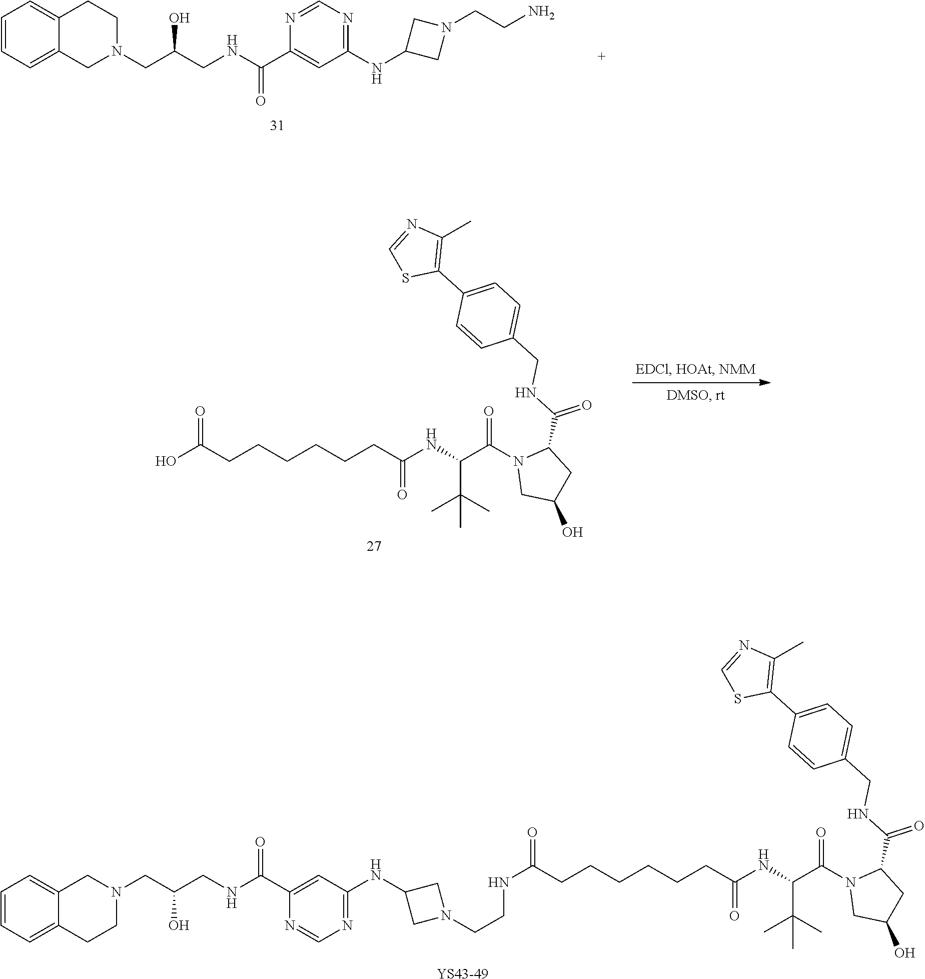 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCCCCCC(=O)O)C(C)(C)C)cc1 ; "27" ; Cc1ncsc1-c1ccc(CNC(=O)[C@@H]2C[C@@H](O)CN2C(=O)[C@@H](NC(=O)CCCCCCC(=O)NCCN2CC(Nc3cc(C(=O)NC[C@H](O)CN4CCc5ccccc5C4)ncn3)C2)C(C)(C)C)cc1 ; "YS43-49"]

To a solution of Intermediate 31 in TFA salt form (5 mg, 0.01 mmol), intermediate 27 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-49 as white solid in TFA salt form (7 mg, yield 71%). ¹H NMR (600 MHz, Methanol-d₄) δ 8.93-8.84 (m, 2H), 7.65 (s, 1H), 7.51-7.36 (m, 4H), 7.34-7.24 (m, 3H), 7.19 (d, J=7.5 Hz, 1H), 4.83-4.46 (m, 10H), 4.37 (s, 5H), 3.94-3.65 (m, 5H), 3.62-3.43 (m, 5H), 3.33-3.18 (m, 2H), 2.47 (s, 3H), 2.33-2.15 (m, 5H), 2.08 (ddd, J=13.3, 9.1, 4.5 Hz, 1H), 1.63-1.56 (m, 4H), 1.39-1.27 (m, 4H), 1.02 (s, 9H). HRMS (m/z) for C₅₂H₇₂N₁₁O₇S⁺ [M+H]⁺: molecular weight calculated 994.5331, found 994.5299.

### Example 55—Synthesis of YS43-50

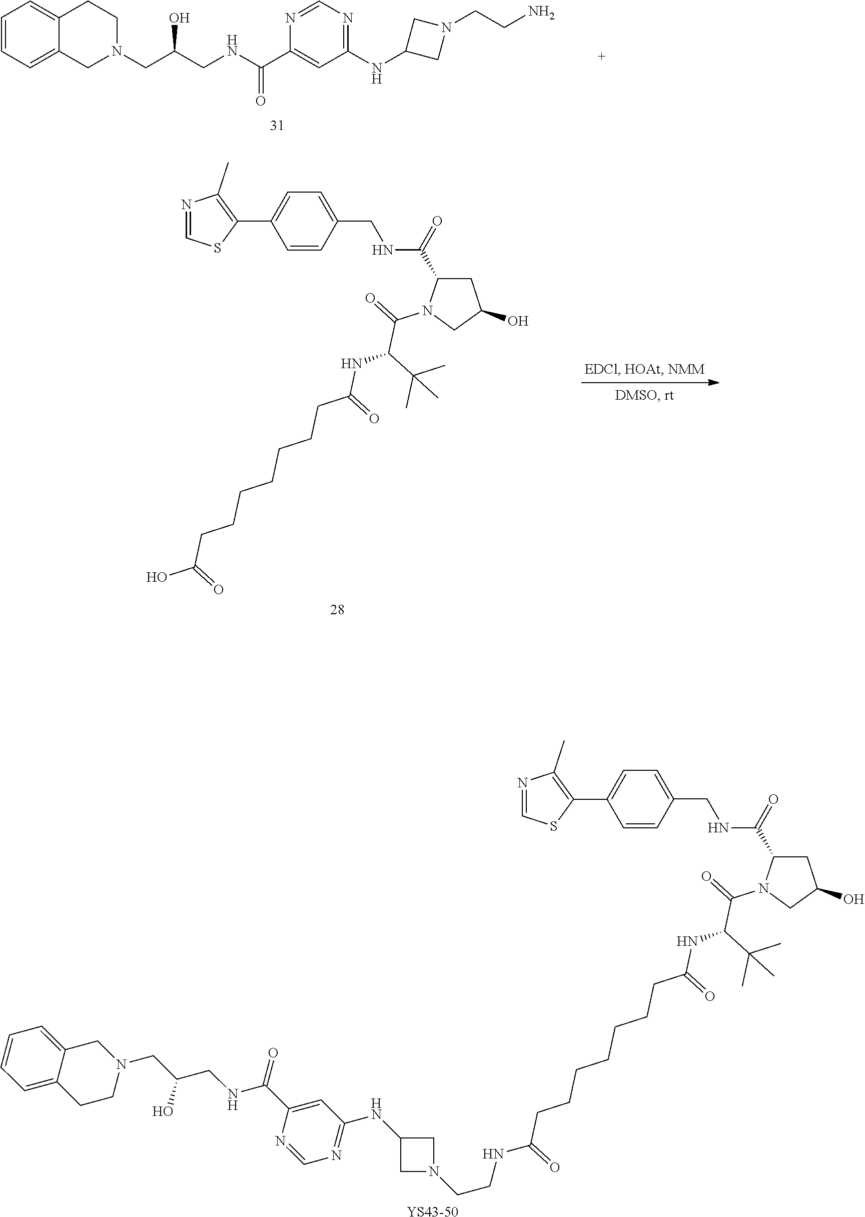

To a solution of Intermediate 31 in TFA salt form (5 mg, 0.01 mmol), intermediate 28 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-50 as white solid in TFA salt form (6 mg, yield 60%). ¹H NMR (600 MHz, Methanol-d₄) δ 8.88 (s, 1H), 8.86 (s, 1H), 7.61 (s, 1H), 7.48-7.40 (m, 4H), 7.34-7.24 (m, 3H), 7.19 (d, J=7.5 Hz, 1H), 4.98-4.89 (m, 1H), 4.71-4.47 (m, 9H), 4.36 (d, J=15.4 Hz, 5H), 3.92-3.75 (m, 5H), 3.58-3.46 (m, 5H), 3.40-3.20 (m, 2H), 2.47 (s, 3H), 2.25 (dh, J=28.9, 7.1 Hz, 5H), 2.11-2.04 (m, 1H), 1.66-1.53 (m, 4H), 1.36-1.24 (m, 6H), 1.03 (s, 9H). HRMS (m/z) for C₅₃H₇₄N₁₁O₇S⁺ [M+H]⁺: molecular weight calculated 1008.5488, found 1008.5465.

### Example 56—Synthesis of YS43-51

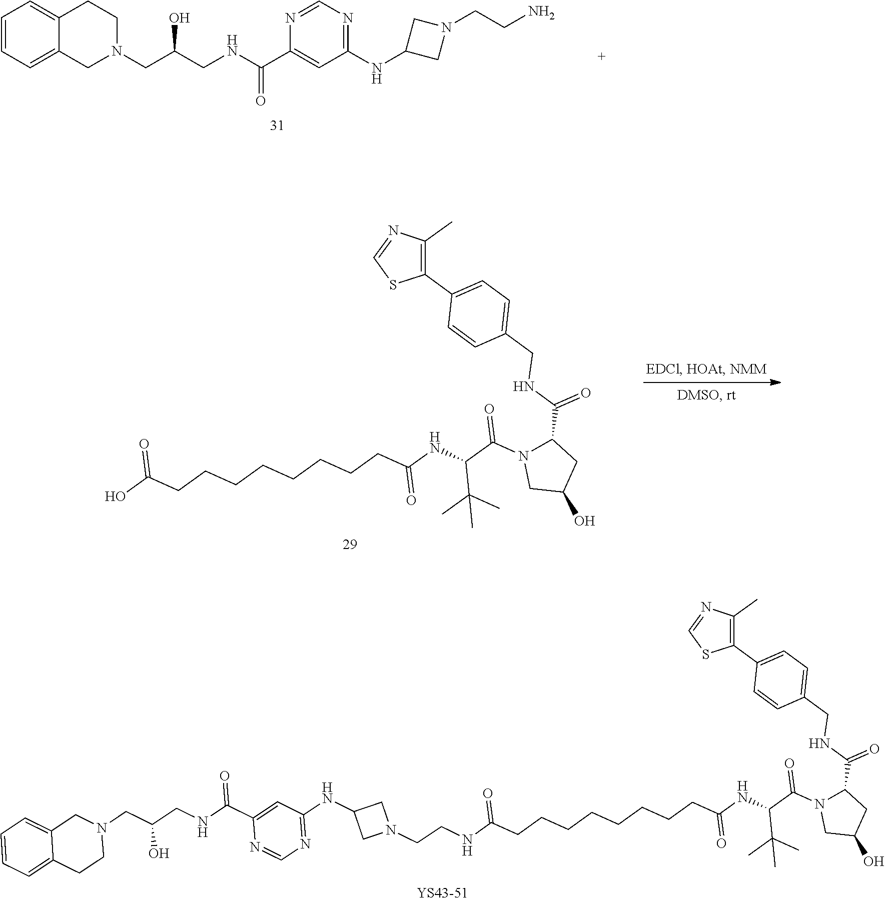 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCCCCCCCC(=O)O)C(C)(C)C)cc1 ; "29" ; Cc1ncsc1-c1ccc(CNC(=O)[C@@H]2C[C@@H](O)CN2C(=O)[C@@H](NC(=O)CCCCCCCCC(=O)NCCN2CC(Nc3cc(C(=O)NC[C@H](O)CN4CCc5ccccc5C4)ncn3)C2)C(C)(C)C)cc1 ; "YS43-51"]

To a solution of Intermediate 31 in TFA salt form (5 mg, 0.01 mmol), intermediate 29 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-51 as white solid in TFA salt form (6 mg, yield 49%). ¹H NMR (600 MHz, Methanol-d₄) δ 8.88 (d, J=2.5 Hz, 1H), 8.85 (s, 1H), 7.60 (s, 1H), 7.48-7.40 (m, 4H), 7.32-7.24 (m, 3H), 7.19 (d, J=7.7 Hz, 1H), 4.88-4.76 (m, 1H), 4.70-4.47 (m, 9H), 4.36 (d, J=14.1 Hz, 5H), 3.94-3.73 (m, 5H), 3.60-3.47 (m, 5H), 3.42-3.19 (m, 2H), 2.47 (dd, J=4.0, 2.7 Hz, 3H), 2.35-2.18 (m, 5H), 2.13-2.02 (m, 1H), 1.65-1.53 (m, 4H), 1.35-1.22 (m, 8H), 1.03 (s, 9H). HRMS (m/z) for C₅₄H₇₆N₁₁O₇S⁺ [M+H]⁺: molecular weight calculated 1022.5644, found 1022.5639.

### Example 57—Synthesis of YS43-52

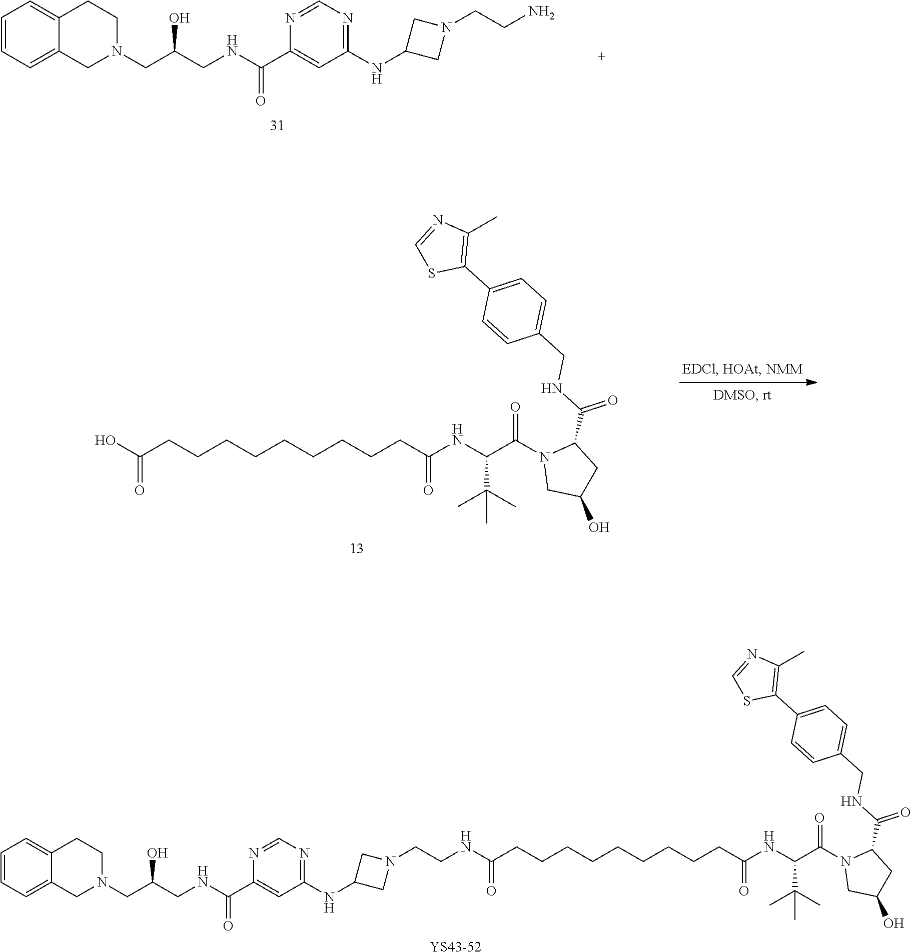 [SMILES: NCCN1CC(Nc2cc(C(=O)NC[C@H](O)CN3CCc4ccccc4C3)ncn2)C1 ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; "13" ; Cc1ncsc1-c1ccc(CNC(=O)[C@@H]2C[C@@H](O)CN2C(=O)[C@@H](NC(=O)CCCCCCCCCC(=O)NCCN2CC(Nc3cc(C(=O)NC[C@H](O)CN4CCc5ccccc5C4)ncn3)C2)C(C)(C)C)cc1 ; "YS43-52"]

To a solution of Intermediate 31 in TFA salt form (5 mg, 0.01 mmol), intermediate 13 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-52 as white solid in TFA salt form (3 mg, yield 29%). ¹H NMR (600 MHz, Methanol-d₄) δ 8.88 (s, 1H), 8.79 (s, 1H), 7.54 (s, 1H), 7.46 (d, J=7.9 Hz, 2H), 7.41 (d, J=8.0 Hz, 2H), 7.33-7.25 (m, 3H), 7.19 (d, J=7.7 Hz, 1H), 4.88-4.81 (m, 1H), 4.65-4.47 (m, 9H), 4.45-4.32 (m, 5H), 3.92-3.73 (m, 5H), 3.59-3.41 (m, 5H), 3.40-3.17 (m, 2H), 2.47 (s, 3H), 2.33-2.26 (m, 1H), 2.26-2.17 (m, 4H), 2.12-2.05 (m, 1H), 1.63-1.53 (m, 4H), 1.35-1.25 (m, 10H), 1.03 (s, 9H). HRMS (m/z) for C₅₅H₇₈N₁₁O₇S⁺ [M+H]⁺: molecular weight calculated 1036.5801, found 1036.5822.

### Example 58—Synthesis of YS43-53

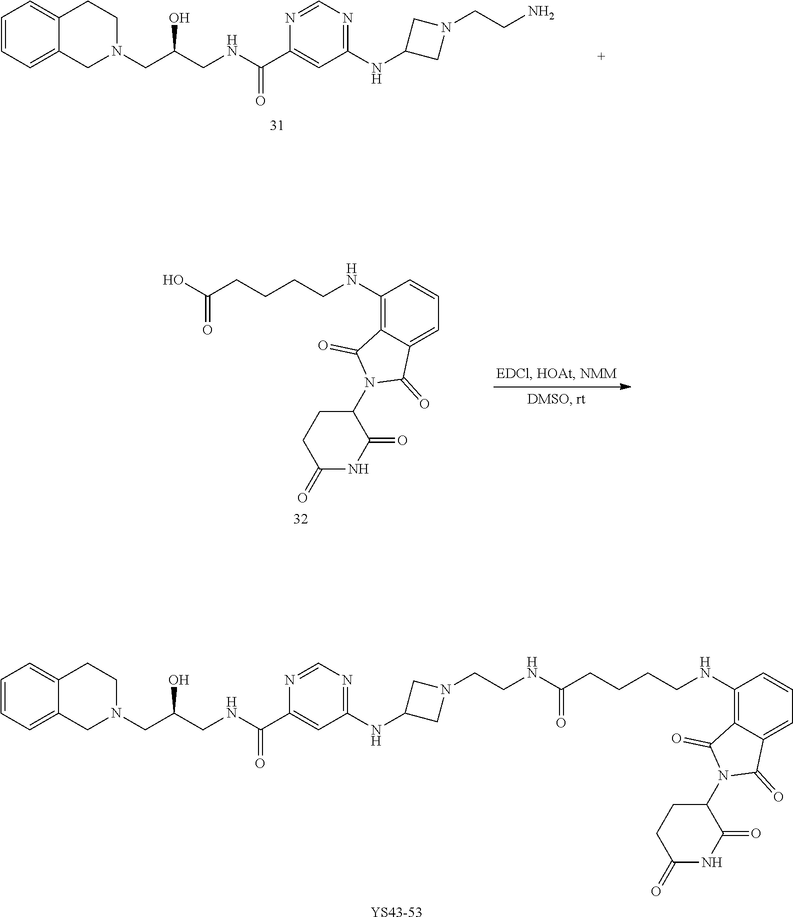

To a solution of Intermediate 31 in TFA salt form (5 mg, 0.01 mmol), intermediate 32 (5 mg, 0.01 mmol, 1 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-53 as yellow solid in TFA salt form (2 mg, yield 26%). ¹H NMR (600 MHz, Methanol-d₄) δ 8.78 (s, 1H), 7.58-7.51 (m, 2H), 7.34-7.17 (m, 4H), 7.04 (t, J=6.8 Hz, 2H), 5.05 (dd, J=12.7, 5.5 Hz, 1H), 4.96-4.86 (m, 2H), 4.66-4.56 (m, 3H), 4.38 (d, J=41.5 Hz, 3H), 3.87-3.80 (m, 1H), 3.57-3.41 (m, 6H), 3.37-3.19 (m, 6H), 2.88-2.62 (m, 4H), 2.34-2.28 (m, 2H), 2.13-2.08 (m, 1H), 1.76-1.65 (m, 4H). HRMS (m/z) for C₄₀H₄₉N₁₀O₇⁺ [M+H]⁺: molecular weight calculated 781.3780, found 781.3763.

### Example 59—Synthesis of YS43-54

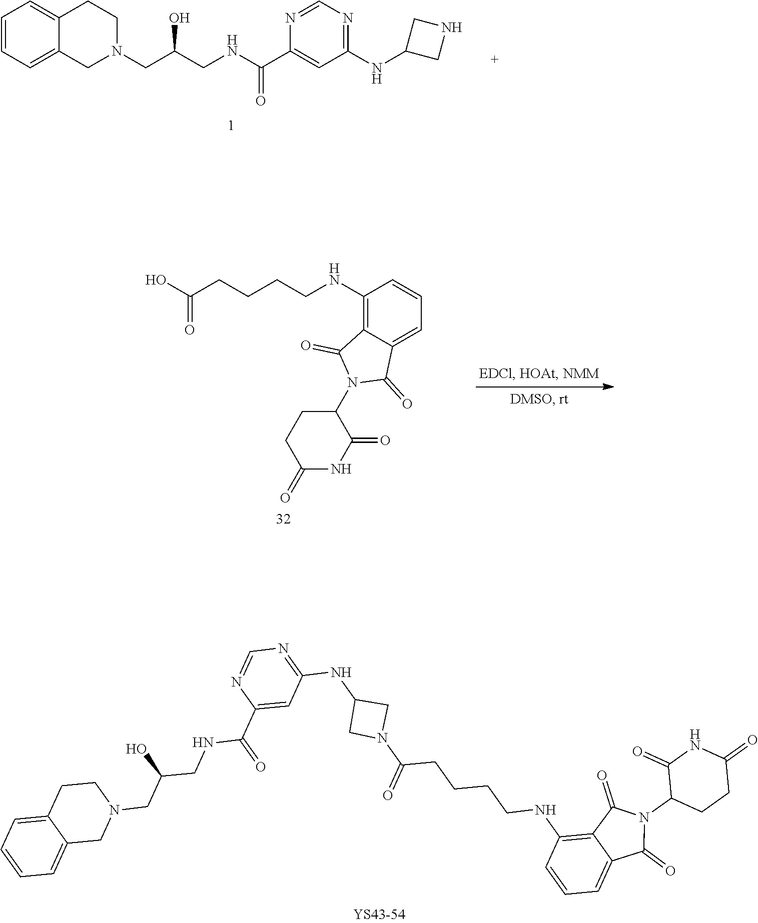

To a solution of Intermediate 1 in TFA salt form (10 mg, 0.01 mmol), intermediate 32 mg, 0.03 mmol, 3 equiv), EDCI (1-ethyl-3-(3-dimethylaminopropyl)carbodiimide) (6 mg, 0.03 mmol, 3 equiv), and HOAt (1-hydroxy-7-azabenzo-triazole) (4 mg, 0.03 mmol, 3 equiv) in 1 mL of DMSO, was added NMM (N-Methylmorpholine) (15 mg, 0.15 mmol, 15 equiv). After stirring overnight at room temperature, the resulting mixture was purified by preparative HPLC (10%-100% methanol/0.1% TFA in H₂O) to afford YS43-54 as yellow solid in TFA salt form (5 mg, yield 68%). ¹H NMR (600 MHz, Methanol-d₄) δ 8.53 (s, 1H), 7.53 (dt, J=10.6, 6.8 Hz, 1H), 7.34-7.12 (m, 5H), 7.02 (dt, J=16.8, 7.0 Hz, 2H), 5.04 (dd, J=11.6 Hz, 1H), 4.83-4.73 (m, 1H), 4.64-4.53 (m, 2H), 4.46-4.29 (m, 3H), 4.11-4.06 (m, 1H), 3.87 (d, J=34.9 Hz, 2H), 3.59-3.46 (m, 2H), 3.45-3.19 (m, 6H), 2.89-2.66 (m, 4H), 2.23 (q, J=6.8 Hz, 2H), 2.13-2.05 (m, 1H), 1.79-1.64 (m, 4H). HRMS (m/z) for C₃₈H₄₄N₉O₇⁺ [M+H]⁺: molecular weight calculated 738.3358, found 738.3354.

### Example 60: Synthesis of YS43-88

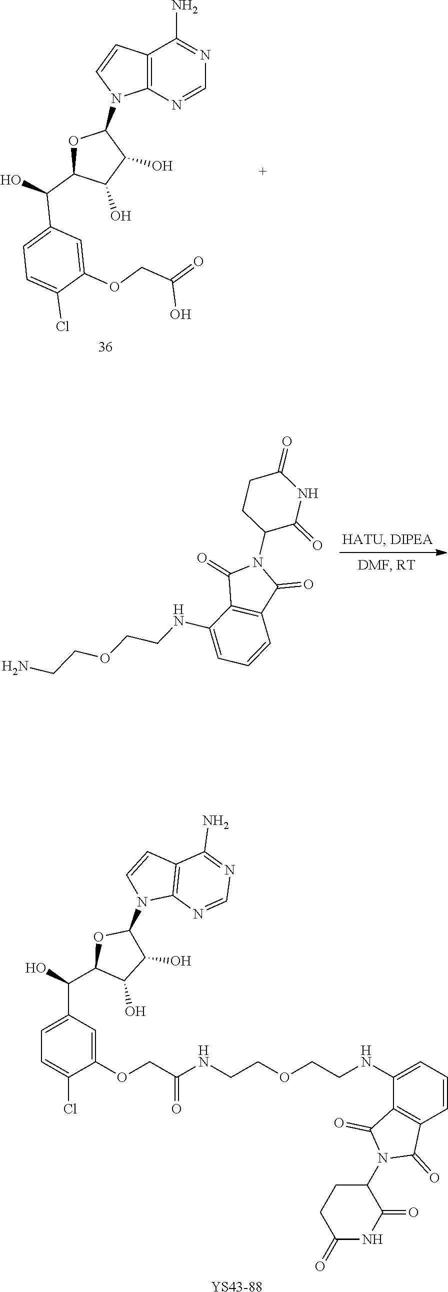 [SMILES: Nc1ncnc2c1ccn2[C@@H]1O[C@H]([C@H](O)c2ccc(Cl)c(OCC(=O)O)c2)[C@@H](O)[C@H]1O ; "36" ; NCCOCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCOCCNc3cccc4c3C(=O)N(C3CCC(=O)NC3=O)C4=O)c2)[C@@H](O)[C@H]1O ; "YS43-88"]

To a solution of intermediate 36 (9 mg, 0.02 mmol), 4-((2-(2-aminoethoxy)ethyl)amino)-2-(2,6-dioxopiperidin-3-yl)isoindoline-1,3-dione (7.4 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-88 (11 mg, yield 68%). ¹H NMR (800 MHz, Methanol-d₄) δ 8.26 (s, 1H), 7.65 (s, 1H), 7.49 (t, J=7.8 Hz, 1H), 7.35 (d, J=8.3 Hz, 1H), 7.13 (s, 1H), 7.09 (dd, J=8.5, 6.0 Hz, 1H), 7.05 (d, J=8.7 Hz, 1H), 6.99 (d, J=7.2 Hz, 1H), 6.92 (d, J=4.0 Hz, 1H), 6.21 (d, J=6.8 Hz, 1H), 5.01-4.96 (m, 1H), 4.93-4.91 (m, 1H), 4.62 (t, J=6.0 Hz, 1H), 4.57-4.55 (m, 2H), 4.30 (d, J=5.8 Hz, 1H), 4.24 (d, J=3.4 Hz, 1H), 3.73 (q, J=5.0, 4.2 Hz, 2H), 3.65 (t, J=5.2 Hz, 2H), 3.54 (t, J=5.3 Hz, 2H), 3.49 (t, J=5.2 Hz, 2H), 2.84-2.76 (m, 1H), 2.74-2.68 (m, 1H), 2.68-2.62 (m, 1H), 2.04 (dt, J=13.1, 5.4 Hz, 1H). MS (ESI) m/z 793.2 [M+H]⁺.

### Example 61: Synthesis of YS43-89

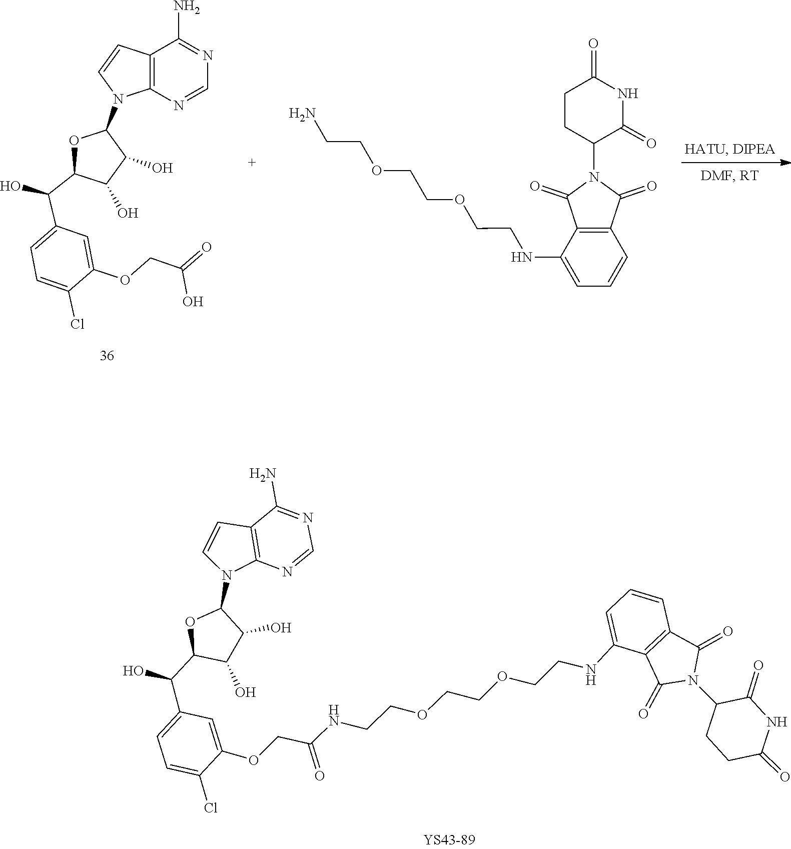 [SMILES: Nc1ncnc2c1ccn2[C@@H]1O[C@H]([C@H](O)c2ccc(Cl)c(OCC(=O)O)c2)[C@@H](O)[C@H]1O ; NCCOCCOCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCOCCOCCNc3cccc4c3C(=O)N(C3CCC(=O)NC3=O)C4=O)c2)[C@@H](O)[C@H]1O]

To a solution of intermediate 36 (9 mg, 0.02 mmol), 4-((2-(2-(2-aminoethoxy)ethoxy)ethyl)amino)-2-(2,6-dioxopiperidin-3-yl)isoindoline-1,3-dione (8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-89 (9.7 mg, yield 58%). ¹H NMR (800 MHz, Methanol-d₄) δ 8.25 (s, 1H), 7.64-7.62 (m, 1H), 7.48 (t, J=7.8 Hz, 1H), 7.34 (d, J=8.0 Hz, 1H), 7.14-7.07 (m, 2H), 7.03-6.96 (m, 2H), 6.92 (d, J=3.9 Hz, 1H), 6.26-6.16 (m, 1H), 5.05 (dd, J=12.9, 5.8 Hz, 1H), 4.92-4.90 (m, 1H), 4.62 (t, J=6.0 Hz, 1H), 4.49 (qd, J=14.7, 6.4 Hz, 2H), 4.30 (d, J=5.5 Hz, 1H), 4.27-4.21 (m, 1H), 3.75-3.70 (m, 2H), 3.70-3.61 (m, 6H), 3.54-3.47 (m, 2H), 3.45 (t, J=5.4 Hz, 2H), 2.87-2.81 (m, 1H), 2.76-2.66 (m, 2H), 2.13-2.06 (m, 1H). MS (ESI) m/z 837.1 [M+H]⁺.

### Example 62: Synthesis of YS43-90

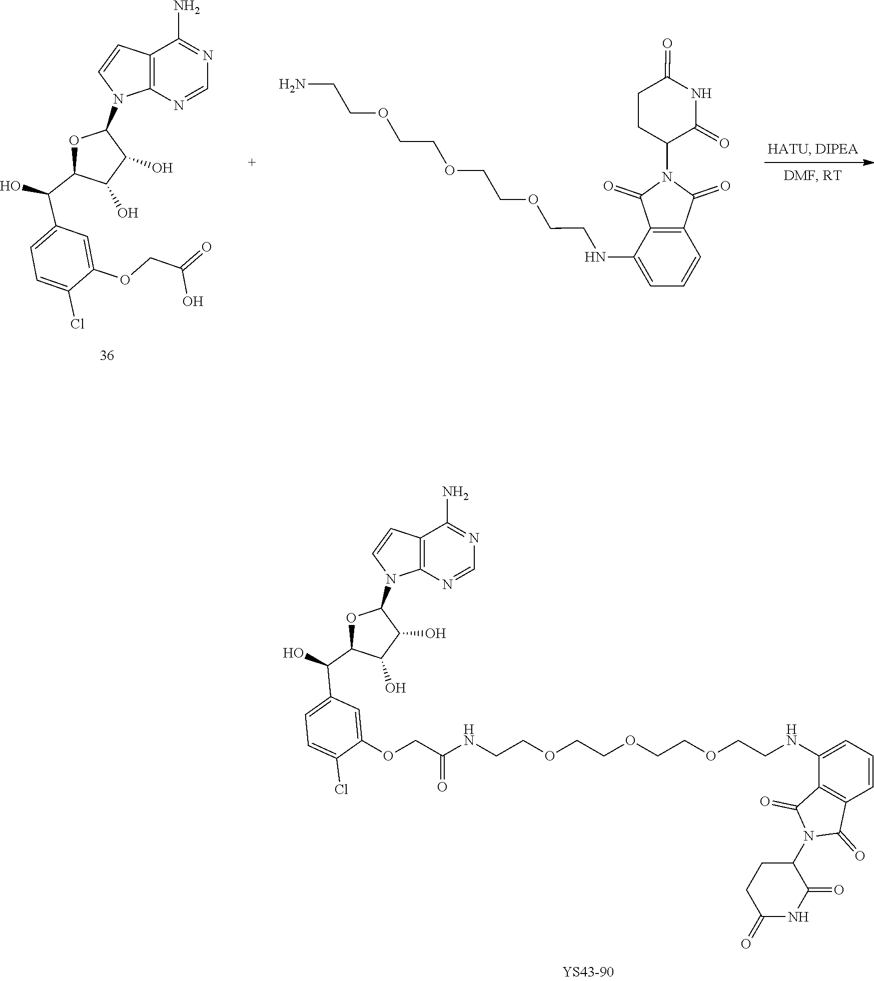 [SMILES: Nc1ncnc2c1ccn2[C@@H]1O[C@H]([C@H](O)c2ccc(Cl)c(OCC(=O)O)c2)[C@@H](O)[C@H]1O ; NCCOCCOCCOCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCOCCOCCOCCNc3cccc4c3C(=O)N(C3CCC(=O)NC3=O)C4=O)c2)[C@@H](O)[C@H]1O]

To a solution of intermediate 36 (9 mg, 0.02 mmol), 4-((2-(2-(2-(2-aminoethoxy)ethoxy)ethoxy)ethyl)amino)-2-(2,6-dioxopiperidin-3-yl)isoindoline-1,3-dione (9 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-90 (10 mg, yield 60%). ¹H NMR (800 MHz, Methanol-d₄) δ 8.25 (s, 1H), 7.63 (d, J=3.9 Hz, 1H), 7.51 (t, J=7.8 Hz, 1H), 7.37 (d, J=8.0 Hz, 1H), 7.14-7.07 (m, 2H), 7.03 (d, J=7.9 Hz, 2H), 6.92 (d, J=3.9 Hz, 1H), 6.22 (d, J=6.5 Hz, 1H), 5.05 (dd, J=12.7, 5.5 Hz, 1H), 4.92 (d, J=4.1 Hz, 1H), 4.62 (t, J=5.9 Hz, 1H), 4.56-4.46 (m, 2H), 4.34-4.29 (m, 1H), 4.25 (d, J=3.6 Hz, 1H), 3.69 (t, J=5.2 Hz, 2H), 3.67-3.54 (m, 10H), 3.50-3.44 (m, 4H), 2.89-2.83 (m, 1H), 2.76-2.68 (m, 2H), 2.15-2.08 (m, 1H). MS (ESI) m/z 881.3 [M+H]⁺.

### Example 63: Synthesis of YS43-91

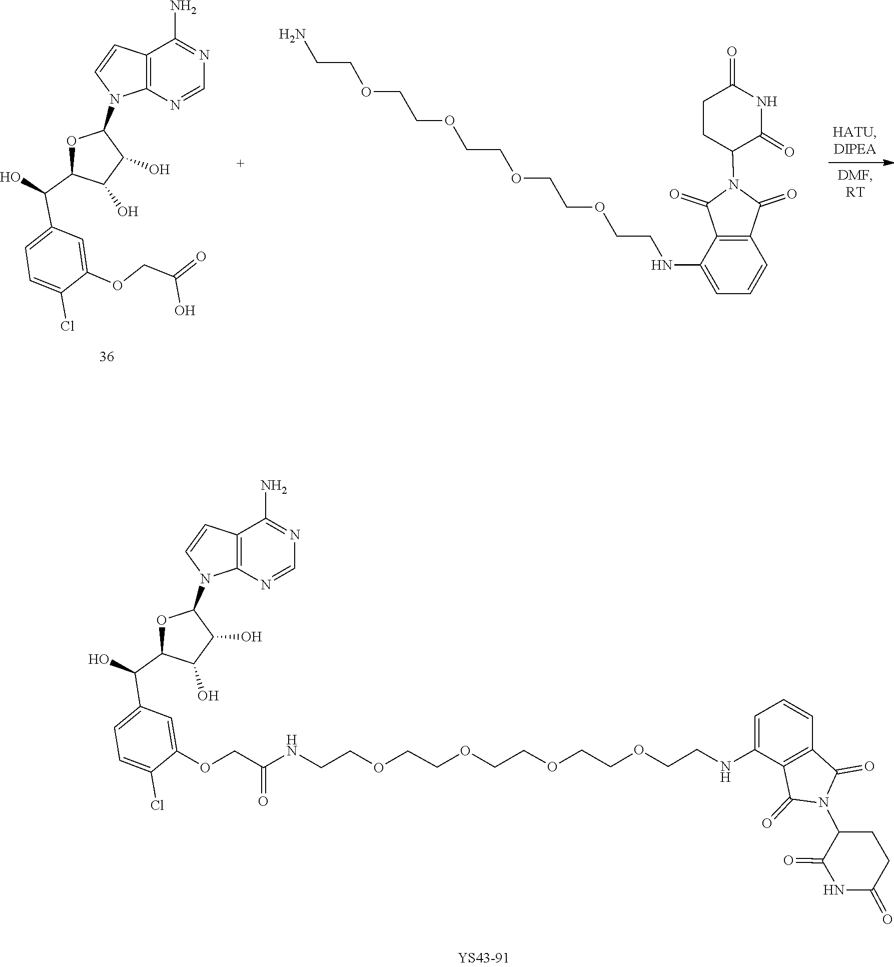 [SMILES: Nc1ncnc2c1ccn2[C@@H]1O[C@H]([C@H](O)c2ccc(Cl)c(OCC(=O)O)c2)[C@@H](O)[C@H]1O ; NCCOCCOCCOCCOCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCOCCOCCOCCOCCNc3cccc4c3C(=O)N(C3CCC(=O)NC3=O)C4=O)c2)[C@@H](O)[C@H]1O]

To a solution of intermediate 36 (9 mg, 0.02 mmol), 4-((14-amino-3,6,9,12-tetraoxatetradecyl)amino)-2-(2,6-dioxopiperidin-3-yl)isoindoline-1,3-dione (9.8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-91 (10.2 mg, yield 55%). ¹H NMR (800 MHz, Methanol-d₄) δ 8.25 (s, 1H), 7.63 (d, J=3.9 Hz, 1H), 7.53 (t, J=7.8 Hz, 1H), 7.38 (d, J=8.1 Hz, 1H), 7.14-7.09 (m, 2H), 7.05 (dd, J=15.8, 7.8 Hz, 2H), 6.92 (d, J=3.9 Hz, 1H), 6.22 (d, J=6.6 Hz, 1H), 5.09-5.01 (m, 1H), 4.92 (d, J=4.1 Hz, 1H), 4.62 (t, J=5.9 Hz, 1H), 4.58-4.47 (m, 2H), 4.33-4.28 (m, 1H), 4.27-4.23 (m, 1H), 3.71 (t, J=5.2 Hz, 2H), 3.66-3.59 (m, 14H), 3.52-3.45 (m, 4H), 2.89-2.81 (m, 1H), 2.78-2.71 (m, 2H), 2.14-2.09 (m, 1H). MS (ESI) m/z 925.3 [M+H]⁺.

### Example 64: Synthesis of YS43-92

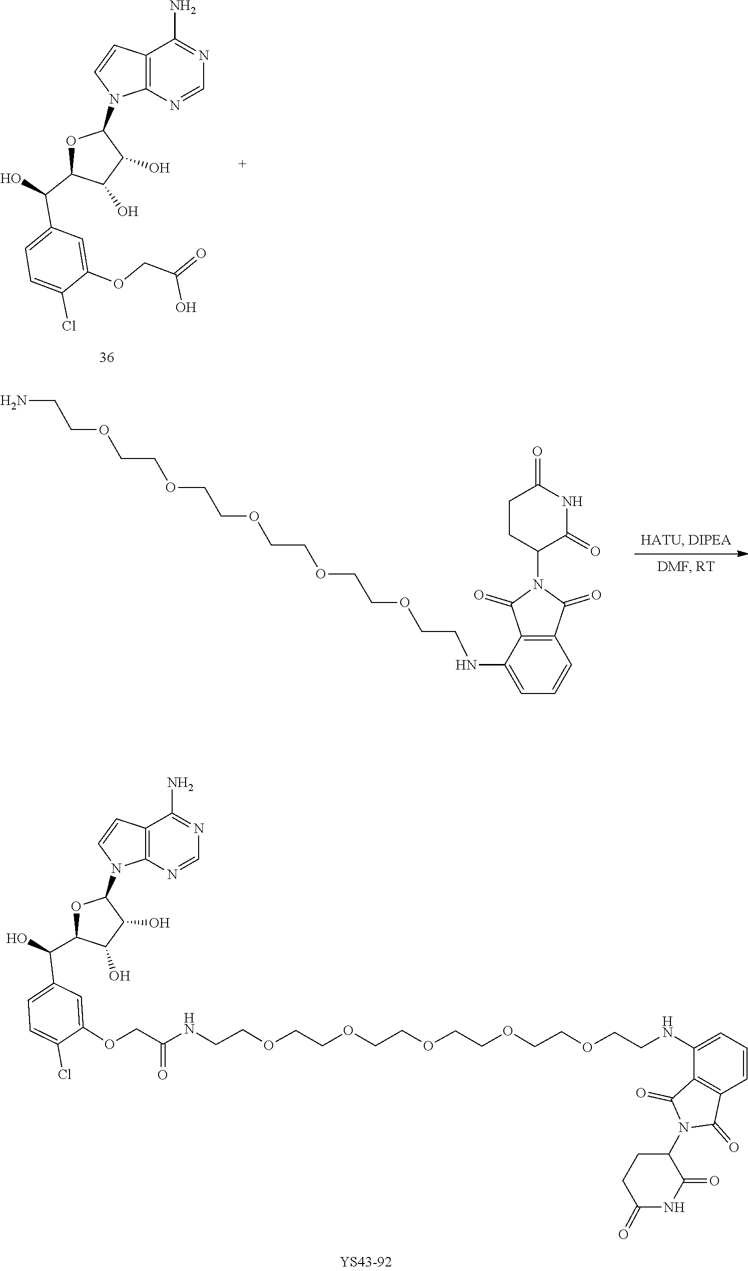 [SMILES: Nc1ncnc2c1ccn2[C@@H]1O[C@H]([C@H](O)c2ccc(Cl)c(OCC(=O)O)c2)[C@@H](O)[C@H]1O ; NCCOCCOCCOCCOCCOCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCOCCOCCOCCOCCOCCNc3cccc4c3C(=O)N(C3CCC(=O)NC3=O)C4=O)c2)[C@@H](O)[C@H]1O]

To a solution of intermediate 36 (9 mg, 0.02 mmol), 4-((17-amino-3,6,9,12,15-pentaoxaheptadecyl)amino)-2-(2,6-dioxopiperidin-3-yl)isoindoline-1,3-dione (10.8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-92 (10 mg, yield 52%). ¹H NMR (800 MHz, Methanol-d₄) δ 8.26 (s, 1H), 7.63 (d, J=3.9 Hz, 1H), 7.53 (t, J=7.8 Hz, 1H), 7.39 (d, J=8.1 Hz, 1H), 7.12-7.09 (m, 2H), 7.08-7.02 (m, 2H), 6.92 (d, J=3.9 Hz, 1H), 6.22 (d, J=6.6 Hz, 1H), 5.05 (dd, J=12.7, 5.4 Hz, 1H), 4.93 (d, J=4.2 Hz, 1H), 4.62 (t, J=5.9 Hz, 1H), 4.59-4.48 (m, 2H), 4.33-4.29 (m, 1H), 4.25 (d, J=3.7 Hz, 1H), 3.72 (t, J=5.1 Hz, 2H), 3.70-3.55 (m, 18H), 3.53-3.45 (m, 4H), 2.91-2.81 (m, 1H), 2.78-2.67 (m, 2H), 2.17-2.08 (m, 1H). MS (ESI) m/z 969.3 [M+H]⁺.

### Example 65: Synthesis of YS43-93

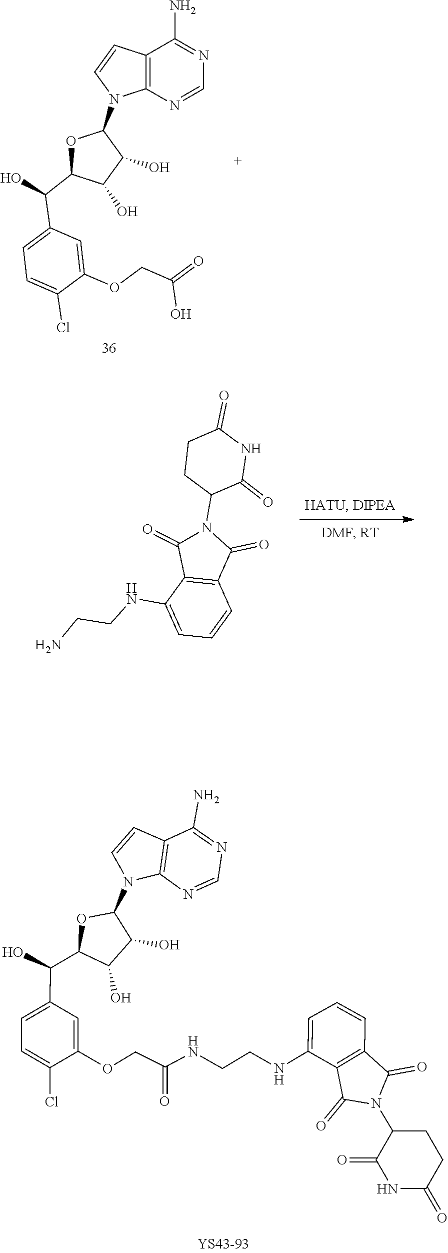 [SMILES: Nc1ncnc2c1ccn2[C@@H]1O[C@H]([C@H](O)c2ccc(Cl)c(OCC(=O)O)c2)[C@@H](O)[C@H]1O ; NCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCNc3cccc4c3C(=O)N(C3CCC(=O)NC3=O)C4=O)c2)[C@@H](O)[C@H]1O]

To a solution of intermediate 36 (9 mg, 0.02 mmol), 4-((2-aminoethyl)amino)-2-(2,6-dioxopiperidin-3-yl)isoindoline-1,3-dione (6.2 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-93 (4.5 mg, yield 30%). ¹H NMR (800 MHz, Methanol-d₄) δ 8.25 (d, J=4.3 Hz, 1H), 7.64 (d, J=3.9 Hz, 1H), 7.50 (t, J=7.8 Hz, 1H), 7.38 (d, J=8.1 Hz, 1H), 7.16-7.09 (m, 3H), 7.01 (d, J=7.1 Hz, 1H), 6.93 (d, J=3.8 Hz, 1H), 6.20 (dd, J=7.0, 4.4 Hz, 1H), 5.05 (dd, J=12.7, 5.3 Hz, 1H), 4.92-4.88 (m, 1H), 4.64-4.60 (m, 1H), 4.56 (s, 2H), 4.31-4.27 (m, 1H), 4.23 (d, 1H), 3.57 (t, J=6.2 Hz, 2H), 3.52 (t, J=6.3, 5.6 Hz, 2H), 2.85 (ddd, J=18.2, 13.7, 5.3 Hz, 1H), 2.77-2.67 (m, 2H), 2.13-2.08 (m, 1H). MS (ESI) m/z 749.2 [M+H]⁺.

### Example 66: Synthesis of YS43-94

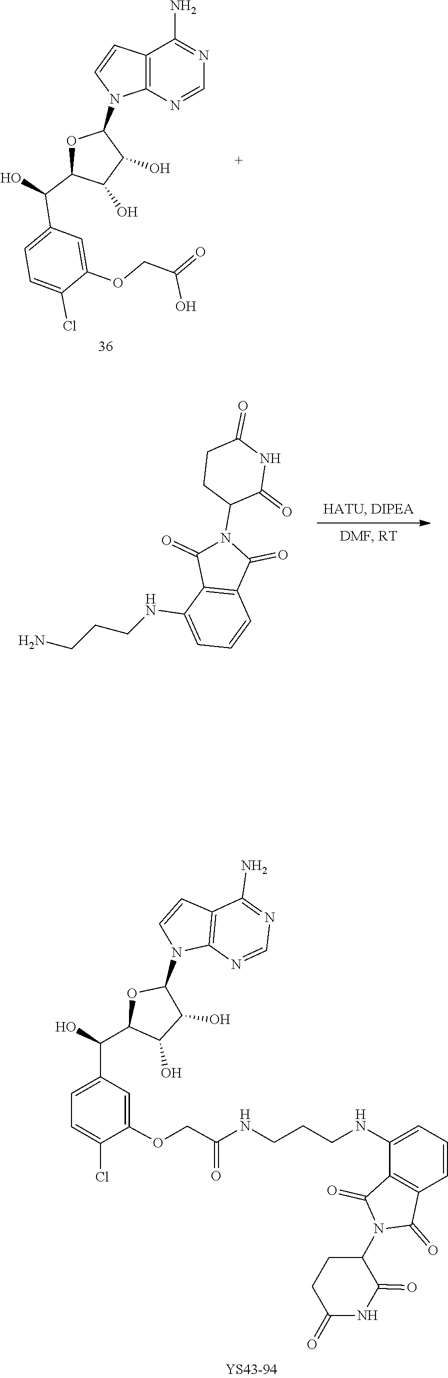 [SMILES: Nc1ncnc2c1ccn2[C@@H]1O[C@H]([C@H](O)c2ccc(Cl)c(OCC(=O)O)c2)[C@@H](O)[C@H]1O ; NCCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCCNc3cccc4c3C(=O)N(C3CCC(=O)NC3=O)C4=O)c2)[C@@H](O)[C@H]1O]

To a solution of intermediate 36 (9 mg, 0.02 mmol), 4-((3-aminopropyl)amino)-2-(2,6-dioxopiperidin-3-yl)isoindoline-1,3-dione (6.6 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-94 (6.3 mg, yield 41%). ¹H NMR (800 MHz, Methanol-d₄) δ 8.24 (s, 1H), 7.63 (d, J=3.9 Hz, 1H), 7.50 (t, J=7.8 Hz, 1H), 7.39 (d, J=8.1 Hz, 1H), 7.15 (d, J=4.2 Hz, 1H), 7.11 (d, J=4.3 Hz, 1H), 7.02 (d, J=7.2 Hz, 1H), 6.98 (d, J=8.5 Hz, 1H), 6.91 (d, J=3.9 Hz, 1H), 6.20 (d, J=6.9 Hz, 1H), 5.04-4.99 (m, 1H), 4.93-4.91 (m, 1H), 4.63 (t, J=6.0 Hz, 1H), 4.58 (s, 2H), 4.30 (d, J=5.3 Hz, 1H), 4.23 (d, J=3.7 Hz, 1H), 3.49-3.42 (m, 2H), 3.38-3.36 (m, 2H), 2.87-2.81 (m, 1H), 2.77-2.71 (m, 1H), 2.70-2.65 (m, 1H), 2.11-2.00 (m, 1H), 1.94-1.88 (m, 2H). MS (ESI) m/z 763.4 [M+H]⁺.

### Example 67: Synthesis of YS43-95

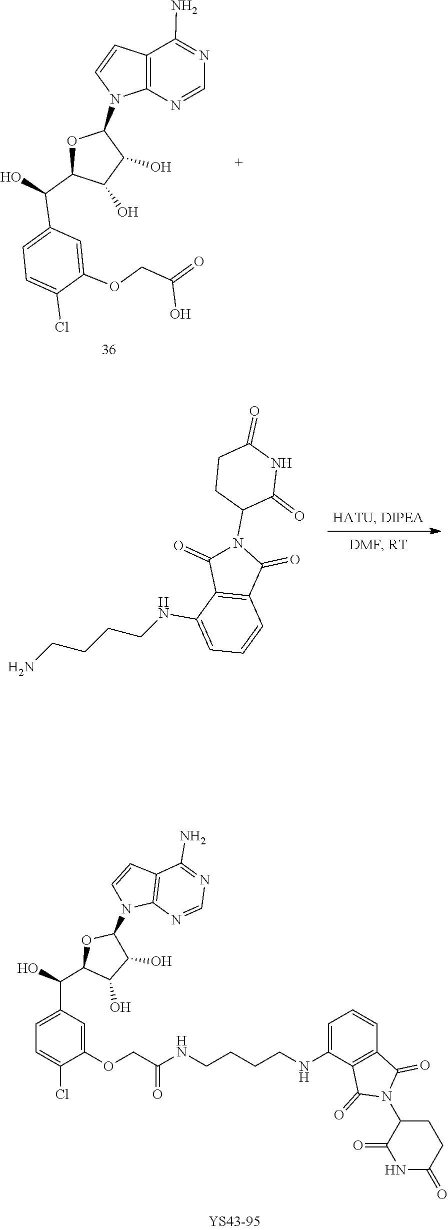 [SMILES: Nc1ncnc2c1ccn2[C@@H]1O[C@H]([C@H](O)c2ccc(Cl)c(OCC(=O)O)c2)[C@@H](O)[C@H]1O ; NCCCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCCCNc3cccc4c3C(=O)N(C3CCC(=O)NC3=O)C4=O)c2)[C@@H](O)[C@H]1O]

To a solution of intermediate 36 (9 mg, 0.02 mmol), 4-((4-aminobutyl)amino)-2-(2,6-dioxopiperidin-3-yl)isoindoline-1,3-dione (6.8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-95 (6.5 mg, yield 42%). ¹H NMR (800 MHz, Methanol-d₄) δ 8.23 (d, J=3.5 Hz, 1H), 7.64 (d, J=3.2 Hz, 1H), 7.50 (t, J=7.8 Hz, 1H), 7.39 (d, J=8.1 Hz, 1H), 7.20-7.08 (m, 2H), 7.00 (dd, J=8.1, 4.3 Hz, 2H), 6.90 (d, J=3.8 Hz, 1H), 6.21 (d, J=7.0 Hz, 1H), 5.05 (dd, J=12.7, 5.4 Hz, 1H), 4.96-4.89 (m, 1H), 4.70-4.60 (m, 1H), 4.57 (s, 2H), 4.33-4.28 (m, 1H), 4.28-4.17 (m, 1H), 3.41-3.34 (m, 4H), 2.91-2.82 (m, 1H), 2.77-2.69 (m, 2H), 2.15-2.05 (m, 1H), 1.68 (t, J=4.1 Hz, 4H). MS (ESI) m/z 777.3 [M+H]⁺.

### Example 68: Synthesis of YS43-96

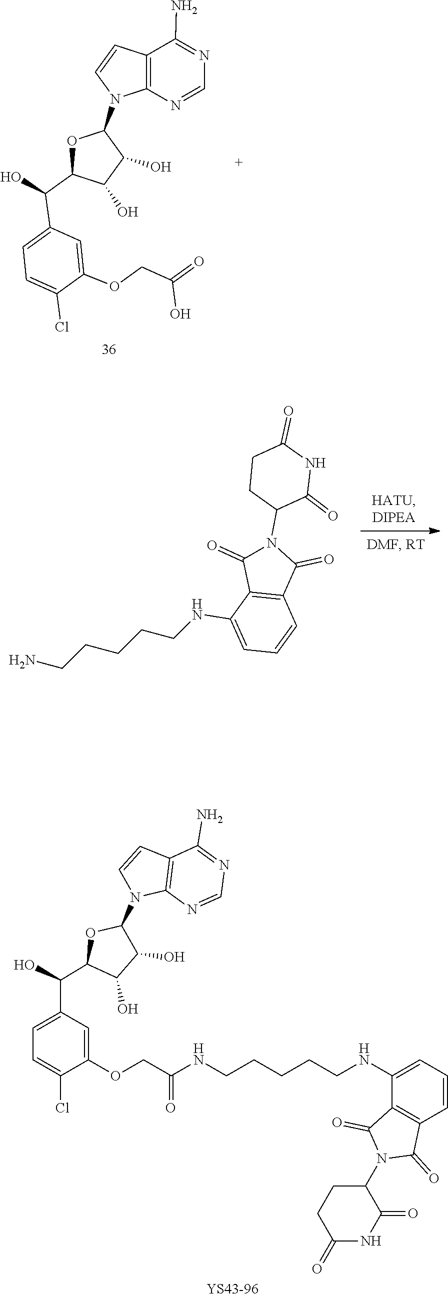 [SMILES: Nc1ncnc2c1ccn2[C@@H]1O[C@H]([C@H](O)c2ccc(Cl)c(OCC(=O)O)c2)[C@@H](O)[C@H]1O ; NCCCCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCCCCNc3cccc4c3C(=O)N(C3CCC(=O)NC3=O)C4=O)c2)[C@@H](O)[C@H]1O]

To a solution of intermediate 36 (9 mg, 0.02 mmol), 4-((5-aminopentyl)amino)-2-(2,6-dioxopiperidin-3-yl)isoindoline-1,3-dione (7.2 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-96 (6.8 mg, yield 43%). ¹H NMR (800 MHz, Methanol-d₄) δ 8.24 (d, J=4.9 Hz, 1H), 7.65 (q, J=3.7 Hz, 1H), 7.50 (d, J=7.1 Hz, 1H), 7.40 (t, J=6.8 Hz, 1H), 7.13 (dd, J=16.5, 5.9 Hz, 2H), 7.00 (dt, J=6.9, 4.1 Hz, 2H), 6.90 (q, J=3.7 Hz, 1H), 6.22 (d, J=6.3 Hz, 1H), 5.05 (dd, J=11.9, 5.6 Hz, 1H), 4.97-4.93 (m, 1H), 4.66-4.53 (m, 3H), 4.36-4.20 (m, 2H), 3.41-3.32 (m, 4H), 2.91-2.81 (m, 1H), 2.78-2.70 (m, 2H), 2.15-2.05 (m, 1H), 1.74-1.67 (m, 2H), 1.67-1.61 (m, 2H), 1.55-1.42 (m, 2H). MS (ESI) m/z 791.3 [M+H]⁺.

### Example 69: Synthesis of YS43-97

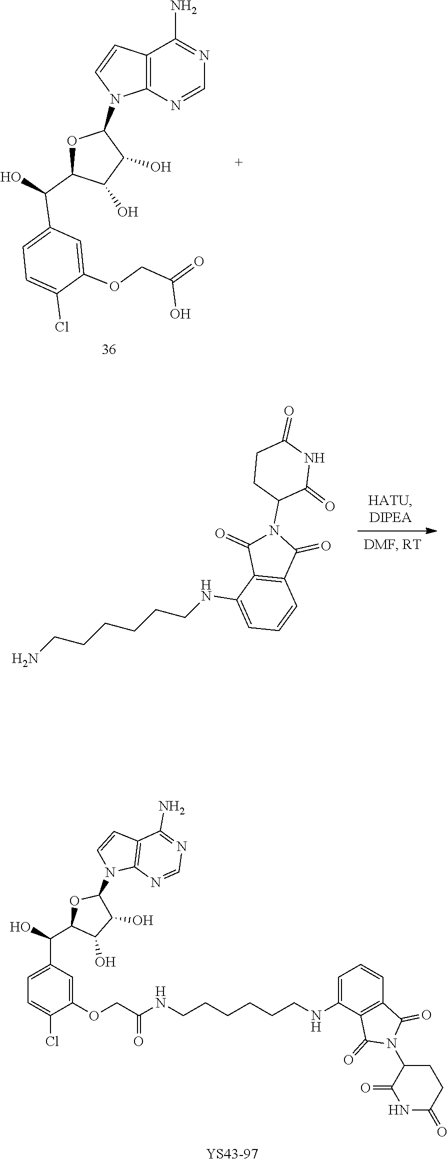 [SMILES: Nc1ncnc2c1ccn2[C@@H]1O[C@H]([C@H](O)c2ccc(Cl)c(OCC(=O)O)c2)[C@@H](O)[C@H]1O ; NCCCCCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCCCCCNc3cccc4c3C(=O)N(C3CCC(=O)NC3=O)C4=O)c2)[C@@H](O)[C@H]1O]

To a solution of intermediate 36 (9 mg, 0.02 mmol), 4-((6-aminohexyl)amino)-2-(2,6-dioxopiperidin-3-yl)isoindoline-1,3-dione (7.4 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-97 (11 mg, yield 69%). ¹H NMR (800 MHz, Methanol-d₄) δ 8.24 (s, 1H), 7.67 (d, J=4.1 Hz, 1H), 7.52 (t, J=7.8 Hz, 1H), 7.40 (d, J=8.1 Hz, 1H), 7.18-7.08 (m, 2H), 7.01 (dd, J=22.0, 7.8 Hz, 2H), 6.91 (d, J=3.9 Hz, 1H), 6.23 (d, J=6.9 Hz, 1H), 5.06 (dd, J=12.7, 5.3 Hz, 1H), 4.97-4.87 (m, 1H), 4.62 (t, J=6.1 Hz, 1H), 4.56 (s, 2H), 4.30 (d, J=5.4 Hz, 1H), 4.28-4.22 (m, 1H), 3.32 (d, J=29.6 Hz, 4H), 2.92-2.84 (m, 1H), 2.81-2.68 (m, 2H), 2.17-2.06 (m, 1H), 1.72-1.62 (m, 2H), 1.62-1.50 (m, 2H), 1.52-1.37 (m, 4H). MS (ESI) m/z 805.3 [M+H]⁺.

### Example 70: Synthesis of YS43-98

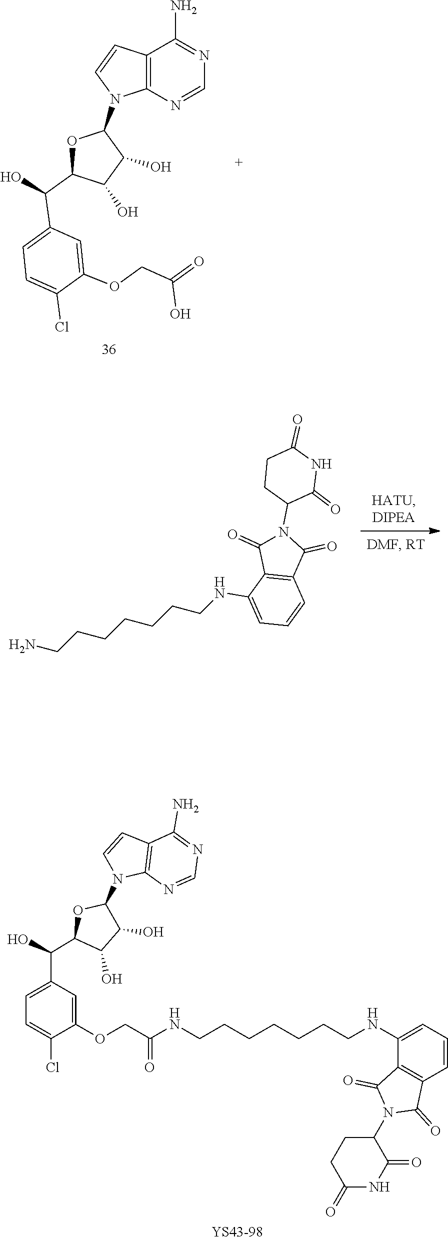 [SMILES: Nc1ncnc2c1ccn2[C@@H]1O[C@H]([C@H](O)c2ccc(Cl)c(OCC(=O)O)c2)[C@@H](O)[C@H]1O ; NCCCCCCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCCCCCCNc3cccc4c3C(=O)N(C3CCC(=O)NC3=O)C4=O)c2)[C@@H](O)[C@H]1O]

To a solution of intermediate 36 (9 mg, 0.02 mmol), 4-((7-aminoheptyl)amino)-2-(2,6-dioxopiperidin-3-yl)isoindoline-1,3-dione (7.8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-98 (6.7 mg, yield 41%). ¹H NMR (800 MHz, Methanol-d₄) δ 8.24 (s, 1H), 7.67 (d, J=3.9 Hz, 1H), 7.52 (t, J=7.8 Hz, 1H), 7.40 (d, J=7.9 Hz, 1H), 7.13 (d, J=9.6 Hz, 2H), 7.02 (d, J=7.2 Hz, 1H), 6.99 (d, J=8.5 Hz, 1H), 6.91 (d, J=3.9 Hz, 1H), 6.23 (d, J=6.9 Hz, 1H), 5.07 (dd, J=12.9, 5.5 Hz, 1H), 4.97-4.89 (m, 1H), 4.63 (t, J=6.1 Hz, 1H), 4.56 (s, 2H), 4.32-4.27 (m, 1H), 4.27-4.23 (m, 1H), 3.29 (s, 4H), 2.92-2.84 (m, 1H), 2.79-2.71 (m, 2H), 2.16-2.09 (m, 1H), 1.66 (p, J=7.2 Hz, 2H), 1.58 (p, J=7.2 Hz, 2H), 1.50-1.34 (m, 6H). MS (ESI) m/z 819.3 [M+H]⁺.

### Example 71: Synthesis of YS43-99

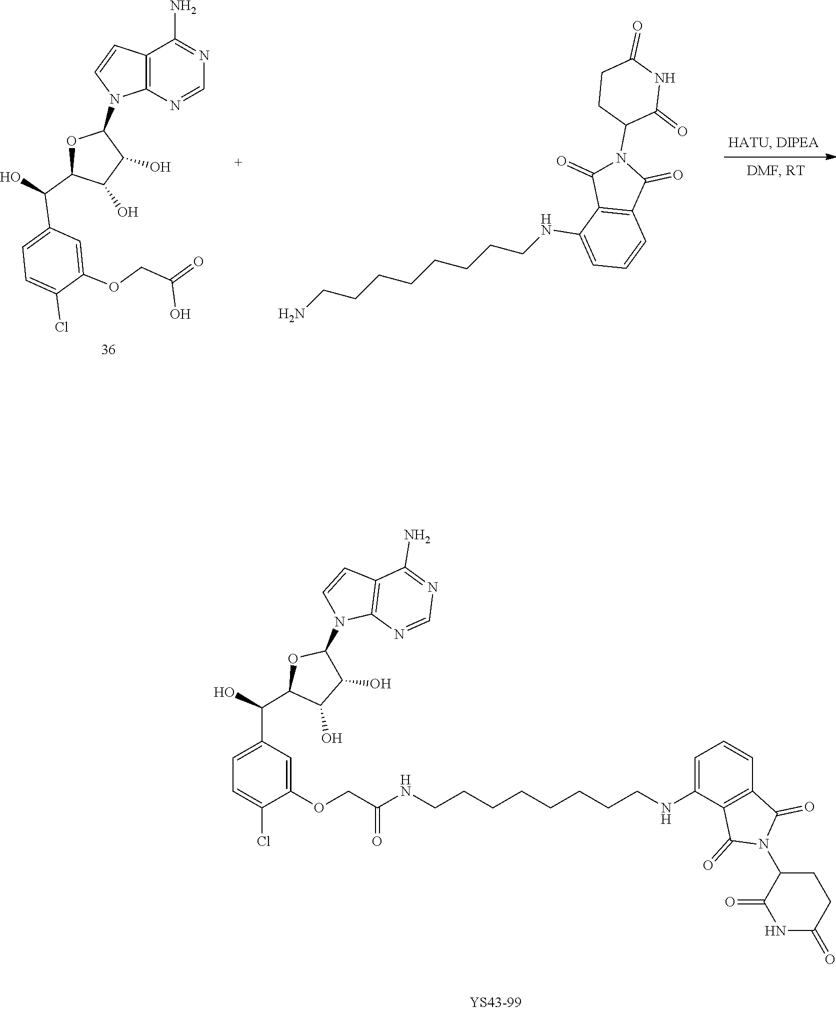 [SMILES: Nc1ncnc2c1ccn2[C@@H]1O[C@H]([C@H](O)c2ccc(Cl)c(OCC(=O)O)c2)[C@@H](O)[C@H]1O ; NCCCCCCCCNc1cccc2c1C(=O)N(C1CCC(=O)NC1=O)C2=O ; Nc1ncnc2c1ccn2[C@@H]1O[C@H]([C@H](O)c2ccc(Cl)c(OCC(=O)NCCCCCCCCNc3cccc4c3C(=O)N(C3CCC(=O)NC3=O)C4=O)c2)[C@@H](O)[C@H]1O]

To a solution of intermediate 36 (9 mg, 0.02 mmol), 4-((8-aminooctyl)amino)-2-(2,6-dioxopiperidin-3-yl)isoindoline-1,3-dione (8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-99 (3.5 mg, yield 21%). ¹H NMR (800 MHz, Methanol-d₄)¹H NMR (800 MHz, Methanol-d₄) δ 8.25 (s, 1H), 7.65 (d, J=3.9 Hz, 1H), 7.54 (t, J=7.8 Hz, 1H), 7.40 (d, J=8.1 Hz, 1H), 7.20-7.09 (m, 2H), 7.02 (dd, J=21.1, 7.9 Hz, 2H), 6.91 (d, J=3.9 Hz, 1H), 6.23 (d, J=7.0 Hz, 1H), 5.07 (dd, J=12.9, 5.4 Hz, 1H), 4.94-4.95 (m, 1H), 4.64 (t, J=6.1 Hz, 1H), 4.61-4.50 (m, 2H), 4.29 (d, J=5.6 Hz, 1H), 4.25 (d, J=3.3 Hz, 1H), 3.37-3.27 (m, 4H), 2.92-2.80 (m, 1H), 2.80-2.70 (m, 2H), 2.17-2.10 (m, 1H), 1.70-1.65 (m, 2H), 1.60-1.55 (m, 2H), 1.49-1.34 (m, 8H). MS (ESI) m/z 833.4 [M+H]⁺.

### Example 72: Synthesis of YS43-100

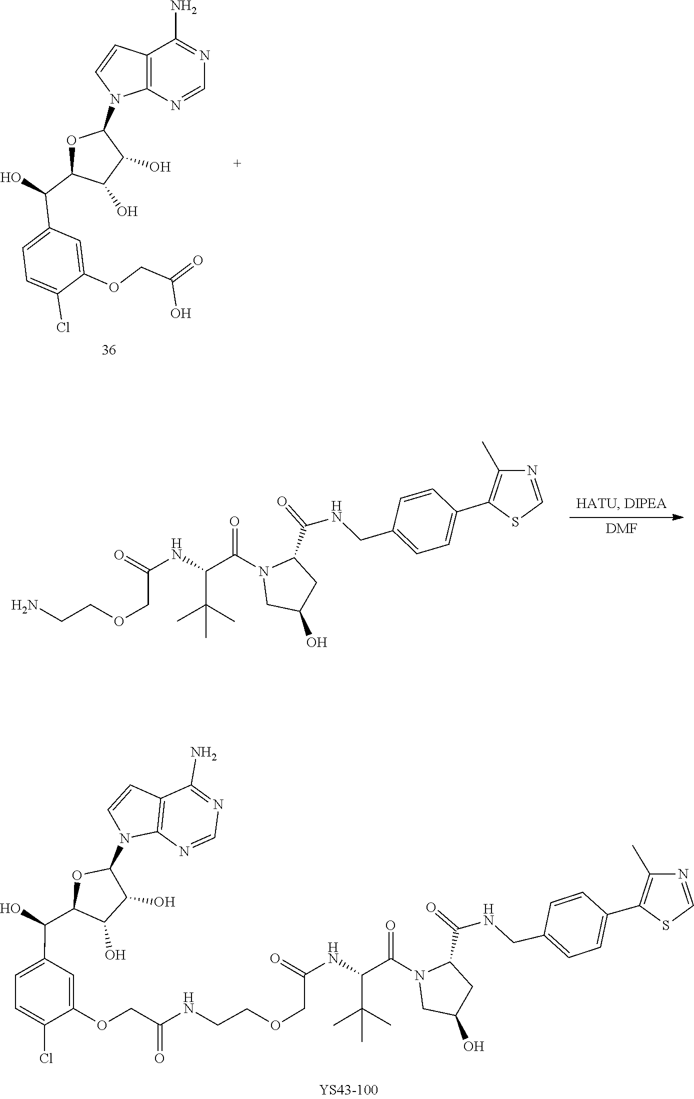

To a solution of intermediate 36 (9 mg, 0.02 mmol), (2S,4R)-1-((S)-2-(2-(2-aminoethoxy)acetamido)-3,3-dimethylbutanoyl)-4-hydroxy-N-(4-(4-methylthiazol-5-yl)benzyl)pyrrolidine-2-carboxamide (10.6 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-100 (8.1 mg, yield 43%). ¹H NMR (800 MHz, Methanol-d₄) δ 8.89 (s, 1H), 8.24 (s, 1H), 7.59 (d, J=4.0 Hz, 1H), 7.48-7.46 (m, 2H), 7.44-7.41 (m, 2H), 7.38-7.36 (m, 1H), 7.13-7.08 (m, 2H), 6.92 (d, J=3.9 Hz, 1H), 6.21 (d, J=6.5 Hz, 1H), 4.91-4.93 (m, 1H), 4.71 (s, 1H), 4.65-4.51 (m, 6H), 4.41-4.33 (m, 1H), 4.30-4.27 (m, 1H), 4.24 (q, J=3.9 Hz, 1H), 4.12-3.99 (m, 2H), 3.89-3.80 (m, 2H), 3.72 -3.65 (m, 2H), 3.63-3.57 (m, 2H), 2.47 (s, 3H), 2.27-2.23 (m, 1H), 2.13-2.09 (m, 1H), 1.04 (s, 9H). MS (ESI) m/z 964.3 [M+H]⁺.

### Example 73: Synthesis of YS43-101

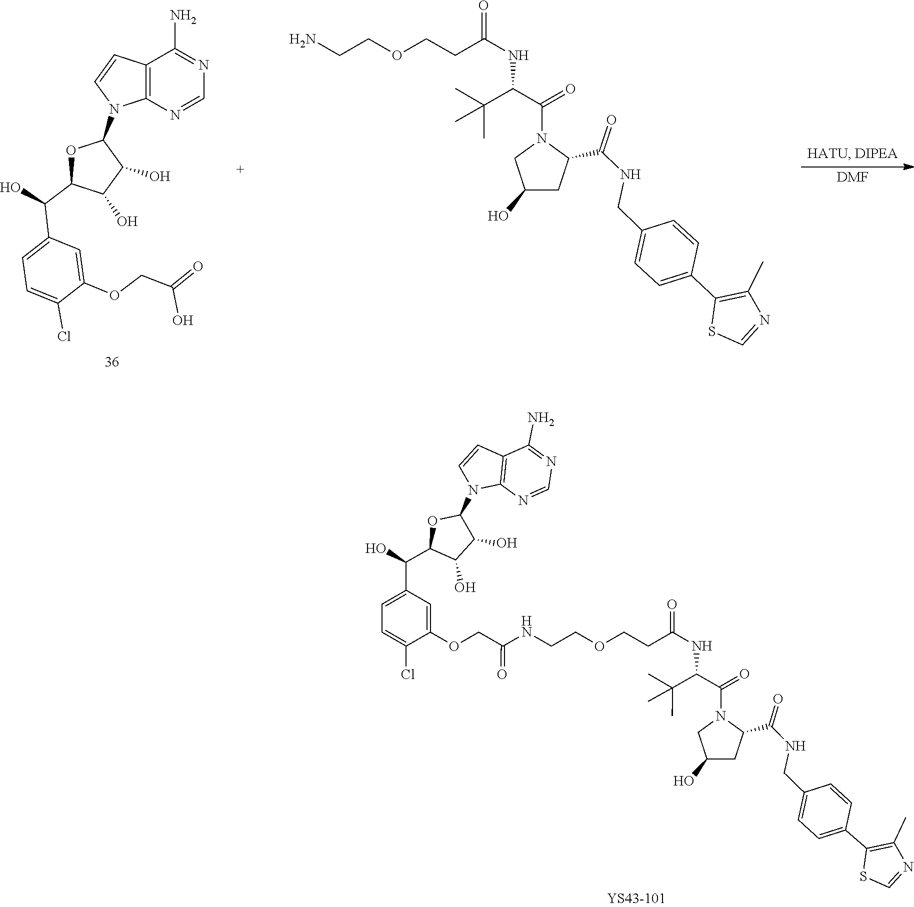

To a solution of intermediate 36 (9 mg, 0.02 mmol), (2S,4R)-1-((S)-2-(3-(2-aminoethoxy)propanamido)-3,3-dimethylbutanoyl)-4-hydroxy-N-(4-(4-methylthiazol-5-yl)benzyl)pyrrolidine-2-carboxamide (10.9 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-101 (9.5 mg, yield 49%). ¹H NMR (800 MHz, Methanol-d₄) δ 8.92 (s, 1H), 8.26 (s, 1H), 7.67 (d, J=3.9 Hz, 1H), 7.46 (d, J=8.0 Hz, 2H), 7.42 (d, J=7.9 Hz, 2H), 7.40-7.38 (m, 1H), 7.15-7.08 (m, 2H), 6.97-6.92 (m, 1H), 6.25 (d, J=6.6 Hz, 1H), 4.94 (d, J=4.0 Hz, 1H), 4.66 (s, 1H), 4.65-4.55 (m, 3H), 4.55-4.49 (m, 3H), 4.39-4.35 (m, 1H), 4.32-4.28 (m, 1H), 4.28-4.23 (m, 1H), 3.94-3.79 (m, 2H), 3.78-3.69 (m, 2H), 3.60 (t, J=5.3 Hz, 2H), 3.51 (t, J=5.0 Hz, 2H), 2.58-2.50 (m, 2H), 2.48 (s, 3H), 2.24 (dd, J=13.3, 7.7 Hz, 1H), 2.10 (ddd, J=13.2, 8.9, 4.5 Hz, 1H), 1.05 (s, 9H). MS (ESI) m/z 978.4 [M+H]⁺.

### Example 74: Synthesis of YS43-102

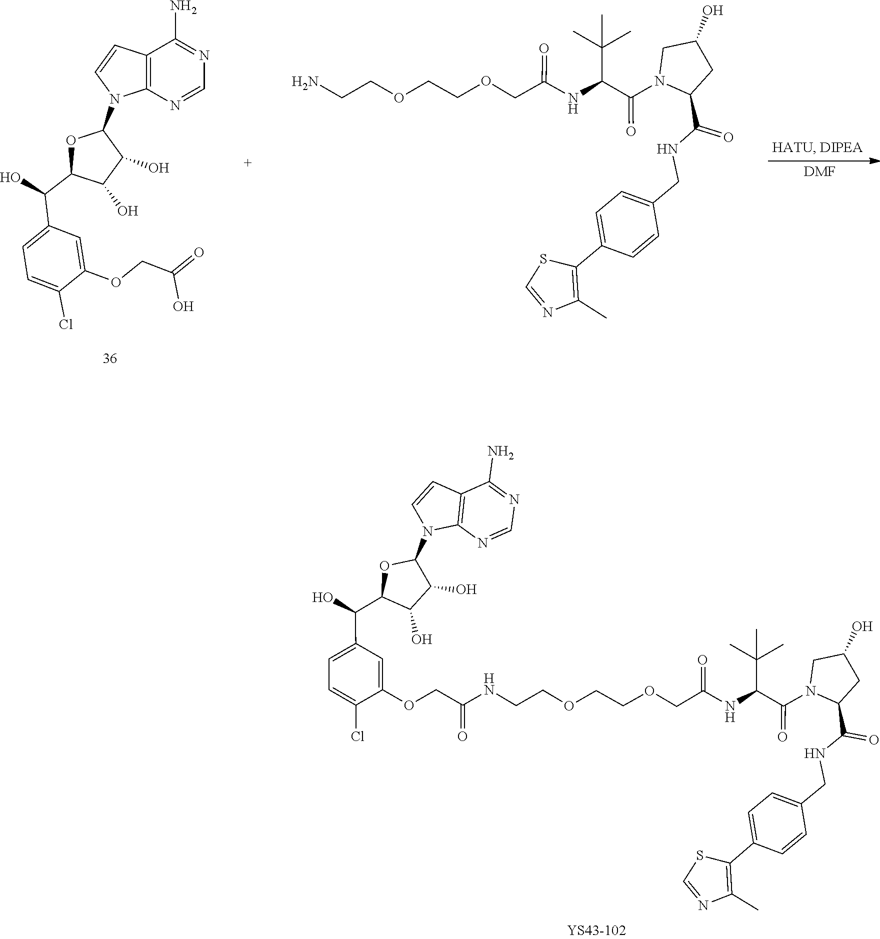

To a solution of intermediate 36 (9 mg, 0.02 mmol), (2S,4R)-1-((S)-2-(2-(2-(2-aminoethoxy)ethoxy)acetamido)-3,3-dimethylbutanoyl)-4-hydroxy-N-(4-(4-methylthiazol-5-yl)benzyl)pyrrolidine-2-carboxamide (11.5 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-102 (12.3 mg, yield 62%). ¹H NMR (800 MHz, Methanol-d₄) δ 8.95 (s, 1H), 8.26 (s, 1H), 7.65 (s, 1H), 7.48-7.41 (m, 4H), 7.38 (d, J=8.2 Hz, 1H), 7.14-7.09 (m, 2H), 6.94 (d, J=4.1 Hz, 1H), 6.25 (d, J=6.7 Hz, 1H), 4.93 (d, J=4.0 Hz, 1H), 4.74 (s, 1H), 4.65-4.56 (m, 3H), 4.56-4.48 (m, 3H), 4.39-4.36 (m, 1H), 4.32-4.30 (m, 1H), 4.26 (d, J=3.5 Hz, 1H), 4.07-3.96 (m, 2H), 3.91-3.81 (m, 2H), 3.75-3.54 (m, 7H), 3.53-3.46 (m, 1H), 2.49 (s, 3H), 2.24 (dd, J=13.3, 7.8 Hz, 1H), 2.10 (ddd, J=13.3, 8.8, 4.4 Hz, 1H), 1.05 (s, 9H). MS (ESI) m/z 1008.4 [M+H]⁺.

### Example 75: Synthesis of YS43-103

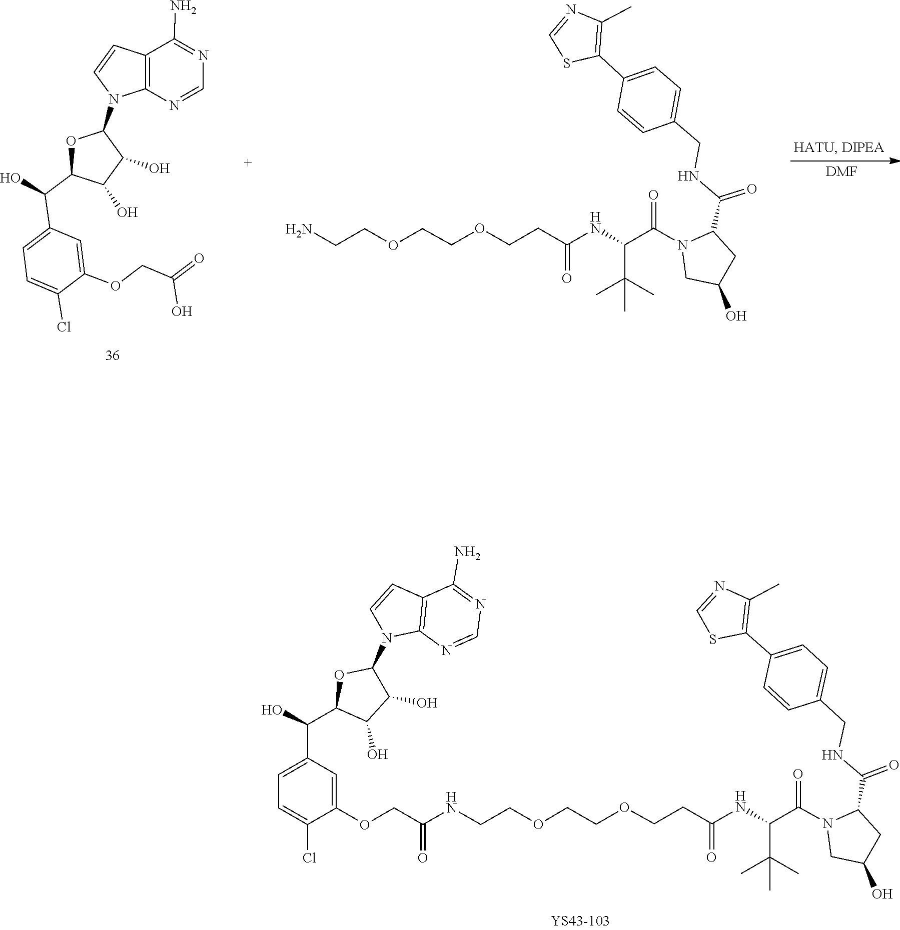

To a solution of intermediate 36 (9 mg, 0.02 mmol), (2S,4R)-1-((S)-2-(3-(2-(2-aminoethoxy)ethoxy)propanamido)-3,3-dimethylbutanoyl)-4-hydroxy-N-(4-(4-methylthiazol-5-yl)benzyl)pyrrolidine-2-carboxamide (11.8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-103 (8 mg, yield 39%). ¹H NMR (800 MHz, Methanol-d₄) δ 8.93 (s, 1H), 8.26 (s, 1H), 7.66 (d, J=4.0 Hz, 1H), 7.48 (d, J=7.8 Hz, 2H), 7.43 (d, J=7.9 Hz, 2H), 7.40 (d, J=8.0 Hz, 1H), 7.15-7.09 (m, 2H), 6.94 (d, J=4.0 Hz, 1H), 6.25 (d, J=6.7 Hz, 1H), 4.94 (d, J=4.1 Hz, 1H), 4.66 (s, 1H), 4.64-4.48 (m, 6H), 4.40-4.36 (m, 1H), 4.31 (d, J=5.9 Hz, 1H), 4.26 (d, J=3.5 Hz, 1H), 3.93-3.89 (m, 1H), 3.84-3.79 (m, 1H), 3.76-3.70 (m, 2H), 3.67-3.57 (m, 6H), 3.50 (t, J=5.3 Hz, 2H), 2.58-2.44 (m, 5H), 2.27-2.22 (m, 1H), 2.13-2.08 (m, 1H), 1.05 (s, 9H). MS (ESI) m/z 1022.4 [M+H]⁺.

### Example 76: Synthesis of YS43-104

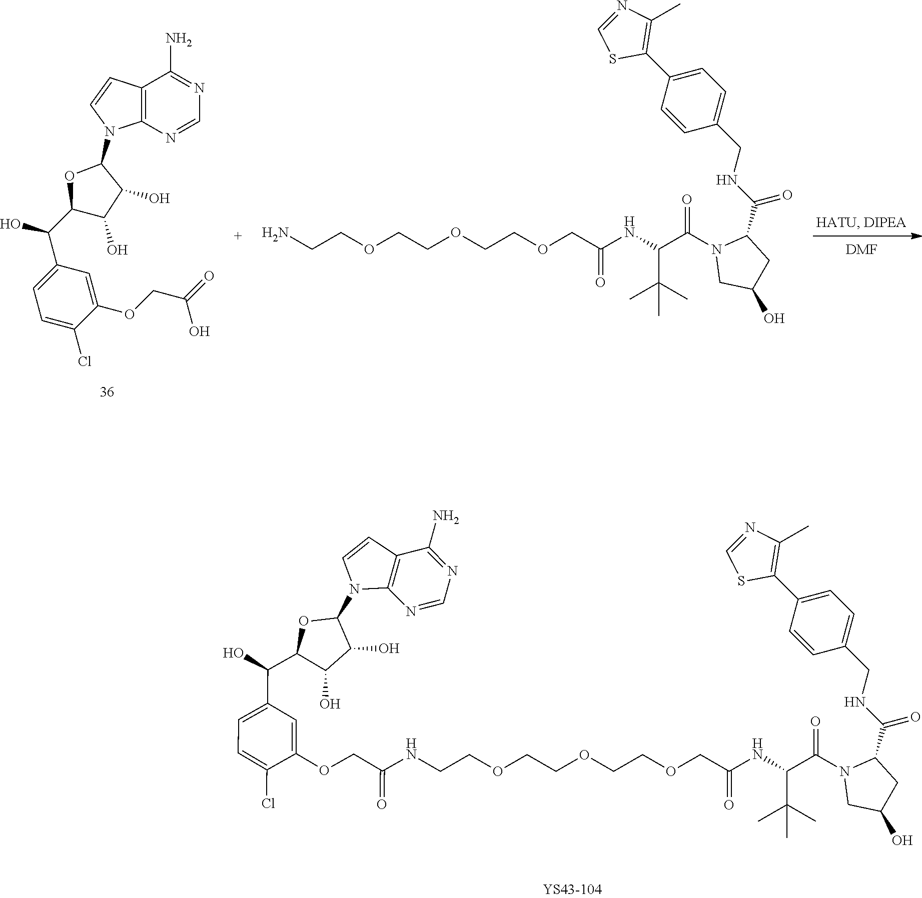

To a solution of intermediate 36 (9 mg, 0.02 mmol), (2S,4R)-1-((S)-14-amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (12.4 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-104 (10 mg, yield 48%). ¹H NMR (800 MHz, Methanol-d₄) δ 8.93 (s, 1H), 8.26 (s, 1H), 7.66 (d, J=4.0 Hz, 1H), 7.53-7.35 (m, 5H), 7.19-7.06 (m, 2H), 6.94 (d, J=3.9 Hz, 1H), 6.25 (d, J=6.7 Hz, 1H), 4.93 (d, J=4.0 Hz, 1H), 4.71 (s, 1H), 4.65-4.45 (m, 6H), 4.38 (d, J=15.4 Hz, 1H), 4.31 (s, 1H), 4.26 (d, J=3.6 Hz, 1H), 4.06-3.98 (m, 2H), 3.90 (d, J=11.0 Hz, 1H), 3.82 (dd, J=11.1, 4.0 Hz, 1H), 3.70-3.63 (m, 8H), 3.60 (d, J=5.3 Hz, 2H), 3.49 (t, J=5.3 Hz, 2H), 2.49 (s, 3H), 2.26 (dd, J=13.3, 7.7 Hz, 1H), 2.11 (ddd, J=13.3, 9.0, 4.5 Hz, 1H), 1.06 (s, 9H). MS (ESI) m/z 1052.4 [M+H]⁺.

### Example 77: Synthesis of YS43-105

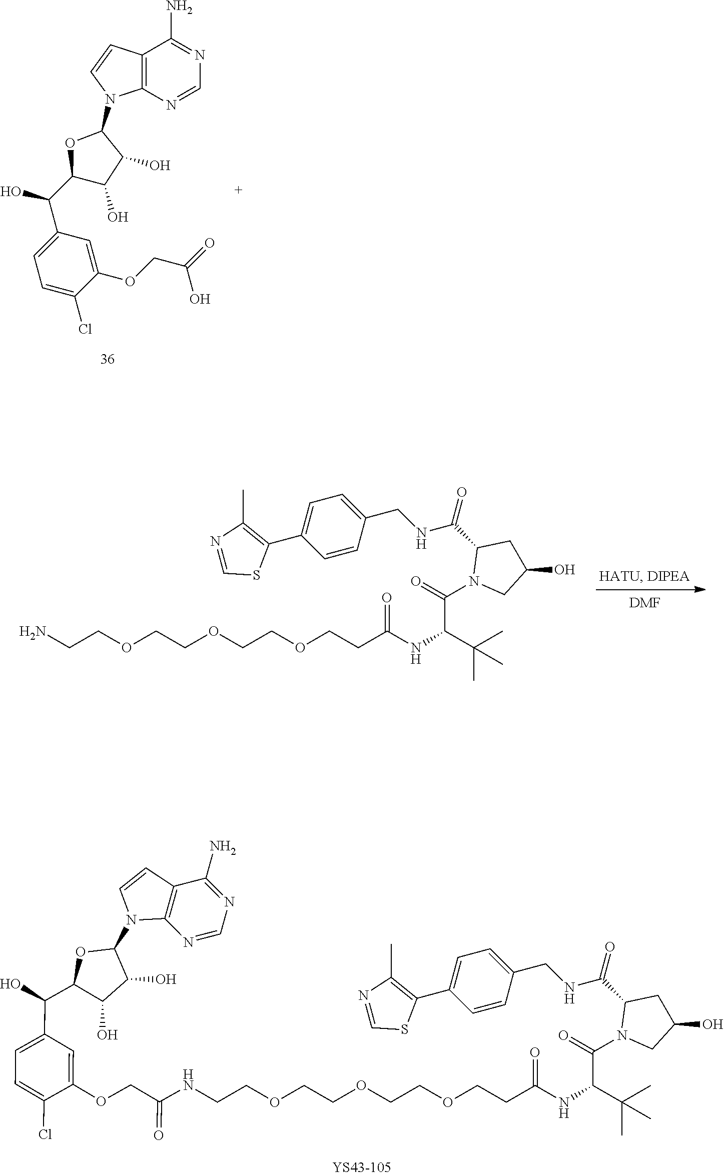

To a solution of intermediate 36 (9 mg, 0.02 mmol), (2S,4R)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (12.6 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-105 (6.9 mg, yield 32%). H NMR (800 MHz, Methanol-d₄) δ 8.93 (s, 1H), 8.26 (s, 1H), 7.65 (d, J=4.0 Hz, 1H), 7.48 (d, J=7.9 Hz, 2H), 7.43 (d, J=7.9 Hz, 2H), 7.40 (d, J=8.1 Hz, 1H), 7.17-7.06 (m, 2H), 6.94 (d, J=4.0 Hz, 1H), 6.25 (d, J=6.7 Hz, 1H), 4.94 (d, J=4.0 Hz, 1H), 4.66 (s, 1H), 4.64-4.49 (m, 6H), 4.37 (d, J=15.4 Hz, 1H), 4.34-4.29 (m, 1H), 4.26 (d, J=3.6 Hz, 1H), 3.91 (d, J=10.9 Hz, 1H), 3.82 (dd, J=11.2, 4.1 Hz, 1H), 3.72 (ddt, J=22.1, 9.9, 4.6 Hz, 2H), 3.66-3.56 (m, 10H), 3.50 (t, J=5.6 Hz, 2H), 2.58-2.54 (m, 1H), 2.51-2.44 (m, 4H), 2.24 (dd, J=13.4, 7.6 Hz, 1H), 2.11 (td, J=8.8, 4.4 Hz, 1H), 1.05 (s, 9H). MS (ESI) m/z 1066.4 [M+H]⁺.

### Example 78: Synthesis of YS43-106

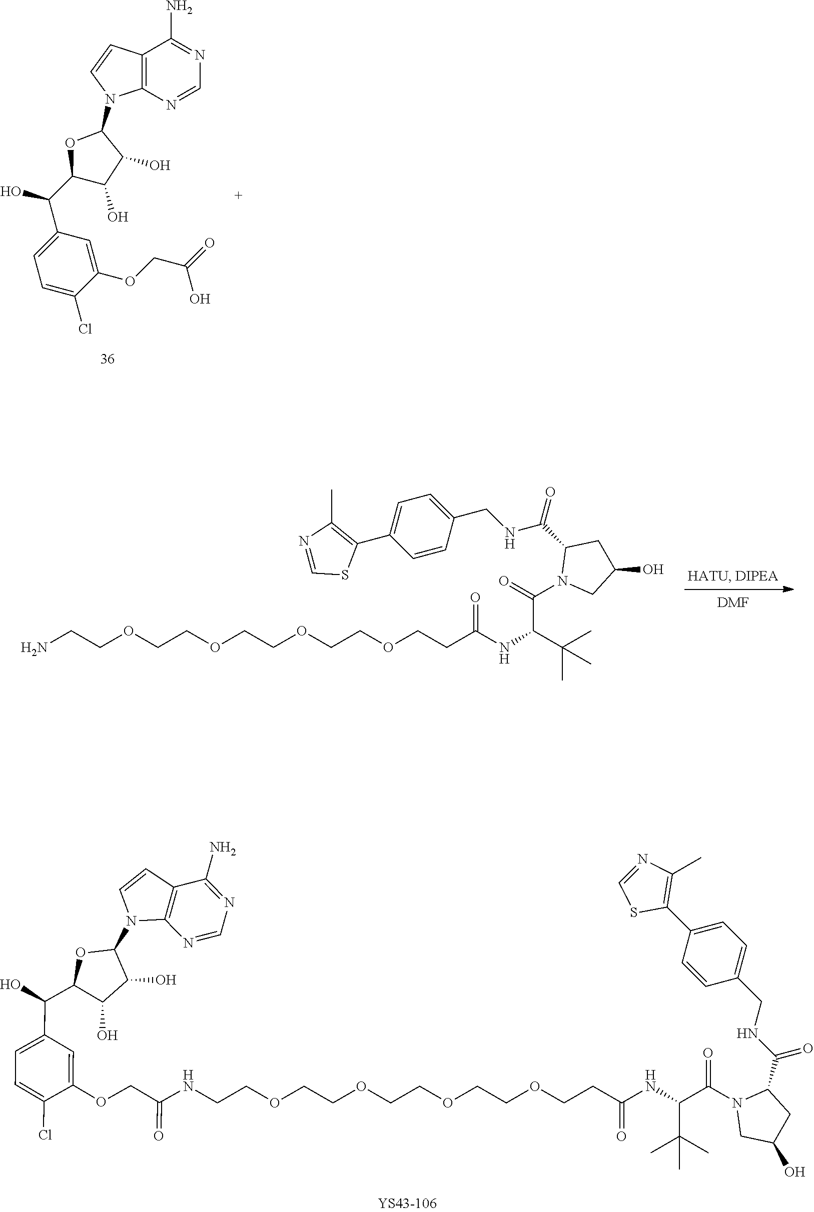

To a solution of intermediate 36 (9 mg, 0.02 mmol), (2S,4R)-1-((S)-1-amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (13.6 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-106 (6.4 mg, yield 29%). ¹H NMR (800 MHz, Methanol-d₄) δ 8.94 (s, 1H), 8.27 (s, 1H), 7.65 (d, J=3.9 Hz, 1H), 7.53-7.36 (m, 5H), 7.15-7.06 (m, 2H), 6.94 (d, J=4.0 Hz, 1H), 6.25 (d, J=6.7 Hz, 1H), 4.93 (d, J=4.0 Hz, 1H), 4.67 (s, 1H), 4.64-4.45 (m, 6H), 4.38 (d, J=15.3 Hz, 1H), 4.35-4.29 (m, 1H), 4.29-4.24 (m, 1H), 3.91 (d, J=10.9 Hz, 1H), 3.85-3.81 (m, 1H), 3.77-3.69 (m, 2H), 3.67-3.58 (m, 14H), 3.50 (t, J=4.8 Hz, 2H), 2.61-2.52 (m, 1H), 2.52-2.42 (m, 4H), 2.24 (dd, J=13.2, 7.7 Hz, 1H), 2.11 (td, J=9.0, 4.6 Hz, 1H), 1.05 (s, 9H). MS (ESI) m/z 1110.4 [M+H]⁺.

### Example 79: Synthesis of YS43-107

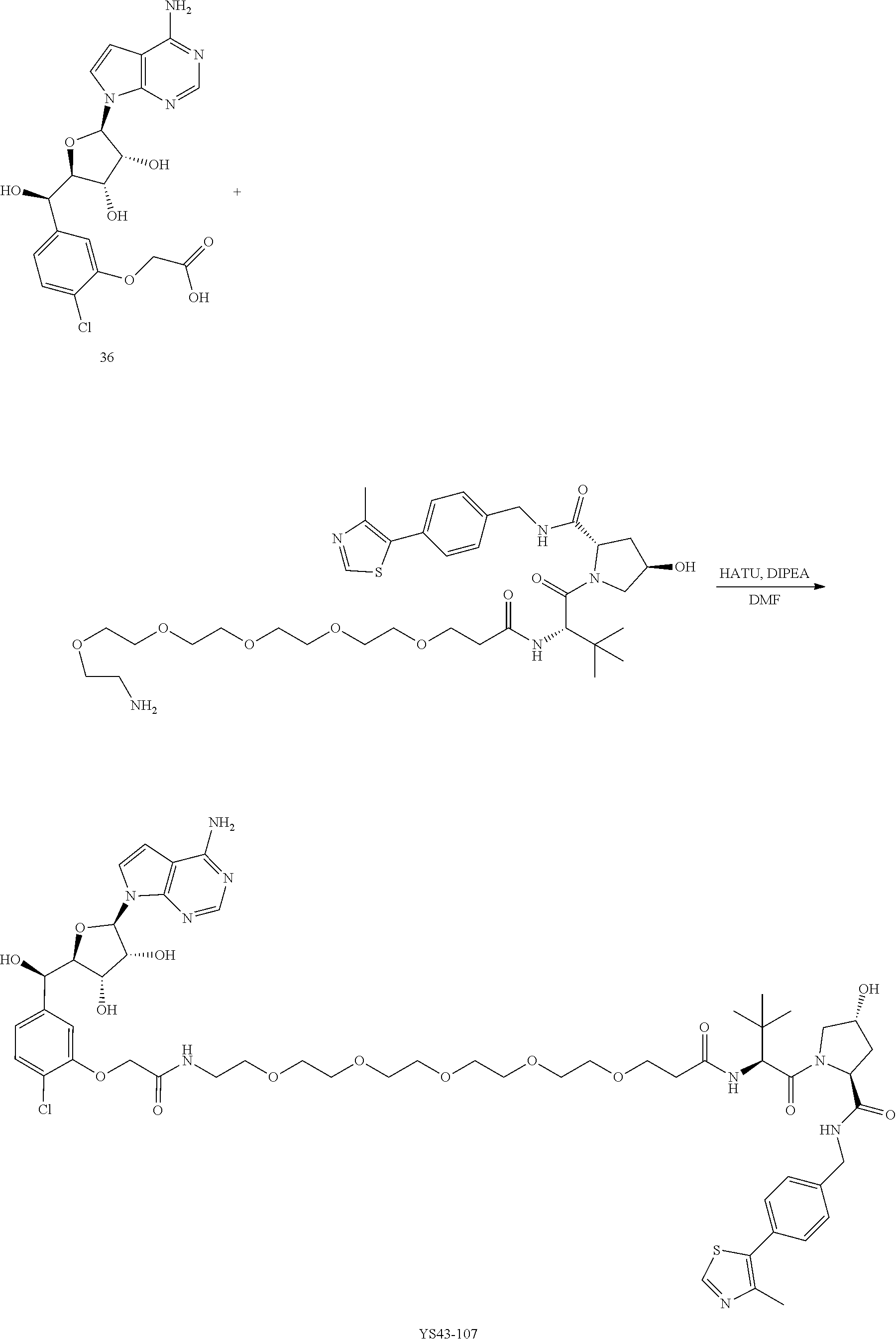

To a solution of intermediate 36 (9 mg, 0.02 mmol), (2S,4R)-1-((S)-1-amino-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (14 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-107 (6.1 mg, yield 26%). ¹H NMR (800 MHz, Methanol-d₄) δ 8.93 (s, 1H), 8.27 (s, 1H), 7.65 (d, J=3.9 Hz, 1H), 7.52-7.36 (m, 5H), 7.16-7.05 (m, 2H), 6.94 (d, J=4.0 Hz, 1H), 6.25 (d, J=6.6 Hz, 1H), 4.97-4.89 (m, 1H), 4.67 (s, 1H), 4.64-4.45 (m, 6H), 4.38 (d, J=15.4 Hz, 1H), 4.34-4.29 (m, 1H), 4.26 (d, J=3.6 Hz, 1H), 3.91 (d, J=10.9 Hz, 1H), 3.82 (dd, J=11.1, 4.0 Hz, 1H), 3.76-3.66 (m, 2H), 3.65-3.54 (m, 18H), 3.50 (q, J=5.2 Hz, 2H), 2.58 (dt, J=13.6, 6.5 Hz, 1H), 2.52-2.41 (m, 4H), 2.24 (dd, J=13.3, 7.6 Hz, 1H), 2.11 (ddd, J=13.3, 9.0, 4.6 Hz, 1H), 1.06 (s, 9H). MS (ESI) m/z 1154.6 [M+H]⁺.

### Example 80: Synthesis of YS43-108

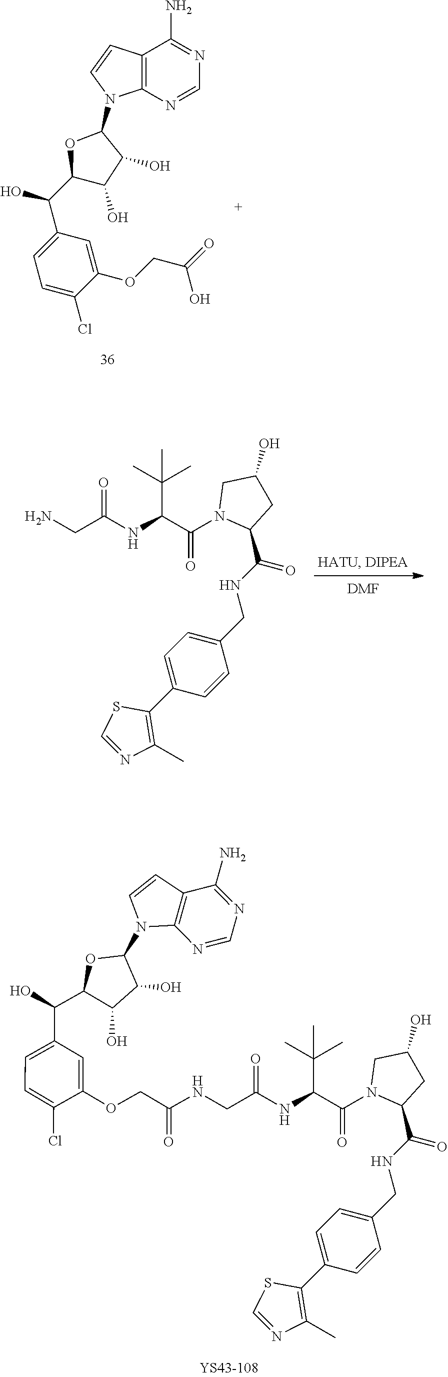

To a solution of intermediate 36 (9 mg, 0.02 mmol), (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide (9.8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-108 (4.2 mg, yield 23%). ¹H NMR (800 MHz, Methanol-d₄) δ 8.94 (s, 1H), 8.24 (s, 1H), 7.64 (d, J=3.9 Hz, 1H), 7.48 (d, J=7.8 Hz, 2H), 7.43 (d, J=8.0 Hz, 2H), 7.37 (d, J=8.1 Hz, 1H), 7.15 (s, 1H), 7.10 (d, J=8.2 Hz, 1H), 6.94 (d, J=3.9 Hz, 1H), 6.24 (d, J=6.1 Hz, 1H), 4.95 (d, J=3.8 Hz, 1H), 4.69 (s, 1H), 4.66-4.47 (m, 6H), 4.37 (d, J=15.4 Hz, 1H), 4.33 (q, J=4.1 Hz, 1H), 4.28 (q, J=3.9 Hz, 1H), 4.14 (d, J=16.8 Hz, 1H), 4.03 (d, J=16.8 Hz, 1H), 3.93 (d, J=11.0 Hz, 1H), 3.80 (dd, J=11.2, 4.0 Hz, 1H), 2.49 (s, 3H), 2.24 (dd, J=13.2, 7.6 Hz, 1H), 2.10 (ddd, J=13.3, 9.0, 4.6 Hz, 1H), 1.07 (s, 9H). MS (ESI) m/z 920.2 [M+H]⁺.

### Example 81: Synthesis of YS43-109

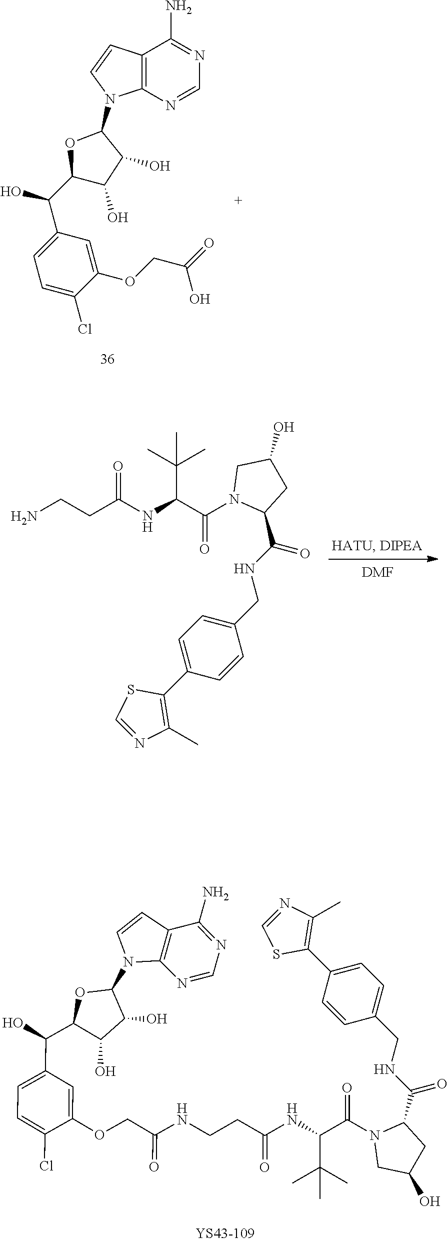

To a solution of intermediate 36 (9 mg, 0.02 mmol), (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide (10 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-109 (6.6 mg, yield 35%). ¹H NMR (800 MHz, Methanol-d₄) δ 8.94 (s, 1H), 8.25 (s, 1H), 7.66 (d, J=3.9 Hz, 1H), 7.55-7.28 (m, 5H), 7.15-7.04 (m, 2H), 6.94 (d, J=4.0 Hz, 1H), 6.24 (d, J=6.5 Hz, 1H), 4.94 (d, J=3.9 Hz, 1H), 4.68-4.43 (m, 7H), 4.37 (d, J=15.4 Hz, 1H), 4.32-4.28 (m, 1H), 4.28-4.22 (m, 1H), 3.92 (d, J=11.0 Hz, 1H), 3.81 (dd, J=11.1, 4.0 Hz, 1H), 3.58 (t, J=6.7 Hz, 2H), 2.63-2.43 (m, 5H), 2.27-2.18 (m, 1H), 2.12-2.07 (m, 1H), 1.04 (s, 9H). MS (ESI) m/z 934.3 [M+H]⁺.

### Example 82: Synthesis of YS43-110

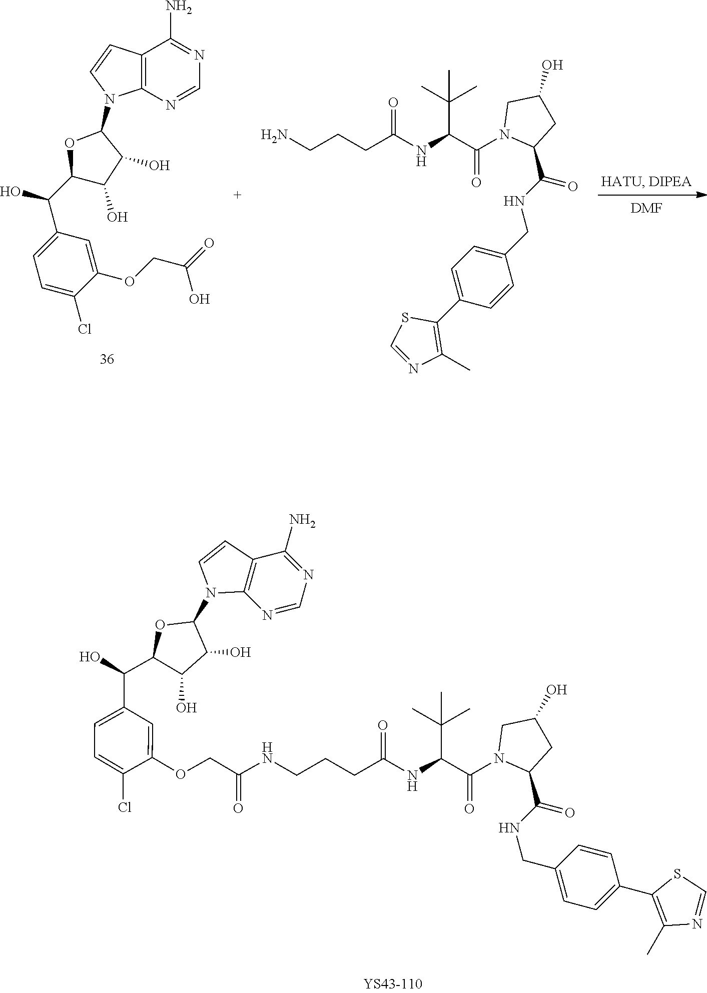

To a solution of intermediate 36 (9 mg, 0.02 mmol), (2S,4R)-1-((S)-2-(4-aminobutanamido)-3,3-dimethylbutanoyl)-4-hydroxy-N-(4-(4-methylthiazol-5-yl)benzyl)pyrrolidine-2-carboxamide (10.3 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-110 (5 mg, yield 26%). ¹H NMR (800 MHz, Methanol-d₄) δ 8.95 (s, 1H), 8.26 (s, 1H), 7.66 (d, J=3.9 Hz, 1H), 7.53-7.31 (m, 5H), 7.25-7.06 (m, 2H), 6.94 (d, J=4.0 Hz, 1H), 6.25 (d, J=6.8 Hz, 1H), 4.94 (d, J=3.9 Hz, 1H), 4.64-4.48 (m, 7H), 4.37 (d, J=15.3 Hz, 1H), 4.30-4.28 (m, 1H), 4.25 (d, J=3.4 Hz, 1H), 3.93 (d, J=10.9 Hz, 1H), 3.82 (dd, J=11.1, 4.1 Hz, 1H), 3.36-3.32 (m, 2H), 2.49 (s, 3H), 2.31 (p, J=7.6 Hz, 2H), 2.24 (dd, J=13.7, 7.5 Hz, 1H), 2.15-2.06 (m, 1H), 1.85 (tt, J=14.8, 7.0 Hz, 2H), 1.06 (s, 9H). MS (ESI) m/z 948.3 [M+H]⁺.

### Example 83: Synthesis of YS43-111

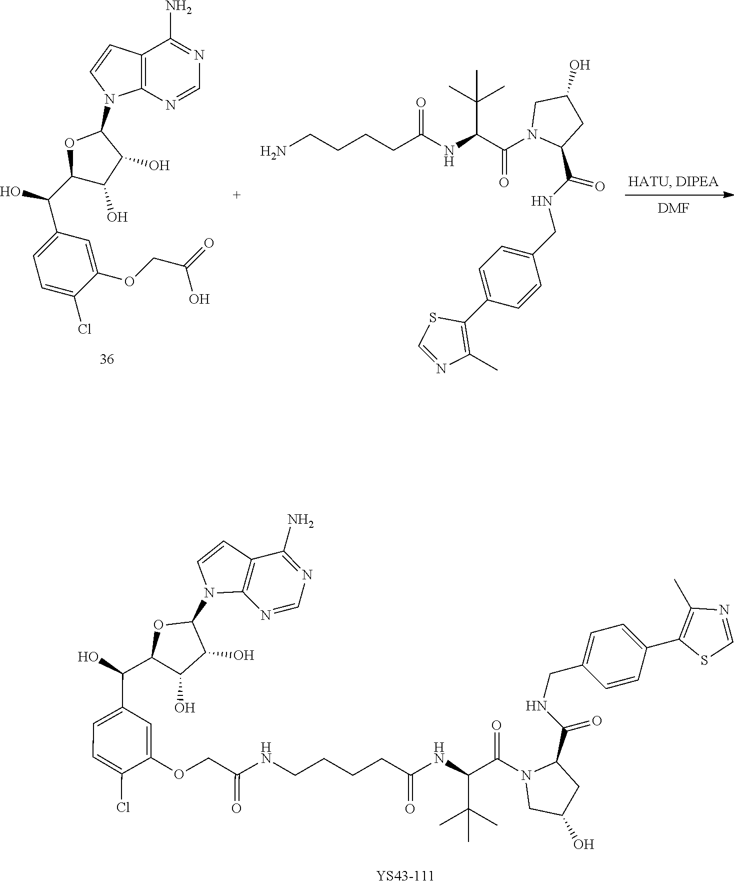

To a solution of intermediate 36 (9 mg, 0.02 mmol), (2S,4R)-1-((S)-2-(5-aminopentanamido)-3,3-dimethylbutanoyl)-4-hydroxy-N-(4-(4-methylthiazol-5-yl)benzyl)pyrrolidine-2-carboxamide (10.6 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-111 (8.8 mg, yield 46%). ¹H NMR (800 MHz, Methanol-d₄) δ 8.93 (s, 1H), 8.26 (s, 1H), 7.67 (d, J=3.9 Hz, 1H), 7.53-7.33 (m, 5H), 7.18-7.03 (m, 2H), 6.95 (d, J=3.9 Hz, 1H), 6.25 (d, J=6.6 Hz, 1H), 4.98-4.84 (m, 1H), 4.66-4.46 (m, 7H), 4.38 (d, J=15.5 Hz, 1H), 4.33-4.27 (m, 1H), 4.25 (t, J=3.6 Hz, 1H), 3.92 (d, J=10.9 Hz, 1H), 3.81 (dd, J=11.1, 4.3 Hz, 1H), 3.33-3.25 (m, 2H), 2.49 (s, 3H), 2.33 (dtd, J=28.7, 14.5, 7.4 Hz, 2H), 2.27-2.20 (m, 1H), 2.11 (ddd, J=13.2, 8.9, 4.5 Hz, 1H), 1.69-1.51 (m, 4H), 1.05 (s, 9H). MS (ESI) m/z 962.4 [M+H]⁺.

### Example 84: Synthesis of YS43-112

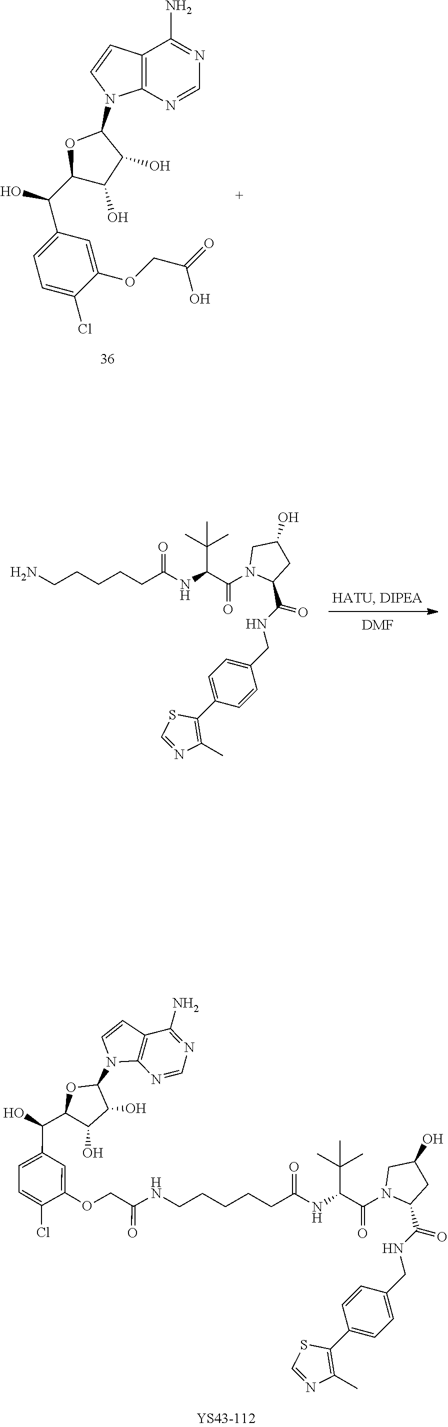

To a solution of intermediate 36 (9 mg, 0.02 mmol), (2S,4R)-1-((S)-2-(6-aminohexanamido)-3,3-dimethylbutanoyl)-4-hydroxy-N-(4-(4-methylthiazol-5-yl)benzyl)pyrrolidine-2-carboxamide (10.8 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-112 (5.6 mg, yield 29%). ¹H NMR (800 MHz, Methanol-d₄) δ 8.93 (s, 1H), 8.27 (s, 1H), 7.67 (d, J=3.9 Hz, 1H), 7.54-7.37 (m, 5H), 7.19-7.05 (m, 2H), 6.95 (d, J=4.0 Hz, 1H), 6.25 (d, J=6.8 Hz, 1H), 4.97-4.89 (m, 1H), 4.70-4.46 (m, 7H), 4.38 (d, J=15.4 Hz, 1H), 4.31-4.27 (m, 1H), 4.25 (d, J=3.4 Hz, 1H), 3.93 (d, J=10.9 Hz, 1H), 3.82 (dd, J=11.1, 4.1 Hz, 1H), 3.34-3.23 (m, 2H), 2.49 (s, 3H), 2.32-2.21 (m, 3H), 2.10 (td, J=9.0, 4.6 Hz, 1H), 1.71-1.53 (m, 4H), 1.37 (p, J=8.1 Hz, 2H), 1.05 (s, 9H). MS (ESI) m/z 976.5 [M+H]⁺.

### Example 85: Synthesis of YS43-113

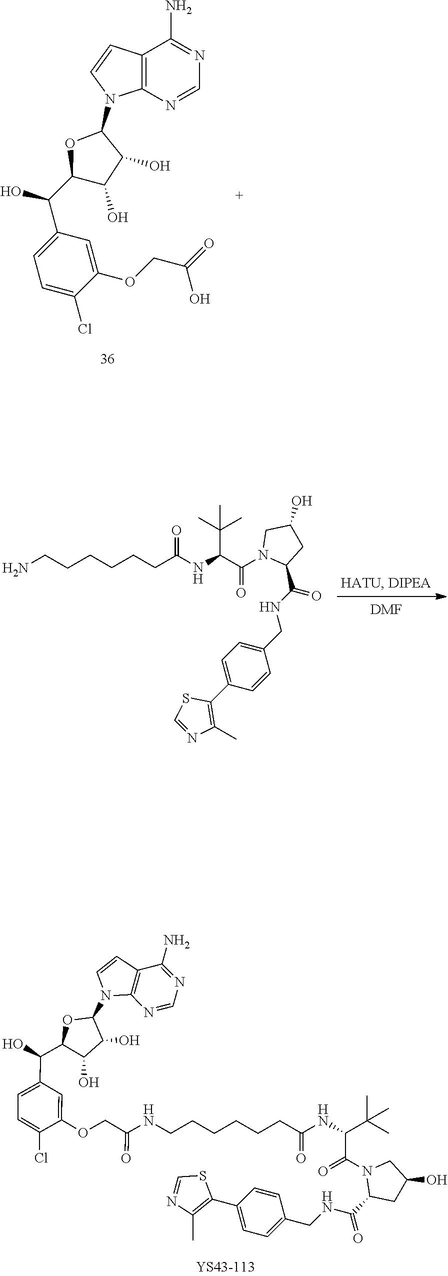

To a solution of intermediate 36 (9 mg, 0.02 mmol), (2S,4R)-1-((S)-2-(7-aminoheptanamido)-3,3-dimethylbutanoyl)-4-hydroxy-N-(4-(4-methylthiazol-5-yl)benzyl)pyrrolidine-2-carboxamide (11 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-113 (10 mg, yield 51%). ¹H NMR (800 MHz, Methanol-d₄) δ 8.97 (s, 1H), 8.27 (s, 1H), 7.67 (d, J=3.9 Hz, 1H), 7.56-7.35 (m, 5H), 7.21-7.07 (m, 2H), 6.95 (d, J=3.9 Hz, 1H), 6.25 (d, J=6.7 Hz, 1H), 4.99-4.90 (m, 1H), 4.71-4.46 (m, 7H), 4.38 (d, J=15.5 Hz, 1H), 4.30 (d, J=5.3 Hz, 1H), 4.25 (d, J=3.4 Hz, 1H), 3.93 (d, J=10.9 Hz, 1H), 3.82 (dd, J=11.1, 4.0 Hz, 1H), 3.30 (d, J=7.0 Hz, 2H), 2.49 (s, 3H), 2.33-2.21 (m, 3H), 2.11-2.06 (m, 1H), 1.67-1.53 (m, 4H), 1.41-1.31 (m, 4H), 1.05 (s, 9H). MS (ESI) m/z 990.4 [M+H]⁺.

### Example 86: Synthesis of YS43-114

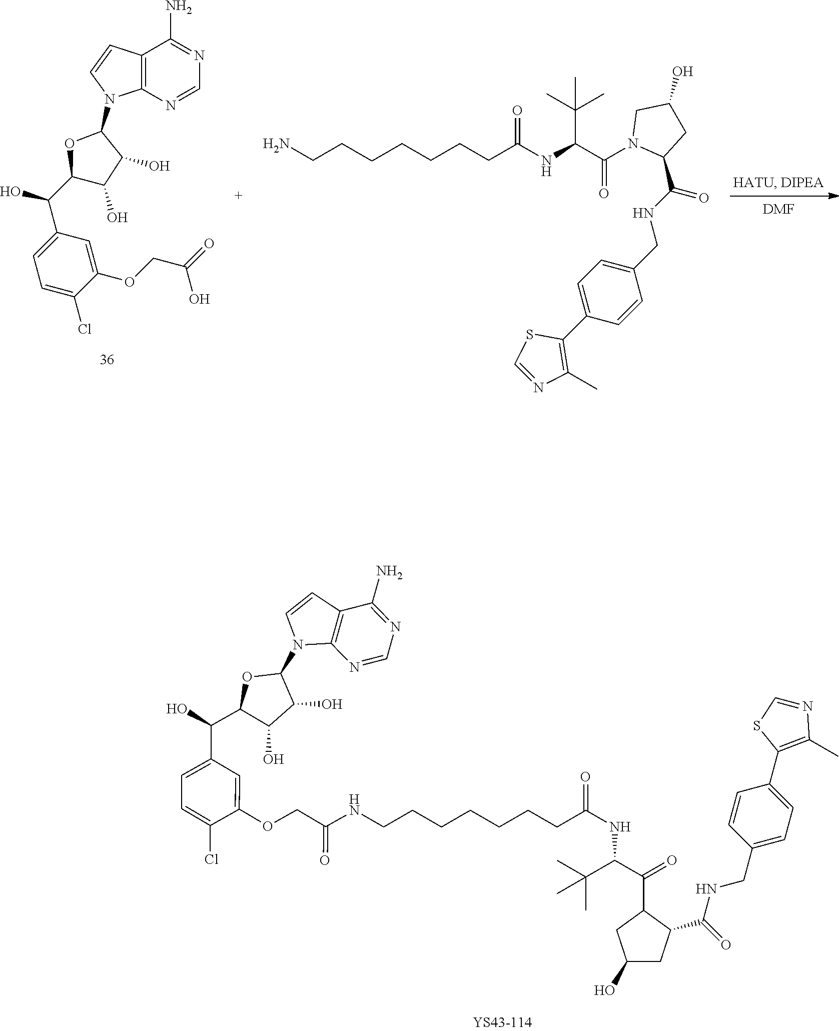

To a solution of intermediate 36 (9 mg, 0.02 mmol), (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide (11.4 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-114 (5 mg, yield 25%). ¹H NMR (800 MHz, Methanol-d₄) δ 8.94 (s, 1H), 8.27 (s, 1H), 7.68 (d, J=3.8 Hz, 1H), 7.54-7.32 (m, 5H), 7.18-7.04 (m, 2H), 6.95 (d, J=3.7 Hz, 1H), 6.25 (d, J=6.8 Hz, 1H), 4.97-4.91 (m, 1H), 4.69-4.47 (m, 7H), 4.38 (d, J=15.4 Hz, 1H), 4.30 (d, J=5.3 Hz, 1H), 4.28-4.22 (m, 1H), 3.93 (d, J=10.9 Hz, 1H), 3.82 (dd, J=11.1, 3.9 Hz, 1H), 3.32 (s, 2H), 2.49 (s, 3H), 2.33-2.22 (m, 3H), 2.11 (ddd, J=13.3, 9.0, 4.6 Hz, 1H), 1.67-1.50 (m, 4H), 1.41-1.32 (m, 6H), 1.05 (s, 9H). MS (ESI) m/z 1004.4[M+H]⁺.

### Example 87: Synthesis of YS43-115

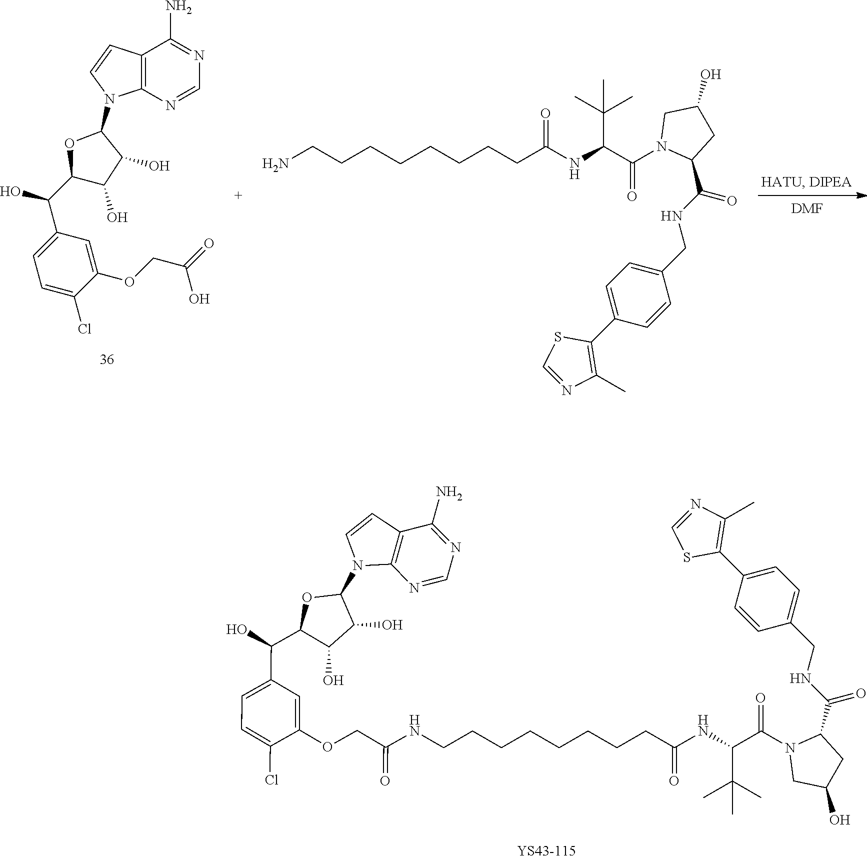

To a solution of intermediate 36 (9 mg, 0.02 mmol), (2S,4R)-1-((S)-2-(9-aminononanamido)-3,3-dimethylbutanoyl)-4-hydroxy-N-(4-(4-methylthiazol-5-yl)benzyl)pyrrolidine-2-carboxamide (11.7 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-115 (6.4 mg, yield 31%). ¹H NMR (800 MHz, Methanol-d₄) δ 8.95 (s, 1H), 8.27 (s, 1H), 7.68 (d, J=3.9 Hz, 1H), 7.55-7.35 (m, 5H), 7.31-7.04 (m, 2H), 6.95 (d, J=4.0 Hz, 1H), 6.26 (d, J=6.7 Hz, 1H), 4.94-4.88 (m, 1H), 4.71-4.47 (m, 7H), 4.38 (d, J=15.4 Hz, 1H), 4.30 (d, J=5.7 Hz, 1H), 4.25 (d, J=3.4 Hz, 1H), 3.93 (d, J=10.9 Hz, 1H), 3.83 (dd, J=11.1, 4.1 Hz, 1H), 3.31-3.25 (m, 2H), 2.50 (s, 3H), 2.34-2.21 (m, 3H), 2.15-2.06 (m, 1H), 1.70-1.49 (m, 4H), 1.43-1.25 (m, 8H), 1.05 (s, 9H). MS (ESI) m/z 1018.4 [M+H]⁺.

### Example 88: Synthesis of YS43-116

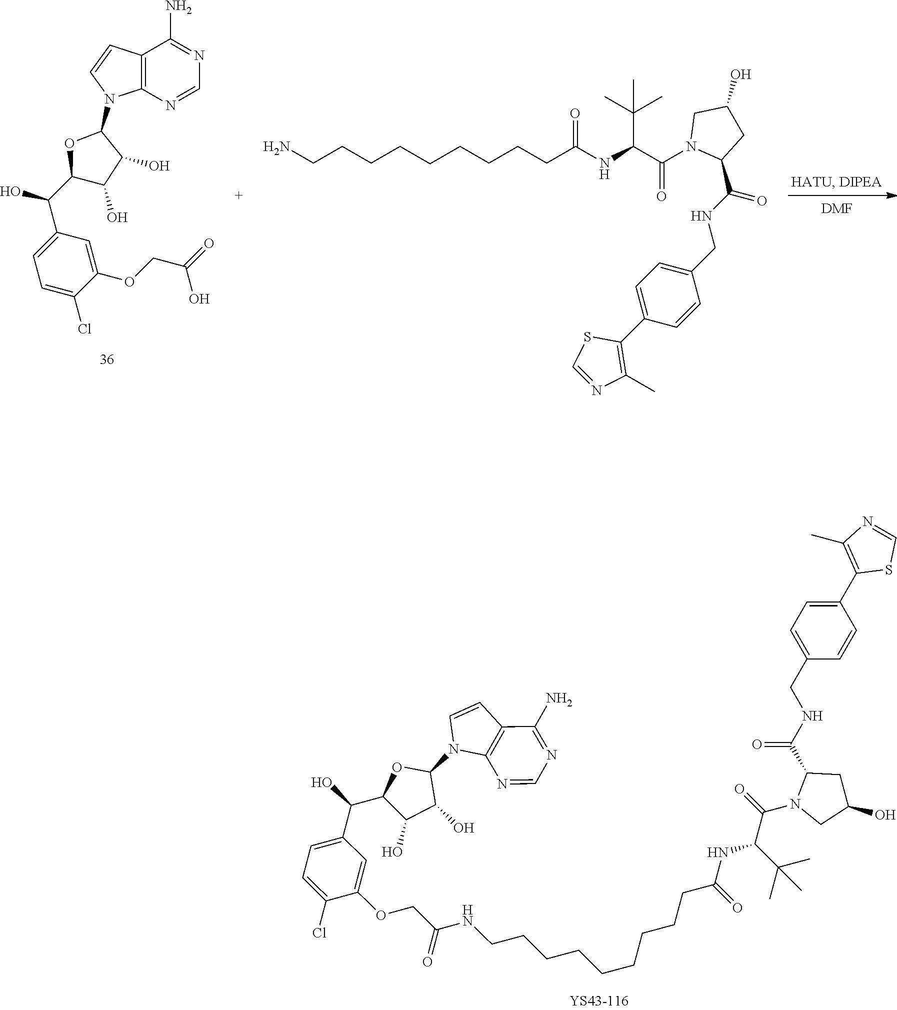

To a solution of intermediate 36 (9 mg, 0.02 mmol), (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide (12 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-116 (2.7 mg, yield 13%). ¹H NMR (800 MHz, Methanol-d₄) δ 8.94 (s, 1H), 8.27 (s, 1H), 7.68 (d, J=3.9 Hz, 1H), 7.52-7.35 (m, 5H), 7.22-7.06 (m, 2H), 6.95 (d, J=4.0 Hz, 1H), 6.26 (d, J=6.7 Hz, 1H), 4.94 (d, J=3.8 Hz, 1H), 4.68-4.48 (m, 7H), 4.38 (d, J=15.3 Hz, 1H), 4.33-4.27 (m, 1H), 4.25 (d, J=3.6 Hz, 1H), 3.93 (d, J=10.9 Hz, 1H), 3.83 (dd, J=11.1, 4.1 Hz, 1H), 3.33-3.24 (m, 2H), 2.50 (s, 3H), 2.34-2.22 (m, 3H), 2.13-2.06 (m, 1H), 1.72-1.51 (m, 4H), 1.40-1.21 (m, 10H), 1.06 (s, 9H). MS (ESI) m/z 1032.4[M+H]⁺.

### Example 89: Synthesis of YS43-117

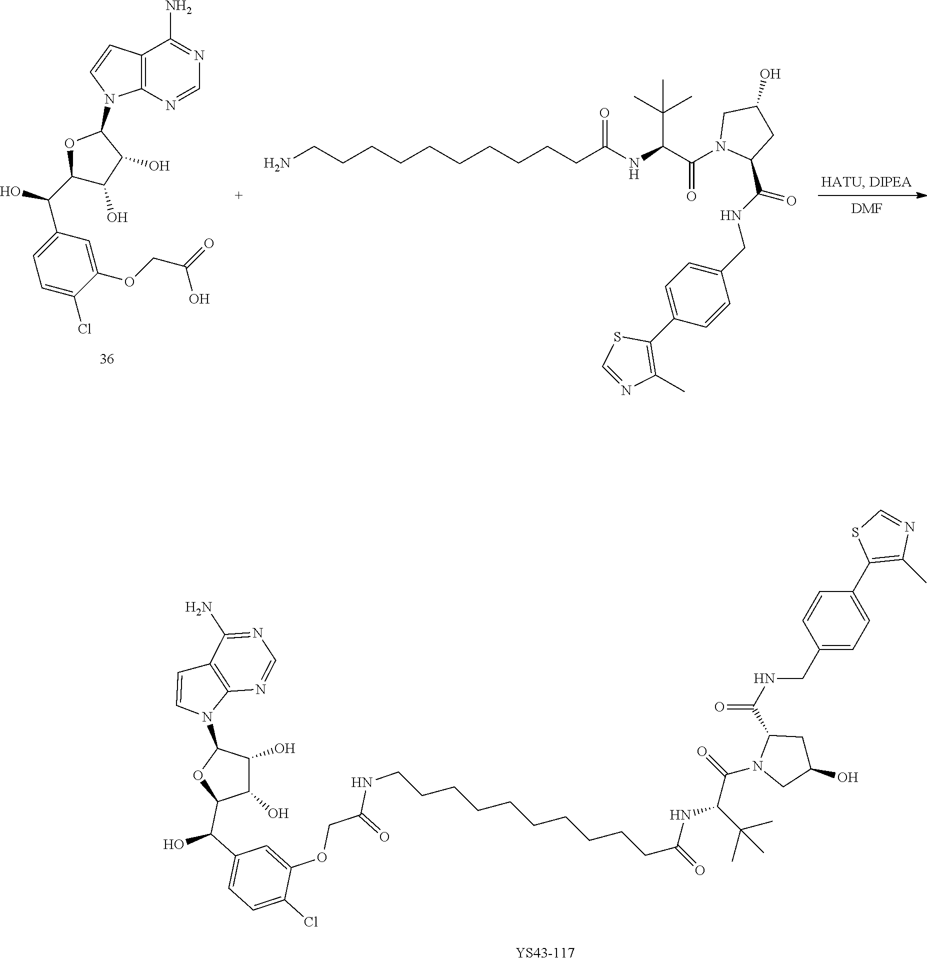

To a solution of intermediate 36 (9 mg, 0.02 mmol), (2S,4R)-1-((S)-2-(11-aminodecanamido)-3,3-dimethylbutanoyl)-4-hydroxy-N-(4-(4-methylthiazol-5-yl)benzyl)pyrrolidine-2-carboxamide (12.2 mg, 0.02 mmol), and HATU (11.4 mg, 0.03 mmol) in DMF (1 mL), was added DIPEA (12.9 mg, 0.1 mmol). After the solution was stirred at room temperature for 1 h, it was purified by preparative HPLC (10%-100% methanol/0.1% TFA in H₂O) to afford YS43-117 (5.6 mg, yield 27%). ¹H NMR (800 MHz, Methanol-d₄) δ 8.93 (s, 1H), 8.27 (s, 1H), 7.68 (d, J=3.9 Hz, 1H), 7.53-7.37 (m, 5H), 7.22-7.04 (m, 2H), 6.95 (d, J=3.9 Hz, 1H), 6.26 (d, J=6.7 Hz, 1H), 4.94 (d, J=4.2 Hz, 1H), 4.69-4.47 (m, 7H), 4.38 (d, J=15.3 Hz, 1H), 4.31-4.28 (m, 1H), 4.25 (d, J=3.5 Hz, 1H), 3.93 (d, J=10.9 Hz, 1H), 3.83 (dd, J=11.1, 4.1 Hz, 1H), 3.31 (t, J=7.1 Hz, 2H), 2.50 (s, 3H), 2.36-2.21 (m, 3H), 2.16-2.08 (m, 1H), 1.69-1.50 (m, 4H), 1.33 (s, 12H), 1.06 (s, 9H). MS (ESI) m/z 1046.4 [M+H]⁺.

Certain compounds disclosed herein have the structures shown in Table 1.

TABLE 1

| Com- | | |
|---|---|---|
| pound | | |
| ID | Structure | Chemical Name |
| YS31- 58 | 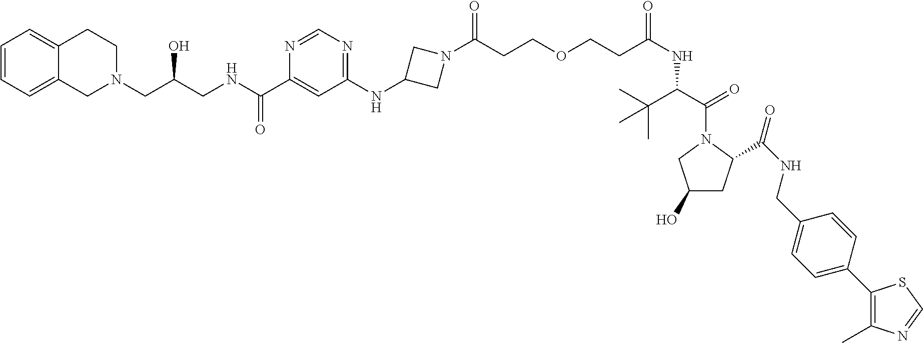 [SMILES: Cc1ncsc1-c1ccc(CNC(=O)[C@@H]2C[C@@H](O)CN2C(=O)[C@@H](NC(=O)CCOCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(3- (3-(((5)-1-((2S,4R)-4- hydroxy-2-((4-(4- methylthiazol-5- yl)benzyl)carbamoyl) pyrrolidin- 1-yl)-3,3-dimethyl- 1-oxobutan-2- yl)amino)-3- oxopropoxy) propanoyl) azetidin-3- yl)amino)pyrimidine- 4-carboxamide |
| YS31- 59 | 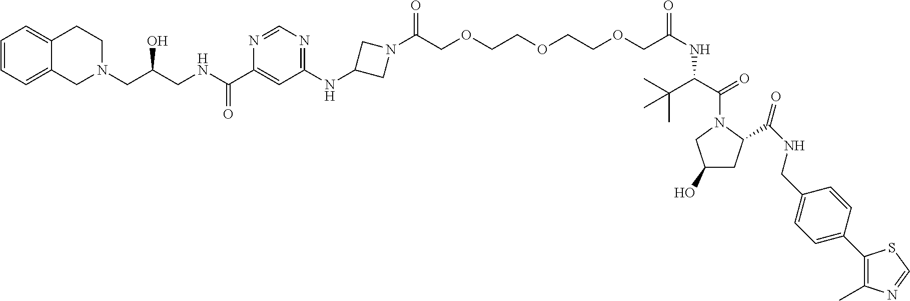 [SMILES: Cc1ncsc1-c1ccc(CNC(=O)[C@@H]2C[C@@H](O)CN2C(=O)[C@@H](NC(=O)COCCOCCOCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 13-((2S,4R)- 4-hydroxy- 2-((4-(4- methylthiazol-5- yl)benzyl)carbamoyl) pyrrolidine- 1-carbonyl)-14,14- dimethyl-11-oxo- 3,6,9-trioxa-12- azapentadecanoyl) azetidin-3- yl)amino)pyrimidine- 4-carboxamide |
| YS31- 60 | 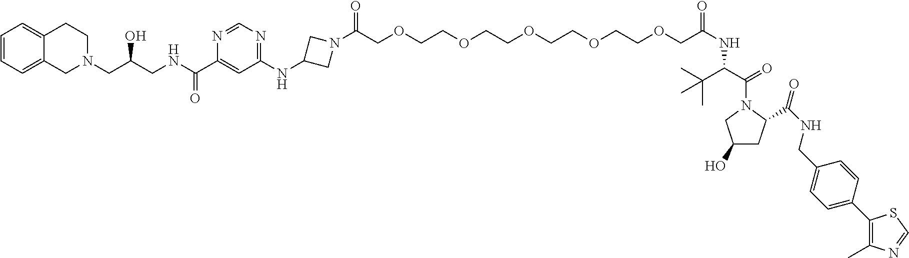 [SMILES: Cc1ncsc1-c1ccc(CNC(=O)[C@@H]2C[C@@H](O)CN2C(=O)[C@@H](NC(=O)COCCOCCOCCOCCOCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)-19- ((2S,4R)-4-hydroxy- 2-((4-(4- methylthiazol-5- yl)benzyl)carbamoyl) pyrrolidine- 1-carbonyl)-20,20- dimethyl-17-oxo- 3,6,9,12,15- pentaoxa-18- azahenicosanoyl) azetidin-3- yl)amino)pyrimidine- 4-carboxamide |
| YS31- 61 | 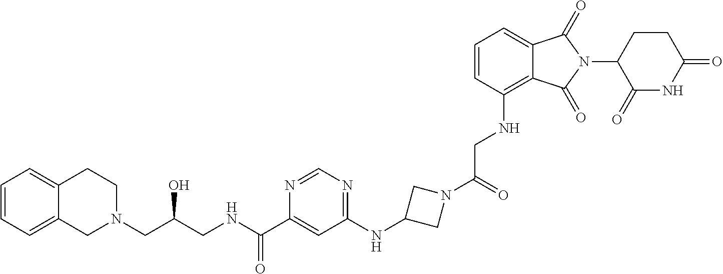 [SMILES: O=C1CCC(N2C(=O)c3cccc(NCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-((2- (2,6-dioxopiperidin- 3-yl)- 1,3-dioxoisoindolin- 4-yl) glycyl)azetidin-3- yl)amino)pyrimidine- 4-carboxamide |
| YS31- 62 | 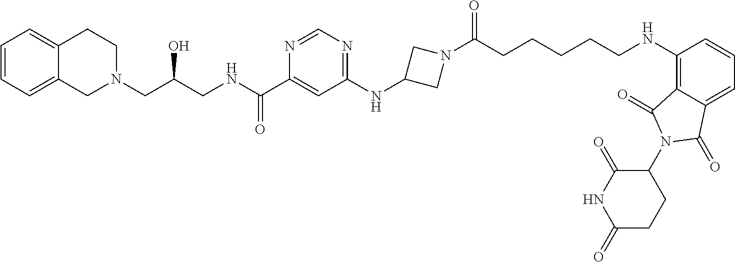 [SMILES: O=C1CCC(N2C(=O)c3cccc(NCCCCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(6- ((2-(2,6- dioxopiperidin-3- yl)-1,3- dioxoisoindolin-4- yl)amino)hexanoyl) azetidin- 3-yl)amino) pyrimidine- 4-carboxamide |
| YS31- 63 | 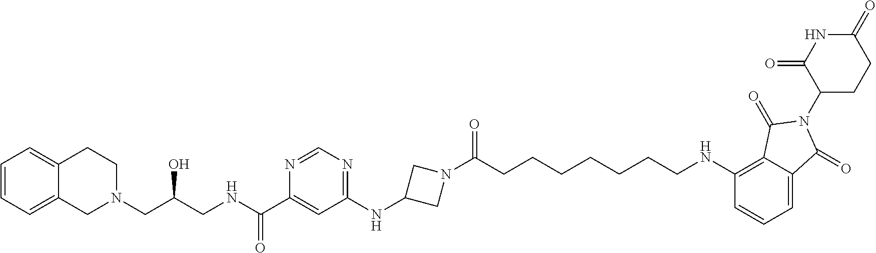 [SMILES: O=C1CCC(N2C(=O)c3cccc(NCCCCCCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(8-((2- (2,6-dioxopiperidin-3- yl)-1,3- dioxoisoindolin-4- yl)amino)octanoyl) azetidin- 3-yl)amino) pyrimidine- 4-carboxamide |
| YS31- 64 | 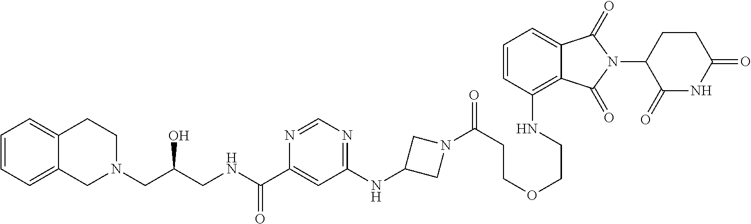 [SMILES: O=C1CCC(N2C(=O)c3cccc(NCCOCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(3-(2- ((2-(2,6- dioxopiperidin- 3-yl)-1,3- dioxoisoindolin-4- yl)amino)ethoxy) propanoyl)azetidin-3- yl)amino)pyrimidine- 4-carboxamide |
| YS31- 65 | 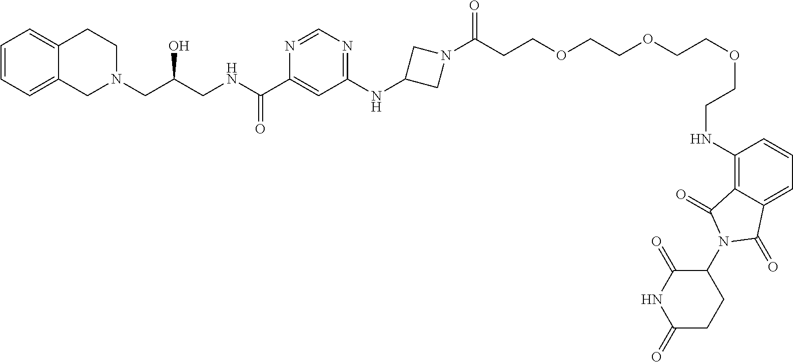 [SMILES: O=C1CCC(N2C(=O)c3cccc(NCCOCCOCCOCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)-6-((1- (3-(2-(2-(2-((2-(2,6- dioxopiperidin- 3-yl)-1,3- dioxoisoindolin-4- yl)amino) ethoxy)ethoxy) ethoxy)propanoyl) azetidin- 3-yl)amino) pyrimidine- 4-carboxamide |
| YS31- 66 | 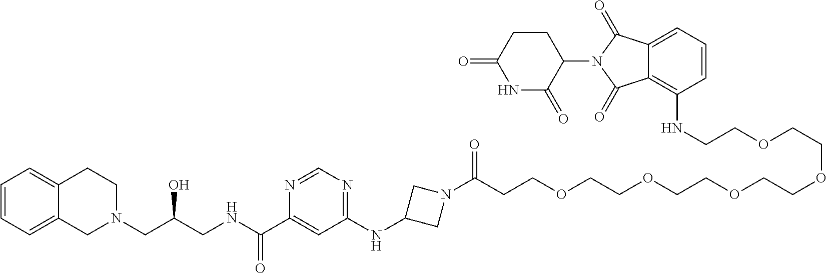 [SMILES: O=C1CCC(N2C(=O)c3cccc(NCCOCCOCCOCCOCCOCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(1- ((2-(2,6- dioxopiperidin-3- yl)-1,3- dioxoisoindolin-4- yl)amino)-3,6,9,12,15- pentaoxaoctadecan- 18- oyl)azetidin-3- yl)amino) pyrimidine-4- carboxamide |
| YS31- 67 | 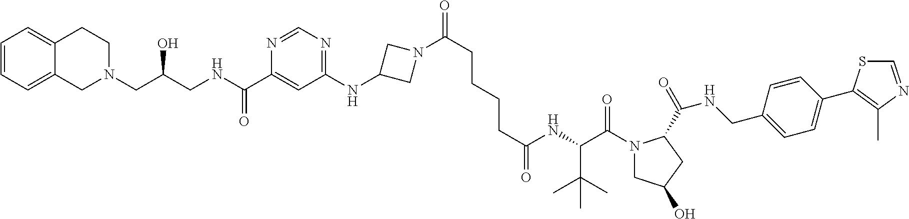 [SMILES: Cc1ncsc1-c1ccc(CNC(=O)[C@@H]2C[C@@H](O)CN2C(=O)[C@@H](NC(=O)CCCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(6- (((S)-1-((2S,4R)-4- hydroxy-2-((4- (4-methylthiazol-5- yl)benzyl)carbamoyl) pyrrolidin- 1-yl)-3,3-dimethyl- 1-oxobutan-2- yl)amino)-6- oxohexanoyl) |
| | | azetidin-3- |
| | | yl)amino)pyrimidine- |
| | | 4-carboxamide |
| YS31- 68 | 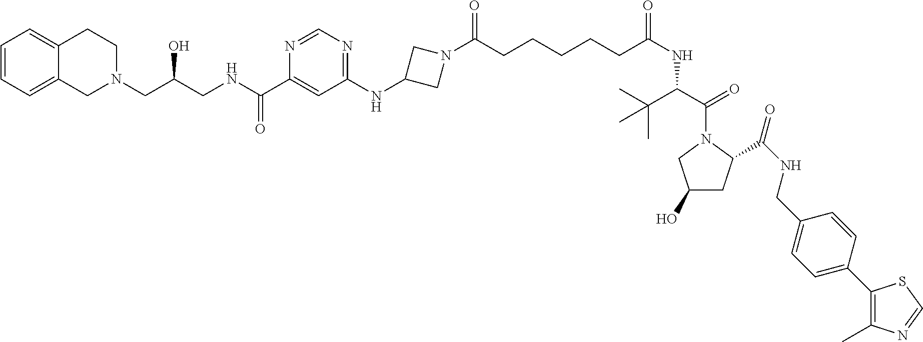 [SMILES: Cc1ncsc1-c1ccc(CNC(=O)[C@@H]2C[C@@H](O)CN2C(=O)[C@@H](NC(=O)CCCCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1- (7-(((S)-1-((2S,4R)-4- hydroxy-2-((4-(4- methylthiazol-5- yl)benzyl)carbamoyl) pyrrolidin- 1-yl)-3,3-dimethyl- 1-oxobutan-2- yl)amino)-7- oxoheptanoyl) azetidin-3- yl)amino)pyrimidine- 4-carboxamide |
| YS31- 69 | 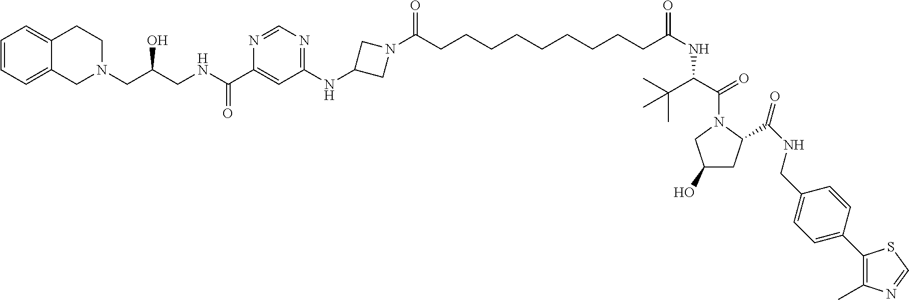 [SMILES: Cc1ncsc1-c1ccc(CNC(=O)[C@@H]2C[C@@H](O)CN2C(=O)[C@@H](NC(=O)CCCCCCCCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(11- (((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-11- oxoundecanoyl) azetidin-3- yl)amino) pyrimidine-4- carboxamide |
| YS43- 6 | 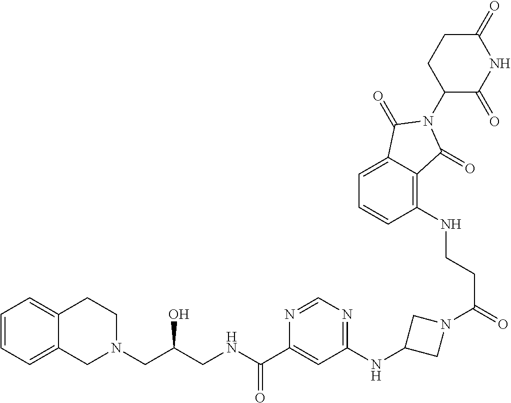 [SMILES: O=C1CCC(N2C(=O)c3cccc(NCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(3- ((2-(2,6- dioxopiperidin-3- yl)-1,3- dioxoisoindolin-4- yl)amino)propanoyl) azetidin-3- yl)amino) pyrimidine- 4-carboxamide |
| YS43- 7 | 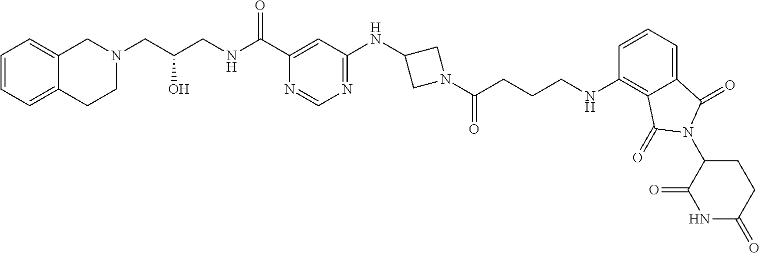 [SMILES: O=C1CCC(N2C(=O)c3cccc(NCCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(4- ((2-(2,6- dioxopiperidin-3- yl)-1,3- dioxoisoindolin-4- yl)amino)butanoyl) azetidin-3- yl)amino) pyrimidine- 4-carboxamide |
| YS43- 8 | 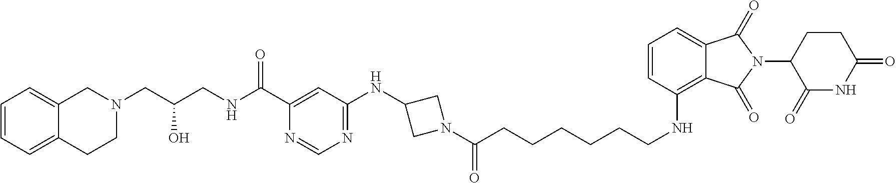 [SMILES: O=C1CCC(N2C(=O)c3cccc(NCCCCCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(7- ((2-(2,6- dioxopiperidin-3- yl)-1,3- dioxoisoindolin-4- yl)amino)heptanoyl) azetidin-3- yl)amino)pyrimidine- 4-carboxamide |
| YS43- 9 | 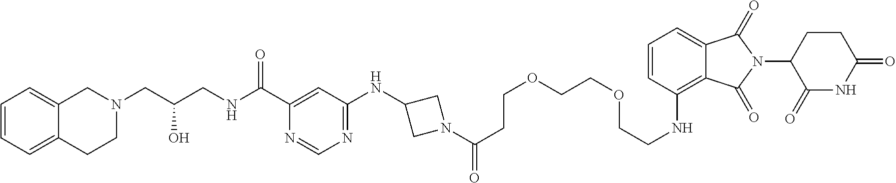 [SMILES: O=C1CCC(N2C(=O)c3cccc(NCCOCCOCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-1- hydroxypropyl)-6-((1- (3-(2-(2-((2-(2,6- dioxopiperidin- 3-yl)-1,3- dioxoisoindolin-4- yl)amino) ethoxy)ethoxy) propanoyl)azetidin-3- yl)amino) |
| | | pyrimidine-4- |
| | | carboxamide |
| YS43- 10 | 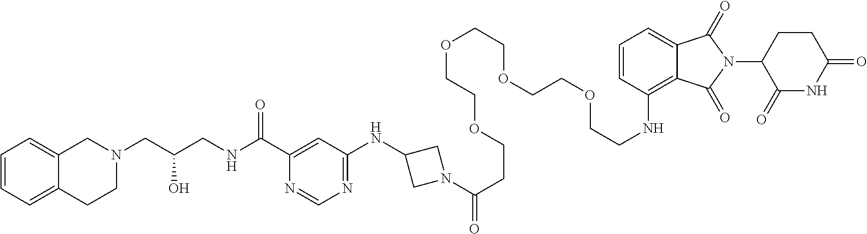 [SMILES: O=C1CCC(N2C(=O)c3cccc(NCCOCCOCCOCCOCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(1- ((2-(2,6- dioxopiperidin-3- yl)-1,3- dioxoisoindolin-4- yl)amino)-3,6,9,12- tetraoxapentadecan- 15- oyl)azetidin-3- yl)amino) pyrimidine-4- carboxamide_ |
| YS43- 11 | 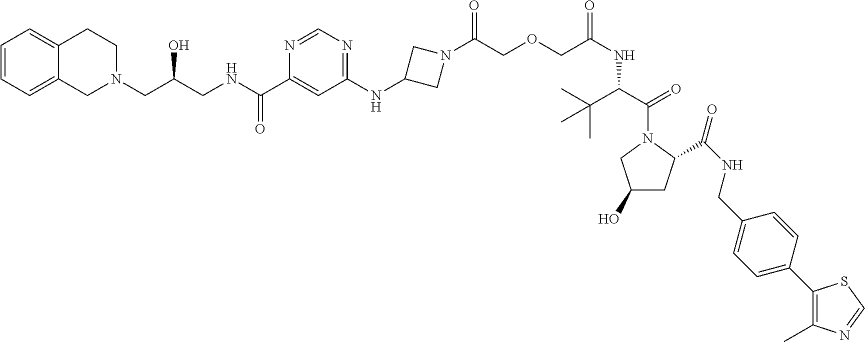 [SMILES: Cc1ncsc1-c1ccc(CNC(=O)[C@@H]2C[C@@H](O)CN2C(=O)[C@@H](NC(=O)COCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(2- (2-(((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-2- oxoethoxy)acetyl) azetidin-3- yl)amino)pyrimidine- 4-carboxaniide |
| YS43- 12 | 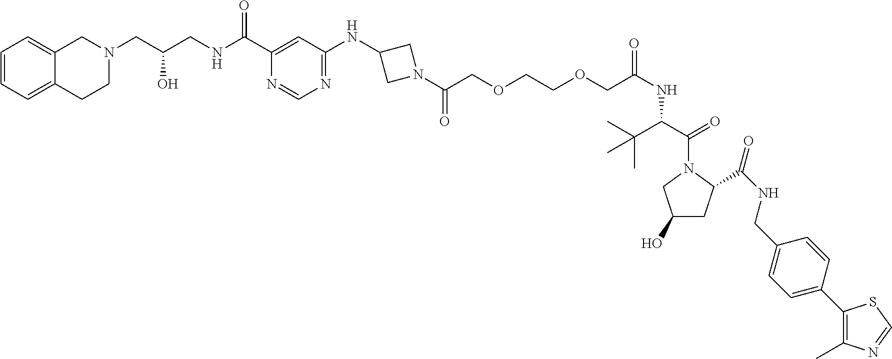 [SMILES: Cc1ncsc1-c1ccc(CNC(=O)[C@@H]2C[C@@H](O)CN2C(=O)[C@@H](NC(=O)COCCOCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(2-(2- (2-(((S)-1-((2S,4R)-4- hydroxy-2-((4-(4- methylthiazol-5- yl)benzyl)caibamoyl) pyrrolidin- 1-yl)-3,3-dimethyl- 1-oxobutan-2-yl) amino)-2- oxoethoxy)ethoxy) acetyl)azetidin-3- yl)amino)pyrimidine- 4-carboxamide |
| YS43- 13 | 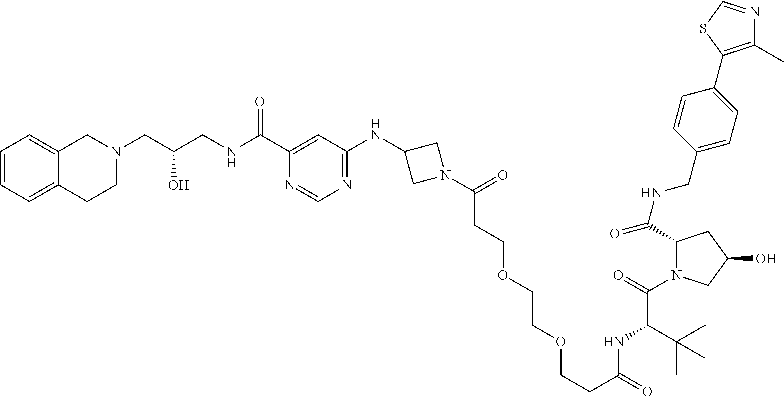 [SMILES: Cc1ncsc1-c1ccc(CNC(=O)[C@@H]2C[C@@H](O)CN2C(=O)[C@@H](NC(=O)CCOCCOCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(3-(2- (3-(((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-3- oxopropoxy)ethoxy) propanoyl)azetidin-3- yl)amino)pyrimidine- 4-carboxamide |
| YS43- 14 | 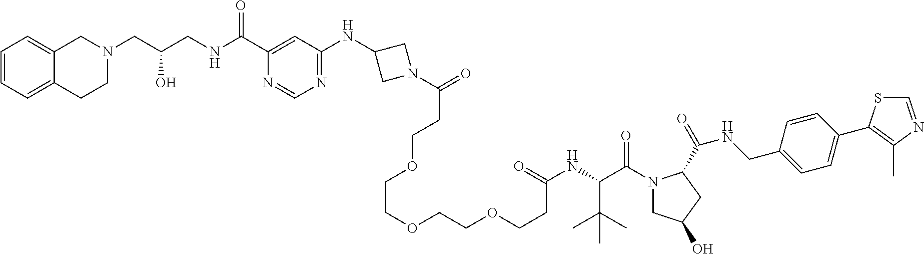 [SMILES: Cc1ncsc1-c1ccc(CNC(=O)[C@@H]2C[C@@H](O)CN2C(=O)[C@@H](NC(=O)CCOCCOCCOCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 15-((2S,4R)- 4-hydroxy- 2-((4-(4- methylthiazol-5- yl)benzyl)carbamoyl) pyrrolidine- 1-carbonyl)-16,16- dimethyl-13-oxo- 4,7,10-trioxa-14- azaheptadecanoyl) azetidin-3- yl)amino)pyrimidine- 4-carboxamide |
| YS43- 15 | 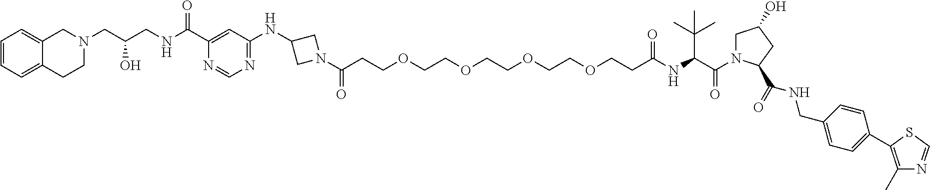 [SMILES: Cc1ncsc1-c1ccc(CNC(=O)[C@@H]2C[C@@H](O)CN2C(=O)[C@@H](NC(=O)CCOCCOCCOCCOCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 18-((2S,4R)- 4-hydroxy- 2-((4-(4- methylthiazol-5- yl)benzyl)carbamoyl) pyrrolidine- 1-carbonyl)-19,19- dimethyl-16-oxo- |
| | | 4,7,10,13- |
| | | tetraoxa-17- |
| | | azaicosanoyl) |
| | | azetidin-3- |
| | | yl)amino) |
| | | pyrimidine-4- |
| | | carboxamide |
| YS43- 16 | 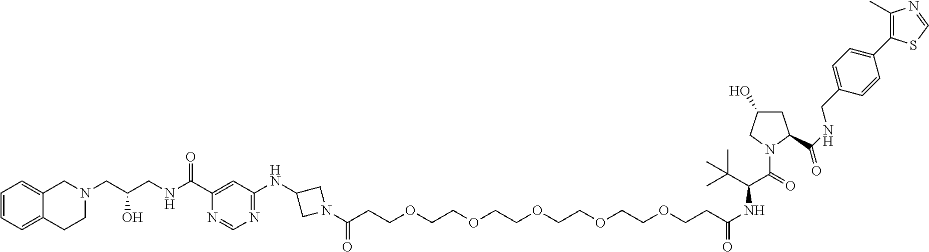 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 21-((2S,4R)- 4-hydroxy-2- ((4-(4- methylthiazol-5- yl)benzyl)carbamoyl) pyrrolidine- 1-carbonyl)-22,22- dimethyl-19-oxo- 4,7,10,13,16- pentaoxa-20- azatricosanoyl) |
| | | azetidin-3-yl) |
| | | amino)pyrimidine-4- |
| | | carboxamide |
| YS43- 17 | 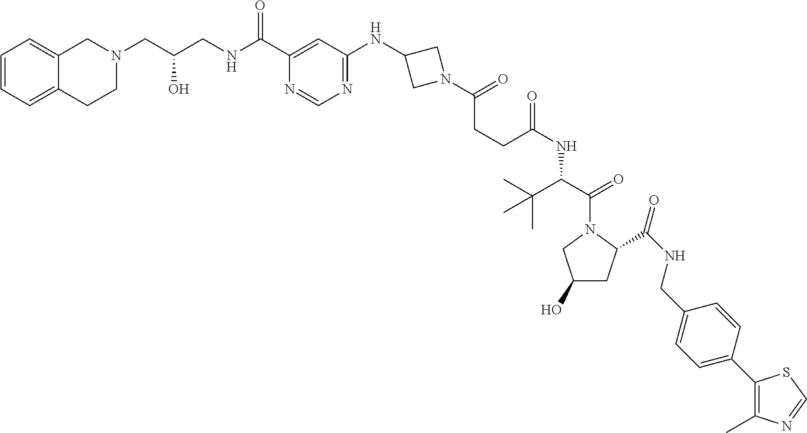 [SMILES: Cc1ncsc1-c1ccc(CNC(=O)[C@@H]2C[C@@H](O)CN2C(=O)[C@@H](NC(=O)CCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(4- (((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-4- oxobutanoyl) azetidin-3- yl)amino) pyrimidine-4- carboxamide |
| YS43- 18 | 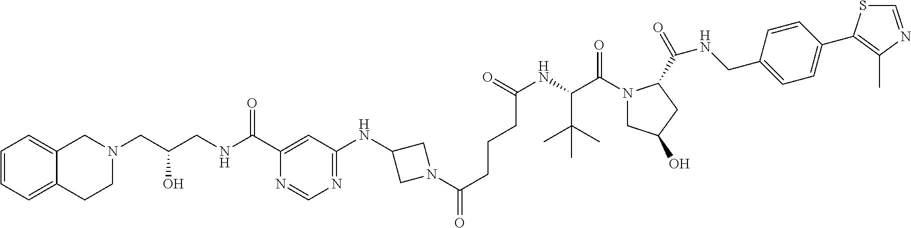 [SMILES: Cc1ncsc1-c1ccc(CNC(=O)[C@@H]2C[C@@H](O)CN2C(=O)[C@@H](NC(=O)CCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(5- (((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-5- oxopentanoyl) azetidin-3- |
| | | yl)amino) |
| | | pyrimidine-4- |
| | | carboxamide |
| YS43- 19 | 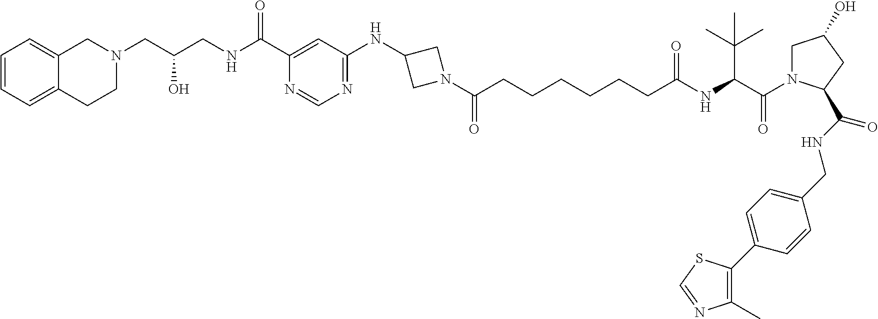 [SMILES: Cc1ncsc1-c1ccc(CNC(=O)[C@@H]2C[C@@H](O)CN2C(=O)[C@@H](NC(=O)CCCCCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(8- (((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-8- oxooctanoyl) azetidin-3- yl)amino) pyrimidine-4- carboxamide |
| YS43- 20 | 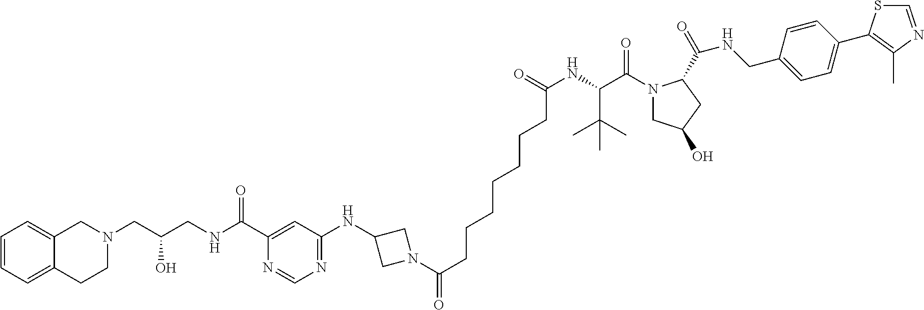 [SMILES: Cc1ncsc1-c1ccc(CNC(=O)[C@@H]2C[C@@H](O)CN2C(=O)[C@@H](NC(=O)CCCCCCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-(9- (((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-9- oxononanoyl) azetidin-3- yl)amino) pyrimidine-4- carboxamide |
| YS43- 21 | 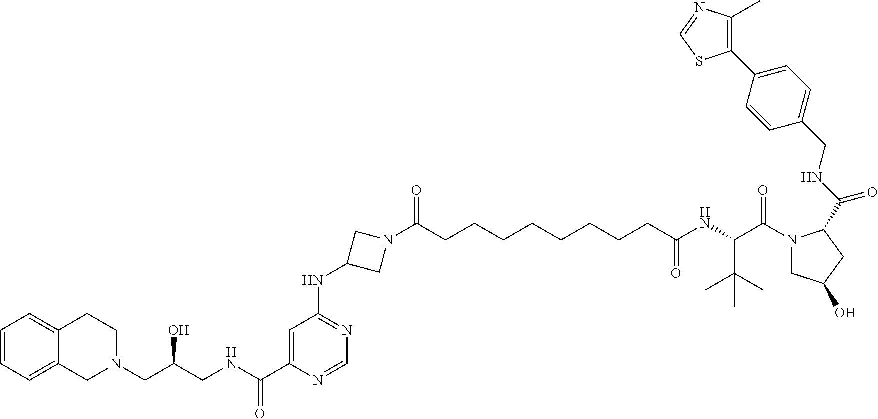 [SMILES: Cc1ncsc1-c1ccc(CNC(=O)[C@@H]2C[C@@H](O)CN2C(=O)[C@@H](NC(=O)CCCCCCCCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)-6-((1- (10-(((S)-1- ((2S,4R)-4- hydroxy-2-((4-(4- methylthiazol-5- yl)benzyl)carbamoyl) pyrrolidin- 1-yl)-3,3-dimethyl- 1-oxobutan-2-yl) amino)-10- oxodecanoyl)azetidin- 3-yl)amino) pyrimidine-4- carboxamide |
| YS43- 22 | 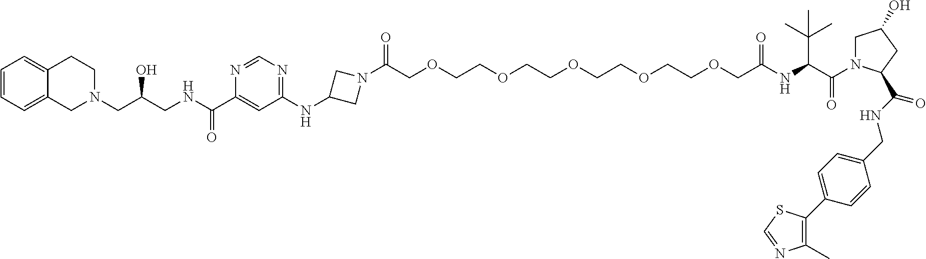 [SMILES: Cc1ncsc1-c1ccc(CNC(=O)[C@@H]2C[C@@H](O)CN2C(=O)[C@@H](NC(=O)COCCOCCOCCOCCOCC(=O)N2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)-19- ((2S,4R)-4-hydroxy-2- (((S)-1-(4-(4- methylthiazol-5- yl)phenyl)ethyl) carbamoyl) pyrrolidine-1- carbonyl)- 20,20-dimethyl- 17-oxo- 3,6,9,12,15- pentaoxa-18- |
| | | azahenicosanoyl) |
| | | azetidin-3- |
| | | yl)amino)pyrimidine- |
| | | 4-carboxamide |
| YS43- 25 | 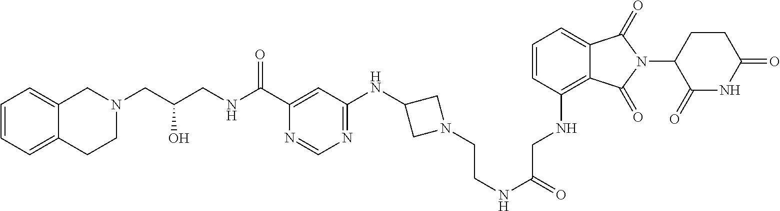 [SMILES: O=C(CNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(2-(2- ((2-(2,6- dioxopiperidin- 3-yl)-1,3- dioxoisoindolin-4- yl)amino)acetamido) ethyl)azetidin-3- yl)amino)pyrimidine- 4-carboxamide |
| YS43- 26 | 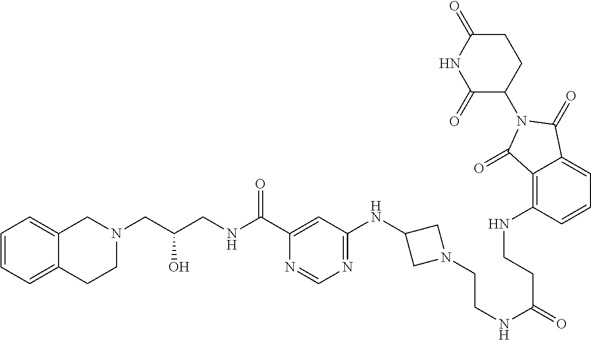 [SMILES: O=C(CCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypnopyl)- 6-((1-(2-(3- ((2-(2,6- dioxopiperidin- 3-yl)-1,3- dioxoisoindolin-4- yl)amino) propanamido) ethyl)azetidin-3- yl)amino) pyrimidine-4- carboxamide |
| YS43- 27 | 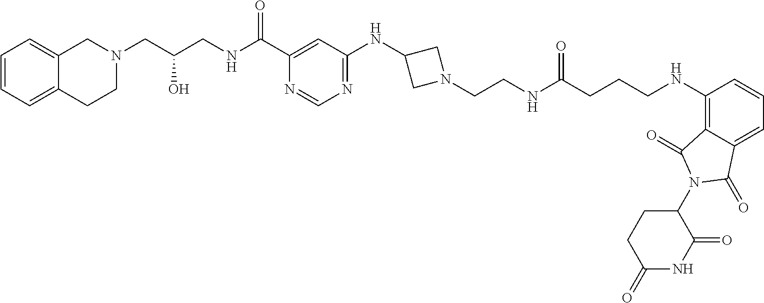 [SMILES: O=C(CCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(2-(2- ((2-(2,6- dioxopiperidin- 3-yl)-1,3- dioxoisoindolin-4- yl)amino)butanamido) ethyl)azetidin-3- yl)amino) pyrimidine-4- carboxamide |
| YS43- 28 | 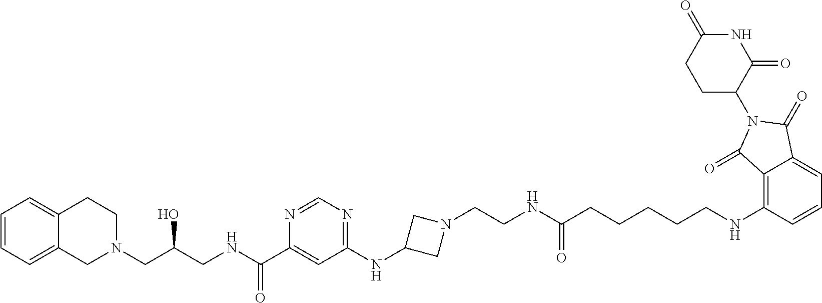 [SMILES: O=C(CCCCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(2-(6- ((2-(2,6- dioxopiperidin- 3-yl)-1,3- dioxoisoindolin-4- yl)amino) hexanamido) ethyl)azetidin-3- yl)amino) pyrimidine-4- carboxamide |
| YS43- 29 | 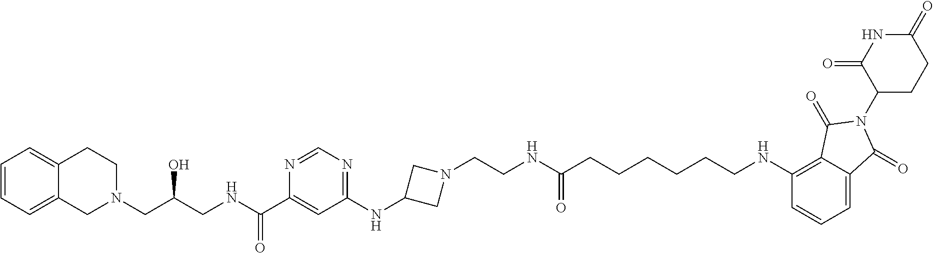 [SMILES: O=C(CCCCCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(2-(7- ((2-(2,6- dioxopiperidin- 3-yl)-1,3- dioxoisoindolin-4- yl)amino)heptanamido) ethyl)azetidin-3- yl)amino) pyrimidine-4- carboxamide |
| YS43- 30 | 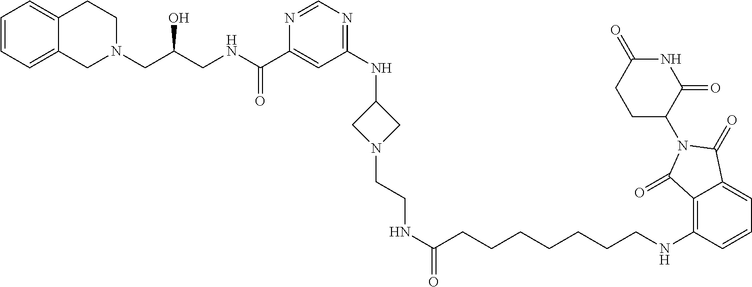 [SMILES: O=C(CCCCCCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(2-(8- ((2-(2,6- dioxopiperidin- 3-yl)-1,3- dioxoisoindolin-4- yl)amino)octanamido) ethyl)azetidin-3- yl)amino) pyrimidine-4- carboxamide |
| YS43- 31 | 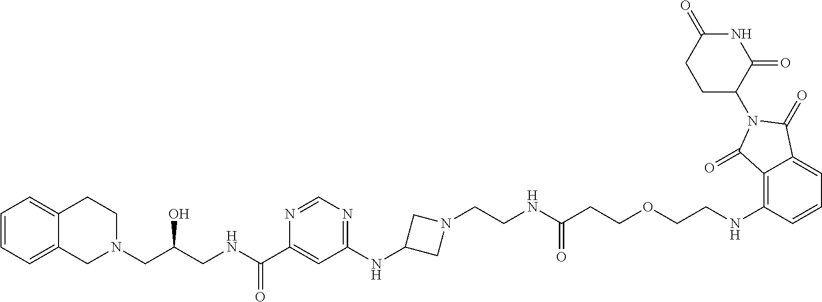 [SMILES: O=C(CCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)-6-((1- (2-(3-(2-((2-(2,6- dioxopiperidin- 3-yl)-1,3- dioxoisoindolin-4- yl)amino)ethoxy) propanamido) ethyl)azetidin-3- yl)amino) pyrimidine-4- carboxamide |
| YS43- 32 | 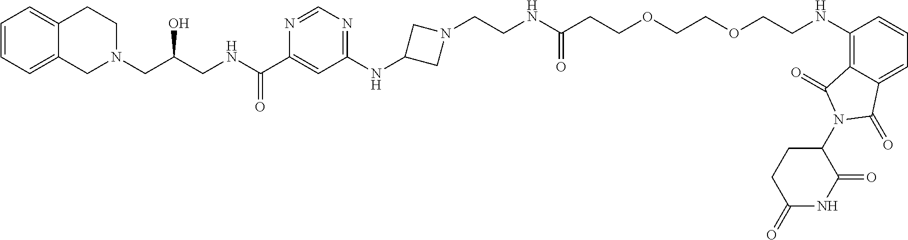 [SMILES: O=C(CCOCCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(2- (3-(2-(2-((2-(2,6- dioxopiperidin- 3-yl)-1,3- dioxoisoindolin-4- yl)amino) ethoxy)ethoxy) propanamido) ethyl)azetidin- 3-yl)amino) pyrimidine- 4-carboxamide |
| YS43- 33 | 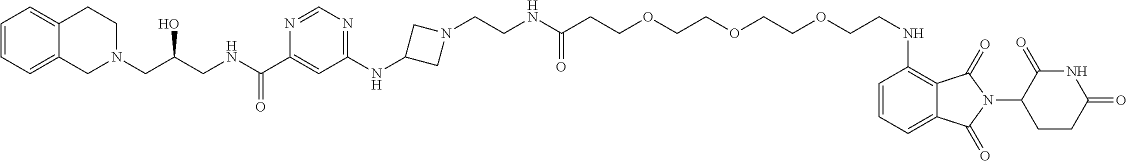 [SMILES: O=C(CCOCCOCCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(1- ((2-(2,6- dioxopiperidin-3- yl)-1,3- dioxoisoindolin-4- yl)amino)-12- oxo-3,6,9- |
| | | trioxa-13- |
| | | azapentadecan- |
| | | 15-yl)azetidin-3- |
| | | yl)amino)pyrimidine- |
| | | 4-carboxamide |
| YS43- 34 | 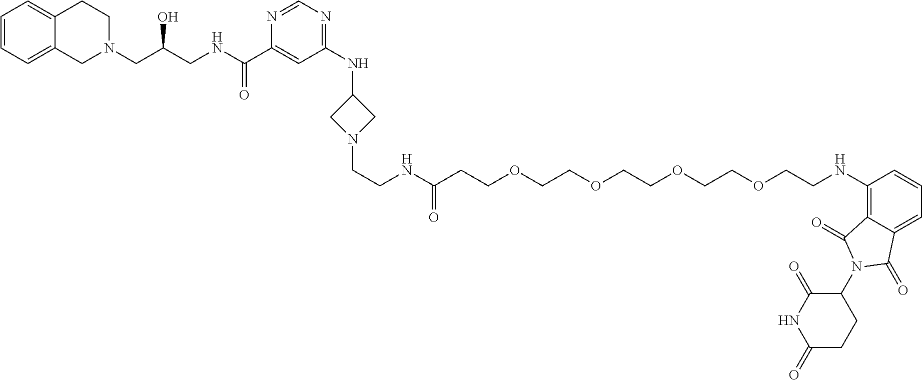 [SMILES: O=C(CCOCCOCCOCCOCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(1- ((2-(2,6- dioxopiperidin-3- yl)-1,3- dioxoisoindolin- 4-yl)amino)-15-oxo- 3,6,9,12- tetraoxa-16- azaoctadecan- 18-yl)azetidin-3- yl)amino)pyrimidine- 4-carboxamide |
| YS43- 35 | 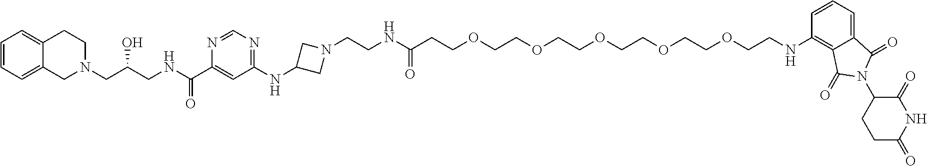 [SMILES: O=C(CCOCCOCCOCCOCCOCCNc1cccc2c1C(=O)N(C1CCC(=O)NC1=O)C2=O)NCCN1CC(Nc2cc(C(=O)NC[C@@H](O)CN3CCc4ccccc4C3)ncn2)C1] | N-((S)-3-(3,4- dihydroisoquinolin- 2(1H)-yl)-2- hydroxypropyl)- 6-((1-(1- ((2-(2,6- dioxopiperidin-3- yl)-1,3- dioxoisoindolin- 4-yl)amino)-18-oxo- 3,6,9,12,15- |
| | | pentaoxa-19- |
| | | azahenicosan-21- |
| | | yl)azetidin- |
| | | 3-yl)amino) |
| | | pyrimidine-4- |
| | | carboxamide |
| YS43- 36 | 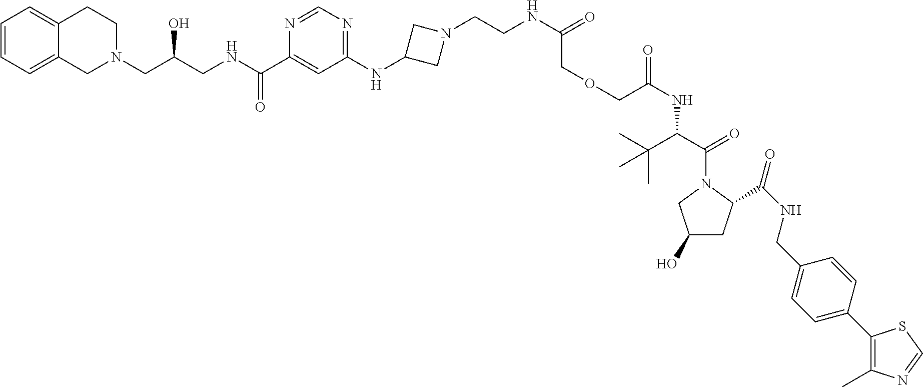 [SMILES: Cc1ncsc1-c1ccc(CNC(=O)[C@@H]2C[C@@H](O)CN2C(=O)[C@@H](NC(=O)COCC(=O)NCCN2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypnopyl)- 6-((1-(2- (2-(2-(((S)-1- ((2S,4R)-4- hydroxy-2-((4-(4- methylthiazol-5- yl)benzyl)carbamoyl) pyrrolidin- 1-yl)-3,3-dimethyl- 1-oxobutan-2- yl)amino)-2- oxoethoxy)acetamido) ethyl)azetidin-3- yl)amino)pyrimidine- 4-carboxamide |
| YS43- 37 | 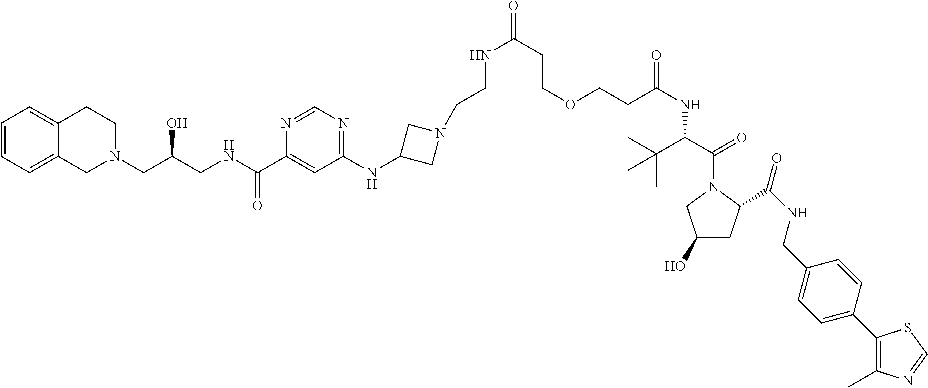 [SMILES: Cc1ncsc1-c1ccc(CNC(=O)[C@@H]2C[C@@H](O)CN2C(=O)[C@@H](NC(=O)CCOCCC(=O)NCCN2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypnopyl)- 6-((1-(2-(3- (3-(((S)-1-((2S,4R)-4- hydroxy-2-((4-(4- methylthiazol-5- yl)benzyl)carbamoyl) pyrrolidin- 1-yl)-3,3-dimethyl- 1-oxobutan-2- yl)amino)-3- oxopropoxy) propanamido) ethyl)azetidin-3- yl)amino)pyrimidine- 4-carboxamide |
| YS43- 38 | 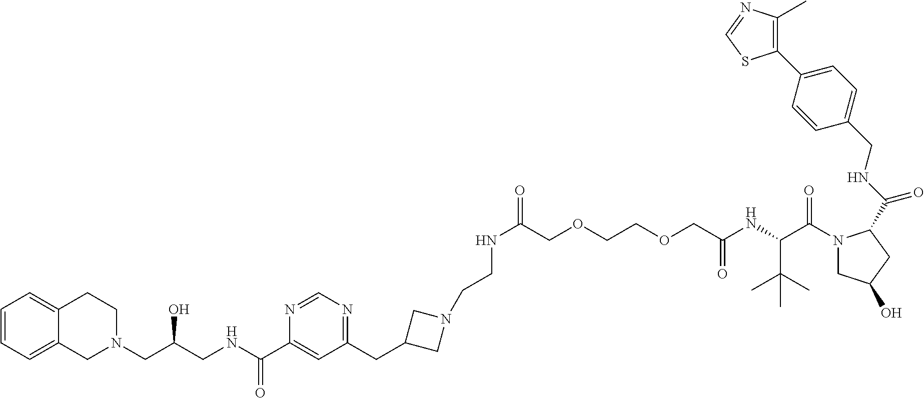 [SMILES: Cc1ncsc1-c1ccc(CNC(=O)[C@@H]2C[C@@H](O)CN2C(=O)[C@@H](NC(=O)COCCOCC(=O)NCCN2CC(Cc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 13-((2S,4R)- 4-hydroxy- 2-((4-(4- methylthiazol-5- yl)benzyl)carbamoyl) pyrrolidine- 1-carbonyl)-14,14- dimethyl-4,11-dioxo- 6,9-dioxa-3,12- diazapentadecyl) azetidin-3-yl) amino)pyrimidine-4- carboxamide |
| YS43- 39 | 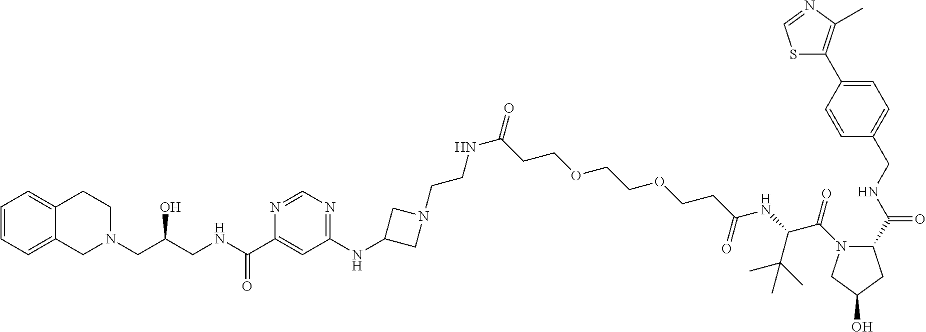 [SMILES: Cc1ncsc1-c1ccc(CNC(=O)[C@@H]2C[C@@H](O)CN2C(=O)[C@@H](NC(=O)CCOCCOCCC(=O)NCCN2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 15-((2S,4R)- 4-hydroxy- 2-((4-(4- methylthiazol-5- yl)benzyl)carbamoyl) pyrrolidine- 1-carbonyl)-16,16- dimethyl-4,13-dioxo- 7,10-dioxa-3,14- diazaheptadecyl) azetidin-3- yl)amino)pyrimidine- 4-carboxamide |
| YS43- 40 | 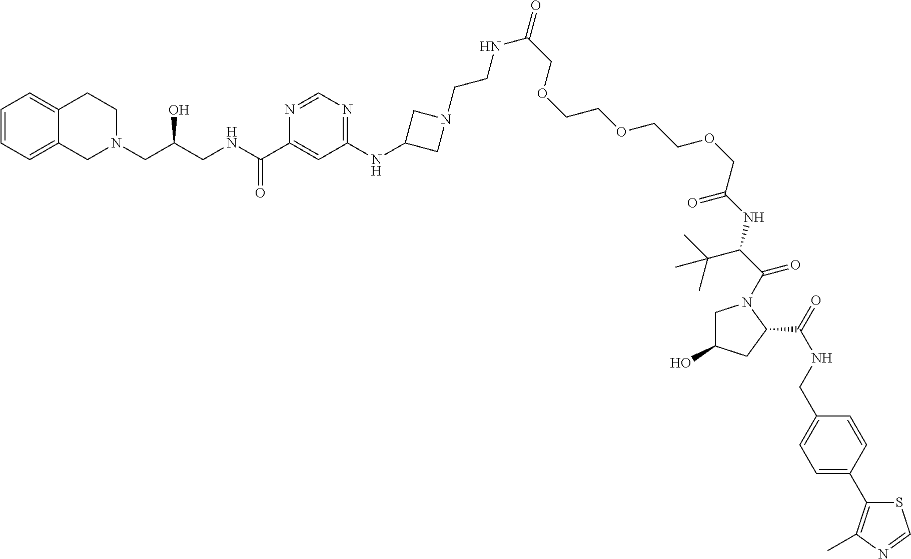 [SMILES: Cc1ncsc1-c1ccc(CNC(=O)[C@@H]2C[C@@H](O)CN2C(=O)[C@@H](NC(=O)COCCOCCOCC(=O)NCCN2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 16-((2S,4R)- 4-hydroxy- 2-((4-(4- methylthiazol-5- yl)benzyl)carbamoyl) pyrrolidine- 1-carbonyl)-17,17- dimethyl-4,14-dioxo- 6,9,12-trioxa-3,15- diazaoctadecyl) azetidin-3- yl)amino)pyrimidine- 4-carboxamide |
| YS43- 41 | 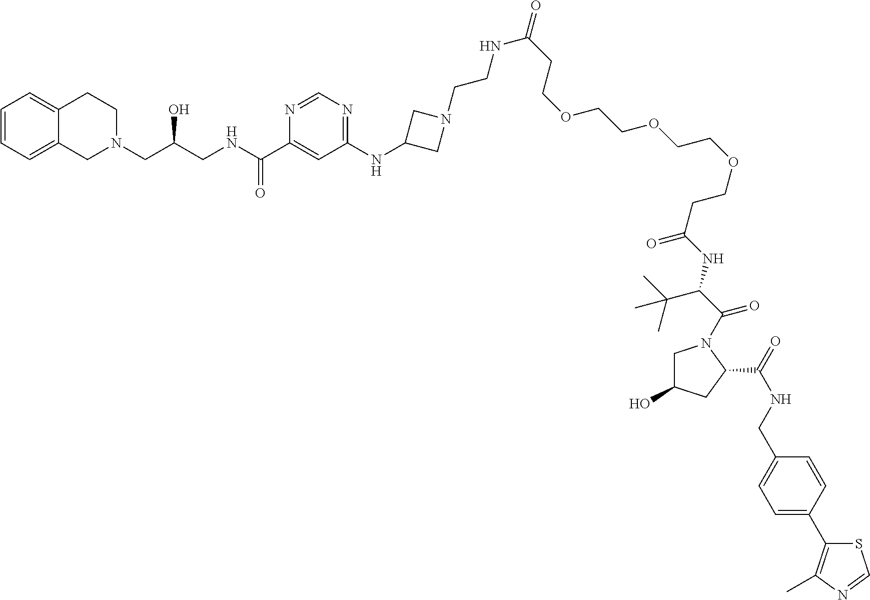 [SMILES: Cc1ncsc1-c1ccc(CNC(=O)[C@@H]2C[C@@H](O)CN2C(=O)[C@@H](NC(=O)CCOCCOCCOCCC(=O)NCCN2CC(Nc3cc(C(=O)NC[C@H](O)CN4CCc5ccccc5C4)ncn3)C2)C(C)(C)C)cc1] | N-((S)-3-(3,4- dihydroisoquinolin- 2(1H)-yl)-2- hydroxypropyl)- 6-((1-((S)- 18-((2S,4R)- 4-hydroxy- 2-((4-(4- methylthiazol-5- yl)benzyl)carbamoyl) pyrrolidine- 1-carbonyl)-19,19- dimethyl-4,16-dioxo- 7,10,13-trioxa-3,17- diazaicosyl) azetidin-3-yl) amino)pyrimidine-4- carboxamide |
| YS43- 42 | 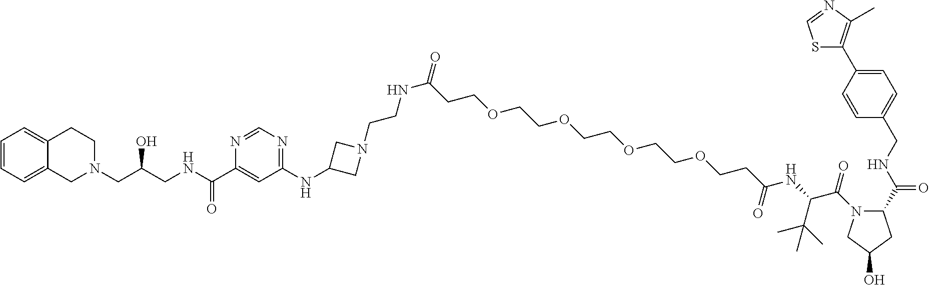 [SMILES: Cc1ncsc1-c1ccc(CNC(=O)[C@@H]2C[C@@H](O)CN2C(=O)[C@@H](NC(=O)CCOCCOCCOCCOCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N¹⁶-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl)- 4,7,10,13- tetraoxahexa- decanediamide |
| YS43- 43 | 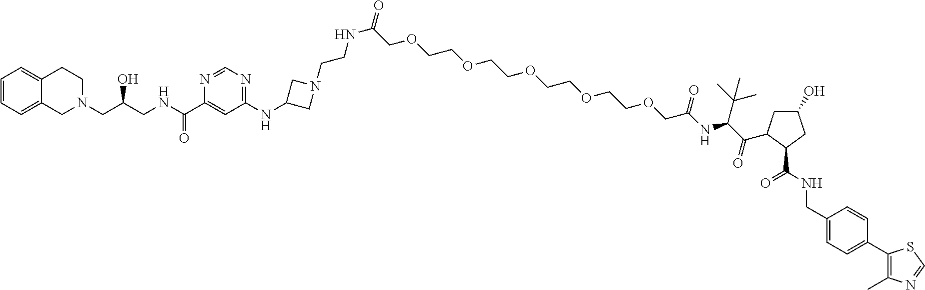 [SMILES: Cc1ncsc1-c1ccc(CNC(=O)[C@@H]2C[C@@H](O)CC2C(=O)[C@@H](NC(=O)COCCOCCOCCOCCOCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N¹⁷-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl)- 3,6,9,12,15- pentaoxahepta- decanediamide |
| YS43- 44 | 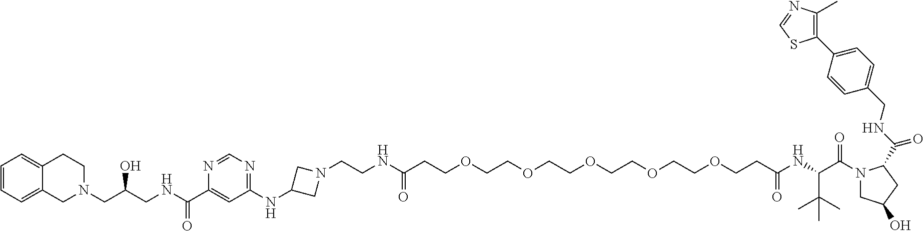 [SMILES: Cc1ncsc1-c1ccc(CNC(=O)[C@@H]2C[C@@H](O)CN2C(=O)[C@@H](NC(=O)CCOCCOCCOCCOCCOCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N¹⁹-((S)-1-((2S,4R)-4- hydroxy-2-((4-(4- methylthiazol-5- yl)benzyl)carbamoyl) pyrrolidin- 1-yl)-3,3-dimethyl- |
| | | 1-oxobutan-2-yl)- |
| | | 4,7,10,13,16- |
| | | pentaoxanona- |
| | | decanediamide |
| YS43- 45 | 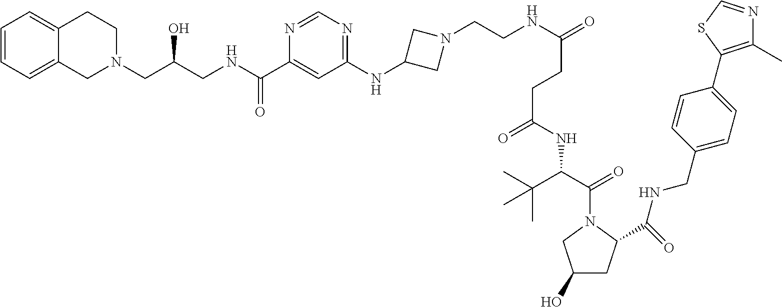 [SMILES: Cc1ncsc1-c1ccc(CNC(=O)[C@@H]2C[C@@H](O)CN2C(=O)[C@@H](NC(=O)CCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N⁴-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) succinamide |
| YS43- 46 | 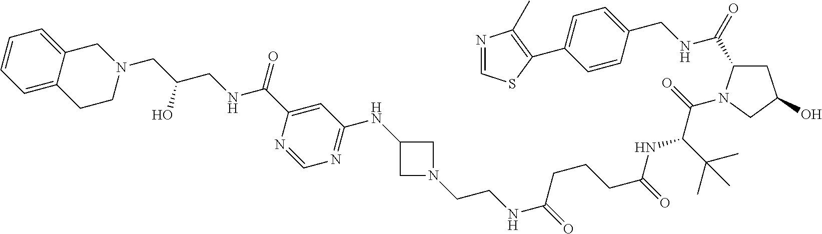 [SMILES: Cc1ncsc1-c1ccc(CNC(=O)[C@@H]2C[C@@H](O)CN2C(=O)[C@@H](NC(=O)CCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N⁵-((S)-1-((2S,4R)-4- hydroxy-2-((4-(4- methylthiazol-5- yl)benzyl)carbamoyl) pyrrolidin- 1-yl)-3,3-dimethyl- |
| | | 1-oxobutan-2-yl) |
| | | glutaramide |
| YS43- 47 | 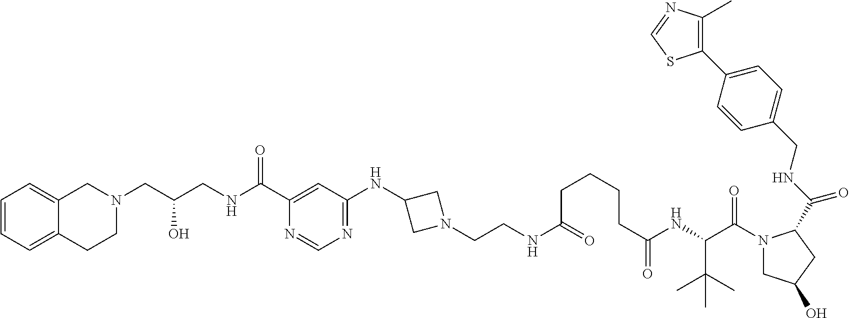 [SMILES: Cc1ncsc1-c1ccc(CNC(=O)[C@@H]2C[C@@H](O)CN2C(=O)[C@@H](NC(=O)CCCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N⁶-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) adipamide |
| YS43- 48 | 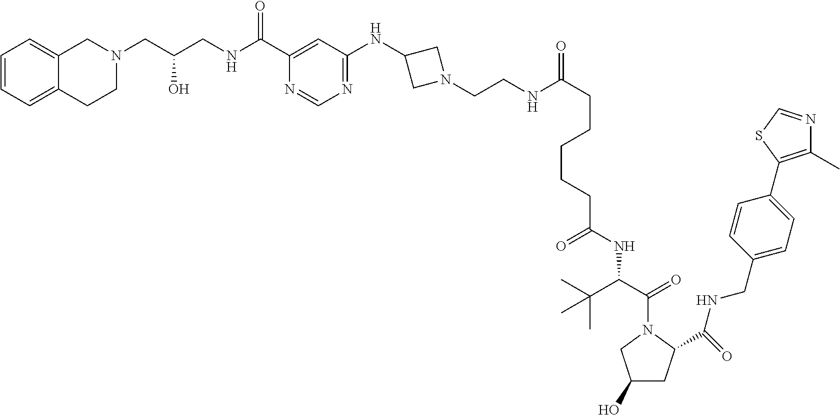 [SMILES: Cc1ncsc1-c1ccc(CNC(=O)[C@@H]2C[C@@H](O)CN2C(=O)[C@@H](NC(=O)CCCCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N⁷-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) heptanediamide |
| YS43- 49 | 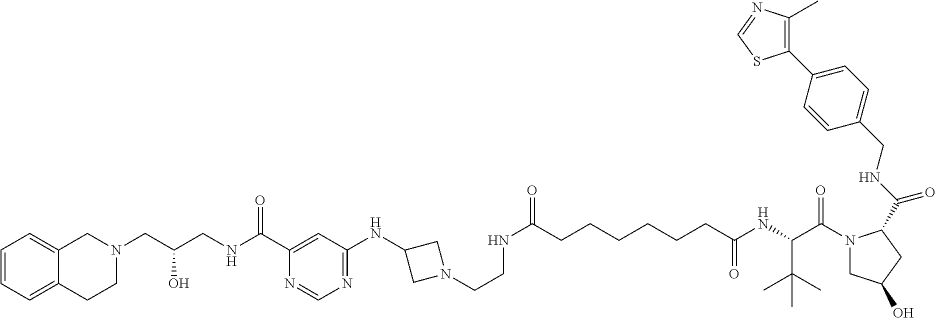 [SMILES: Cc1ncsc1-c1ccc(CNC(=O)[C@@H]2C[C@@H](O)CN2C(=O)[C@@H](NC(=O)CCCCCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypnopyl) carbamoyl) pyrimidin-4- yl)amino)azetidin- 1-yl)ethyl)- N⁸-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) octanediamide |
| YS43- 50 | 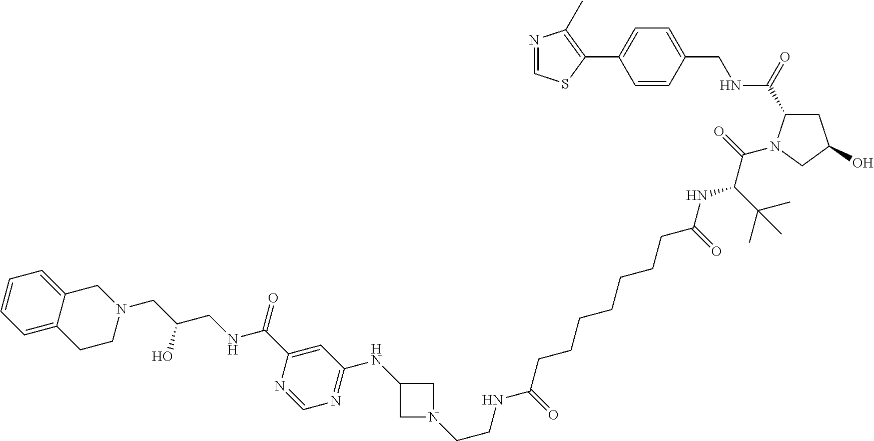 [SMILES: Cc1ncsc1-c1ccc(CNC(=O)[C@@H]2C[C@@H](O)CN2C(=O)[C@@H](NC(=O)CCCCCCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N⁹-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) nonanediamide |
| YS43- 51 | 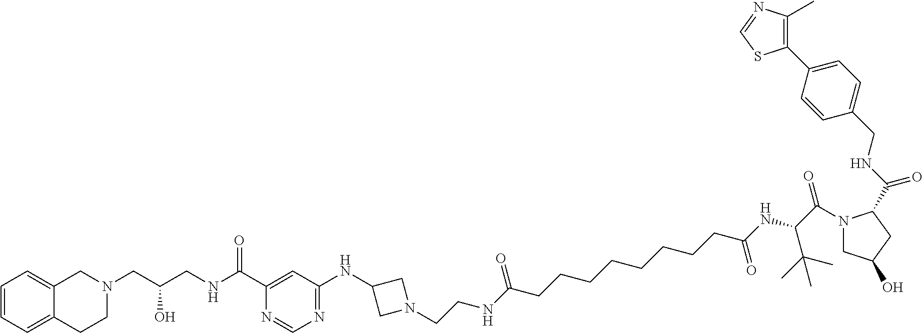 [SMILES: Cc1ncsc1-c1ccc(CNC(=O)[C@@H]2C[C@@H](O)CN2C(=O)[C@@H](NC(=O)CCCCCCCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N¹⁰-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) decanediamide |
| YS43- 52 | 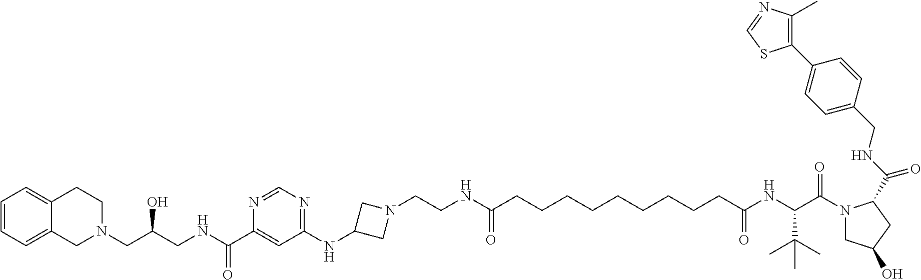 [SMILES: Cc1ncsc1-c1ccc(CNC(=O)[C@@H]2C[C@@H](O)CN2C(=O)[C@@H](NC(=O)CCCCCCCCCC(=O)NCCN2CC(Nc3cc(C(=O)NC[C@H](O)CN4CCc5ccccc5C4)ncn3)C2)C(C)(C)C)cc1] | N¹-(2-(3-((6- (((S)-3-(3,4- dihydroisoquinolin- 2(1H)- yl)-2-hydroxypropyl) carbamoyl) pyrimidin-4- yl)amino)azetidin- 1-yl)ethyl)- N¹¹-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) undecanediamide |
| YS43- 53 | 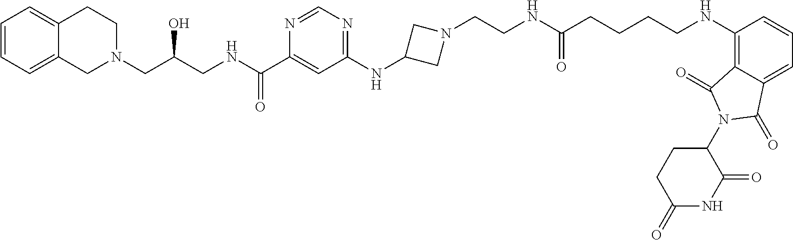 [SMILES: O=C(CCCCNc1cccc2c1C(=O)N(C1CCC(=O)NC1=O)C2=O)NCCN1CC(Nc2cc(C(=O)NC[C@H](O)CN3CCc4ccccc4C3)ncn2)C1] | N-((S)-3-(3,4- dihydroisoquinolin- 2(1H)-yl)-2- hydroxypropyl)- 6-((1-(2-(5- ((2-(2,6-dioxopiperidin- 3-yl)-1,3- dioxoisoindolin-4- yl)amino)pentanamido) ethyl)azetidin-3- yl)amino) pyrimidine-4- carboxamide |
| YS43- 54 | 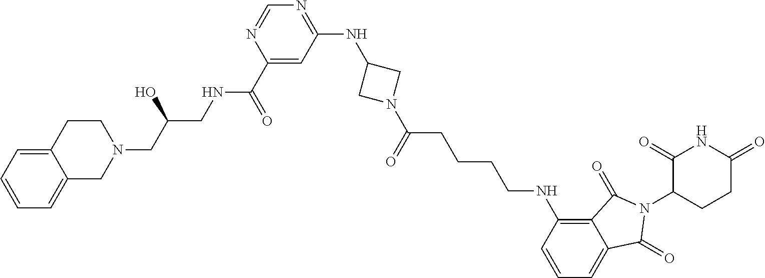 [SMILES: O=C1CCC(N2C(=O)c3cccc(NCCCCC(=O)N4CC(Nc5cc(C(=O)NC[C@H](O)CN6CCc7ccccc7C6)ncn5)C4)c3C2=O)C(=O)N1] | N-((S)-3-(3,4- dihydroisoquinolin- 2(1H)-yl)-2- hydroxypropyl)- 6-((1-(5- ((2-(2,6- dioxopiperidin-3- yl)-1,3- dioxoisoindolin-4- yl)amino)pentanoyl) azetidin-3- yl)amino)pyrimidine- 4-carboxamide |
| YS43- 88 | 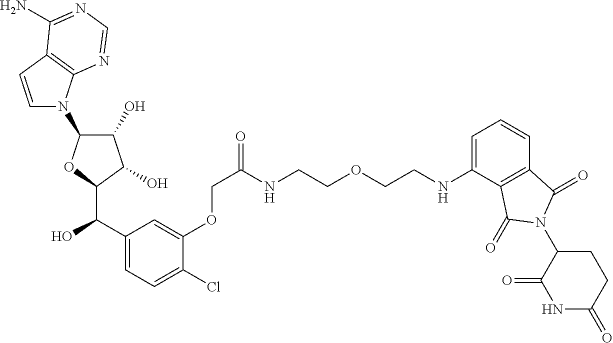 [SMILES: Nc1ncnc2c1ccn2[C@@H]1O[C@H]([C@H](O)c2ccc(Cl)c(OCC(=O)NCCOCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(2-(2- ((2-(2,6- dioxopiperidin-3- yl)-1,3- dioxoisoindolin-4- yl)amino) ethoxy)ethyl) acetamide |
| YS43- 89 | 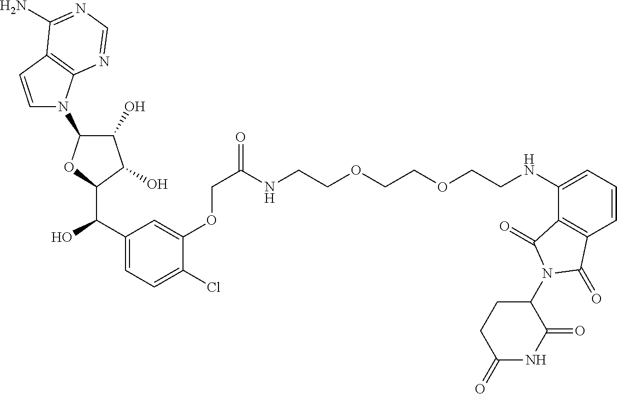 [SMILES: Nc1ncnc2c1ccn2[C@@H]1O[C@H]([C@H](O)c2ccc(Cl)c(OCC(=O)NCCOCCOCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(2-(2-(2- ((2-(2,6- dioxopiperidin- 3-yl)-1,3- dioxoisoindolin-4- yl)amino)ethoxy) ethoxy)ethyl) acetamide |
| YS43- 90 | 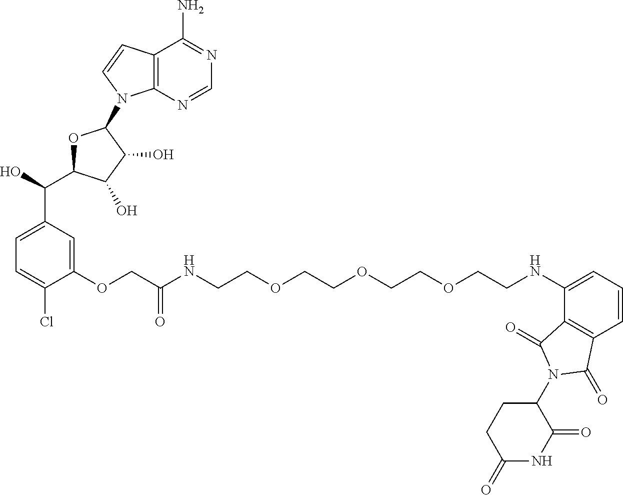 [SMILES: Nc1ncnc2c1ccn2[C@@H]1O[C@H]([C@H](O)c2ccc(Cl)c(OCC(=O)NCCOCCOCCOCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran- 2-yl)(hydroxy) methyl)-2- chlorophenoxy)-N-(2- (2-(2-(2-((2-(2,6- dioxopiperidin- 3-yl)-1,3- dioxoisoindolin-4- yl)amino) ethoxy)ethoxy) ethoxy)ethyl) acetamide |
| YS43- 91 | 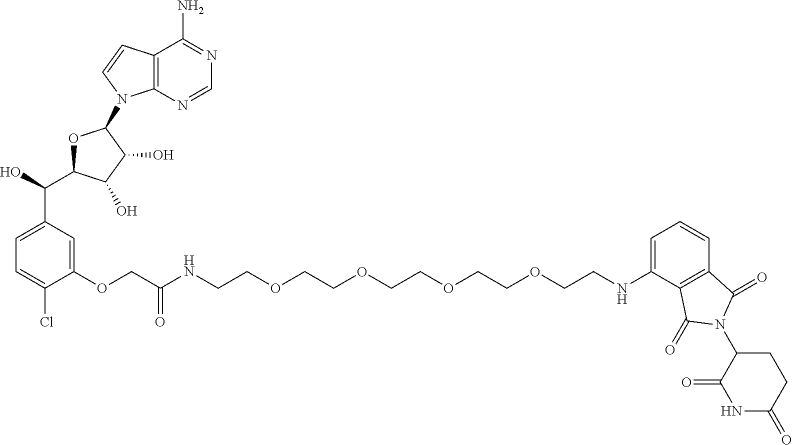 [SMILES: Nc1ncnc2c1ccn2[C@@H]1O[C@H]([C@H](O)c2ccc(Cl)c(OCC(=O)NCCOCCOCCOCCOCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran- 2-yl) (hydroxy)methyl)-2- chlorophenoxy)- N-(14-((2- (2,6- dioxopiperidin-3- yl)-1,3- dioxoisoindolin-4- yl)amino)-3,6,9,12- tetraoxatetradecyl) acetamide |
| YS43- 92 | 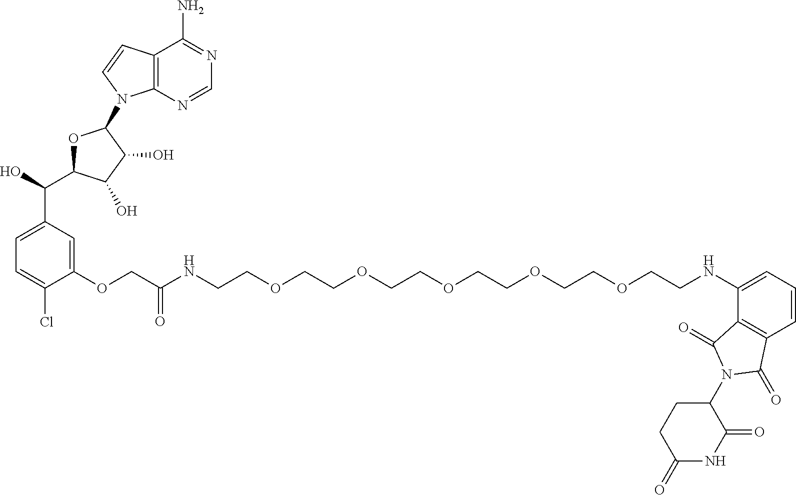 [SMILES: Nc1ncnc2c1ccn2[C@@H]1O[C@H]([C@H](O)c2ccc(Cl)c(OCC(=O)NCCOCCOCCOCCOCCOCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(17-((2- (2,6- dioxopiperidin-3- yl)-1,3- dioxoisoindolin-4-yl) amino)-3,6,9,12,15- pentaoxaheptadecyl) acetamide |
| YS43- 93 | 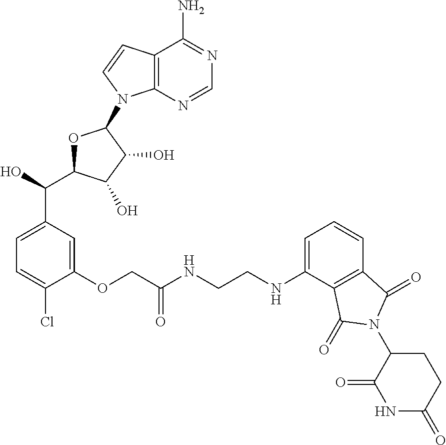 [SMILES: Nc1ncnc2c1ccn2[C@@H]1O[C@H]([C@H](O)c2ccc(Cl)c(OCC(=O)NCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran- 2-yl) (hydroxy)methyl)-2- chlorophenoxy)- N-(2-((2- (2,6- dioxopiperidin-3-yl)- 1,3-dioxoisoindolin- 4-yl) amino)ethyl) acetamide |
| YS43- 94 | 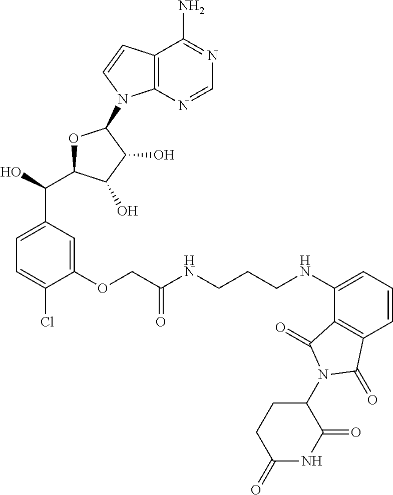 [SMILES: Nc1ncnc2c1ccn2[C@@H]1O[C@H]([C@H](O)c2ccc(Cl)c(OCC(=O)NCCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(3-((2- (2,6- dioxopiperidin-3-yl)- 1,3-dioxoisoindolin- 4-yl) amino)propyl) acetamide |
| YS43- 95 | 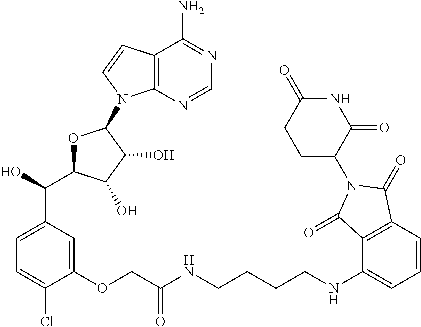 [SMILES: Nc1ncnc2c1ccn2[C@@H]1O[C@H]([C@H](O)c2ccc(Cl)c(OCC(=O)NCCCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3.4- dihydroxytetra- hydrofuran-2- yl)(hydroxy) methyl)-2- chlorophenoxy)- N-(4-((2- (2,6- dioxopiperidin-3-yl)- 1,3-dioxoisoindolin- 4-yl) amino)butyl) acetamide |
| YS43- 96 | 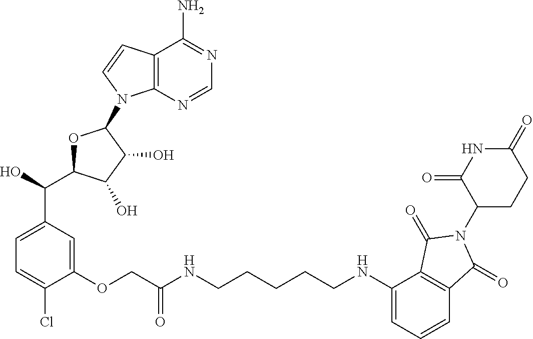 [SMILES: Nc1ncnc2c1ccn2[C@@H]1O[C@H]([C@H](O)c2ccc(Cl)c(OCC(=O)NCCCCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(5-((2- (2,6- dioxopiperidin-3-yl)- 1,3- dioxoisoindolin-4-yl) amino)pentyl) acetamide |
| YS43- 97 | 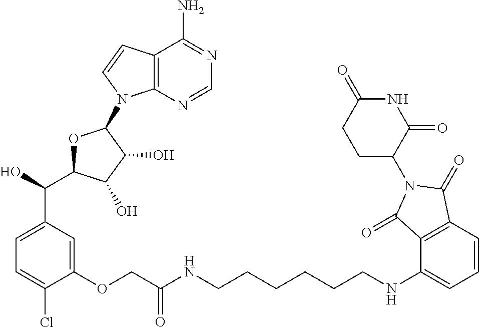 [SMILES: Nc1ncnc2c1ccn2[C@@H]1O[C@H]([C@H](O)c2ccc(Cl)c(OCC(=O)NCCCCCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(6-((2- (2,6- dioxopiperidin-3-yl)- 1,3-dioxoisoindolin- 4-yl) amino)hexyl) acetamide |
| YS43- 98 | 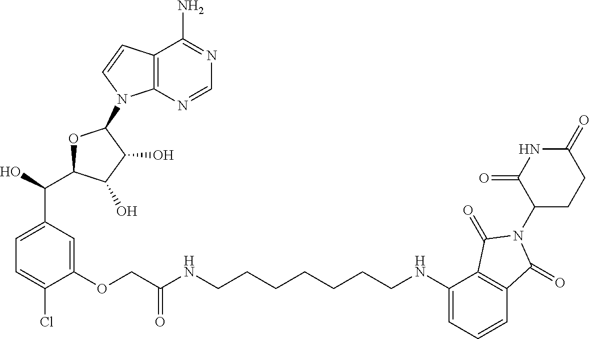 [SMILES: Nc1ncnc2c1ccn2[C@@H]1O[C@H]([C@H](O)c2ccc(Cl)c(OCC(=O)NCCCCCCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(7-((2- (2,6- dioxopiperidin-3-yl)- 1,3- dioxoisoindolin-4-yl) amino)heptyl) acetamide |
| YS43- 99 | 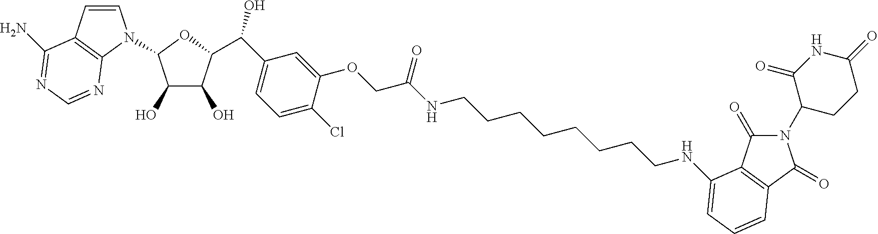 [SMILES: Nc1ncnc2c1ccn2[C@@H]1O[C@H]([C@H](O)c2ccc(Cl)c(OCC(=O)NCCCCCCCCNc3cccc4c3C(=O)N(C3CCC(=O)NC3=O)C4=O)c2)[C@@H](O)[C@H]1O] | 2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)- N-(8-((2-(2,6- dioxopiperidin-3-yl)- 1,3-dioxoisoindolin- 4-yl) amino)octyl) acetamide |
| YS43- 100 | 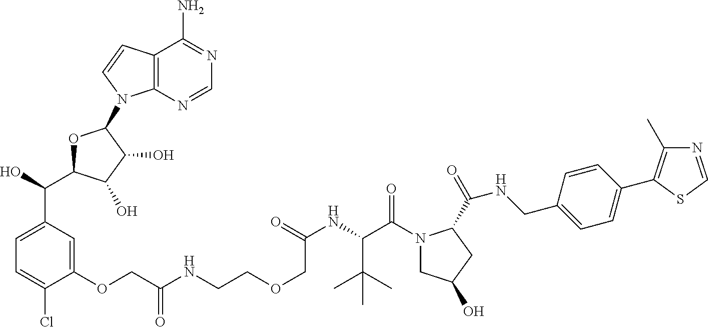 [SMILES: Cc1ncsc1-c1ccc(CNC(=O)[C@@H]2C[C@@H](O)CN2C(=O)[C@@H](NC(=O)COCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-2-(2-(2-(2- (5-((R)- ((2R,3S,4R,5R)-5- (4-amino-7H- pyrrolo[2,3-d] pyrimidin-7-yl)-3,4- dihydroxytetra- hydrofuran-2- yl)(hydroxy) methyl)-2- chlorophenoxy) acetamido) ethoxy) acetamido)-3,3- dimethylbutanoyl)-4- hydroxy-N-(4-(4- methyithiazol-5-yl) benzyl)pyrrolidine-2- carboxamide |
| YS43- 101 | 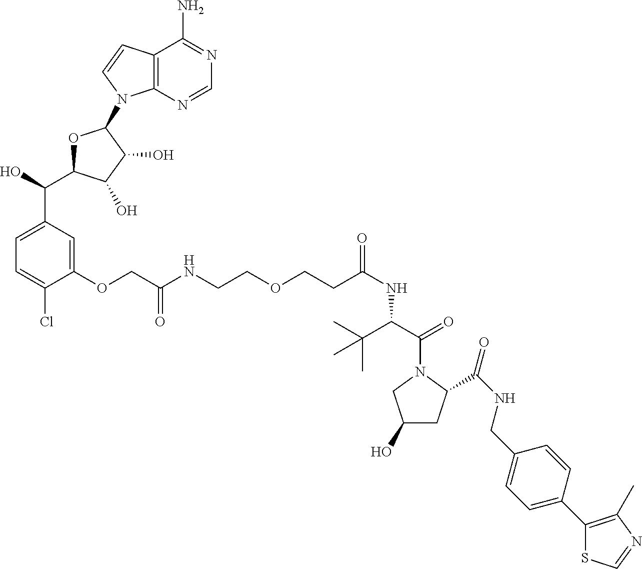 [SMILES: Cc1ncsc1-c1ccc(CNC(=O)[C@@H]2C[C@@H](O)CN2C(=O)[C@@H](NC(=O)CCOCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-2-(3-(2-(2- (5-((R)- ((2R,3S,4R,5R)-5- (4-amino-7H- pyrrolo[2,3-d] pyrimidin-7-yl)-3,4- dihydroxytetra- hydrofnran- 2-yl)(hydroxy) methyl)-2- chlorophenoxy) acetamido) ethoxy) propanamido)- 3,3- dimethylbutanoyl)- 4-hydroxy-N-(4-(4- methylthiazol-5-yl) benzyl)pyrrolidine-2- carboxamide |
| YS43- 102 | 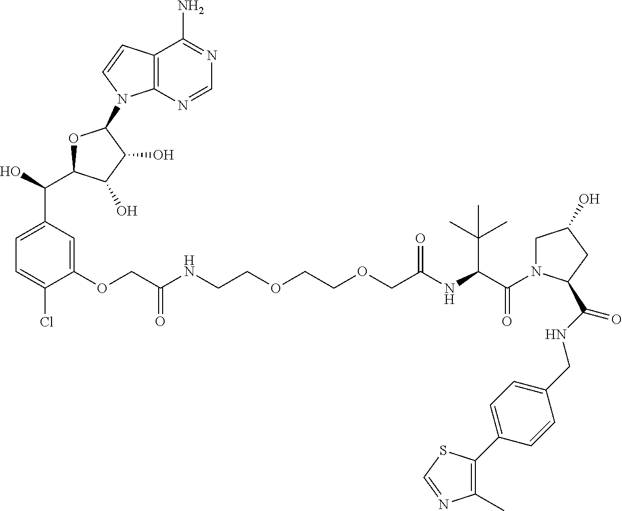 [SMILES: Cc1ncsc1-c1ccc(CNC(=O)[C@@H]2C[C@@H](O)CN2C(=O)[C@@H](NC(=O)COCCOCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-14-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 2-(tert- butyl)-4,13- dioxo-6,9- dioxa-3,12- diazatetradecanoyl)- 4-hydroxy-N-(4-(4- methylthiazol- 5-yl)benzyl) pyrrolidine-2- carboxamide |
| YS43- 103 | 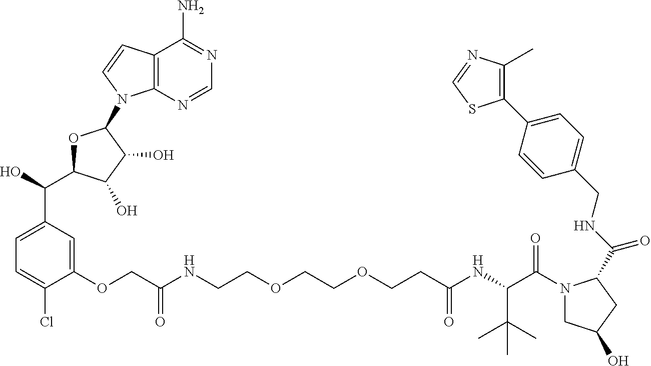 [SMILES: Cc1ncsc1-c1ccc(CNC(=O)[C@@H]2C[C@@H](O)CN2C(=O)[C@@H](NC(=O)CCOCCOCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-1-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy)methyl)-2- chlorophenoxy)- 14-(tert- butyl)-2,12-dioxo-6,9- dioxa-3,13- diazapentadecan- 15-oyl)-4-hydroxy-N- (4-(4-methylthiazol-5- yl)benzyl) pyrrolidine-2- carboxamide |
| YS43- 104 | 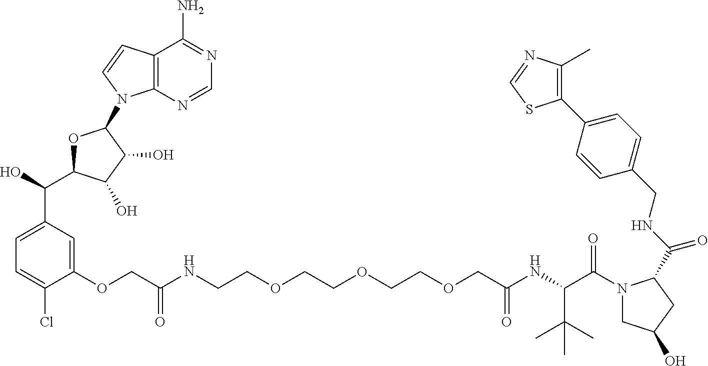 [SMILES: Cc1ncsc1-c1ccc(CNC(=O)[C@@H]2C[C@@H](O)CN2C(=O)[C@@H](NC(=O)COCCOCCOCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2,S,4R)-1- ((S)-17-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 2-(tert- butyl)-4,16- dioxo-6,9,12- trioxa-3,15- diazaheptadecanoyl)- 4-hydroxy-N-(4- (4-methylthiazol-5-yl) benzyl)pyrrolidine-2- carboxamide |
| YS43- 105 | 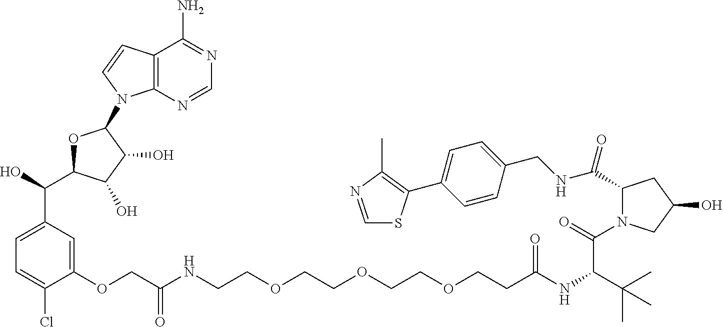 [SMILES: Cc1ncsc1-c1ccc(CNC(=O)[C@@H]2C[C@@H](O)CN2C(=O)[C@@H](NC(=O)CCOCCOCCOCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-1-(5-((R)- ((2R,3S,4R,5R)-5-(4- amino-7H- pyrrolo[2,3-d] pyrimidin-7-yl)-3,4- dihydroxytetra- hydrofuran- 2-yl)(hydroxy) methyl)-2- chlorophenoxy)- 17-(tert-butyl)- 2,15-dioxo-6,9,12- trioxa-3,16- diazaoctadecan- 18-oyl)-4-hydroxy-N- (4-(4-methylthiazol- 5-yl) benzyl)pynolidine-2- carboxamide |
| YS43- 106 | 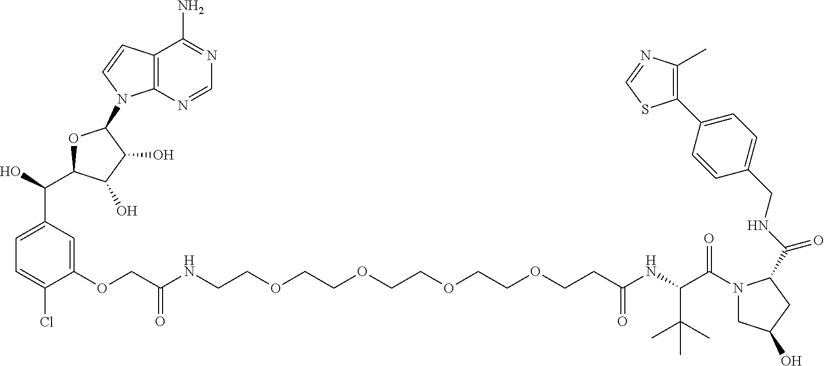 [SMILES: Cc1ncsc1-c1ccc(CNC(=O)[C@@H]2C[C@@H](O)CN2C(=O)[C@@H](NC(=O)CCOCCOCCOCCOCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-1-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 20-(tert- butyl)-2,18-dioxo- 6,9,12,15- tetraoxa-3,19- diazahenicosan- 21-oyl)-4- hydroxy-N-(4-(4- methylthiazol-5- yl)benzyl) pyrrolidine-2- carboxamide |
| YS43- 107 | 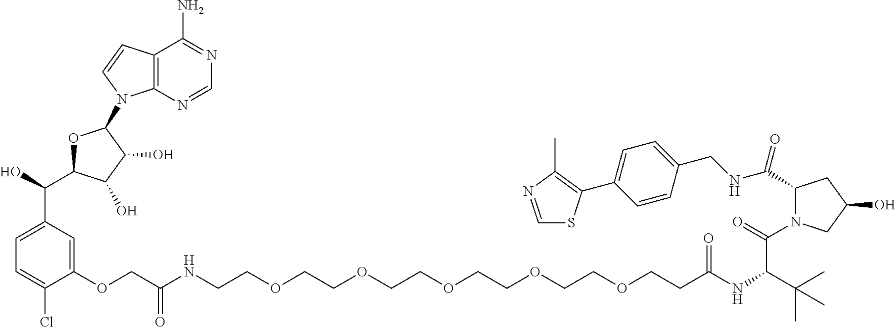 [SMILES: Cc1ncsc1-c1ccc(CNC(=O)[C@@H]2C[C@@H](O)CN2C(=O)[C@@H](NC(=O)CCOCCOCCOCCOCCOCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-1-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 23-(tert- butyl)-2,21-dioxo- 6,9,12,15,18- pentaoxa-3,22- diazatetracosan-24- oyl)-4-hydroxy-N-(4- (4-methylthiazol-5- yl)benzyl) pyrrolidine-2- carboxamide Molecular |
| YS43- 108 | 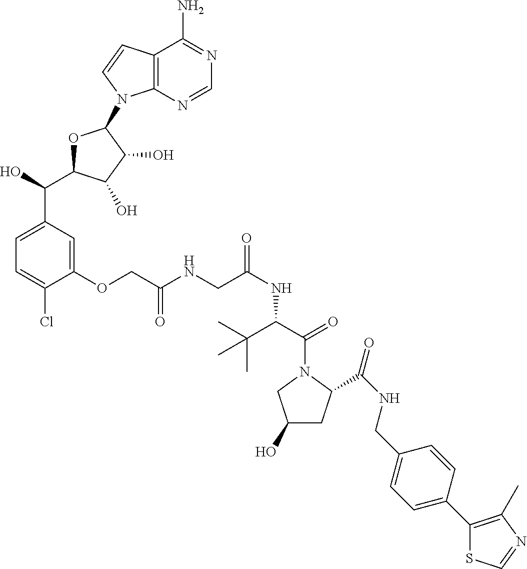 [SMILES: Cc1ncsc1-c1ccc(CNC(=O)[C@@H]2C[C@@H](O)CN2C(=O)[C@@H](NC(=O)CNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-2-(2-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) acetamido)-3,3- dimethylbutanoyl)-4- hydroxy-N-(4-(4- methylthiazol-5- yl)benzyl) pyrrolidine-2- carboxamide |
| YS43- 109 | 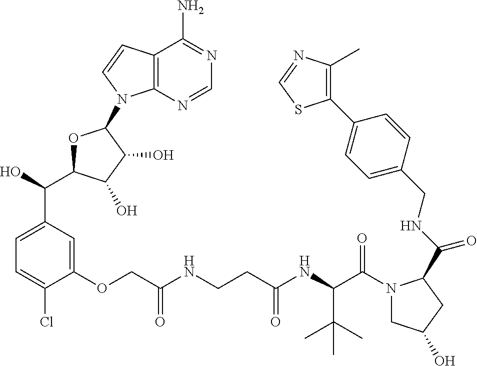 [SMILES: Cc1ncsc1-c1ccc(CNC(=O)[C@H]2C[C@H](O)CN2C(=O)[C@H](NC(=O)CCNC(=O)COc2cc([C@@H](O)[C@H]3O[C@@H](n4ccc5c(N)ncnc54)[C@H](O)[C@@H]3O)ccc2Cl)C(C)(C)C)cc1] | (2R,4S)-1- ((R)-2-(3-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) propanamido)-3,3- dimethylbutanoyl)-4- hydroxy-N-(4-(4- methylthiazol-5-yl) benzyl) pyrrolidine-2- carboxamide |
| YS43- 110 | 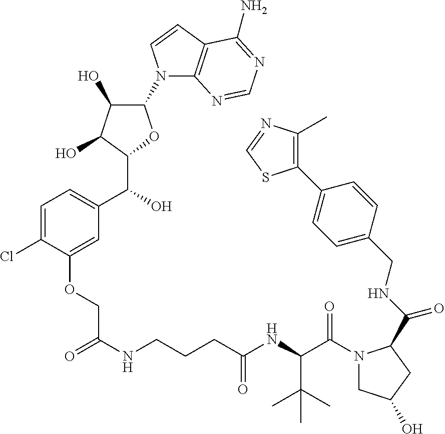 [SMILES: Cc1ncsc1-c1ccc(CNC(=O)[C@H]2C[C@H](O)CN2C(=O)[C@H](NC(=O)CCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2R,4S)-1- ((R)-2-(4-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) butanamido)-3,3- dimethylbutanoyl)-4- hydroxy-N-(4-(4- methylthiazol-5- yl)benzyl) pyrrolidine-2- carboxamide |
| YS43- 111 | 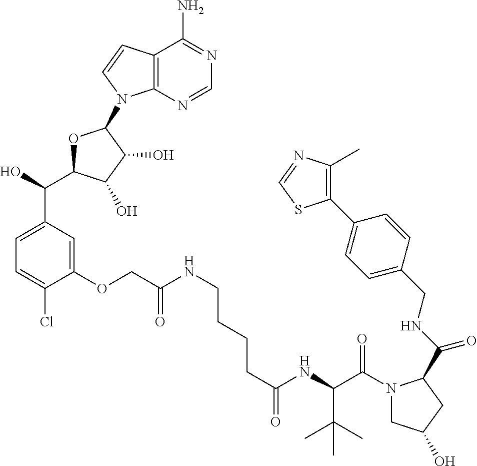 [SMILES: Cc1ncsc1-c1ccc(CNC(=O)[C@H]2C[C@H](O)CN2C(=O)[C@H](NC(=O)CCCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2R,4S)-1- ((R)-2-(5-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) pentanamido)-3,3- dimethylbutanoyl)-4- hydroxy-N-(4-(4- methylthiazol-5-yl) benzyl) pyrrolidine-2- carboxamide |
| YS43- 112 | 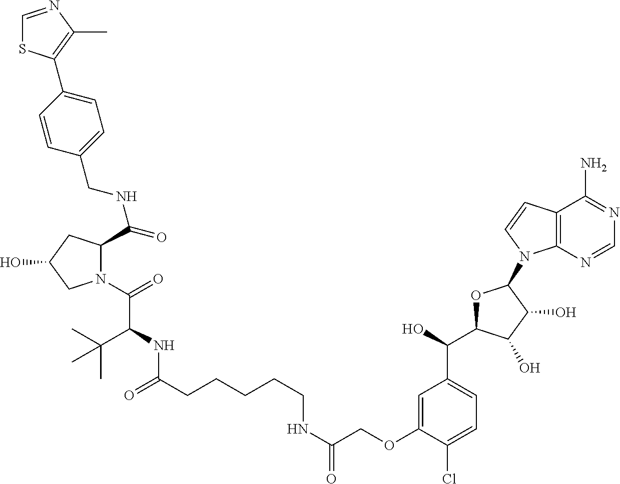 [SMILES: Cc1ncsc1-c1ccc(CNC(=O)[C@@H]2C[C@@H](O)CN2C(=O)[C@@H](NC(=O)CCCCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-2-(6-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) hexanamido)-3,3- dimethylbutanoyl)-4- hydroxy-N-(4-(4- methylthiazol-5-yl) benzyl) pyrrolidine-2- carboxamide |
| YS43- 113 | 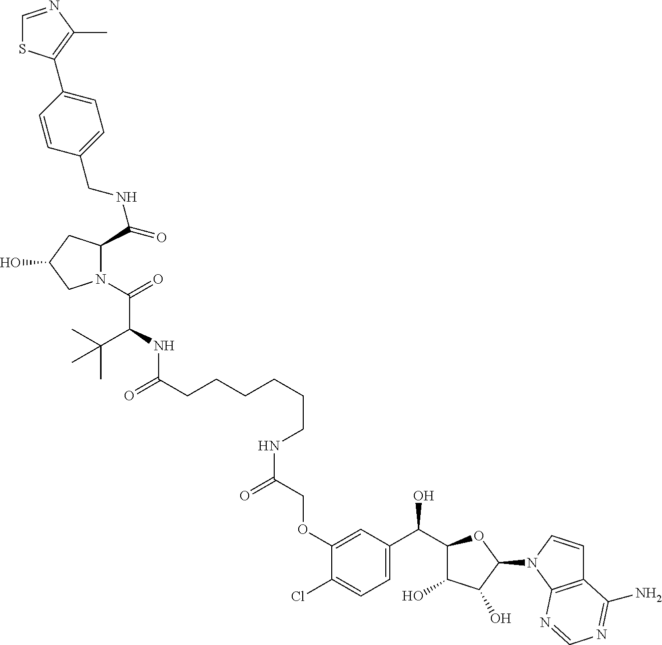 [SMILES: Cc1ncsc1-c1ccc(CNC(=O)[C@@H]2C[C@@H](O)CN2C(=O)[C@@H](NC(=O)CCCCCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-2-(7-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) heptanamido)-3,3- dimethylbutanoyl)-4- hydroxy-N-(4-(4- methylthiazol-5-yl) benzyl) pyrrolidine-2- carboxamide |
| YS43- 114 | 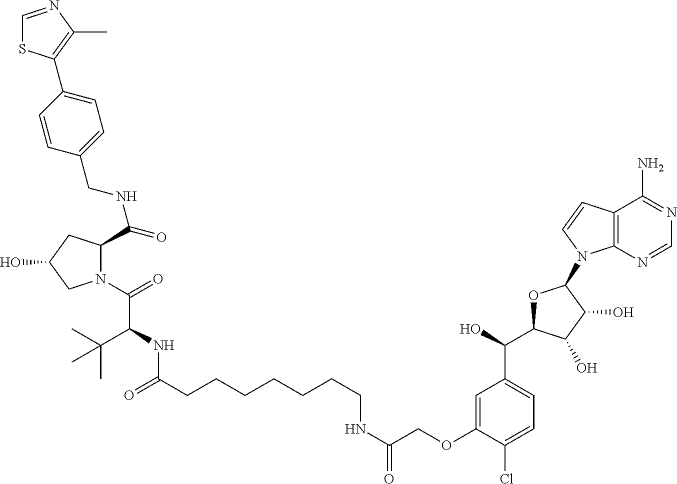 [SMILES: Cc1ncsc1-c1ccc(CNC(=O)[C@@H]2C[C@@H](O)CN2C(=O)[C@@H](NC(=O)CCCCCCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-2-(8-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) octanamido)-3,3- dimethylbutanoyl)-4- hydroxy-N-(4-(4- methylthiazol-5-yl) benzyl) pyrrolidine-2- carboxamide |
| YS43- 115 | 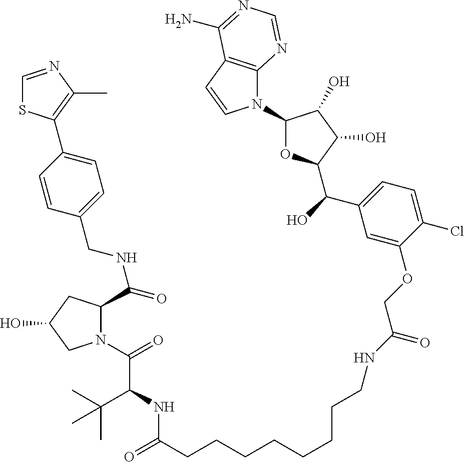 [SMILES: Cc1ncsc1-c1ccc(CNC(=O)[C@@H]2C[C@@H](O)CN2C(=O)[C@@H](NC(=O)CCCCCCCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-2-(9-(2-(5- ((R)-((2R,3S,4R,5R)- 5-(4- amino-7H- pyrrolo[2,3-d] pyrimidin-7-yl)-3,4- dihydroxytetra- hydrofuran-2- yl)(hydroxy) methyl)-2- chlorophenoxy) acetamido) nonanamido)-3,3- dimethylbutanoyl)-4- hydroxy-N-(4-(4- methylthiazol-5-yl) benzyl) pyrrolidine-2- carboxamide |
| YS43- 116 | 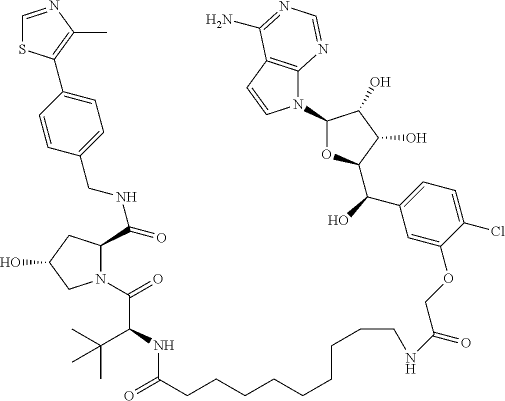 [SMILES: Cc1ncsc1-c1ccc(CNC(=O)[C@@H]2C[C@@H](O)CN2C(=O)[C@@H](NC(=O)CCCCCCCCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1-((S)- 2-(10-(2-(5- ((R)- ((2R,3S,4R,5R)-5- (4-amino-7H- pyrrolo[2,3-d] pyrimidin-7-yl)-3,4- dihydroxytetra- hydrofuran-2- yl)(hydroxy) methyl)-2- chlorophenoxy) acetamido) decanamido)-3,3- dimethylbutanoyl)-4- hydroxy-N-(4-(4- methylthiazol-5-yl) benzyl) pyrrolidine-2- carboxamide |
| YS43- 117 | 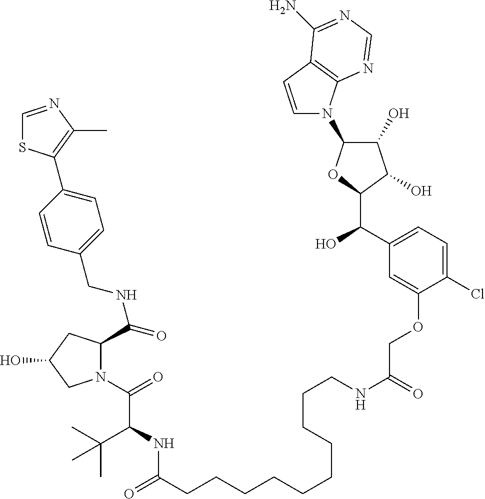 [SMILES: Cc1ncsc1-c1ccc(CNC(=O)[C@@H]2C[C@@H](O)CN2C(=O)[C@@H](NC(=O)CCCCCCCCCCNC(=O)COc2cc([C@@H](O)[C@H]3O[C@@H](n4ccc5c(N)ncnc54)[C@H](O)[C@@H]3O)ccc2Cl)C(C)(C)C)cc1] | (2S,4R)-1-((S)- 2-(11-(2-(5- ((R)- ((2R,3S,4R,5R)-5- (4-amino-7H- pyrrolo[2,3-d] pyrimidin-7-yl)-3,4- dihydroxytetra- hydrofuran-2- yl)(hydroxy) methyl)-2- chlorophenoxy) acetamido) undecanamido)-3,3- dimethylbutanoyl)-4- hydroxy-N-(4-(4- methylthiazol-5- yl)benzyl) pyrrolidine-2- carboxamide |
| CPD- 90 | 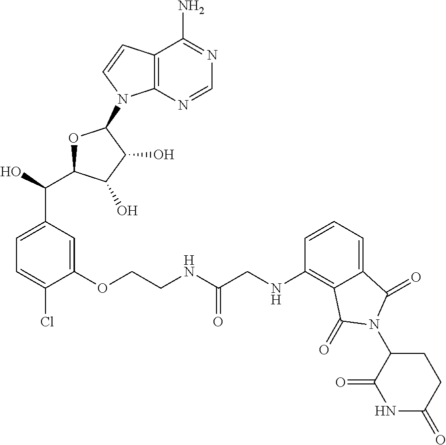 [SMILES: Nc1ncnc2c1ccn2[C@@H]1O[C@H]([C@H](O)c2ccc(Cl)c(OCCNC(=O)CNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3,4- dihydroxytetra- hydrofuran-2-yl) (hydroxy)methyl)-2- chlorophenoxy) ethyl)-2-((2- (2,6- dioxopiperidin-3- yl)-1,3- dioxoisoindolin-4- yl)amino)acetamide |
| CPD- 91 | 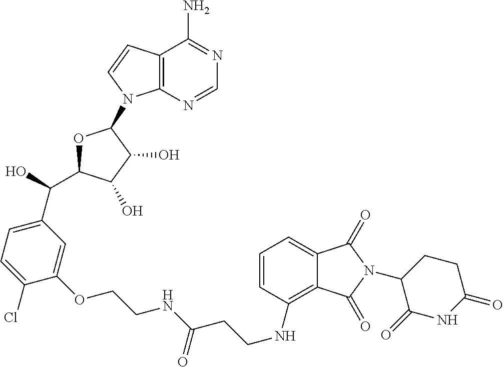 [SMILES: Nc1ncnc2c1ccn2[C@@H]1O[C@H]([C@H](O)c2ccc(Cl)c(OCCNC(=O)CCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin- 7-yl)-3,4- dihydroxytetra- hydrofuran- 2-yl) (hydroxy)methyl)-2- chlorophenoxy) ethyl)-3-((2- (2,6- dioxopiperidin-3- yl)-1,3- dioxoisoindolin- 4-yl) amino)propanamide |
| CPD- 92 | 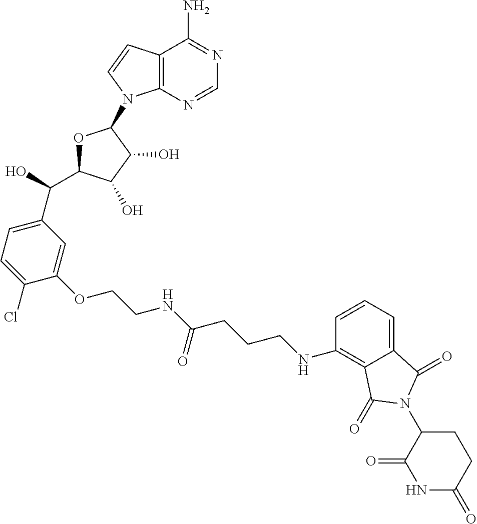 [SMILES: Nc1ncnc2c1ccn2[C@@H]1O[C@H]([C@H](O)c2ccc(Cl)c(OCCNC(=O)CCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin- 7-yl)-3,4- dihydroxytetra- hydrofuran-2- yl)(hydroxy) methyl)-2- chlorophenoxy) ethyl)-4-((2- (2,6- dioxopiperidin-3- yl)-1,3- dioxoisoindolin-4-yl) amino)butanamide |
| CPD- 93 | 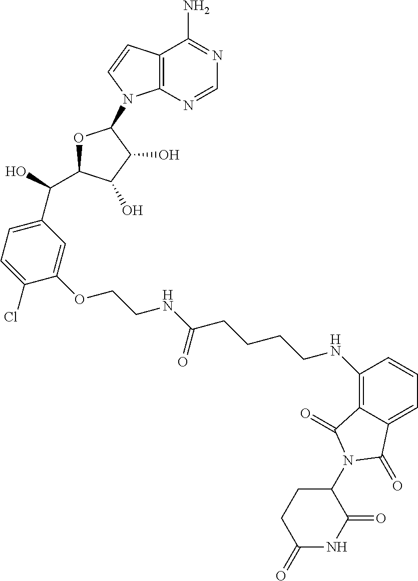 [SMILES: Nc1ncnc2c1ccn2[C@@H]1O[C@H]([C@H](O)c2ccc(Cl)c(OCCNC(=O)CCCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3.4- dihydroxytetra- hydrofuran-2- yl)(hydroxy) methyl)-2- chlorophenoxy) ethyl)-5-((2- (2,6-dioxopiperidin-3- yl)-1,3- dioxoisoindolin-4- yl)amino) pentanamide |
| CPD- 94 | 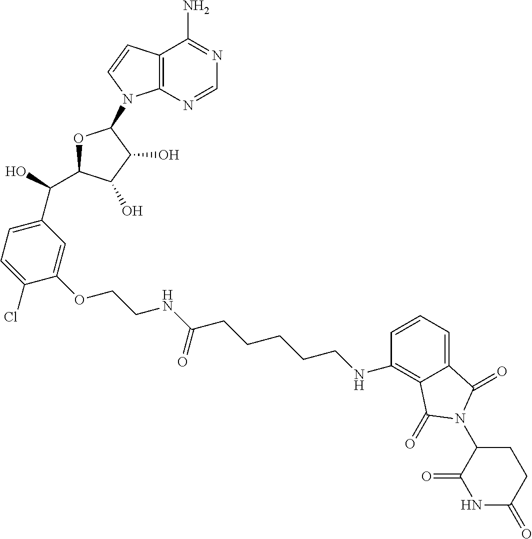 [SMILES: Nc1ncnc2c1ccn2[C@@H]1O[C@H]([C@H](O)c2ccc(Cl)c(OCCNC(=O)CCCCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin- 7-yl)-3,4- dihydroxytetra- hydrofuran-2- yl)(hydroxy) methyl)-2- chlorophenoxy) ethyl)-6-((2- (2,6-dioxopiperidin-3- yl)-1,3- dioxoisoindolin- 4-yl) amino)hexanamide |
| CPD- 95 | 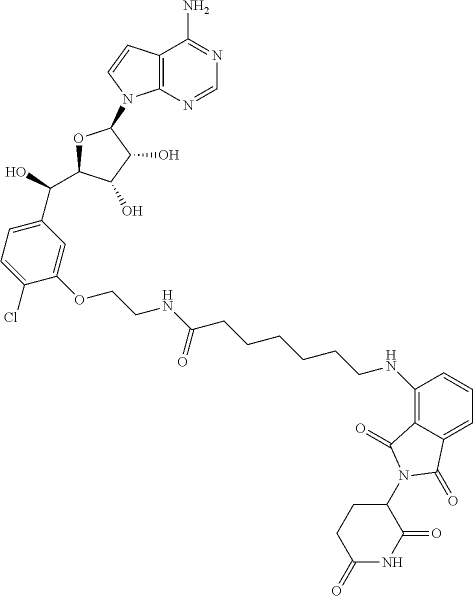 [SMILES: Nc1ncnc2c1ccn2[C@@H]1O[C@H]([C@H](O)c2ccc(Cl)c(OCCNC(=O)CCCCCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-7-((2- (2,6-dioxopiperidin-3- yl)-1,3- dioxoisoindolin- 4-yl) amino)heptanamide |
| CPD- 96 | 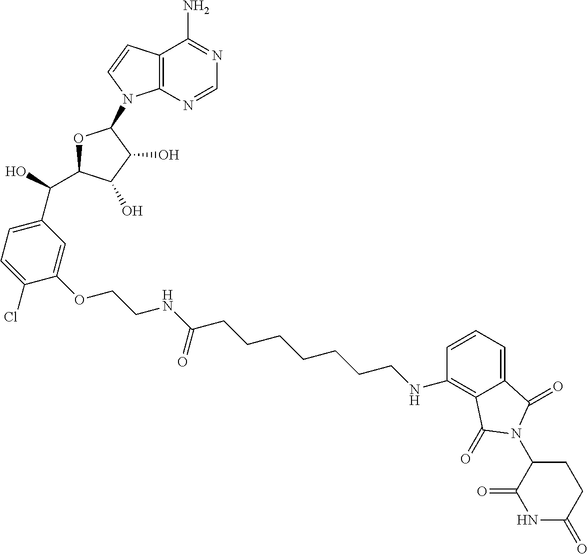 [SMILES: Nc1ncnc2c1ccn2[C@@H]1O[C@H]([C@H](O)c2ccc(Cl)c(OCCNC(=O)CCCCCCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-8-((2- (2,6- dioxopiperidin-3- yl)-1,3- dioxoisoindolin-4- yl)amino) octanamide |
| CPD- 97 | 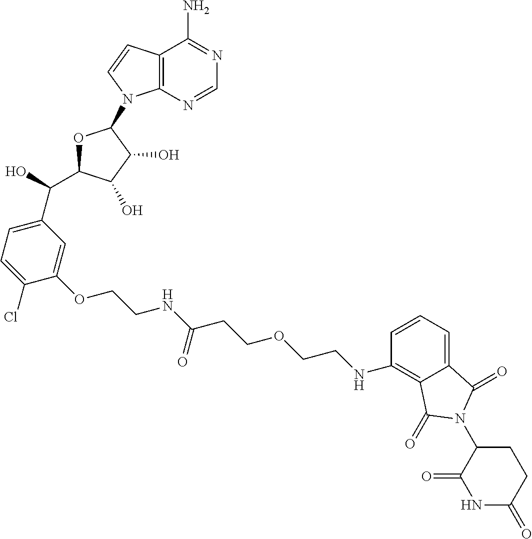 [SMILES: Nc1ncnc2c1ccn2[C@@H]1O[C@H]([C@H](O)c2ccc(Cl)c(OCCNC(=O)CCOCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-3-(2- ((2-(2,6- dioxopiperidin- 3-yl)-1,3- dioxoisoindolin-4- yl)amino)ethoxy) propanamide |
| CPD- 98 | 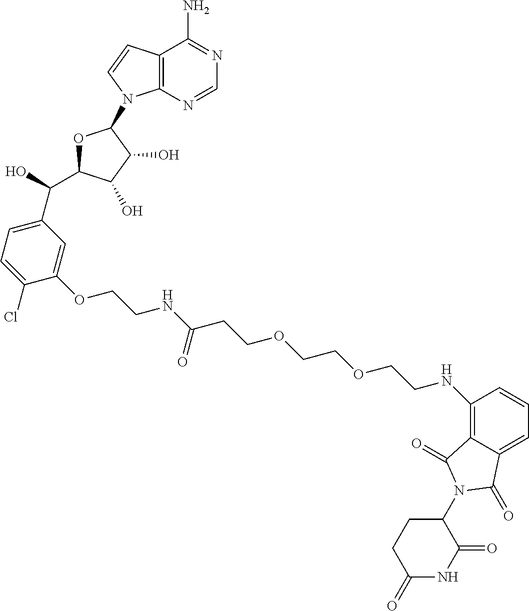 [SMILES: Nc1ncnc2c1ccn2[C@@H]1O[C@H]([C@H](O)c2ccc(Cl)c(OCCNC(=O)CCOCCOCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy)ethyl)- 3-(2-(2-((2-(2,6- dioxopiperidin- 3-yl)-1,3- dioxoisoindolin-4- yl)amino) ethoxy)ethoxy) propanamide |
| CPD- 99 | 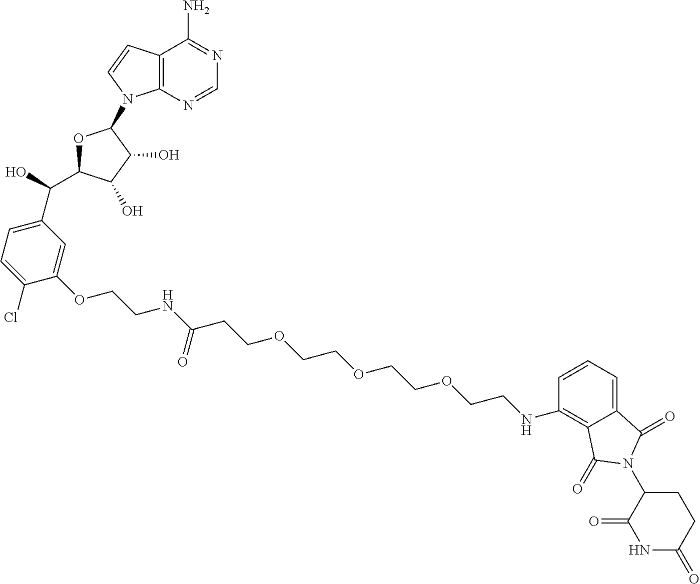 [SMILES: Nc1ncnc2c1ccn2[C@@H]1O[C@H]([C@H](O)c2ccc(Cl)c(OCCNC(=O)CCOCCOCCOCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-3- (2-(2-(2-((2-(2,6- dioxopiperidin- 3-yl)-1,3- dioxoisoindolin-4- yl)amino) ethoxy)ethoxy) ethoxy)propanamide |
| CPD- 100 | 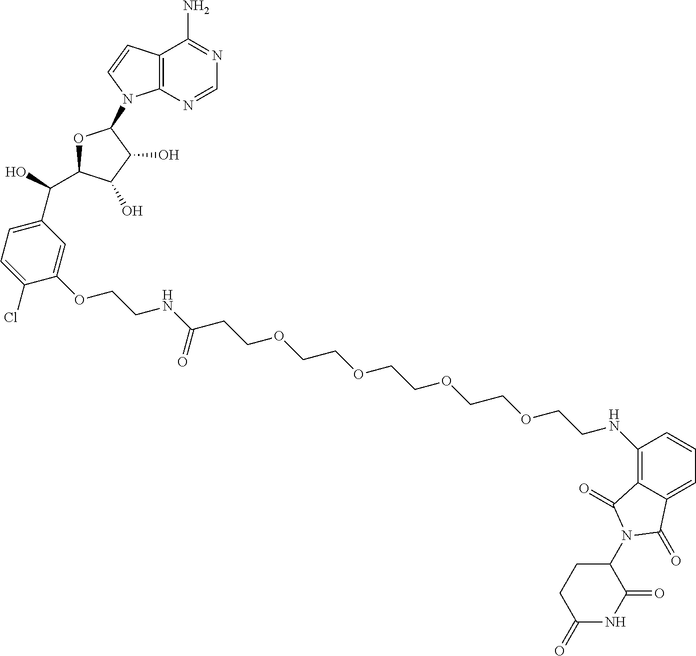 [SMILES: Nc1ncnc2c1ccn2[C@@H]1O[C@H]([C@H](O)c2ccc(Cl)c(OCCNC(=O)CCOCCOCCOCCOCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin-7-yl)-3.4- dihydroxytetra- hydrofuran-2- yl)(hydroxy) methyl)-2- chlorophenoxy) ethyl)-1-((2- (2,6- dioxopiperidin-3- yl)-1,3- dioxoisoindolin-4- yl)amino)-3,6,9,12- tetraoxapentadecan- 15-amide |
| CPD- 101 | 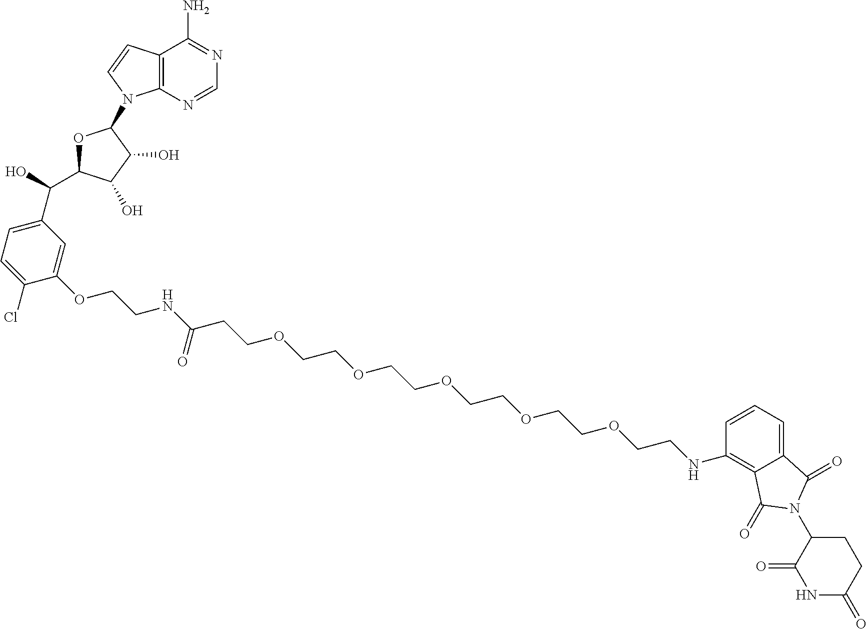 [SMILES: Nc1ncnc2c1ccn2[C@@H]1O[C@H]([C@H](O)c2ccc(Cl)c(OCCNC(=O)CCOCCOCCOCCOCCOCCNc3cccc4c3C(=O)N(C3CCC(=O)NC3=O)C4=O)c2)[C@@H](O)[C@H]1O] | N-(2-(5-((R)- ((2R,3S,4R,5R)- 5-(4-amino-7H- pyrrolo[2,3-d] pyrimidin- 7-yl)-3,4- dihydroxytetra- hydrofuran- 2-yl) (hydroxy)methyl)-2- chlorophenoxy) ethyl)-1-((2- (2,6- dioxopiperidin-3- yl)-1,3- dioxoisoindolin- 4-yl) amino)-3,6,9,12,15- pentaoxaoctadecan- 18-amide |
| CPD- 102 | 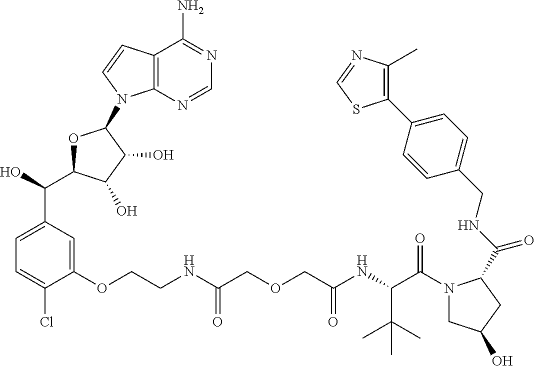 [SMILES: Cc1ncsc1-c1ccc(CNC(=O)[C@@H]2C[C@@H](O)CN2C(=O)[C@@H](NC(=O)COCC(=O)NCCOc2cc([C@@H](O)[C@H]3O[C@@H](n4ccc5c(N)ncnc54)[C@H](O)[C@@H]3O)ccc2Cl)C(C)(C)C)cc1] | (2S,4R)-1-((S)- 2-(2-(2-((2- (5-((R)- ((2R,3S,4R,5R)- 5-(4-amino-7H- pyrrolo[2,3-d] pyrimidin-7-yl)-3,4- dihydroxytetra- hydrofuran- 2-yl) (hydroxy)methyl)-2- chlorophenoxy) ethyl)amino)-2- oxoethoxy) acetamido)-3,3- dimethylbutanoyl)-4- hydroxy-N-(4-(4- methylthiazol-5- yl)benzyl) pyrrolidine- 2-carboxamide |
| CPD- 103 | 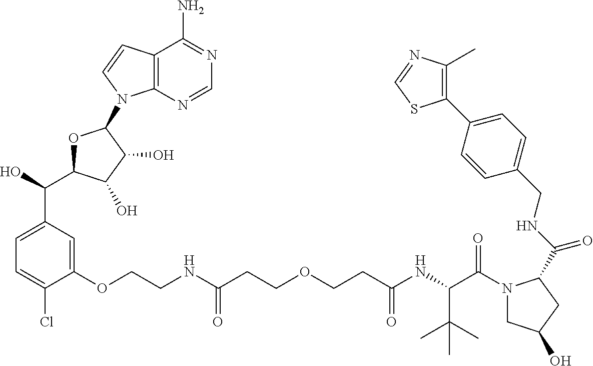 [SMILES: Cc1ncsc1-c1ccc(CNC(=O)[C@@H]2C[C@@H](O)CN2C(=O)[C@@H](NC(=O)CCOCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | (2S,4R)-1-((S)- 2-(3-(3-((2-(5- ((R)-((2R,3S,4R,5R)- 5-(4-amino-7H- pyrrolo[2,3-d] pyrimidin-7-yl)-3,4- dihydroxytetra- hydrofuran- 2-yl) (hydroxy)methyl)-2- chlorophenoxy) ethyl)amino)-3- oxopropoxy) propanamido)-3,3- dimethylbutanoyl)- 4-hydroxy-N-(4-(4- methylthiazol-5- yl)benzyl) pyrrolidine- 2-carboxamide |
| CPD- 104 | 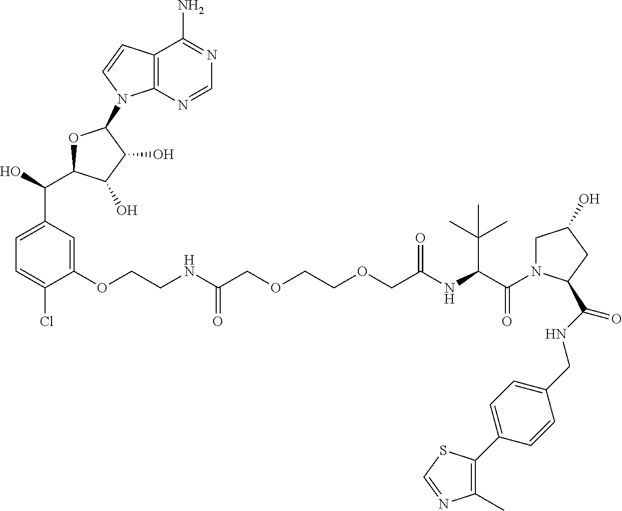 [SMILES: Cc1ncsc1-c1ccc(CNC(=O)[C@@H]2C[C@@H](O)CN2C(=O)[C@@H](NC(=O)COCCOCC(=O)NCCOc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-14-(5-(R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 2-(tert- butyl)-4,11- dioxo-6,9- dioxa-3,12- diazatetradecanoyl)- 4-hydroxy-N-(4-(4- methyithiazol-5- yl)benzyl) pyrrolidine-2- carboxamide |
| CPD- 105 | 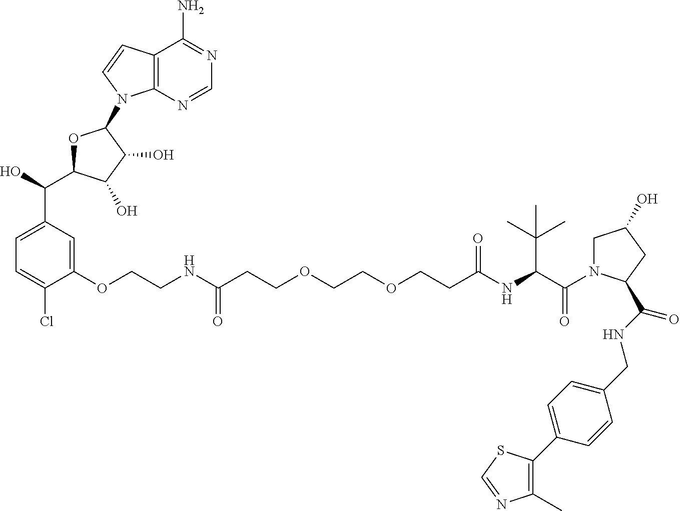 [SMILES: Cc1ncsc1-c1ccc(CNC(=O)[C@@H]2C[C@@H](O)CN2C(=O)[C@@H](NC(=O)CCOCCOCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | (2S,4R)-1- ((S)-16-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 2-(tert- butyl)-4,13- dioxo-7,10- dioxa-3,14- diazahexadecanoyl)- 4-hydroxy-N-(4-(4- methylthiazol- 5-yl)benzyl) pyrrolidine-2- carboxamide |
| CPD- 106 | 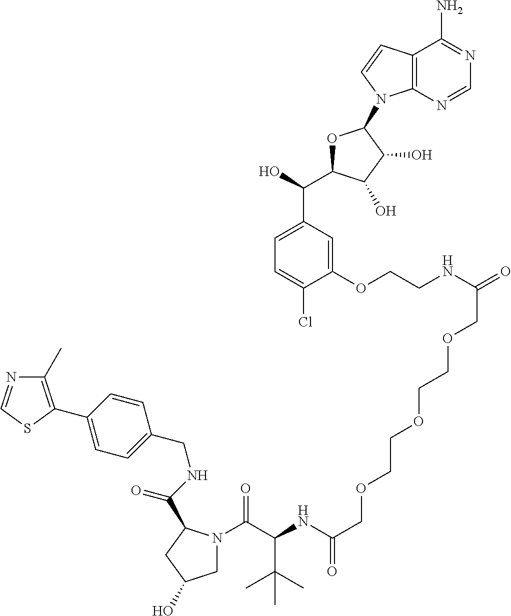 [SMILES: Cc1ncsc1-c1ccc(CNC(=O)[C@@H]2C[C@@H](O)CN2C(=O)[C@@H](NC(=O)COCCOCCOCC(=O)NCCOc2cc([C@@H](O)[C@H]3O[C@@H](n4ccc5c(N)ncnc54)[C@H](O)[C@@H]3O)ccc2Cl)C(C)(C)C)cc1] | (2S,4R)-1-((S)- 17-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 2-(tert-butyl)- 4,14-dioxo-6,9,12- trioxa-3,15- diazaheptadecanoyl)- 4-hydroxy-N-(4- (4-methylthiazol- 5-yl)benzyl) pyrrolidine-2- carboxamide |
| CPD- 107 | 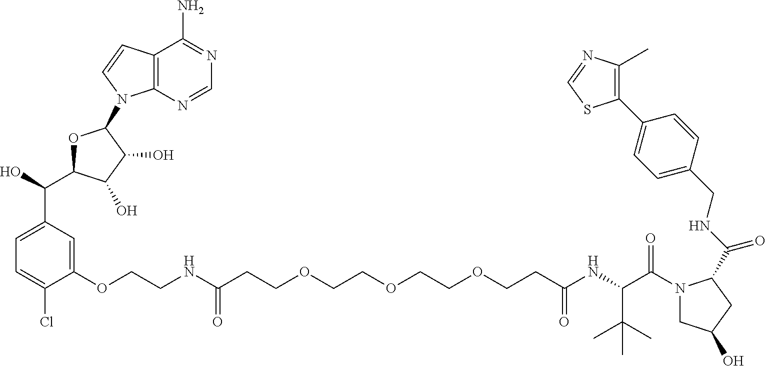 [SMILES: Cc1ncsc1-c1ccc(CNC(=O)[C@@H]2C[C@@H](O)CN2C(=O)[C@@H](NC(=O)CCOCCOCCOCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | (2S,4R)-1-((S)- 19-(5-((R)- ((2R,3S,4R,5R)-5-(4- amino-7H- pyrrolo[2,3-d] pyrimidin-7-yl)-3,4- dihydroxytetra- hydrofuran-2- yl)(hydroxy) methyl)-2- chlorophenoxy)- 2-(tert-butyl)- 4,16-dioxo-7,10,13- trioxa-3,17- diazanonadecanoyl)- 4-hydroxy-N-(4-(4- methylthiazol- 5-yl)benzyl) pyrrolidine-2- carboxamide |
| CPD- 108 | 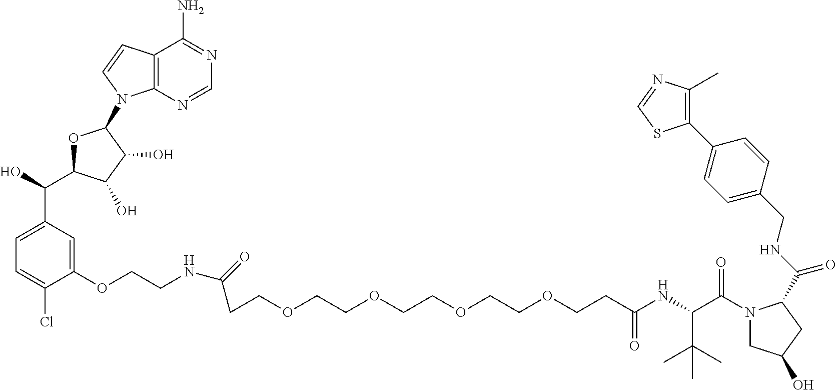 [SMILES: Cc1ncsc1-c1ccc(CNC(=O)[C@@H]2C[C@@H](O)CN2C(=O)[C@@H](NC(=O)CCOCCOCCOCCOCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran- 2-yl) (hydroxy)methyl)-2- chlorophenoxy) ethyl)-N¹⁶- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2- yl)-4,7,10,13- tetraoxahexa- decanediamide |
| CPD- 109 | 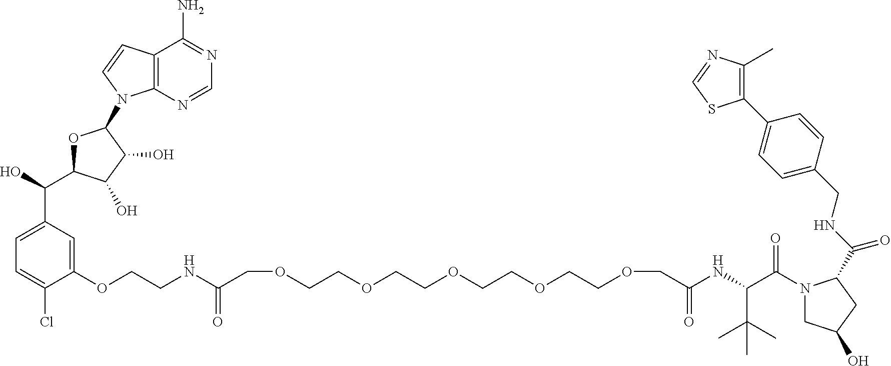 [SMILES: Cc1ncsc1-c1ccc(CNC(=O)[C@@H]2C[C@@H](O)CN2C(=O)[C@@H](NC(=O)COCCOCCOCCOCCOCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3,S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-N¹⁷- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl)- 3,6,9,12,15- pentaoxahepta- decanediamide |
| CPD- 110 | 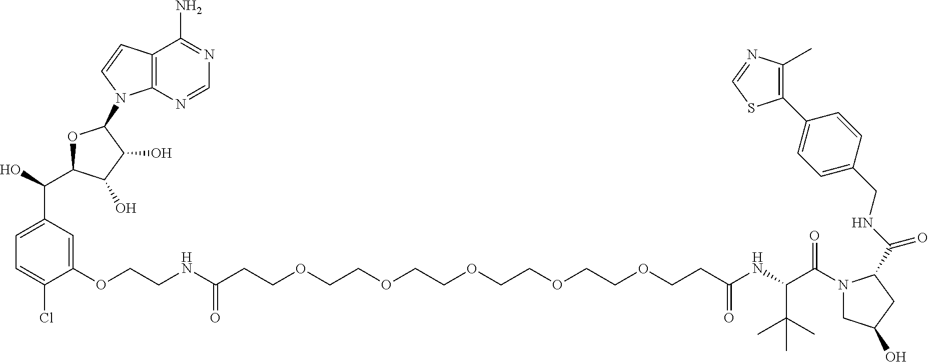 [SMILES: Cc1ncsc1-c1ccc(CNC(=O)[C@@H]2C[C@@H](O)CN2C(=O)[C@@H](NC(=O)CCOCCOCCOCCOCCOCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-N¹⁹- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl)- 4,7,10,13,16- pentaoxanona- decanediamide |
| CPD- 111 | 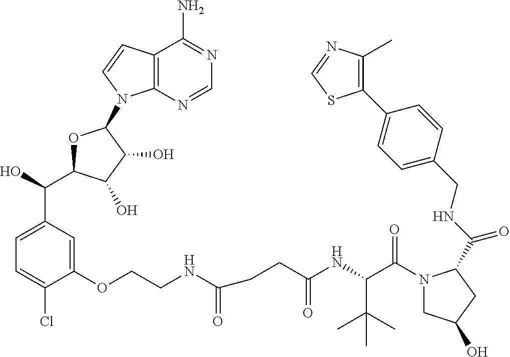 [SMILES: Cc1ncsc1-c1ccc(CNC(=O)[C@@H]2C[C@@H](O)CN2C(=O)[C@@H](NC(=O)CCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-N⁴- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) succinamide |
| CPD- 112 | 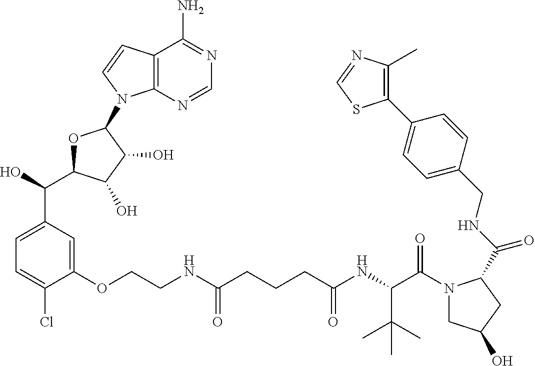 [SMILES: Cc1ncsc1-c1ccc(CNC(=O)[C@@H]2C[C@@H](O)CN2C(=O)[C@@H](NC(=O)CCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-N⁵- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) glutaramide |
| CPD- 113 | 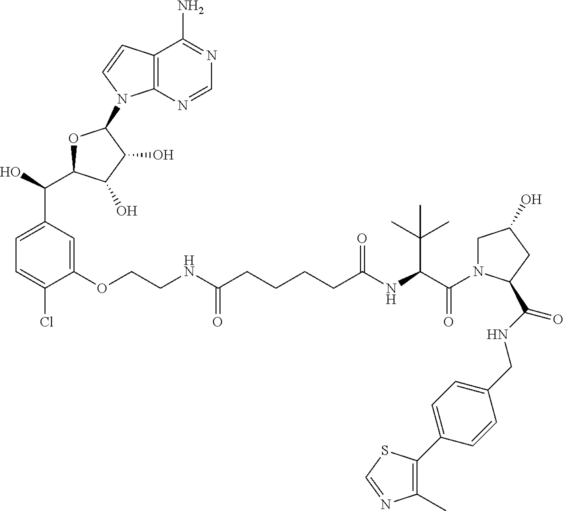 [SMILES: Cc1ncsc1-c1ccc(CNC(=O)[C@@H]2C[C@@H](O)CN2C(=O)[C@@H](NC(=O)CCCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran- 2-yl) (hydroxy)methyl)-2- chlorophenoxy) ethyl)-N⁶- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) adipamide |
| CPD- 114 | 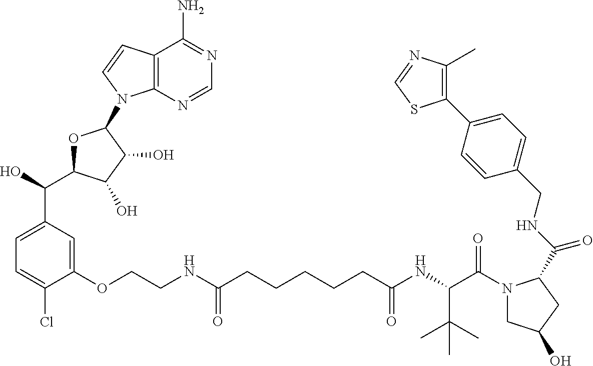 [SMILES: Cc1ncsc1-c1ccc(CNC(=O)[C@@H]2C[C@@H](O)CN2C(=O)[C@@H](NC(=O)CCCCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-N⁷- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) heptanediamide |
| CPD- 115 | 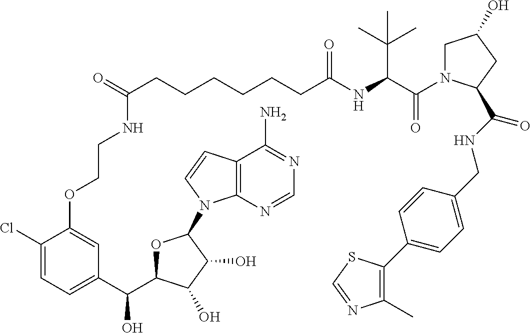 [SMILES: Cc1ncsc1-c1ccc(CNC(=O)[C@@H]2C[C@@H](O)CN2C(=O)[C@@H](NC(=O)CCCCCCC(=O)NCCOc2cc([C@H](O)[C@H]3O[C@@H](n4ccc5c(N)ncnc54)[C@H](O)[C@@H]3O)ccc2Cl)C(C)(C)C)cc1] | N¹-(2-(5-((S)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)- N⁸-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) octanediamide |
| CPD- 116 | 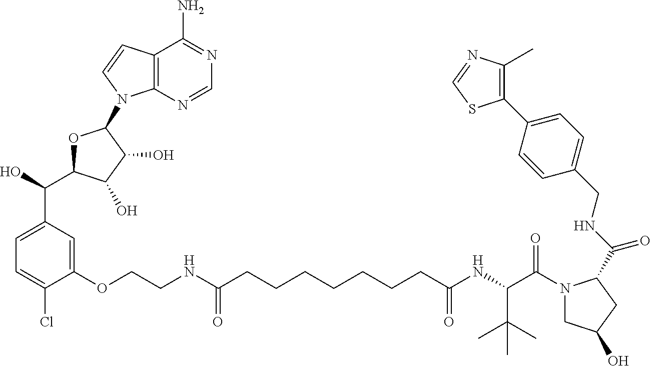 [SMILES: Cc1ncsc1-c1ccc(CNC(=O)[C@@H]2C[C@@H](O)CN2C(=O)[C@@H](NC(=O)CCCCCCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)- N⁹-((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) nonanediamide |
| CPD- 117 | 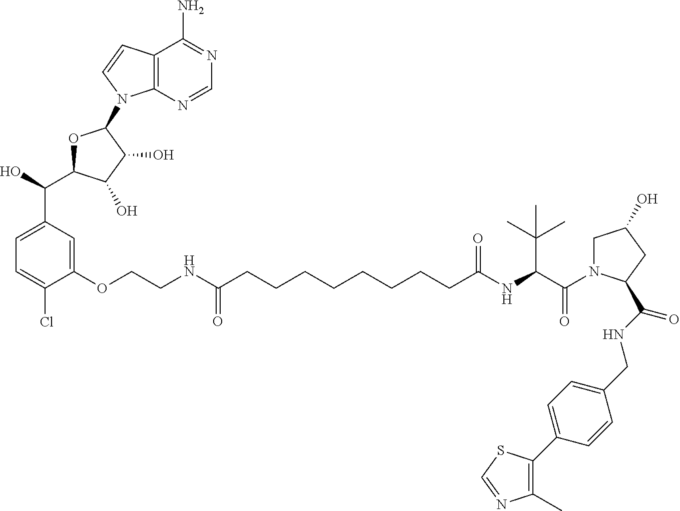 [SMILES: Cc1ncsc1-c1ccc(CNC(=O)[C@@H]2C[C@@H](O)CN2C(=O)[C@@H](NC(=O)CCCCCCCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)-N¹⁰- ((S)-1-((2S,4R)-4- hydroxy-2-((4-(4- methylthiazol-5- yl)benzyl)carbamoyl) pyrrolidin- 1-yl)-3,3-dimethyl- 1-oxobutan-2-yl) decanediamide |
| CPD- 118 | 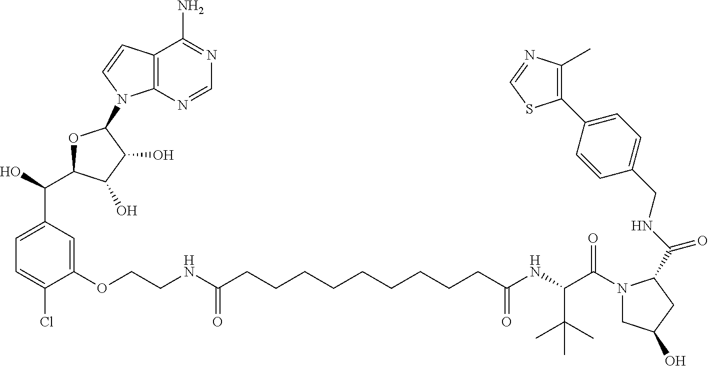 [SMILES: Cc1ncsc1-c1ccc(CNC(=O)[C@@H]2C[C@@H](O)CN2C(=O)[C@@H](NC(=O)CCCCCCCCCC(=O)NCCOc2cc([C@@H](O)[C@H]3O[C@@H](n4ccc5c(N)ncnc54)[C@H](O)[C@@H]3O)ccc2Cl)C(C)(C)C)cc1] | N¹-(2-(5-((R)- ((2R,3S,4R,5R)- 5-(4-amino-7H- pyrrolo[2,3-d] pyrimidin-7-yl)-3,4- dihydroxytetra- hydrofuran-2- yl)(hydroxy) methyl)-2- chlorophenoxy) ethyl)- N¹¹-((S)-1- ((2S,4R)-4- hydroxy-2-((4-(4- methylthiazol-5- yl)benzyl)carbamoyl) pyrrolidin- 1-yl)-3,3-dimethyl- 1-oxobutan-2-yl) undecanediamide |

As used herein, in case of discrepancy between the structure and chemical name provided for a particular compound, the given structure shall control.

### Materials And Methods:

### Cell Lines and Tissue Culture

MCF-7 cells, HEK293T cells and HeLa cells were cultured in Dulbecco's Modified Eagle Medium (DMEM) supplemented with 10% fetal bovine serum (FBS) in 5% CO₂ at 37° C. Jurkat cells were cultured in RPMI 1640 medium supplemented with 10% FBS in 5% CO₂ at 37° C. H2171 cells were cultured in HITES medium supplemented with 10% FBS in 5% CO₂ at 37° C. HCT116 were cultured in McCoy's 5A medium supplemented with 10% fetal bovine serum (FBS) in 5% CO₂ at 37° C. All the cells lines were treated with 5 μM (or indicated concentration) of PRMT5 inhibitor (EPZ015666) or degraders, or equivalent volume of DMSO for 6 days (or indicated time points). Fresh compounds were constituted in medium and used for every 2 days.

### Antibodies and Western Blotting

The antibodies used in this study are: PRMT5 antibody (Active Motif), (3-actin antibody (Sigma), SDMA antibody (collaboration with Cell Signaling Technologies). Cell cultures were harvested at the end of compound treatments and applied subjected to western blotting assay. Briefly, cells were washed with PBS and lysed with RIPA lysis buffer for 20 minutes on ice. Then lysates were centrifuged and the clear supernatant were collected. SDS loading buffer was added to the lysates and then the mixture was boiled at 100° C. for 5 minutes. Samples were loaded to 10% or 12% SDS-PAGE gel and ran at 120V for 1.5 hours. Then the proteins were transferred to PVDF membrane and blocked in 5% milk fat at room temperature for 1 hour. Then the membranes were incubated with indicated antibodies at 4° C. overnight. Membranes were washed with PBST buffer three times for 10 minutes each time and incubated with HRP-conjugated (horse radish peroxidase) secondary antibodies at room temperature for 1 hour. Again the membranes were washed with PBST buffer three times for 10 minutes each time. Finally, the indicated proteins on the membranes were visualized with ECL (enhanced chemiluminescence) reagents.

### Cell Proliferation Assay

The same starting number of MCF-7 cells were treated with DMSO, 5 μM EPZ015666 or YS43-22 for up to 10 days. Every two days, fresh compounds were constituted in DMEM medium and added to the cell culture. Relative cell numbers were counted every two days (i.e. day 0, 2, 4, 6, 8 and 10) using Cell-Titer Glo kit (Promega).

### Example 90—PRMT5 Degraders Reduced PRMT5 Protein Levels in MCF Cells at 5 μM (FIG. 1)

MCF-7 cells were treated with DMSO or indicated compounds at 5 μM for 6 days. Western blot results showed that various PRMT5 degraders significantly reduced PRMT5 protein levels while the PRMT5 inhibitor, EPZ015666 had no effect on PRMT5 protein levels.

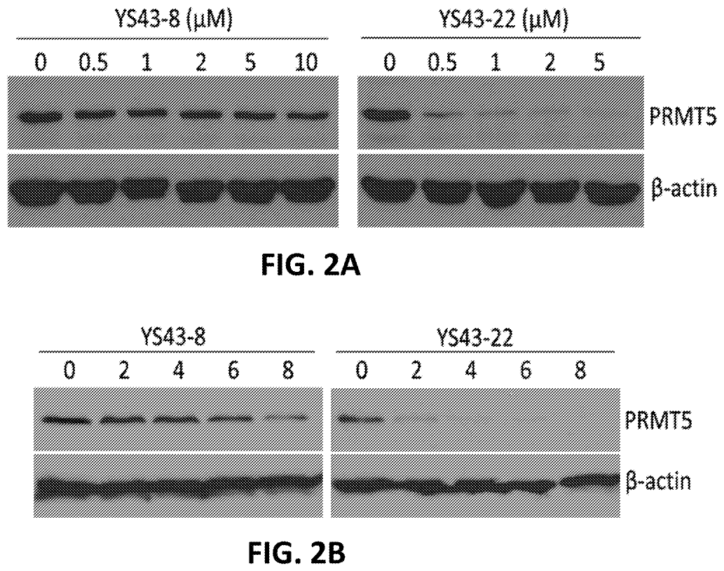

### Example 91—PRMT5 Degraders Reduced PRMT5 Protein Levels in MCF-7 Cells in a Concentration- and Time-Dependent Manner (FIG. 2)

MCF-7 cells were treated with DMSO or indicated serial dilutions of compounds for 6 days. The PRMT5 protein levels were determined by Western blot. The results showed that PRMT5 degrader YS43-22 significantly reduced PRMT5 protein levels in a concentration and time-dependent manner.

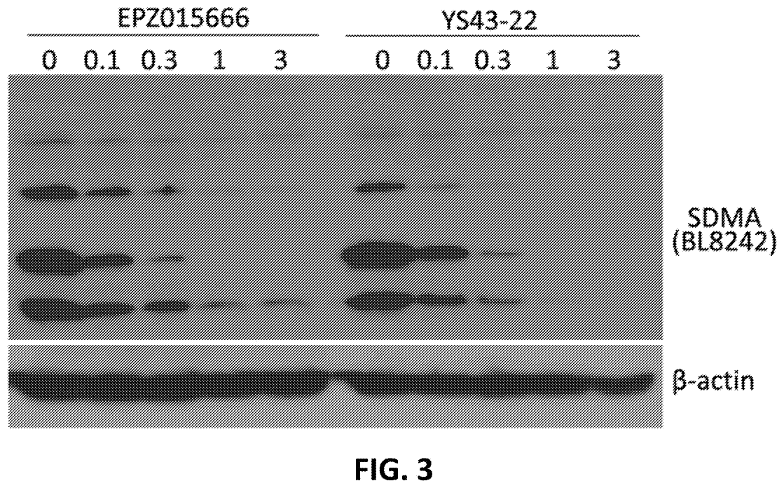

### Example 92—Effect of YS43-22 on Arginine Symmetric Dimethylation (FIG. 3)

MCF-7 cells were treated with EPZ015666 or YS43-22 at indicated compound concentrations (μM) for 6 days. YS43-22 showed significant inhibition of arginine symmetric dimethylation.

### Example 93—PRMT5 Degraders Reduced PRMT5 Protein Levels in Multiple Cell Lines (FIG. 4)

Example 93 demonstrates that PRMT5 degraders reduced PRMT5 protein levels in multiple cell lines. MCF-7, Hela, Jurkat, HCT116, 293T, and H₂₁₇₁ cells were treated with DMSO, YS43-8 (5 μM), or YS43-22 (5 μM) for 6 days. YS43-22 was more potent than YS43-8 in the down-regulation of (i.e., reducing) PRMT5 protein levels in these tested cell lines. YS43-22 degraded PRMT5 very well in MCF-7 and Jurkat cells; moderately in HeLa cells; slightly in HCT116 and 293T cells; not obviously in H₂₁₇₁ cells.

### Example 94—YS43-22 Significantly Inhibited Cell Growth of MCF-7 Cells (FIG. 5)

MCF-7 cells were treated with DMSO, EPZ015666 (5 μM), or YS43-22 (5 μM) for 10 days. Similar to EPZ015666, YS43-22 significantly inhibited cell growth of MCF-7 cells.

### Example 95—YS43-22 was Bioavailable in Mice (FIG. 6)

Standard PK studies were conducted using male Swiss Albino mice. A single 150 mg/kg intraperitoneal (IP) injection of YS43-22 was evaluated. Plasma concentrations of YS43-22 reported at each of the six time points (15 min, 30 min, 1 h, 2 h, 6 h, and 12 h post dosing) are the average values from 3 test animals. There were no abnormal clinical observations noted during the course of the study.

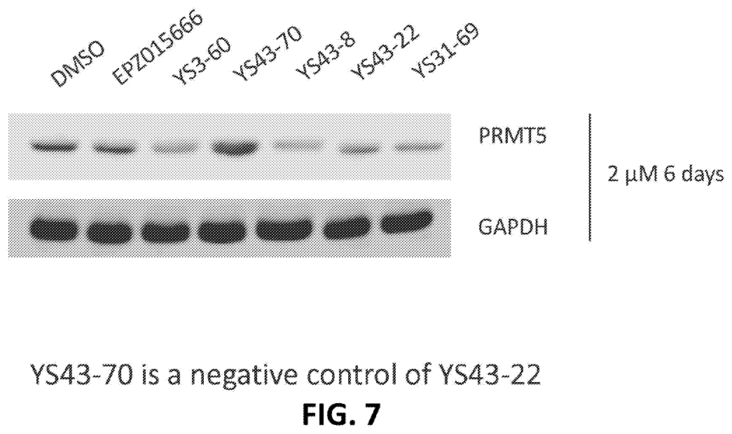

### Example 96—PRMT5 Degraders Reduced PRMT5 Protein Levels in MDA-MB-231 Cells (FIG. 7)

2.5×10⁵ MDA-MB-231 cells were seeded in 10 mL of DMEM 1× (Invitrogen) supplemented with 10% heat inactivated FBS (cellgro) and treated with 20 μL of 1 mM test compounds resuspended in DMSO (YS3-60, YS43-8, YS43-70, YS43-22, YS31-69 and EPZ015666) for 6 days. Western Blot was performed with standard procedures by using anti-PRMT5 antibody from Epigentek (A-3005-50). YS43-70 is a negative control of YS43-22.

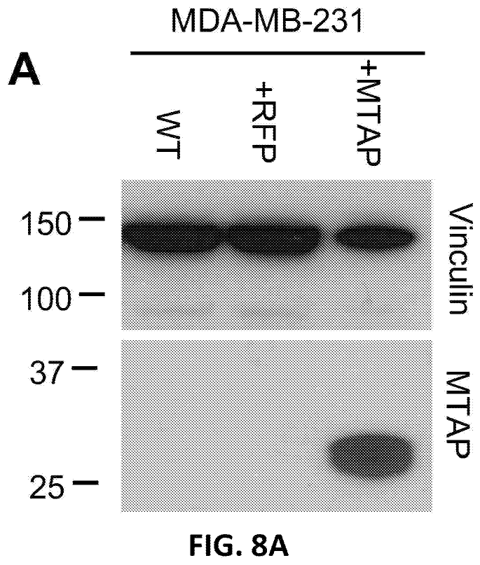

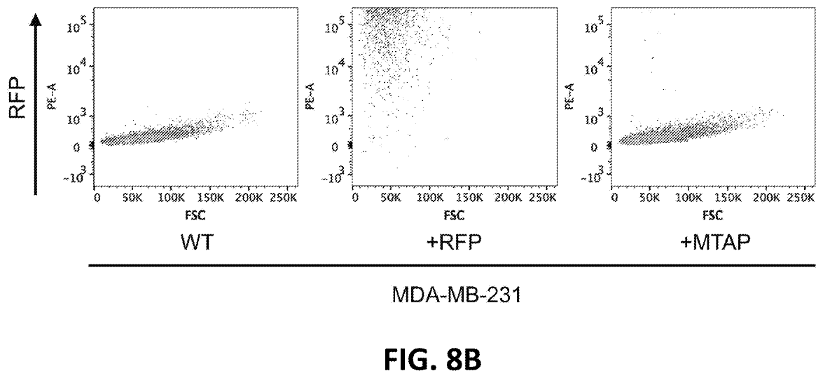

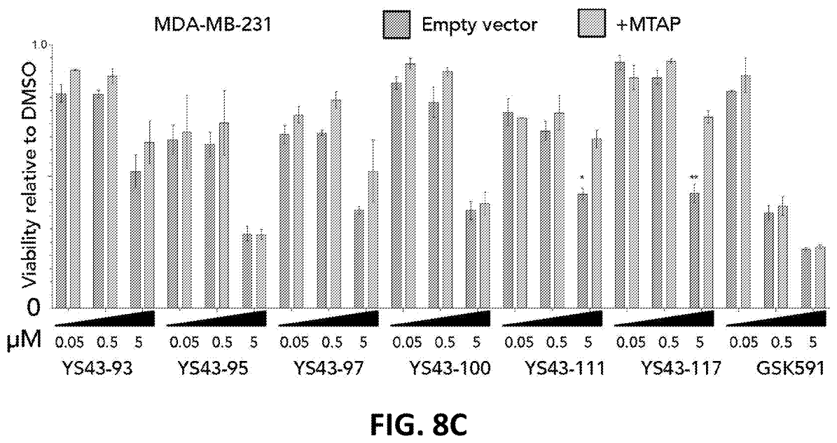

### Example 97—MTAP Deletion in MDA-MB-231 Cells Confers Higher Susceptibility to PRMT5 Degraders (FIG. 8)

### Lentivirus Production

293T cells were seeded at 1×10⁶ one day prior to being transfected with pLVX-RFP or pLVX-MTAP using the protocol for Lipofectamine 3000 Transfection Reagent (Invitrogen) in 10% FBS 1% glutamine Dulbecco's Modified Eagle Medium (DMEM). At 24 hours post transfection, their media was aspirated and replaced with 10 mL 10% FBS 1% glutamine DMEM. At 48 hours post transfection, lentiviral supernatants were collected and mixed in a 1:4 ratio with a 20% sucrose buffer then centrifuged for 4 hours at maximum speed to concentrate the virus. After 4 hours the supernatant was aspirated and the lentiviral pellet was resuspended in 200 uL 10% FBS 1% glutamine DMEM, aliquoted, and stored at −80 degrees Celsius. Titers were determined using Lenti-X GoStix (Clontech).

### Lentiviral Infection

MDA-MB-231 cells were seeded at 3×10⁵ in 6-well plates one day prior to infection. Cells were infected at an MOI of 5 with pLVX-MTAP lentivirus to over-express MTAP, or pLVX-RFP as a control. Two days post infection, the virus was removed and replaced with complete DMEM with 2 ug/mL puromycin for selection over three days.

### Western Blot

Cells were trypsinized, resuspended, and counted using trypan blue staining. 2×10⁶ cells were lysed with 2× Laemmeli buffer and quantified using RC-DC Protein Assay (Bio-Rad). 10 ug of protein were loaded per well. Membranes were blocked in 5% milk PBS-T for 1 hour at room temperature and incubated over night with antibodies against vinculin at 1:10,000 (abcam) or MTAP at 1:1,000 (Cell Signaling Technology) in 0.5% milk PBS-T. An anti-rabbit HRP secondary antibody (GE Healthcare) was used at 1:10,000 for 1 hour at room temperature in 0.5% milk PBS-T.

### Flow Cytometry

To confirm RFP expression, cells were trypsinized and fixed in 4% paraformaldehyde for 10 minutes at room temperature. Fluorescence was measured using the BD LSRFortessa (BD Bioscences).

### Cell Viability Assay

Cells were seeded at 3×10⁴ in 96-well plates one day prior to treatment with each of 6 PRMT5 degraders (YS43-93, YS43-95, YS43-97, YS43-100, YS43-111 and YS43-117) or GSK591 at 50 nM, 0.5 uM, and 5 uM or 5 uM DMSO. 7 days post treatment, 10 uL of 0.15 mg/mL resazurin sodium salt in PBS was added to each well and incubated at 37 degrees Celsius for 2 hours. Fluorescent was then read at 560 nM excitation/590 nM emission.

### OTHER ASPECTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Aguilar, A., Lu, J., Liu, L., Du, D., Bernard, D., McEachern, D., Przybranowski, S., Li, X., Luo, R., Wen, B., et al. (2017). Discovery of 4-((3′R,4'S,5′R)-6″-Chloro-4′-(3-chloro-2-fluorophenyl)-1′-ethyl-2″-oxodispiro[cyclohexane-1,2′-pyrrolidine-3′,3″-indoline]-5′-carboxamido)bicyclo[2.2.2]octane-1-carboxylic Acid (AA-115/APG-115): A Potent and Orally Active Murine Double Minute 2 (MDM2) Inhibitor in Clinical Development. J Med Chem 60, 2819-2839.
Alinari, L., Mahasenan, K. V., Yan, F., Karkhanis, V., Chung, J. H., Smith, E. M., Quinion, C., Smith, P. L., Kim, L., Patton, J. T., et al. (2015). Selective inhibition of protein arginine methyltransferase 5 blocks initiation and maintenance of B-cell transformation. Blood 125, 2530-2543.
Bondeson, D. P., Mares, A., Smith, I. E., Ko, E., Campos, S., Miah, A. H., Mulholland, K. E., Routly, N., Buckley, D. L., Gustafson, J. L., et al. (2015). Catalytic in vivo protein knockdown by small-molecule PROTACs. Nat Chem Biol 11, 611-617.
Braun, C. J., Stanciu, M., Boutz, P. L., Patterson, J. C., Calligaris, D., Higuchi, F., Neupane, R., Fenoglio, S., Cahill, D. P., Wakimoto, H., et al. (2017). Coordinated Splicing of Regulatory Detained Introns within Oncogenic Transcripts Creates an Exploitable Vulnerability in Malignant Glioma. Cancer Cell 32, 411-+.
Buckley, D. L., and Crews, C. M. (2014). Small-molecule control of intracellular protein levels through modulation of the ubiquitin proteasome system. Angew Chem Int Ed Engl 53, 2312-2330.
Buckley, D. L., Gustafson, J. L., Van Molle, I., Roth, A. G., Tae, H. S., Gareiss, P. C., Jorgensen, W. L., Ciulli, A., and Crews, C. M. (2012a). Small-molecule inhibitors of the interaction between the E3 ligase VHL and HIF1alpha. Angew Chem Int Ed Engl 51, 11463-11467.
Buckley, D. L., Raina, K., Darricarrere, N., Hines, J., Gustafson, J. L., Smith, I. E., Miah, A. H., Harling, J. D., and Crews, C. M. (2015). HaloPROTACS: Use of Small Molecule PROTACs to Induce Degradation of HaloTag Fusion Proteins. ACS Chem Biol 10, 1831-1837.
Buckley, D. L., Van Molle, I., Gareiss, P. C., Tae, H. S., Michel, J., Noblin, D. J., Jorgensen, W. L., Ciulli, A., and Crews, C. M. (2012b). Targeting the von Hippel-Lindau E3 ubiquitin ligase using small molecules to disrupt the VHL/HIF-1alpha interaction. J Am Chem Soc 134, 4465-4468.
Chamberlain, P. P., Lopez-Girona, A., Miller, K., Carmel, G., Pagarigan, B., Chie-Leon, B., Rychak, E., Corral, L. G., Ren, Y. J., Wang, M., et al. (2014). Structure of the human Cereblon-DDB1-lenalidomide complex reveals basis for responsiveness to thalidomide analogs. Nat Struct Mol Biol 21, 803-809.
Chan-Penebre, E., Kuplast, K. G., Majer, C. R., Boriack-Sjodin, P. A., Wigle, T. J., Johnston, L. D., Rioux, N., Munchhof, M. J., Jin, L., Jacques, S. L., et al. (2015). A selective inhibitor of PRMT5 with in vivo and in vitro potency in MCL models. Nat Chem Biol 11, 432-437.
Deng, X., Shao, G., Zhang, H. T., Li, C., Zhang, D., Cheng, L., Elzey, B. D., Pili, R., Ratliff, T. L., Huang, J., et al. (2017). Protein arginine methyltransferase 5 functions as an epigenetic activator of the androgen receptor to promote prostate cancer cell growth. Oncogene 36, 1223-1231.
E. Wakeling, A. (1995). Use of pure antioestrogens to elucidate the mode of action of oestrogens. Biochem Pharmacol 49, 1545-1549.
Fischer, E. S., Bohm, K., Lydeard, J. R., Yang, H., Stadler, M. B., Cavadini, S., Nagel, J., Serluca, F., Acker, V., Lingaraju, G. M., et al. (2014). Structure of the DDB1-CRBN E3 ubiquitin ligase in complex with thalidomide. Nature 512, 49-53.
Galdeano, C., Gadd, M. S., Soares, P., Scaffidi, S., Van Molle, I., Birced, I., Hewitt, S., Dias, D. M., and Ciulli, A. (2014). Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663.
Gulla, A., Hideshima, T., Bianchi, G., Fulciniti, M., Kemal Samur, M., Qi, J., Tai, Y. T., Harada, T., Morelli, E., Amodio, N., et al. (2017). Protein arginine methyltransferase 5 has prognostic relevance and is a druggable target in multiple myeloma. Leukemia.
Hiroyuki Suda, Tomohisa Takita, Takaaki Aoyagi, and Umezawa, H. (1976). The structure of bestatin. The Journal of Antibiotic 20, 100-101.
Hu, H., Qian, K., Ho, M. C., and Zheng, Y. G. (2016). Small Molecule Inhibitors of Protein Arginine Methyltransferases. Expert Opin Investig Drugs 25, 335-358.
Indrawan J. Mcalpine, John Tatlock, Joseph Billitti, John Braganza, Alexei Brooun, Deng Ya-Li, Brad Hirakawa, Kristen Jensen-Pergakes, Robert Kumpf, Wei Liu, et al. (2018). Abstract 4857: Discovery of PF-06855800, a SAM competitive PRMT5 inhibitor with potent antitumor activity. Paper presented at: AACR Annual Meeting (Chicago, Ill.).
Ito, T., Ando, H., Suzuki, T., Ogura, T., Hotta, K., Imamura, Y., Yamaguchi, Y., and Handa, H. (2010). Identification of a primary target of thalidomide teratogenicity. Science 327, 1345-1350.
Jin, Y., Zhou, J., Xu, F., Jin, B., Cui, L., Wang, Y., Du, X., Li, J., Li, P., Ren, R., et al. (2016). Targeting methyltransferase PRMT5 eliminates leukemia stem cells in chronic myelogenous leukemia. J Clin Invest 126, 3961-3980.
Kanda, M., Shimizu, D., Fujii, T., Tanaka, H., Shibata, M., Iwata, N., Hayashi, M., Kobayashi, D., Tanaka, C., Yamada, S., et al. (2016). Protein arginine methyltransferase 5 is associated with malignant phenotype and peritoneal metastasis in gastric cancer. Int J Oncol 49, 1195-1202.
Kaniskan, H. U., Konze, K. D., and Jin, J. (2015). Selective inhibitors of protein methyltransferases. J Med Chem 58, 1596-1629.
Kaniskan, H. U., Martini, M. L., and Jin, J. (2017). Inhibitors of Protein Methyltransferases and Demethylases. Chem Rev.
Kryukov, G. V., Wilson, F. H., Ruth, J. R., Paulk, J., Tsherniak, A., Marlow, S. E., Vazquez, F., Weir, B. A., Fitzgerald, M. E., Tanaka, M., et al. (2016). MTAP deletion confers enhanced dependency on the PRMT5 arginine methyltransferase in cancer cells. Science 351, 1214-1218.
Lai, A. C., Toure, M., Hellerschmied, D., Salami, J., Jaime-Figueroa, S., Ko, E., Hines, J., and Crews, C. M. (2016). Modular PROTAC Design for the Degradation of Oncogenic BCR-ABL. Angew Chem Int Ed Engl 55, 807-810.
Lu, J., Qian, Y., Altieri, M., Dong, H., Wang, J., Raina, K., Hines, J., Winkler, J. D., Crew, A. P., Coleman, K., et al. (2015). Hijacking the E3 Ubiquitin Ligase Cereblon to Efficiently Target BRD4. Chemistry & biology 22, 755-763.
Maniaci, C., Hughes, S. J., Testa, A., Chen, W., Lamont, D. J., Rocha, S., Alessi, D. R., Romeo, R., and Ciulli, A. (2017). Homo-PROTACs: bivalent small-molecule dimerizers of the VHL E3 ubiquitin ligase to induce self-degradation. Nat Commun 8, 830.
Marjon, K., Cameron, M. J., Quang, P., Clasquin, M. F., Mandley, E., Kunii, K., McVay, M., Choe, S., Kernytsky, A., Gross, S., et al. (2016). MTAP Deletions in Cancer Create Vulnerability to Targeting of the MAT2A/PRMT5/RIOK1 Axis. Cell Rep 15, 574-587.
Mavrakis, K. J., McDonald, E. R., 3rd, Schlabach, M. R., Billy, E., Hoffman, G. R., deWeck, A., Ruddy, D. A., Venkatesan, K., Yu, J., McAllister, G., et al. (2016). Disordered methionine metabolism in MTAP/CDKN2A-deleted cancers leads to dependence on PRMT5. Science 351, 1208-1213.
Prabhu, L., Wei, H., Chen, L., Demir, O., Sandusky, G., Sun, E., Wang, J., Mo, J., Zeng, L., Fishel, M., et al. (2017). Adapting AlphaLISA high throughput screen to discover a novel small-molecule inhibitor targeting protein arginine methyltransferase 5 in pancreatic and colorectal cancers. Oncotarget 8, 39963-39977.
Shimizu, D., Kanda, M., Sugimoto, H., Shibata, M., Tanaka, H., Takami, H., Iwata, N., Hayashi, M., Tanaka, C., Kobayashi, D., et al. (2017). The protein arginine methyltransferase promotes malignant phenotype of hepatocellular carcinoma cells and is associated with adverse patient outcomes after curative hepatectomy. Int J Oncol 50, 381-386.
Sun, D., Li, Z., Rew, Y., Gribble, M., Bartberger, M. D., Beck, H. P., Canon, J., Chen, A., Chen, X., Chow, D., et al. (2014). Discovery of AMG 232, a potent, selective, and orally bioavailable MDM2-p53 inhibitor in clinical development. J Med Chem 57, 1454-1472.
Tarighat, S. S., Santhanam, R., Frankhouser, D., Radomska, H. S., Lai, H., Anghelina, M., Wang, H., Huang, X., Alinari, L., Walker, A., et al. (2016). The dual epigenetic role of PRMT5 in acute myeloid leukemia: gene activation and repression via histone arginine methylation. Leukemia 30, 789-799.
Varfolomeev, E., Blankenship, J. W., Wayson, S. M., Fedorova, A. V., Kayagaki, N., Garg, P., Zobel, K., Dynek, J. N., Elliott, L. O., Wallweber, H. J., et al. (2007). IAP antagonists induce autoubiquitination of c-IAPs, NF-kappaB activation, and TNFalpha-dependent apoptosis. Cell 131, 669-681.
Vassilev, L. T., Vu, B. T., Graves, B., Carvajal, D., Podlaski, F., Filipovic, Z., Kong, N., Kammlott, U., Lukacs, C., Klein, C., et al. (2004). In vivo activation of the p53 pathway by small-molecule antagonists of MDM2. Science 303, 844-848.
Vu, B., Wovkulich, P., Pizzolato, G., Lovey, A., Ding, Q., Jiang, N., Liu, J. J., Zhao, C., Glenn, K., Wen, Y., et al. (2013). Discovery of RG7112: A Small-Molecule MDM2 Inhibitor in Clinical Development. ACS Med Chem Lett 4, 466-469.
Wei, H., Mundade, R., Lange, K. C., and Lu, T. (2014). Protein arginine methylation of non-histone proteins and its role in diseases. Cell Cycle 13, 32-41.
Weisberg, E., Ray, A., Barrett, R., Nelson, E., Christie, A. L., Porter, D., Straub, C., Zawel, L., Daley, J. F., Lazo-Kallanian, S., et al. (2010). Smac mimetics: implications for enhancement of targeted therapies in leukemia. Leukemia 24, 2100-2109.
Winter, G. E., Buckley, D. L., Paulk, J., Roberts, J. M., Souza, A., Dhe-Paganon, S., and Bradner, J. E. (2015). Phthalimide conjugation as a strategy for in vivo target protein degradation. Science 348, 1376-1381.
Xie, T., Lim, S. M., Westover, K. D., Dodge, M. E., Ercan, D., Ficarro, S. B., Udayakumar, D., Gurbani, D., Tae, H. S., Riddle, S. M., et al. (2014). Pharmacological targeting of the pseudokinase Her3. Nat Chem Biol 10, 1006-1012.
Zengerle, M., Chan, K. H., and Ciulli, A. (2015). Selective Small Molecule Induced Degradation of the BET Bromodomain Protein BRD4. ACS Chem Biol 10, 1770-1777.
Raina, K., Lu, J., Qian, Y., Altieri, M., Gordon, D., Rossi, A. M., Wang, J., Chen, X., Dong, H., Siu, K., Winkler, J. D., Crew, A. P., Crews, C. M., and Coleman, K. G. (2016). PROTAC-induced BET protein degradation as a therapy for castration-resistant prostate cancer. Proc. Natl. Acad. Sci. U.S.A 113, 7124-7129.

1.-63. (canceled)

64. A bivalent compound comprising a protein arginine methyltransferase 5 (PRMT5) ligand conjugated to a degradation/disruption tag.

65. (canceled)

66. The bivalent compound of claim 64, wherein the PRMT5 ligand is EPZ015666, GSK591, EPZ015938, BLL-1, HLCL-61, LLY-283, or PF-06855800.

67. (canceled)

68. The bivalent compound of claim 64, wherein the degradation/disruption tag is pomalidomide, thalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, or LCL161.

69. The bivalent compound of claim 64, wherein the degradation/disruption tag binds to a ubiquitin ligase or serves as a hydrophobic group that leads to PRMT5 protein misfolding.

70. The bivalent compound of claim 64, wherein the PRMT5 ligand is conjugated to the degradation/disruptor tag through a linker.

71. The bivalent compound of claim 64, wherein the bivalent compound has the form 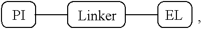
wherein PI comprises an PRMT5 ligand and EL comprises a degradation/disruption tag.

72. The bivalent compound of claim 71, wherein PI comprises: 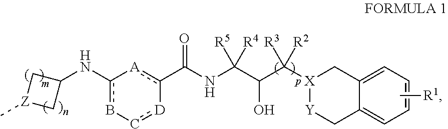
wherein A, B, C, and D are independently a bond, CR⁶, N, O, or S,
X and Z are independently CR⁷ or N,
Y is a bond, CR⁸, or N,
R¹, R², R³, R⁴, R⁵, R⁶, R⁷, and R⁸ are independently hydrogen, halogen, C₁-C₈ alkyl, C₁-C₈ alkoxy, or C₁-C₈ alkoxyalkyl,
m and n are independently 0-3, and
p is 0 or 1.

73. The bivalent compound of claim 71, wherein PI comprises: 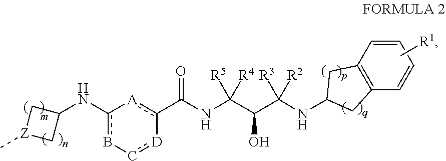
wherein A, B, C, and D are independently a bond, CR⁶, N, O, or S,
Z is CR⁷ or N,
R¹, R², R³, R⁴, R⁵, R⁶, R⁷, and R⁸ are independently hydrogen, halogen, C₁-C₈ alkyl, C₁-C₈ alkoxy, or C₁-C₈ alkoxyalkyl, and
m, n, p, and q are independently 0-3.

74. The bivalent compound of claim 71, wherein PI comprises: 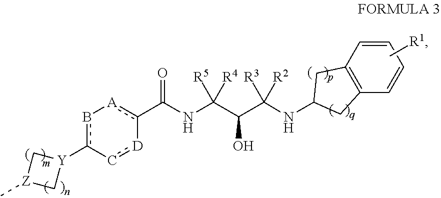
wherein A, B, C, and D are independently a bond, CR⁶, N, O, or S,
Y and Z are independently CR⁷ or N,
R¹, R², R³, R⁴, R⁵, R⁶, R⁷, and R⁸ are independently hydrogen, halogen, C₁-C₈ alkyl, C₁-C₈ alkoxy, or C₁-C₈ alkoxyalkyl, and
m, n, p, and q are independently 0-3.

75. The bivalent compound of claim 71, wherein PI is

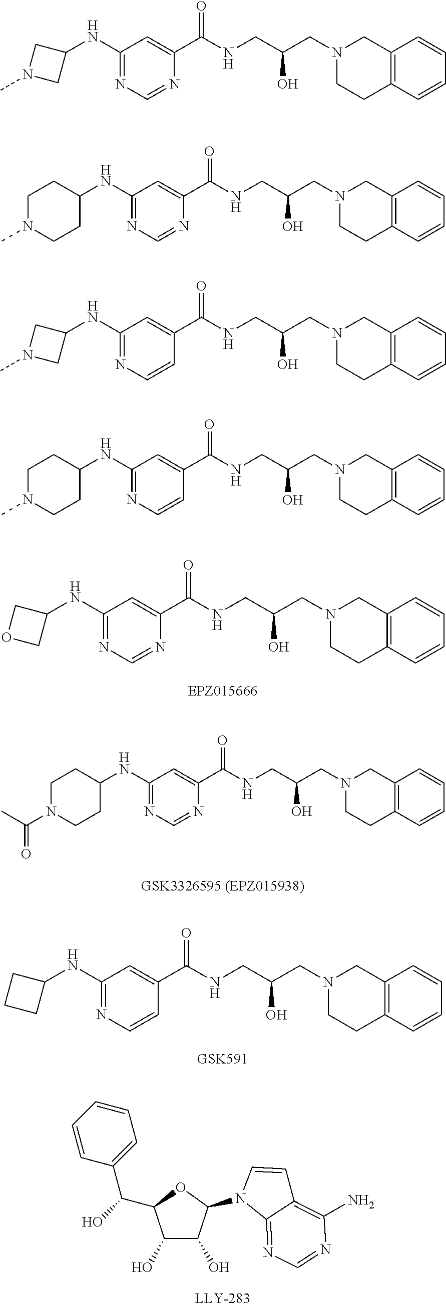

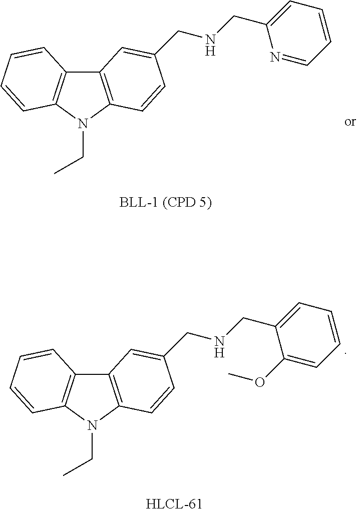 [SMILES: CCn1c2ccccc2c2cc(CNCc3ccccn3)ccc21 ; CCn1c2ccccc2c2cc(CNCc3ccccc3OC)ccc21]

76. The bivalent compound of claim 71, wherein EL 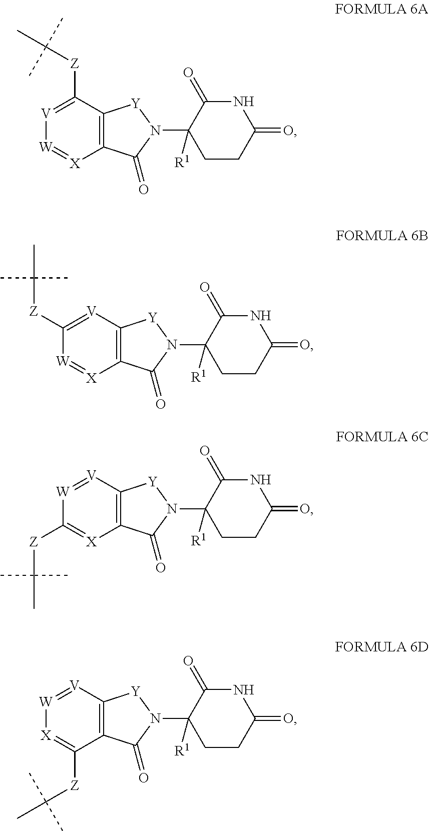
wherein V, W, and X are independently CR² or N, Y is CO or CH₂, Z is CH₂, NH, or O, R¹ is hydrogen, methyl, or fluoro, and R² is hydrogen, halogen, or C₁-C₅ alkyl; 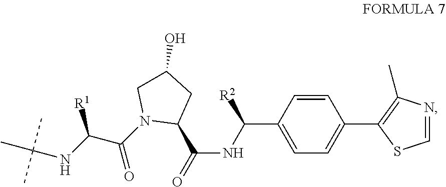
wherein R¹ and R² are independently hydrogen, C₁-C₈ alkyl, C₁-C₈ alkoxyalkyl, C₁-C₈ haloalkyl, C₁-C₈ hydroxyalkyl, C₃-C₇ cycloalkyl, C₃-C₇ heterocyclyl, C₂-C₈ alkenyl, or C₂-C₈ alkynyl; or 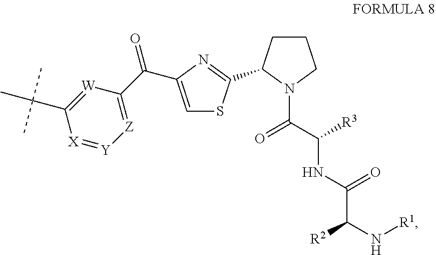
wherein R¹, R², R³, and R⁴ are independently hydrogen, C₁-C₈ alkyl, C₁-C₈ alkoxyalkyl, C₁-C₈ haloalkyl, C₁-C₈ hydroxyalkyl, C₃-C₇ cycloalkyl, C₃-C₇ heterocyclyl, C₂-C₈ alkenyl, or C₂-C₈ alkynyl, and V, W, X, and Z are independently CR⁴ or N.

77. The bivalent compound of claim 71, wherein EL is

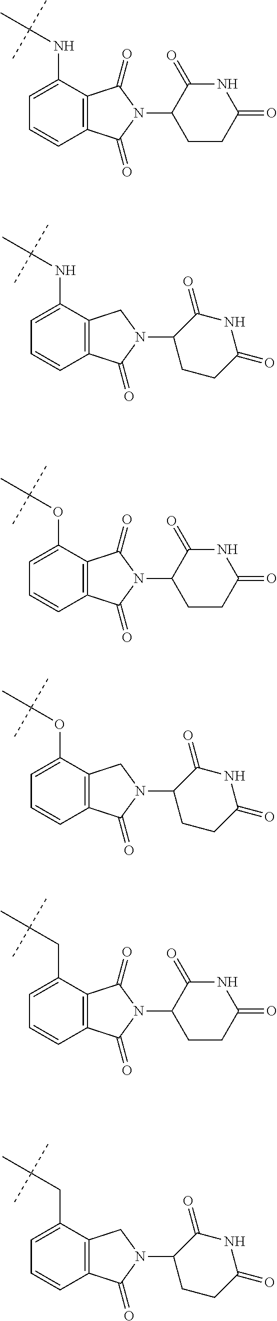

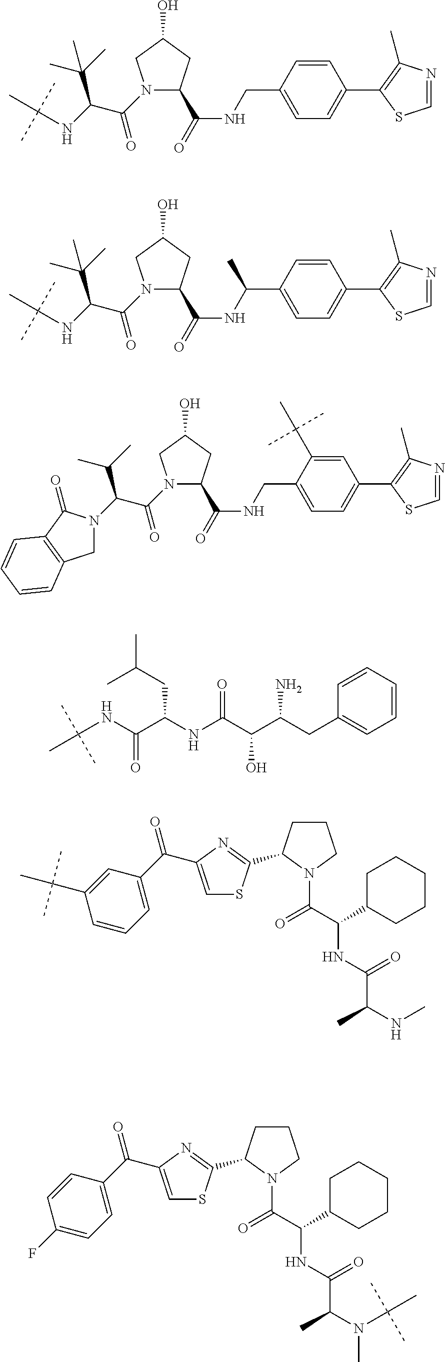

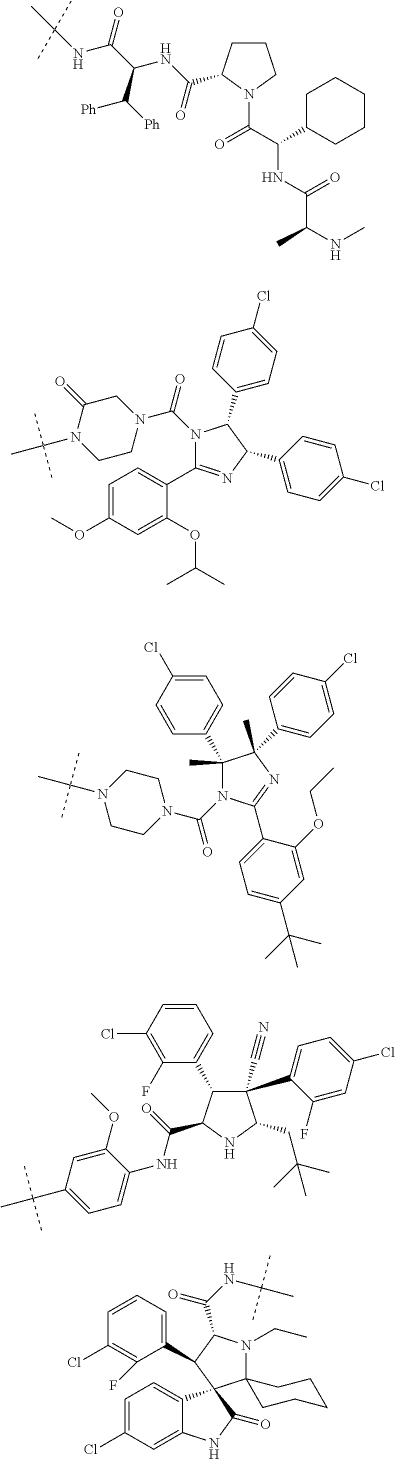

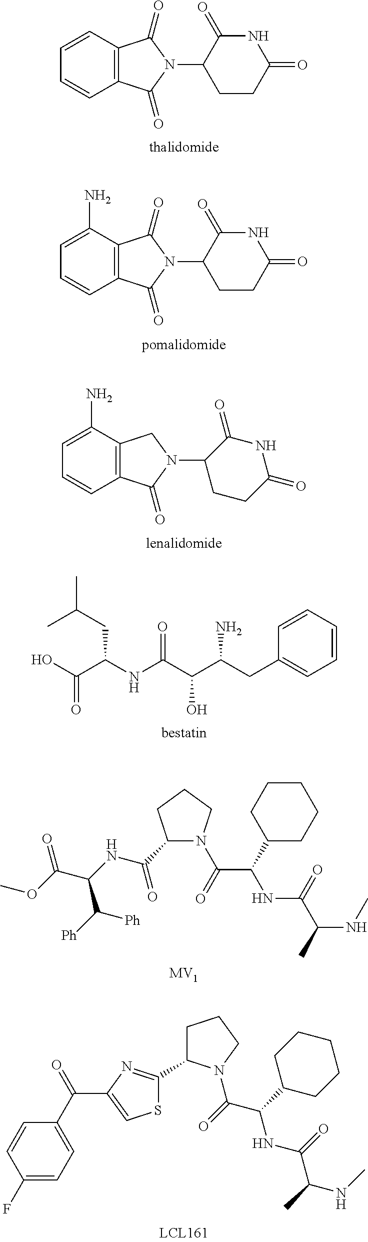

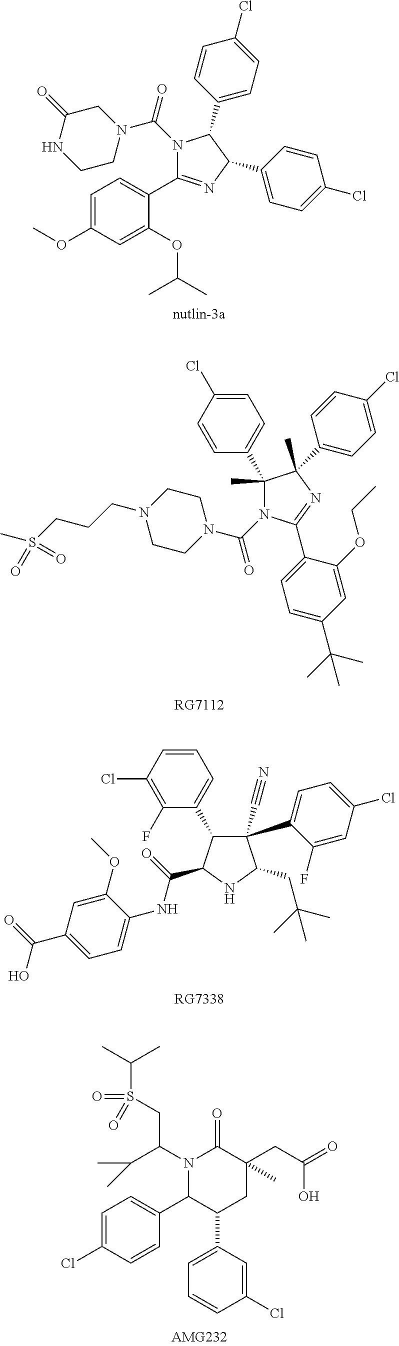 [SMILES: COc1ccc(C2=N[C@@H](c3ccc(Cl)cc3)[C@@H](c3ccc(Cl)cc3)N2C(=O)N2CCNC(=O)C2)c(OC(C)C)c1 ; CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1 ; COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F ; CC(C)C(CS(=O)(=O)C(C)C)N1C(=O)[C@@](C)(CC(=O)O)C[C@H](c2cccc(Cl)c2)C1c1ccc(Cl)cc1]

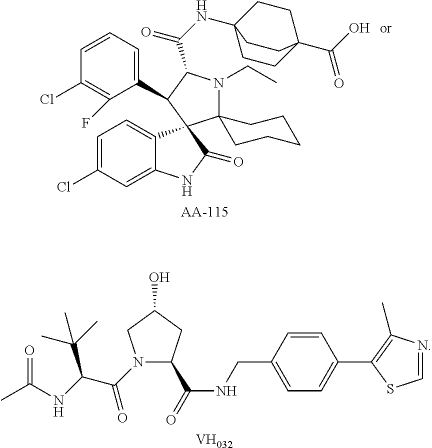

78.-79. (canceled)

80. The bivalent compound of claim 71, wherein the linker comprises acyclic or cyclic saturated or unsaturated carbon, ethylene glycol, amide, amino, ether, urea, carbamate, aromatic, heteroaromatic, heterocyclic or carbonyl containing groups with different lengths.

81. The bivalent compound of claim 71, wherein the linker is 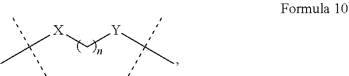
wherein X is C═O or CH₂, Y is C═O or CH₂, and n is 0-15; 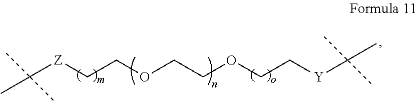
wherein X is C═O or CH₂, Y is C═O or CH₂, m is 0-15, n is 0-6, and o is 0-15; or 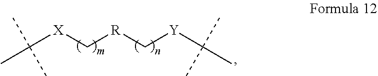
wherein X is C═O or CH₂, Y is C═O or CH₂, R is —CH₂—, —CF₂—, —CH(C₁₋₃ alkyl)-, —C(C₁₋₃ alkyl)(C₁₋₃ alkyl)-, —CH═CH—, —C(C₁₋₃ alkyl)═C(C₁₋₃ alkyl), —C═C—, —O—, —NH—, —N(C₁₋₃ alkyl)-, —C(O)NH—, —C(O)N(C₁₋₃ alkyl)-, a 3-13 membered ring, a 3-13 membered fused ring, a 3-13 membered bridged ring, or a 3-13 membered spiro ring, m is 0-15, and n is 0-15.

82. The bivalent compound of claim 81, wherein the linker is Formula 12 and R is selected from the group consisting of 3-13 membered rings, 3-13 membered fused rings, 3-13 membered bridged rings, and 3-13 membered spiro rings, wherein R contains one or more heteroatoms.

83. The bivalent compound of claim 81, wherein the linker is Formula 12 and R is

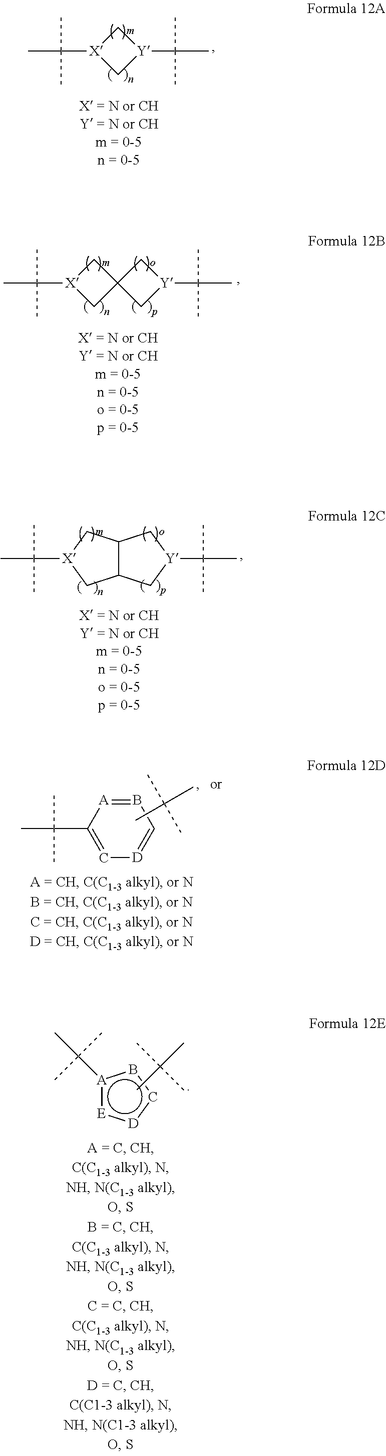

84. A method for identifying a bivalent compound which mediates degradation/disruption of PRMT5, the method comprising:
providing a heterobifunctional test compound comprising a PRMT5 ligand conjugated to a degradation/disruption tag;
contacting the heterobifunctional test compound with a cell comprising a ubiquitin ligase and PRMT5;
determining whether PRMT5 levels decrease in the cell; and
identifying the heterobifunctional test compound as a bivalent compound which mediates degradation/reduction of PRMT5 levels decrease in the cell.

85.-86. (canceled)

87. A bifunctional compound having the formula corresponding to YS31-58, YS31-60, YS31-61, YS31-62, YS31-63, YS31-64, YS31-67, YS31-68, YS31-69, YS43-6, YS43-7, YS43-8, YS43-16, YS43-19, YS43-20, YS43-21, YS43-22, YS43-25, YS43-28, YS43-29, YS43-30, YS43-31, YS43-32, YS43-33, YS43-34, YS43-37, YS-43-45, YS43-52, YS43-53 or YS43-54.

88. A bifunctional compound according to claim 87, having the formula corresponding to YS31-60, YS31-61, YS31-62, YS31-63, YS31-67, YS31-69, YS43-7, YS43-8, YS43-16, YS43-20, YS43-21, YS43-22, YS43-25, YS43-29, YS43-30, YS43-31, YS43-32, YS43-33, YS43-34, YS43-37 or YS-43-45.

89. A bifunctional compound according to claim 88, having the formula corresponding to YS31-60, YS31-69, YS43-8, YS43-16, YS43-20, YS43-21 or YS43-22.
